(19) United States
(12) Patent Application Publication
(10) Pub. No.: US 2024/0141435 A1
(43) Pub. Date: May 2, 2024

(54) Methods for detecting and predicting cancer

(71) Applicant: UCL Business Ltd, London (GB)

(72) Inventors: Martin Widschwendter, London (GB); James Barrett, London (GB)

(73) Assignee: UCL Business Ltd, London (GB)

(21) Appl. No.: 18/009,943

(22) PCT Filed: Jun. 17, 2021
(86) PCT No.: PCT/GB2021/051538

§ 371 Date: Dec. 12, 2022

(30) Foreign Application Priority Data
Jun. 17, 2020 (GB) 2009222.7

(51) Int. Cl. C12Q 1/6886; A61K 45/06; C12Q 1/6806; C12Q 1/686; C12Q 1/6874

(52) U.S. Cl. CPC C12Q 1/6886; A61K 45/06; C12Q 1/6806; C12Q 1/686; C12Q 1/6874; C12Q 2600/154

(57) ABSTRACT

The present invention relates to assays for predicting the presence, absence or development of cancer in an individual, particularly ovarian and endometrial cancer, by determining the methylation status of certain CpGs in a population of DNA molecules in a sample which has been taken from the individual, deriving an index value based on the methylation status of the certain CpGs, and predicting the presence, absence or development of cancer in the individual based on the cancer index value. The invention further relates to a method of treating and/or prevention of cancer in an individual, particularly ovarian and endometrial cancer, the method comprising assessing the presence, absence or development of cancer in an individual by performing the assays of the invention, followed by administering one or more therapeutic treatments or measures to the individual based on the assessment. The invention further provides a method of monitoring the cancer status of an individual according to changes in the individual's cancer index value over the course of time. The invention further relates to arrays which are suitable for performing the assays of the invention.

### SEQUENCE LISTING

The instant application contains a Sequence Listing which has been submitted electronically in ASCII format and is hereby incorporated by reference in its entirety. Said ASCII copy, created on Jun. 9, 2021, is named ‘Seq listing as filed 17 Jun. 2021 N417233WO MGW JAS.txt’ and is 7,370,039 bytes in size.

### FIELD OF THE INVENTION

The present invention relates to assays for predicting the presence, absence or development of cancer in an individual, particularly ovarian and endometrial cancer, by determining the methylation status of certain CpGs in a population of DNA molecules in a sample which has been taken from the individual, deriving an index value based on the methylation status of the certain CpGs, and predicting the presence, absence or development of cancer in the individual based on the cancer index value. The invention further relates to a method of treating and/or preventing cancer in an individual, particularly ovarian and endometrial cancer, the method comprising assessing the presence, absence or development of cancer in an individual by performing the assays of the invention, followed by administering one or more therapeutic or preventative treatments or measures to the individual based on the assessment. The invention further provides a method of monitoring the cancer status of an individual according to changes in the individual's cancer index value over the course of time. The invention further relates to arrays which are suitable for performing the assays of the invention.

The project leading to this application has been funded by the European Commission's Horizon 2020 Research and Innovation Action, H2020 FORECEE under Grant Agreement No. 634570, the European Commission's Horizon 2020 European Research Council Executive Agency, H2020 BRCA-ERC under Grant Agreement No. 742432 as well as the charity, The Eve Appeal.

### BACKGROUND TO THE INVENTION

Epithelial ovarian cancer is by far the most common cause of gynecological cancer-associated death. In order to improve this, the biggest challenge has been to identify the group of women at the highest risk of developing this devastating disease for which measures to diagnose early and/or prevent the disease can be offered. Currently, the best performing model has used a combination of single nucleotide polymorphisms (SNPs) as well as a large number of epidemiological parameters and has achieved (although only internally validated) a Receiver Operating Characteristic (ROC) Area Under the Curve (AUC) of 0.66. More recent data show that women with the highest and lowest 5% of the resulting polygenic risk score (PRS) have a 2.8% and a 0.9% risk of developing ovarian cancer until 80 years of age (the general risk for ovarian cancer in this population was 1.86%). Hence, there is a clear unmet need to identify women at risk of developing ovarian cancer in order to offer women at high risk those strategies which have been shown to early detect ovarian cancer or recommend to remove the Fallopian Tubes, the organ from which the majority of ovarian cancers originate.

Recent evidence has shown that an assay (called PapSEEK) for mutations in 18 genes as well as an assay for aneuploidy in cervical brush samples is able to identify 33% of women presenting with ovarian cancer (although the majority of these women have advanced stage III/IV cancers). In this study, the average age of cases and controls was 58 and 34 years, respectively. The consistent observation of a high allele frequency of pathogenic driver mutations in DNA from non-malignant normal endometrium with increasing age in light of the fact that the cases were almost twice as old as the controls makes it impossible to judge the true specificity of the PapSEEK test. The PapSEEK test includes steps which assess the presence or absence of tumour-derived DNA in cervical smear samples. In this test the tumour-derived DNA in the cervical smear sample may originate from an anatomical site other than the cervix. For example, the tumour-derived DNA may originate from an ovary, and may arrive at the cervix having drained through the uterus via a fallopian tube.

Epigenetic (i.e. DNA methylation, DNAme) changes have been identified in normal fimbriae from women with BRCA1/2 germline mutations and could potentially serve as a surrogate for both genetic and non-genetic factors including lifestyle, reproductive and environmental exposures contributing to ovarian cancer development. A number of proof of principle studies, so far exclusively performed in blood, have demonstrated that certain DNAme changes are associated with ovarian cancer predisposition. Sample heterogeneity and the choice of surrogate tissue are deemed to be among the most important factors impeding clinical implementation. Thus, the inventors aimed to assess whether DNAme profiles derived from cervical smear samples (containing hormone sensitive epithelial cells capable of recording ovarian cancer-predisposing factors at the level of the epigenome and arising from the Müllerian Duct, the same embryological structure from which the vast majority of ovarian cancers arise) are capable of identifying women with primary epithelial ovarian cancer.

### SUMMARY OF THE INVENTION

The current inventors set out to understand whether DNAme (DNA methylation) profiles may be used to detect the presence or absence of cancer, particularly ovarian and endometrial cancer. The inventors also set out to understand whether said DNAme profiles may be associated with the development of cancer, and therefore whether such profiles may be capable of functioning as surrogate markers for individual stratification purposes in connection with cancer.

In this regard the inventors have succeeded in developing assays involving “cancer index values” which are derived from and associated with DNAme profiles established from a sample comprising epithelial cells from a given individual. The sample may particularly be derived from the cervix, the vagina, the buccal area, blood and/or urine. The sample is preferably a cervical liquid-based cytology sample, and more preferably a cervical smear sample. Subsequent DNAme profiles from a given individual, and which values can be used to stratify the individual in connection with cancer. A preferred sample for use in any of the assays described and defined herein is a cervical liquid-based cytology sample. A particularly preferred sample for use in any of the assays described and defined herein is a cervical smear sample.

Accordingly, in contrast to prior art assays, the inventors have surprisingly determined that it is possible to derive “cancer index values” derived from and associated with DNAme profiles established from samples, wherein the samples are free of tumor-derived DNA. Thus the tissue(s) from which DNAme profiles of the present assays are established may act to provide surrogate markers for the presence, absence or development of cancer, wherein tumor cells, if present, or cells at risk of transforming into tumor cells, are located at an anatomical site distinct from the site from which the sample was taken.

The cancer index value is determined from data relating to the methylation status of one or more CpGs in a panel of CpGs as further defined and described herein. CpGs of the panel are methylation sites in DNA from cells derived from/obtained from samples comprising epithelial cells. The sample may particularly be derived from the cervix, the vagina, the buccal area, blood and/or urine. The sample is preferably a cervical liquid-based cytology sample, and more preferably a cervical smear sample.

For the purposes of the present invention, the cancer index value may be used interchangeably herein with “WID-OC-Index”, “WID-Index”, “cancer index”, “index” or “index value” (WID=women's risk identification). Furthermore, any reference to a cancer index value in the context of the present invention, may be equally used for the assessment of the presence, absence or development of ovarian cancer and/or endometrial cancer in an individual.

Based on studies with patients known to be free of ovarian cancer, the inventors have established cancer index values, using specific panels of CpGs, which have been determined to be associated with/characteristic of ovarian tissue which is negative for ovarian cancer, i.e. normal ovarian tissue which is free of ovarian cancer. Based on studies with patients known to possess ovarian cancer, the inventors have established cancer index values which have been determined to be associated with/characteristic of ovarian tissue which is positive for ovarian cancer.

The inventors have further established that the same specific panels of CpGs that associate with ovarian tissue which is negative or positive for ovarian cancer may likewise be associated with endometrial tissue that is negative or positive for endometrial cancer. Based on studies with patients known to be free of endometrial cancer, the inventors have established cancer index values, using specific panels of CpGs, which have been determined to be associated with/characteristic of endometrial tissue which is negative for endometrial cancer, i.e. normal endometrial tissue which is free of endometrial cancer. Based on studies with patients known to possess endometrial cancer, the inventors have established cancer index values which have been determined to be associated with/characteristic of endometrial tissue which is positive for endometrial cancer.

Thus, the inventors have been able to establish cancer index values, using specific panels of CpGs, which can characterize an individual as having cancer or not having cancer, or having a high risk of cancer development.

By determining the methylation profile-based cancer index value from a sample derived from the individual, the individual may be seen to possess a cancer index value which correlates with those possessed by individuals which are known, via the inventor's studies described herein, to be cancer positive or negative. Such correlations have been determined with a high degree of statistical accuracy, particularly with respect to parameters relevant to biological assays such as receiver operating characteristics (ROC) sensitivity and specificity, as well as area under the curve (AUC). Accordingly, by determining the cancer index value from a sample from a given individual, the individual may be determined to possess ovarian and/or endometrial tissue which is positive for cancer, i.e. the individual is diagnosed as having ovarian and/or endometrial cancer. Conversely, by determining the cancer index value from a sample from a given individual, the individual may be determined to possess ovarian and/or endometrial tissue which is negative for cancer, i.e. the individual is diagnosed as not having ovarian and/or endometrial cancer.

By determining the methylation profile-based cancer index value from samples derived from women known to possess a BRCA1 germline mutation, but are otherwise cancer negative, these women have an increased cancer index value relative to healthy women that do not possess a BRCA1 germline mutation. Women with a BRCA1 germline mutation are known to be at high risk of developing ovarian cancer. Accordingly, an increased cancer index value in women that are, at the time of testing, cancer negative can indicate a high risk of said women developing cancer, particularly ovarian and/or endometrial cancer, most preferably ovarian cancer. Preventative therapies and intensified screening as described herein may be particularly suitable for these women that have a high risk of developing cancer, particularly ovarian and/or endometrial cancer, most preferably ovarian cancer.

The inventors have determined that the cancer index value can vary depending on whether the women from which a sample has been obtained has, for example, a serous or mucinous cancer. A lower cancer index value is observed in samples obtained from women with mucinous cancer relative to serous cancer. Serous and mucinous cancers both arise as a result of fallopian tube epithelial cell differentiation, however serous cancers are further differentiated than mucinous cancers, and therefore tend to be of a higher grade and thus more dangerous. The observation that the cancer index value discussed herein correlates with the grade and severity of cancer in women indicates that the cancer index value can act as a surrogate marker for indicating the severity of cancer in an individual. These observations from the current inventors further establish that the cancer index value, as further described and defined herein, is dynamic and can change according to genetic and environmental conditions including the grade and severity of the cancer. The cancer index value may therefore be used to monitor an individual's cancer status and risk of cancer development. Moreover, the cancer index value may be used to monitor the efficacy of cancer treatments being administered to an individual, including therapeutic treatments and preventative treatments.

Accordingly, in the context of the present invention, by determining the cancer index value from a sample from a given individual it is possible to assess the presence, absence or development of cancer in an individual, or in other words to stratify the individual for cancer. In the context of the present invention, stratification for cancer is the process of categorizing the individual as being a member of a group of individuals who possess a phenotype in connection with cancer, including the presence or absence of cancer in the individual, or the development of cancer, i.e. by having epithelial cells, particularly derived from the cervix, the vagina, the buccal area, blood and/or urine which is more characteristic of ovarian or endometrial tissue which is cancer positive than ovarian or endometrial tissue which is cancer negative. The sample is preferably a cervical liquid-based cytology sample, and more preferably a cervical smear sample.

As explained herein, the assays methods of the invention are based on a cancer index value derived from a methylation profile from DNA originating from samples comprising epithelial cells. The sample may particularly be derived from the cervix, the vagina, the buccal area, blood and/or urine. The sample is preferably a cervical liquid-based cytology sample, and more preferably a cervical smear sample. Accordingly, the assays provide means for correlating samples derived from the cervix, the vagina, the buccal area, blood and/or urine-derived DNA methylation profile with a status connected with ovarian or endometrial cancer ranging from the individual being cancer negative, to the individual being cancer positive, with high statistical accuracy. Because the assays of the invention provide a correlation between the methylation profile and the disease status, the skilled person will appreciate that as part of the stratification process and outcome, disease status is assigned on the basis of a likelihood. As such, the methods of the invention provide assays which are predictive of an individual's status with respect to cancer. The assays of the invention accordingly provide means for predicting the presence or absence of cancer in an individual. The assays of the invention accordingly also provide means for predicting the development of cancer in an individual. The assays of the invention can provide means for predicting the development of cancer in an individual since the inventors have demonstrated that specific cancer index values can define ovarian and endometrial tissue which is cancer negative, whilst others can define ovarian and endometrial tissue which is cancer positive, and since the specific cancer index values may be dynamic and thereby increased in association with tumour grade and further increased cancer risk factors such as germline BRCA1 mutation, the values may be subject to change along a scale of cancer risk.

Whilst disease status may be assigned on the basis of a likelihood, the inventors have demonstrated herein that correlations between DNA methylation profile and cancer status using cancer index values can be achieved with a very high degree of statistical accuracy using parameters relevant to biological assays, as described further herein. As such, the assays of the invention provide means for predicting the presence or absence of cancer in an individual and for predicting the development of cancer in an individual, and for stratifying an individual for cancer, and wherein the prediction/stratification can be defined to be statistically highly reliable and robust. This in turn means that the prediction/stratification can be made with a high level of confidence. The assays of the invention can be defined to be statistically accurate by means known in the art, as further described and defined herein. The assays of the invention can be defined according to parameters relating to their statistical specificity and sensitivity. These parameters define the likelihood of false positive and false negative test results. The lower the proportion of false positive and false negative test results the more statistically accurate the assay becomes. In this regard the inventors have established CpG panels, as described and defined further herein, wherein the methylation status of CpGs in the panel can be used to establish cancer index values such that the assays produce statistically accurate predictions of cancer status. Accordingly, the inventors have determined that the assays described herein may be defined according to statistical parameters such as percentage specificity and sensitivity and also by receiver operating characteristics (ROC) area under the curve (AUC). All such means are known in the art and are known to be defined measures of statistical accuracy for biological assays such as those described and defined herein.

Thus the methods of the invention provide assays which can be used, with a high degree of statistical accuracy, to predict the presence, absence or development of cancer. The methods of the invention provide assays which can be used, with a high degree of statistical accuracy, to stratify an individual with respect to cancer status. Accordingly, the methods of the invention provide useful information to individuals and their physicians concerning patient cancer status. This information may help inform actual therapeutic treatment measures if the presence of cancer is identified in the individual. The information may help to monitor the progress of therapeutic treatment measures in the individual by monitoring changes in the cancer index value over the course of a period of time. The information may help to monitor the progress of prophylactic or preventative treatment measures in the individual by monitoring changes in the cancer index value over the course of a period of time. As such the methods of the invention offer significant advantages in the personalised prevention and early detection as well as treatment and management of cancer in individuals.

Accordingly, the invention provides an assay for assessing the presence, absence or development of cancer in an individual, the assay comprising:
a. providing a sample which has been taken from the individual, the sample comprising a population of DNA molecules;
b. determining in the population of DNA molecules in the sample the methylation status of a panel of one or more CpGs selected from a panel of CpGs identified at nucleotide positions 61 to 62 in SEQ ID NOs 1 to 14,000;
c. deriving a cancer index value based on the methylation status of the one or more CpGs in the panel; and
d. assessing the presence, absence or development of cancer in the individual based on the cancer index value;
wherein the assay is characterised as having an area under the curve (AUC) of 0.60 or more as determined by receiver operating characteristics (ROC).

The assay of the invention may be performed above and additionally wherein the panel of one or more CpGs comprises at least 500 CpGs selected from the CpGs identified at nucleotide positions 61 to 62 in SEQ ID NOs 1 to 14,000, preferably wherein the assay is characterised as having an AUC of at least 0.67.

The assay of the invention may be performed above and additionally wherein the panel of one or more CpGs comprises at least the CpGs identified in SEQ ID NOs 1 to 500 and identified at nucleotide positions 61 to 62, preferably wherein the assay is characterised as having an AUC of at least 0.74.

The assay of the invention may be performed above and additionally wherein the panel of one or more CpGs comprises at least 1000 CpGs selected from the CpGs identified at nucleotide positions 61 to 62 in SEQ ID NOs 1 to 14,000, preferably wherein the assay is characterised as having an AUC of at least 0.68.

The assay of the invention may be performed above and additionally wherein the panel of one or more CpGs comprises at least the CpGs identified in SEQ ID NOs 1 to 1000 and identified at nucleotide positions 61 to 62, preferably wherein the assay is characterised as having an AUC of at least 0.75.

The assay of the invention may be performed above and additionally wherein the panel of one or more CpGs comprises at least 2000 CpGs selected from the CpGs identified at nucleotide positions 61 to 62 in SEQ ID NOs 1 to 14,000, preferably wherein the assay is characterised as having an AUC of at least 0.68.

The assay of the invention may be performed above and additionally wherein the panel of one or more CpGs comprises at least the CpGs identified in SEQ ID NOs 1 to 2000 and identified at nucleotide positions 61 to 62, preferably wherein the assay is characterised as having an AUC of at least 0.75.

The assay of the invention may be performed above and additionally wherein the panel of one or more CpGs comprises at least 10,000 CpGs selected from the CpGs identified at nucleotide positions 61 to 62 in SEQ ID NOs 1 to 14,000, preferably wherein the assay is characterised as having an AUC of at least 0.73.

The assay of the invention may be performed above and additionally wherein the panel of one or more CpGs comprises at least the CpGs identified in SEQ ID NOs 1 to 10,000 and identified at nucleotide positions 61 to 62, preferably wherein the assay is characterised as having an AUC of at least 0.78.

The assay of the invention may be performed above and additionally wherein the panel of one or more CpGs comprises at least the 14,000 CpGs identified at nucleotide positions 61 to 62 in SEQ ID NOs 1 to 14,000, and further wherein the assay is characterised as having an AUC of at least 0.78.

The assay of the invention may be performed above and additionally wherein the step of determining in the population of DNA molecules in the sample the methylation status of the one or more CpGs in the panel further comprises determining a β value of each CpG.

The assay of the invention may be performed above and additionally wherein the step of deriving the cancer index value based on the methylation status of the one or more CpGs in the panel comprises:
a. providing a methylation 3-value data set comprising the methylation β-values for each CpG in the panel;
b. providing a mathematical model capable of generating the cancer index from the methylation 3-value data set; and
c. applying the mathematical model to the methylation 3-value data set, thereby generating the cancer index.

The assay of the invention may be performed above and additionally wherein the cancer index value is an ovarian cancer index value (WID-OC-Index), and wherein the mathematical model which is applied to the methylation 3-value data set to generate the cancer index is an algorithm according to the following formula:

$$\\ WID - OC - index = {\sum }_{i = 1}^{n} \frac{{w}_{i} {β}_{i} - μ}{σ}$$

wherein:
   a. β₁, . . . , βₙ are methylation beta-values (between 0 and 1);
   b. w₁, . . . , w_{14,000} are real valued coefficients;
   c. p and a are real valued parameters used to scale the index; and
   d. n refers to the number of CpGs in the panel of one or more CpGs;
preferably wherein the cancer is ovarian cancer.

The assay of the invention may be performed above and additionally wherein when the cancer index value for the individual is about −0.056 or more, the individual is assessed as having cancer or as having a high risk of cancer development, or wherein when the cancer index value for the individual is less than about 0.056, the individual is assessed as not having cancer or as having a low risk of cancer development, preferably wherein:
a. the panel of one or more CpGs comprises at least 500 of the CpGs identified at nucleotide positions 61 to 62 in SEQ ID NOs 1 to 14,000, and wherein the sensitivity is at least 64% and the specificity is at least 63%;
b. the panel of one or more CpGs comprises at least the CpGs defined by SEQ ID NOs 1 to 500 and identified at nucleotide positions 61 to 62, and wherein the sensitivity is at least 72% and specificity is at least 62%;
c. the panel of one or more CpGs comprises at least the CpGs defined by SEQ ID NOs 1 to 1000 and identified at nucleotide positions 61 to 62, and wherein the sensitivity is at least 80% and specificity is at least 61%; or
d. the panel of one or more CpGs comprises at least the CpGs defined by SEQ ID NOs 1 to 2000 and identified at nucleotide positions 61 to 62, and
   wherein the sensitivity is at least 76% and specificity is at least 61%; preferably wherein the assay comprises determining methylation β-values for each CpG in the panel of one or more CpGs, more preferably wherein the cancer is ovarian cancer.

The assay of the invention may be performed above and additionally wherein when the cancer index value for the individual is about 0.485 or more, the individual is assessed as having cancer or as having a high risk of cancer development, or wherein when the cancer index value for the individual is less than about 0.485, the individual is assessed as not having cancer or as having a low risk of cancer development, preferably wherein:
a. the panel of one or more CpGs comprises at least 500 of the CpGs identified at nucleotide positions 61 to 62 in SEQ ID NOs 1 to 14,000, and wherein the sensitivity is at least 43% and the specificity is at least 80%;
b. the panel of one or more CpGs comprises at least the CpGs defined by SEQ ID NOs 1 to 500 and identified at nucleotide positions 61 to 62, and wherein the sensitivity is at least 51% and specificity is at least 87%;
c. the pane of one or more CpGs comprises at least the CpGs defined by SEQ ID NOs 1 to 1000 and identified at nucleotide positions 61 to 62, and wherein the sensitivity is at least 45% and specificity is at least 87%; or
d. the panel of one or more CpGs comprises at least the CpGs defined by SEQ ID NOs 1 to 2000 and identified at nucleotide positions 61 to 62, and
   wherein the sensitivity is at least 49% and specificity is at least 89%; preferably wherein the assay comprises determining methylation β-values for each CpG in the panel of one or more CpGs, more preferably wherein the cancer is ovarian cancer.

The assay of the invention may be performed above and additionally wherein when the cancer index value for the individual is about 1.006 or more, the individual is assessed as having cancer or as having a high risk of cancer development, or wherein when the cancer index value for the individual is less than about 1.006, the individual is assessed as not having cancer or as having a low risk of cancer development, preferably wherein:
a. the panel of one or more CpGs comprises at least 500 of the CpGs identified at nucleotide positions 61 to 62 in SEQ ID NOs 1 to 14,000, and wherein the sensitivity is at least 30% and the specificity is at least 87%;
b. the panel of one or more CpGs comprises at least the CpGs defined by SEQ ID NOs 1 to 500 and identified at nucleotide positions 61 to 62, and wherein the sensitivity is at least 24% and specificity is at least 95%;
c. the panel of one or more CpGs comprises at least the CpGs defined by SEQ ID NOs 1 to 1000 and identified at nucleotide positions 61 to 62, and wherein the sensitivity is at least 22% and specificity is at least 95%; or
d. the panel of one or more CpGs comprises at least the CpGs defined by SEQ ID NOs 1 to 2000 and identified at nucleotide positions 61 to 62, and
   wherein the sensitivity is at least 25% and specificity is at least 96%; preferably wherein the assay comprises determining methylation β-values for each CpG in the panel of one or more CpGs, more preferably wherein the cancer is ovarian cancer.

The assay of the invention may be performed above and additionally wherein when the cancer index value for the individual is:
a. less than about −0.570 the individual is assessed as not having cancer;
b. about −0.570 or more and less than about −0.210 the individual is assessed as having a low risk of cancer;
c. about −0.210 or more and less than about 0.170 the individual is assessed as having a moderate risk of cancer;
d. about 0.170 or more the individual is assessed as having a high risk of cancer;
   preferably wherein the assay comprises determining methylation β-values for each CpG in the panel of one or more CpGs, more preferably wherein the cancer is ovarian cancer.

The assay of the invention may be performed above and additionally wherein the step of determining in the population of DNA molecules in the sample the methylation status of each CpG in the panel of one or more CpGs comprises:
a. performing a sequencing step to determine the sequence of each CpG;
b. hybridising DNA to an array comprising probes capable of discriminating between methylated and non-methylated forms of the CpGs and applying a detection system to the array so as to determine the methylation status of each CpG; and/or
c. performing a PCR step using methylation-specific primers, wherein the methylation status of the CpG is determined by the presence or absence of a PCR product.

The assay of the invention may be performed above and additionally wherein the step of determining the methylation status of each CpG in the panel of one or more CpGs comprises:
a. bisulphite converting the DNA; or
b. performing the steps of oxidising 5-methylcytosine bases (5mC) to 5-carboxylcytosine bases (5caC), preferably by ten-eleven translocation (TET), and/or oxidising 5-hydroxymethylcytosine bases (5hmC) to 5-carboxylcytosine bases (5caC), preferably by ten-eleven translocation (TET); followed by reducing 5-carboxylcytosine bases (5caC) to dihydrouracil bases (DHU), optionally with pyridine borane.

The invention also provides a method of treating or preventing cancer in an individual, the method comprising:
a. assessing the cancer status of the individual by assessing the presence, absence or development of cancer in the individual by performing any one of the assays of the invention;
b. administering one or more therapeutic or preventative treatments to the individual based on the assessment.

The method of the invention may be performed above and additionally wherein the individual is assessed as not having cancer or as having a low risk of cancer development, and wherein when the cancer index value is about −0.570 or more and less than about −0.210, and preferably wherein the assay comprises determining methylation β-values for each CpG in the panel of one or more CpGs, the individual is subjected to one or more treatments according to their cancer index value, wherein the one or more treatments comprise intensified screening, preferably wherein the intensified screening comprises any of:
a. a test for a BRCA1 and/or BRCA2 germline mutation;
b. a test for CA125, preferably wherein the test is repeated annually;
c. a test for cell-free tumour DNA methylation in plasma/serum, preferably wherein the test is repeated annually;
d. a test for cell-free tumour DNA methylation in vaginal fluid, preferably wherein the test is repeated annually;
e. a repeat assay according to any one of the assays of the invention, preferably wherein the repeat assay is performed about two years after the previous assay,
   preferably wherein the individual being tested by any one or more of b. to d. is post-menopausal.

The method of the invention may be performed above and additionally wherein the individual is assessed as having a moderate risk of having cancer or as having a moderate risk of cancer development, and wherein when the cancer index value is about −0.210 or more and less than about 0.170, and preferably wherein the assay comprises determining methylation β-values for each CpG in the panel of one or more CpGs, the individual is subjected to one or more treatments according to their cancer index value, wherein the one or more treatments comprise any of:
a. intensified screening, preferably wherein the intensified screening comprises one or more of:
   i. a test for a BRCA1 and/or BRCA2 germline mutation;
   ii. a test for CA125, preferably wherein the test is repeated annually;
   iii. a test for cell-free tumour DNA methylation in plasma/serum, preferably wherein the test is repeated annually;
   iv. a test for cell-free tumour DNA methylation in vaginal fluid, preferably wherein the test is repeated annually
   v. a pelvic MRI scan, preferably wherein the individual being subjected to the pelvic MRI scan is post-menopausal, and preferably wherein the scan is repeated annually;
   vi. a repeat assay according to any one of the assays of the invention, preferably wherein the repeat assay is performed about one year after the previous assay;
b. administration of one or more of Aspirin, oral contraceptive pill, selective estrogen receptor modulators (SERMS), and selective progesterone receptor modulators (SPRMs).

The method of the invention may be performed above and additionally wherein the individual is assessed as having cancer or as having a high risk of cancer development, and wherein when the cancer index value is about 0.170 or more, and preferably wherein the assay comprises determining methylation β-values for each CpG in the panel of one or more CpGs, the individual is subjected to one or more treatments according to their cancer index value, wherein the one or more treatments comprise any of:
a. intensified screening, preferably wherein the intensified screening comprises one or more of:
   i. a test for a BRCA1 and/or BRCA2 germline mutation;
   ii. a test for CA125, preferably wherein the test is repeated three-monthly;
   iii. a test for cell-free tumour DNA methylation in plasma/serum, preferably wherein the test is repeated annually;
   iv. a test for cell-free tumour DNA methylation in vaginal fluid, preferably wherein the test is repeated annually
   v. a pelvic MRI scan, preferably wherein the scan is repeated annually;
   vi. a repeat assay according to any one of the assays of the invention, preferably wherein the repeat assay is performed about one year after the previous assay;
b. administration of one or more of Aspirin, oral contraceptive pill, selective estrogen receptor modulators (SERMS), and selective progesterone receptor modulators (SPRMs); and/or
c. a total hysterectomy and bilateral salpingo-oophorectomy.

The method of the invention may be performed above and additionally wherein the one or more treatments that the individual is subjected to are repeated on a monthly, three monthly, six monthly, yearly or two yearly basis following an initial administration.

The assay of the invention may be performed above and additionally wherein:
a. the SERMs comprise Anordin, Bazedoxifene, Broparestrol, Broparestrol, Clomifene, Cyclofenil, Lasofoxifene, Ormeloxifene, Ospemifene, Raloxifene, Tamoxifen, preferably wherein the SERMs comprise Tamoxifen, Bazedoxifene and Raloxifene; and/or
b. the SPRMs comprise Mifepristone, Ulipristal, Asoprisnil, Proellex, Onapristone, Asoprisnil and Lonaprisan.

The invention additionally provides a method of monitoring the cancer status of an individual according to the individual's cancer index value, the method comprising: (a) assessing the presence, absence or development of cancer in an individual by performing the assay according to any one of the assays of the invention at a first time point; (b) assessing the presence, absence or development of cancer in the individual by performing the assay according to any one of the assays of the invention at one or more further time points; and (c) monitoring any change in the cancer status of the individual between time points.

The method of the invention may be performed above and additionally wherein the further time points are monthly, three monthly, six monthly, yearly or two yearly basis following an initial assessment.

The method of the invention may be performed above and additionally wherein depending on the cancer index value and/or cancer status of the individual, one or more treatments are administered to the individual according to any one of methods of the invention, or wherein when the cancer index value of the individual is less than about −0.570 no treatment is administered to the individual.

The method of the invention may be performed above and additionally wherein an increase in the cancer index value indicates a negative response to the one or more treatments.

The method of the invention may be performed above and additionally wherein changes are made to the one or more treatments if a negative response is identified.

The method of the invention may be performed above and additionally wherein a decrease in the cancer index value indicates a positive response to the one or more treatments.

The method of the invention may be performed above and additionally wherein changes are made to the one or more treatments if a positive response is identified.

The assay of the invention may be performed above and additionally wherein the sample is obtained from a tissue comprising epithelial cells, preferably wherein the sample is not obtained from ovarian or endometrial tissue.

The assay of the invention may be performed above and additionally wherein the sample is obtained from:
a. cervical tissue;
b. vaginal tissue;
c. cervicovaginal tissue; and/or
d. buccal tissue;
preferably wherein the sample is obtained from cervical tissue, most preferably
wherein the sample is obtained from tissue from a cervical smear.

The assay of the invention may be performed above and additionally wherein the assay is for assessing the presence, absence or development of:
a. ovarian cancer, preferably wherein the ovarian cancer is serious carcinoma, mucinous carcinoma, endometrioid carcinoma, clear cell carcinoma, low malignant potential (LMP) tumor, borderline epithelial ovarian cancer, teratoma, dysgerminoma, endodermal sinus tumor, Choriocarcinoma, granulosa-theca tumor, Sertoli-Leydig tumor, granulosa cell tumor, small cell carcinoma of the ovary or primary peritoneal carcinoma; or
b. endometrial cancer, preferably wherein the endometrial cancer is an endometriod cancer, uterine carcinosarcoma, squamous cell carcinoma, small cell carcinoma, transitional carcinoma, serous carcinoma, clear-cell carcinoma, mucinous adenocarcinoma, undifferentiated carcinoma, dedifferentiated carcinoma or serous adenocarcinoma.

The invention also provides an array capable of discriminating between methylated and non-methylated forms of CpGs; the array comprising oligonucleotide probes specific for a methylated form of each CpG in a CpG panel and oligonucleotide probes specific for a non-methylated form of each CpG in the panel; wherein the panel consists of at least 500 CpGs selected from the CpGs identified at nucleotide position 61 to 62 in SEQ ID NOs 1 to 14,000.

The array of the invention may be as above and additionally provided that the array is not an Infinium MethylationEPIC BeadChip array or an Infinium HumanMethylation450, and/or provided that the number of CpG-specific oligonucleotide probes of the array is 482,000 or less, 480,000 or less, 450,000 or less, 440,000 or less, 430,000 or less, 420,000 or less, 410,000 or less, or 400,000 or less.

The array of the invention may be performed as above and additionally wherein the panel comprises any set of CpGs defined in the assays of the invention.

The array of the invention may be as above and additionally further comprising one or more oligonucleotides comprising any set of CpGs defined in the assays of any one of the invention, wherein the one or more oligonucleotides are hybridized to corresponding oligonucleotide probes of the array.

The invention also provides a hybridized array, wherein the array is obtainable by hybridizing to an array of the invention a group of oligonucleotides comprising any panel of one or more CpGs defined in the assays of the invention.

The invention also provides a process for making the hybridized array of the invention, comprising contacting an array of the invention with a group of oligonucleotides comprising any panel of one or more CpGs defined in any one of the assays of the invention.

### BRIEF DESCRIPTION OF THE FIGURES

FIG. 1 shows training of the WID-OC-index classifiers and predictive performance of the WID-OC index, wherein optionally the WID-OC-index controls for immune cell proportion.

Distribution of p-values obtained by comparing cases and controls at each CpG site and after controlling for immune cell proportion and age (A). The distribution of different cell types in the discovery dataset inferred using the EpiDISH algorithm (* p<0.05, ** p<0.01, *** p<0.001) (B). Area under the curve (AUC) values in the internal validation set as a function of the number of CpGs used to train the classifier (C). ROC curves of the WID-OC-index in the internal validation set for samples with an immune cell (IC) proportion ≤0.5 and >0.5 (D). Distribution of the WID-OC-index with respect to immune cell proportion in the internal validation set (E). Distribution of the estimated variance in epitheilal and immune cells across all CpGs used in the WID-OC-index (F). Odds ratios corresponding to the genomic annotation of the CpGs comprising the WID-OC-index (when compared to the overall EPIC array; * p<0.05, ** p<0.01, *** p<0.001) (G). AUC values in the internal validation set after training classifiers on different subsets of the CpGs used in the WID-OC-index. The top n CpGs were either retained or removed. CpGs were also split into separate bins of size 500 (H).

FIG. 2 shows external validation of the WID-OC-index, performance of the WID-OC-index in discriminating cancer-free women and women with endometrial cancer, and association between the WID-OC-index and germline BRCA1 mutation.

The WID-OC-index versus immune cell proportion in an independent external validation set (A). ROC curve from the external validation set (B). The WID-OC-index versus immune cell proportion in a separate cohort of endometrial cancer samples and the same control samples from the internal validation set (c). ROC curve from the endometrial cancer dataset (D). The WID-OC-index versus immune cell proportion in an independent cohort of BRCA1 mutation carriers (E). ROC curve from the BRCA1 dataset (F).

FIG. 3 shows association of WID-OC-index with other clinically relevant factors.

The WID-OC-index versus age in control samples from the internal and external validation datasets (A). ROC curves for women above and below 50 years of age (B). The WID-OC-index versus a 28 SNP ovarian cancer polygeneic risk score (PRS) in the internal validation dataset (C). ROC curves for women above and below 50 years of age (D). The distribution of the WID-OC-index across different histological subtypes (E). The distribution of the WID-OC-index across different cancer grades (* p<0.05, ** p<0.01, *** p<0.001) (F).

FIG. 4 shows WID-OC-index data and its association with tumour cell fraction, known cancer markers, indicators of cell type of origin.

The estimated proportion of tumour DNA in each cervical smear sample as estimated using the EpiDISH algorithm (A). Results from real time PCR to detect ZNF154, a pan cancer marker primarily discovered in ovarian cancer (* p<0.05, ** p<0.01, *** p<0.001) (B). The WID-OC-index evaluated in eight different cell lines (C). a subset of ENCODE tissue samples. The germline mutation proportion refers to the proportion of cancers in each tissue type that have a BRCA1 or BRCA2 mutation (D and E). Top ten tissue-specific patterns enriched in hyper-methylated CpGs (F). Top ten tissue-specific patterns enriched in hypo-methylated CpGs (G).

FIG. 5 shows the experimental design underpinning the discovery and validation of the WID-OC-index.

FIG. 6 shows cell type distributions in different datasets.

Cell type distribution in the external validation set inferred using the EpiDISH algorithm (A). Cell type distribution in the endometrial cancer dataset inferred using the EpiDISH algorithm (B). Cell type distribution in the BRCA1 dataset inferred using the EpiDISH algorithm (C). Cell type distribution in the BRCA2 dataset inferred using the EpiDISH algorithm (* p<0.05, ** p<0.01, *** p<0.001) (D).

FIG. 7 shows the performance of the WID-OC-index in the discriminatory performance for assessing ovarian cancer risk of BRCA2 germline mutation carriers.

The WID-OC-index versus immune cell proportion in an independent cohort of BRCA2 mutation carriers (A). ROC curve from the BRCA2 dataset (B).

FIG. 8 shows variable association of WID-OC-index with other clinically relevant factors.

Association of the WID-OC-index with (A) family history, (B) age at menarche, (C) oral contraceptive pill (OCP) use, (D) ethnicity, (E) last period age, and (F) parity (* p<0.05, ** p<0.01, *** p<0.001).

FIG. 9 shows lack association of WID-OC-index with other technical parameters.

Distribution of the WID-OC-index among various subtypes of control samples (A). Association between the WID-OC-index and time from sample collection to DNA extraction (B).

FIG. 10 shows an evaluation of the WID-OC-index in ENCODE tissue samples. Tissue types at high risk of developing cancer in BRCA carriers are coloured red, low risk tissues are coloured blue.

FIG. 11 shows inferred tumour DNA proportion and epithelial variance, and their effects on the WID-OC index.

The proportions of epithelial and tumour DNA in each sample inferred using the EpiDISH algorithm with a reference panel of epithelial, immune, fibroblast and tumour cell types (A). An example of a CpG with a high epithelial variance and low immune cell variance (B).

FIG. 12 shows cutpoints applied to the patient data, and consequent specificity and sensitivity for cancer status discrimination achieved when these cutpoints are applied.

### DETAILED DESCRIPTION OF THE INVENTION

### Identification of CpGs

The present inventors sought to identify CpG methylation-based assays capable of assessing the presence, absence or development of cancer in an individual. Any of the assays described herein for assessing the presence, absence or development of cancer in an individual are capable of being utilised for assessing the presence, absence or development of ovarian cancer and/or endometrial cancer. The present inventors compared CpG methylation levels in non-cancerous epithelial tissue, particularly from the cervix or vagina across groups of women that were either known to be both ovarian and endometrial cancer negative, or known to be ovarian and/or endometrial cancer positive, or known to have a BRCA1 germline mutation but are as yet ovarian and endometrial cancer free. This led to the surprising establishment of a “cancer index”, used interchangeable herein with “index”, “index value”, “WID-OC-Index” or “WID-Index” (WID=women's risk identification).

A CpG as defined herein refers to the CG dinucleotide motif identified in relation to each SEQ ID NO., wherein the CG dinucleotide of interest is identified at positions 61 to 62. Thus by determining the methylation status of any panel of one or more CpGs defined by a panel of one more of SEQ ID NOs 1 to 14,000, it is meant that a determination is made as to the methylation status of the cytosine of the CG dinucleotide motif identified at positions 61 to 62 in the panel of one or more CpGs of SEQ ID NOs 1 to 14,000, accepting that variations in the sequence upstream and downstream of any given CpG, as identified at positions 61 to 62 of any given SEQ ID NO, may exist due to sequencing errors or variation between individuals.

As set out in more detail in the Examples, the methylation status of sub-selections of the 14,000 CpGs, as identified in SEQ ID NOs 1 to 14,000, may be determined in order to assess an individual for the presence, absence or development of cancer with high sensitivity and specificity. A panel of one or more of the CpGs identified in SEQ ID NOs 1 to 14,000 may be utilised to derive a cancer index for an individual in accordance with the invention described herein.

The methylation status of a panel of one or more CpGs of the 14,000 CpGs defined according to SEQ ID NOs: 1 to 14,000 may be assessed by any suitable technique. As explained in more detail in the Examples below, one particular exemplary technique which the inventors have used is an array-based analysis technique coupled with beta value analysis. SEQ ID NOs 1 to 14,000 correspond to the sequences of commercial probes utilised in said array.

### Cancer-Related CPGs for Analysis

In any of the assays described herein, in a sample which has been taken from an individual, the sample comprises a population of DNA molecules.

The assay of the invention further comprises determining in the population of DNA molecules in the sample the methylation status of a panel of one or more CpGs selected from a panel of CpGs identified at nucleotide positions 61 to 62 in SEQ ID NOs 1 to 14,000. A cancer index value is then derived based on the methylation status of the one or more CpGs in the panel, which is used to assess the presence, absence or development of cancer in the individual based on the cancer index value.

In any of the assays described herein, in DNA derived from cells in the sample the methylation status of each CpG in a panel of one or more CpGs from a panel of CpGs identified at nucleotide positions 61 to 62 in SEQ ID NOs 1 to 14,000, is determined.

In any of the assays described herein, the panel of one or more CpGs may comprise at least 500 CpGs selected from the CpGs identified at nucleotide positions 61 to 62 in SEQ ID NOs 1 to 14,000, preferably wherein the assay is characterised as having a receiver operating characteristics (ROC) area under the curve (AUC) of at least 0.67. The panel of one or more CpGs may comprise at least the CpGs identified in SEQ ID NOs 1 to 500 and identified at nucleotide positions 61 to 62, preferably wherein the assay is characterised as having an ROC AUC of at least 0.74.

In any of the assays described herein, the panel of one or more CpGs may comprise at least 1000 CpGs selected from the CpGs identified at nucleotide positions 61 to 62 in SEQ ID NOs 1 to 14,000, preferably wherein the assay is characterised as having a ROC AUC of at least 0.68. The panel of one or more CpGs may comprise at least the CpGs identified in SEQ ID NOs 1 to 1000 and identified at nucleotide positions 61 to 62, preferably wherein the assay is characterised as having a ROC AUC of at least 0.75.

In any of the assays described herein, the panel of one or more CpGs may comprise at least 2000 CpGs selected from the CpGs identified at nucleotide positions 61 to 62 in SEQ ID NOs 1 to 14,000, preferably wherein the assay is characterised as having a ROC AUC of at least 0.68. The panel of one or more CpGs may comprise at least the CpGs identified in SEQ ID NOs 1 to 2000 and identified at nucleotide positions 61 to 62, preferably wherein the assay is characterised as having a ROC AUC of at least 0.75.

In any of the assays described herein, the panel of one or more CpGs may comprise at least 10,000 CpGs selected from the CpGs identified at nucleotide positions 61 to 62 in SEQ ID NOs 1 to 14,000, preferably wherein the assay is characterised as having an AUC of at least 0.73. The panel of one or more CpGs may comprise at least the CpGs identified in SEQ ID NOs 1 to 10,000 and identified at nucleotide positions 61 to 62, preferably wherein the assay is characterised as having a ROC AUC of at least 0.78.

In any of the assays described herein, the panel of one or more CpGs may comprise at least the 14,000 CpGs identified at nucleotide positions 61 to 62 in SEQ ID NOs 1 to 14,000, and further wherein the assay is characterised as having a ROC AUC of at least 0.78.

In any of the above-described assays, the assay may be characterised as having a ROC AUC of 0.60 or more, 0.61 or more, 0.62 or more, 0.63 or more, 0.64 or more, 0.65 or more, 0.66 or more, 0.67 or more, 0.68 or more, 0.69 or more, 0.70 or more, 0.71 or more, 0.72 or more, 0.73 or more, 0.74 or more, 0.75 or more, 0.76 or more, 0.77 or more, 0.78 or more, 0.79 or more, 0.80 or more, 0.81 or more, 0.82 or more, 0.83 or more, 0.84 or more, 0.85 or more, 0.86 or more, 0.87 or more, 0.88 or more, 0.89 or more or 0.90 or more.

In any of the described assays, the methylation status of the one or more CpGs in the panel is preferably determined by a p-value analysis, and the cancer is ovarian cancer or endometrial cancer. Preferably, the cancer is ovarian cancer.

In any of the assays described herein, the panel of one or more CpGs may comprise at least 500 CpGs selected from the CpGs identified at nucleotide positions 61 to 62 in SEQ ID NOs 1 to 14,000, optionally wherein:
1. the assay is characterised as having an ROC AUC (AUC) of at least 0.74, and more preferably wherein the panel of one or more CpGs comprises at least the CpGs identified in SEQ ID NOs 1 to 500 and identified at nucleotide positions 61 to 62;
2. the assay is characterised as having an AUC of at least 0.74, and more preferably wherein the panel of one or more CpGs comprises at least the CpGs identified in SEQ ID NOs 501 to 1000 and identified at nucleotide positions 61 to 62;
3. the assay is characterised as having an AUC of at least 0.74, and more preferably wherein the panel of one or more CpGs comprises at least the CpGs identified in SEQ ID NOs 1001 to 1500 and identified at nucleotide positions 61 to 62;
4. the assay is characterised as having an AUC of at least 0.70, and more preferably wherein the panel of one or more CpGs comprises at least the CpGs identified in SEQ ID NOs 1501 to 2000 and identified at nucleotide positions 61 to 62;
5. the assay is characterised as having an AUC of at least 0.72, and more preferably wherein the panel of one or more CpGs comprises at least the CpGs identified in SEQ ID NOs 2001 to 2500 and identified at nucleotide positions 61 to 62;
6. the assay is characterised as having an AUC of at least 0.75, and more preferably wherein the panel of one or more CpGs comprises at least the CpGs identified in SEQ ID NOs 2501 to 3000 and identified at nucleotide positions 61 to 62;
7. the assay is characterised as having an AUC of at least 0.74, and more preferably wherein the panel of one or more CpGs comprises at least the CpGs identified in SEQ ID NOs 3001 to 3500 and identified at nucleotide positions 61 to 62;
8. the assay is characterised as having an AUC of at least 0.73, and more preferably wherein the panel of one or more CpGs comprises at least the CpGs identified in SEQ ID NOs 3501 to 4000 and identified at nucleotide positions 61 to 62;
9. the assay is characterised as having an AUC of at least 0.73, and more preferably wherein the panel of one or more CpGs comprises at least the CpGs identified in SEQ ID NOs 4001 to 4500 and identified at nucleotide positions 61 to 62;
10. the assay is characterised as having an AUC of at least 0.75, and more preferably wherein the panel of one or more CpGs comprises at least the CpGs identified in SEQ ID NOs 4501 to 5000 and identified at nucleotide positions 61 to 62;
11. the assay is characterised as having an AUC of at least 0.73, and more preferably wherein the panel of one or more CpGs comprises at least the CpGs identified in SEQ ID NOs 5001 to 5500 and identified at nucleotide positions 61 to 62;
12. the assay is characterised as having an AUC of at least 0.70, and more preferably wherein the panel of one or more CpGs comprises at least the CpGs identified in SEQ ID NOs 5501 to 6000 and identified at nucleotide positions 61 to 62;
13. the assay is characterised as having an AUC of at least 0.70, and more preferably wherein the panel of one or more CpGs comprises at least the CpGs identified in SEQ ID NOs 6001 to 6500 and identified at nucleotide positions 61 to 62;
14. the assay is characterised as having an AUC of at least 0.68, and more preferably wherein the panel of one or more CpGs comprises at least the CpGs identified in SEQ ID NOs 6501 to 7000 and identified at nucleotide positions 61 to 62;
15. the assay is characterised as having an AUC of at least 0.73, and more preferably wherein the panel of one or more CpGs comprises at least the CpGs identified in SEQ ID NOs 7001 to 7500 and identified at nucleotide positions 61 to 62;
16. the assay is characterised as having an AUC of at least 0.69, and more preferably wherein the panel of one or more CpGs comprises at least the CpGs identified in SEQ ID NOs 7501 to 8000 and identified at nucleotide positions 61 to 62;
17. the assay is characterised as having an AUC of at least 0.69, and more preferably wherein the panel of one or more CpGs comprises at least the CpGs identified in SEQ ID NOs 8001 to 8500 and identified at nucleotide positions 61 to 62;
18. the assay is characterised as having an AUC of at least 0.68, and more preferably wherein the panel of one or more CpGs comprises at least the CpGs identified in SEQ ID NOs 8501 to 9000 and identified at nucleotide positions 61 to 62;
19. the assay is characterised as having an AUC of at least 0.66, and more preferably wherein the panel of one or more CpGs comprises at least the CpGs identified in SEQ ID NOs 9001 to 9500 and identified at nucleotide positions 61 to 62;
20. the assay is characterised as having an AUC of at least 0.68, and more preferably wherein the panel of one or more CpGs comprises at least the CpGs identified in SEQ ID NOs 9501 to 10000 and identified at nucleotide positions 61 to 62;
21. the assay is characterised as having an AUC of at least 0.67, and more preferably wherein the panel of one or more CpGs comprises at least the CpGs identified in SEQ ID NOs 10001 to 10500 and identified at nucleotide positions 61 to 62;
22. the assay is characterised as having an AUC of at least 0.68, and more preferably wherein the panel of one or more CpGs comprises at least the CpGs identified in SEQ ID NOs 10501 to 11000 and identified at nucleotide positions 61 to 62;
23. the assay is characterised as having an AUC of at least 0.65, and more preferably wherein the panel of one or more CpGs comprises at least the CpGs identified in SEQ ID NOs 11001 to 11500 and identified at nucleotide positions 61 to 62;
24. the assay is characterised as having an AUC of at least 0.68, and more preferably wherein the panel of one or more CpGs comprises at least the CpGs identified in SEQ ID NOs 11501 to 12000 and identified at nucleotide positions 61 to 62;
25. the assay is characterised as having an AUC of at least 0.67, and more preferably wherein the panel of one or more CpGs comprises at least the CpGs identified in SEQ ID NOs 12001 to 12500 and identified at nucleotide positions 61 to 62;
26. the assay is characterised as having an AUC of at least 0.67, and more preferably wherein the panel of one or more CpGs comprises at least the CpGs identified in SEQ ID NOs 12501 to 13000 and identified at nucleotide positions 61 to 62;
27. the assay is characterised as having an AUC of at least 0.66, and more preferably wherein the panel of one or more CpGs comprises at least the CpGs identified in SEQ ID NOs 13001 to 13500 and identified at nucleotide positions 61 to 62; or
28. the assay is characterised as having an AUC of at least 0.67, and more preferably wherein the panel of one or more CpGs comprises at least the CpGs identified in SEQ ID NOs 13001 to 14000 and identified at nucleotide positions 61 to 62.

In any of the described assays, the methylation status of the one or more CpGs in the panel is preferably determined by a p-value analysis, and the cancer is ovarian cancer or endometrial cancer. Preferably, the cancer is ovarian cancer.

In any of the above-described assays, the assay may be characterised as having a ROC AUC of 0.60 or more, 0.61 or more, 0.62 or more, 0.63 or more, 0.64 or more, 0.65 or more, 0.66 or more, 0.67 or more, 0.68 or more, 0.69 or more, 0.70 or more, 0.71 or more, 0.72 or more, 0.73 or more, 0.74 or more, 0.75 or more, 0.76 or more, 0.77 or more, 0.78 or more, 0.79 or more, 0.80 or more, 0.81 or more, 0.82 or more, 0.83 or more, 0.84 or more, 0.85 or more, 0.86 or more, 0.87 or more, 0.88 or more, 0.89 or more or 0.90 or more.

In any of the assays described herein, the panel of one or more CpGs may comprise:
1. at least 100 CpGs selected from the CpGs identified at nucleotide positions 61 to 62 in SEQ ID NOs 1 to 14,000, preferably wherein the assay is characterised as having an AUC of at least 0.69, and more preferably wherein the panel of one or more CpGs comprises at least the CpGs identified in SEQ ID NOs 1 to 100 and identified at nucleotide positions 61 to 62;
2. at least 500 CpGs selected from the CpGs identified at nucleotide positions 61 to 62 in SEQ ID NOs 1 to 14,000, preferably wherein the assay is characterised as having an AUC of at least 0.74, and more preferably wherein the panel of one or more CpGs comprises at least the CpGs identified in SEQ ID NOs 1 to 500 and identified at nucleotide positions 61 to 62;
3. at least 1000 CpGs selected from the CpGs identified at nucleotide positions 61 to 62 in SEQ ID NOs 1 to 14,000, preferably wherein the assay is characterised as having an AUC of at least 0.75, and more preferably wherein the panel of one or more CpGs comprises at least the CpGs identified in SEQ ID NOs 1 to 1000 and identified at nucleotide positions 61 to 62;
4. at least 2000 CpGs selected from the CpGs identified at nucleotide positions 61 to 62 in SEQ ID NOs 1 to 14,000, preferably wherein the assay is characterised as having an AUC of at least 0.75, and more preferably wherein the panel of one or more CpGs comprises at least the CpGs identified in SEQ ID NOs 1 to 2000 and identified at nucleotide positions 61 to 62;
5. at least 3000 CpGs selected from the CpGs identified at nucleotide positions 61 to 62 in SEQ ID NOs 1 to 14,000, preferably wherein the assay is characterised as having an AUC of at least 0.76, and more preferably wherein the panel of one or more CpGs comprises at least the CpGs identified in SEQ ID NOs 1 to 3000 and identified at nucleotide positions 61 to 62;
6. at least 4000 CpGs selected from the CpGs identified at nucleotide positions 61 to 62 in SEQ ID NOs 1 to 14,000, preferably wherein the assay is characterised as having an AUC of at least 0.77, and more preferably wherein the panel of one or more CpGs comprises at least the CpGs identified in SEQ ID NOs 1 to 4000 and identified at nucleotide positions 61 to 62;
7. at least 5000 CpGs selected from the CpGs identified at nucleotide positions 61 to 62 in SEQ ID NOs 1 to 14,000, preferably wherein the assay is characterised as having an AUC of at least 0.78, and more preferably wherein the panel of one or more CpGs comprises at least the CpGs identified in SEQ ID NOs 1 to 5000 and identified at nucleotide positions 61 to 62;
8. at least 6000 CpGs selected from the CpGs identified at nucleotide positions 61 to 62 in SEQ ID NOs 1 to 14,000, preferably wherein the assay is characterised as having an AUC of at least 0.78, and more preferably wherein the panel of one or more CpGs comprises at least the CpGs identified in SEQ ID NOs 1 to 6000 and identified at nucleotide positions 61 to 62;
9. at least 7000 CpGs selected from the CpGs identified at nucleotide positions 61 to 62 in SEQ ID NOs 1 to 14,000, preferably wherein the assay is characterised as having an AUC of at least 0.78, and more preferably wherein the panel of one or more CpGs comprises at least the CpGs identified in SEQ ID NOs 1 to 7000 and identified at nucleotide positions 61 to 62;
10. at least 8000 CpGs selected from the CpGs identified at nucleotide positions 61 to 62 in SEQ ID NOs 1 to 14,000, preferably wherein the assay is characterised as having an AUC of at least 0.78, and more preferably wherein the panel of one or more CpGs comprises at least the CpGs identified in SEQ ID NOs 1 to 8000 and identified at nucleotide positions 61 to 62;
11. at least 9000 CpGs selected from the CpGs identified at nucleotide positions 61 to 62 in SEQ ID NOs 1 to 14,000, preferably wherein the assay is characterised as having an AUC of at least 0.78, and more preferably wherein the panel of one or more CpGs comprises at least the CpGs identified in SEQ ID NOs 1 to 9000 and identified at nucleotide positions 61 to 62;
12. at least 10000 CpGs selected from the CpGs identified at nucleotide positions 61 to 62 in SEQ ID NOs 1 to 14,000, preferably wherein the assay is characterised as having an AUC of at least 0.78, and more preferably wherein the panel of one or more CpGs comprises at least the CpGs identified in SEQ ID NOs 1 to 10000 and identified at nucleotide positions 61 to 62;
13. at least 11000 CpGs selected from the CpGs identified at nucleotide positions 61 to 62 in SEQ ID NOs 1 to 14,000, preferably wherein the assay is characterised as having an AUC of at least 0.78, and more preferably wherein the panel of one or more CpGs comprises at least the CpGs identified in SEQ ID NOs 1 to 11000 and identified at nucleotide positions 61 to 62;
14. at least 12000 CpGs selected from the CpGs identified at nucleotide positions 61 to 62 in SEQ ID NOs 1 to 14,000, preferably wherein the assay is characterised as having an AUC of at least 0.78, and more preferably wherein the panel of one or more CpGs comprises at least the CpGs identified in SEQ ID NOs 1 to 12000 and identified at nucleotide positions 61 to 62;
15. at least 13000 CpGs selected from the CpGs identified at nucleotide positions 61 to 62 in SEQ ID NOs 1 to 14,000, preferably wherein the assay is characterised as having an AUC of at least 0.78, and more preferably wherein the panel of one or more CpGs comprises at least the CpGs identified in SEQ ID NOs 1 to 13000 and identified at nucleotide positions 61 to 62; or
16. at least 14000 CpGs selected from the CpGs identified at nucleotide positions 61 to 62 in SEQ ID NOs 1 to 14,000, preferably wherein the assay is characterised as having an AUC of at least 0.78, and more preferably wherein the panel of one or more CpGs comprises at least the CpGs identified in SEQ ID NOs 1 to 14000 and identified at nucleotide positions 61 to 62.

In any of the described assays, the methylation status of the one or more CpGs in the panel is preferably determined by a p-value analysis, and the cancer is ovarian cancer or endometrial cancer. Preferably, the cancer is ovarian cancer.

In any of the above-described assays, the assay may be characterised as having a ROC AUC of 0.60 or more, 0.61 or more, 0.62 or more, 0.63 or more, 0.64 or more, 0.65 or more, 0.66 or more, 0.67 or more, 0.68 or more, 0.69 or more, 0.70 or more, 0.71 or more, 0.72 or more, 0.73 or more, 0.74 or more, 0.75 or more, 0.76 or more, 0.77 or more, 0.78 or more, 0.79 or more, 0.80 or more, 0.81 or more, 0.82 or more, 0.83 or more, 0.84 or more, 0.85 or more, 0.86 or more, 0.87 or more, 0.88 or more, 0.89 or more or 0.90 or more.

In any of the assays described herein, the panel of one or more CpGs may comprise:
1. at least 1000 CpGs selected from the CpGs identified at nucleotide positions 61 to 62 in SEQ ID NOs 1 to 14,000, preferably wherein the assay is characterised as having an AUC of at least 0.68, and more preferably wherein the panel of one or more CpGs comprises at least the CpGs identified in SEQ ID NOs 13,001 to 14,000 and identified at nucleotide positions 61 to 62;
2. at least 2000 CpGs selected from the CpGs identified at nucleotide positions 61 to 62 in SEQ ID NOs 1 to 14,000, preferably wherein the assay is characterised as having an AUC of at least 0.68, and more preferably wherein the panel of one or more CpGs comprises at least the CpGs identified in SEQ ID NOs 12,001 to 14,000 and identified at nucleotide positions 61 to 62;
3. at least 3000 CpGs selected from the CpGs identified at nucleotide positions 61 to 62 in SEQ ID NOs 1 to 14,000, preferably wherein the assay is characterised as having an AUC of at least 0.66, and more preferably wherein the panel of one or more CpGs comprises at least the CpGs identified in SEQ ID NOs 11,001 to 14,000 and identified at nucleotide positions 61 to 62;
4. at least 4000 CpGs selected from the CpGs identified at nucleotide positions 61 to 62 in SEQ ID NOs 1 to 14,000, preferably wherein the assay is characterised as having an AUC of at least 0.67, and more preferably wherein the panel of one or more CpGs comprises at least the CpGs identified in SEQ ID NOs 10,001 to 14,000 and identified at nucleotide positions 61 to 62;
5. at least 5000 CpGs selected from the CpGs identified at nucleotide positions 61 to 62 in SEQ ID NOs 1 to 14,000, preferably wherein the assay is characterised as having an AUC of at least 0.67, and more preferably wherein the panel of one or more CpGs comprises at least the CpGs identified in SEQ ID NOs 9,001 to 14,000 and identified at nucleotide positions 61 to 62;
6. at least 6000 CpGs selected from the CpGs identified at nucleotide positions 61 to 62 in SEQ ID NOs 1 to 14,000, preferably wherein the assay is characterised as having an AUC of at least 0.67, and more preferably wherein the panel of one or more CpGs comprises at least the CpGs identified in SEQ ID NOs 8,001 to 14,000 and identified at nucleotide positions 61 to 62;
7. at least 7000 CpGs selected from the CpGs identified at nucleotide positions 61 to 62 in SEQ ID NOs 1 to 14,000, preferably wherein the assay is characterised as having an AUC of at least 0.67, and more preferably wherein the panel of one or more CpGs comprises at least the CpGs identified in SEQ ID NOs 7,001 to 14,000 and identified at nucleotide positions 61 to 62;
8. at least 8000 CpGs selected from the CpGs identified at nucleotide positions 61 to 62 in SEQ ID NOs 1 to 14,000, preferably wherein the assay is characterised as having an AUC of at least 0.68, and more preferably wherein the panel of one or more CpGs comprises at least the CpGs identified in SEQ ID NOs 6,001 to 14,000 and identified at nucleotide positions 61 to 62;
9. at least 9000 CpGs selected from the CpGs identified at nucleotide positions 61 to 62 in SEQ ID NOs 1 to 14,000, preferably wherein the assay is characterised as having an AUC of at least 0.69, and more preferably wherein the panel of one or more CpGs comprises at least the CpGs identified in SEQ ID NOs 5,001 to 14,000 and identified at nucleotide positions 61 to 62;
10. at least 10000 CpGs selected from the CpGs identified at nucleotide positions 61 to 62 in SEQ ID NOs 1 to 14,000, preferably wherein the assay is characterised as having an AUC of at least 0.7, and more preferably wherein the panel of one or more CpGs comprises at least the CpGs identified in SEQ ID NOs 4,001 to 14,000 and identified at nucleotide positions 61 to 62;
11. at least 11000 CpGs selected from the CpGs identified at nucleotide positions 61 to 62 in SEQ ID NOs 1 to 14,000, preferably wherein the assay is characterised as having an AUC of at least 0.72, and more preferably wherein the panel of one or more CpGs comprises at least the CpGs identified in SEQ ID NOs 3,001 to 14,000 and identified at nucleotide positions 61 to 62;
12. at least 12000 CpGs selected from the CpGs identified at nucleotide positions 61 to 62 in SEQ ID NOs 1 to 14,000, preferably wherein the assay is characterised as having an AUC of at least 0.73, and more preferably wherein the panel of one or more CpGs comprises at least the CpGs identified in SEQ ID NOs 2,001 to 14,000 and identified at nucleotide positions 61 to 62;
13. at least 13000 CpGs selected from the CpGs identified at nucleotide positions 61 to 62 in SEQ ID NOs 1 to 14,000, preferably wherein the assay is characterised as having an AUC of at least 0.75, and more preferably wherein the panel of one or more CpGs comprises at least the CpGs identified in SEQ ID NOs 1,001 to 14,000 and identified at nucleotide positions 61 to 62;
14. at least 13500 CpGs selected from the CpGs identified at nucleotide positions 61 to 62 in SEQ ID NOs 1 to 14,000, preferably wherein the assay is characterised as having an AUC of at least 0.75, and more preferably wherein the panel of one or more CpGs comprises at least the CpGs identified in SEQ ID NOs 501 to 14,000 and identified at nucleotide positions 61 to 62;
15. at least 13900 CpGs selected from the CpGs identified at nucleotide positions 61 to 62 in SEQ ID NOs 1 to 14,000, preferably wherein the assay is characterised as having an AUC of at least 0.77, and more preferably wherein the panel of one or more CpGs comprises at least the CpGs identified in SEQ ID NOs 101 to 14,000 and identified at nucleotide positions 61 to 62; or
16. at least 14000 CpGs selected from the CpGs identified at nucleotide positions 61 to 62 in SEQ ID NOs 1 to 14,000, preferably wherein the assay is characterised as having an AUC of at least 0.78, and more preferably wherein the panel of one or more CpGs comprises at least the CpGs identified at nucleotide positions 61 to 62 in SEQ ID NOs 1 to 14,000.

In any of the described assays, the methylation status of the one or more CpGs in the panel is preferably determined by a p-value analysis, and the cancer is ovarian cancer or endometrial cancer. Preferably, the cancer is ovarian cancer.

In any of the above-described assays, the assay may be characterised as having a ROC AUC of 0.60 or more, 0.61 or more, 0.62 or more, 0.63 or more, 0.64 or more, 0.65 or more, 0.66 or more, 0.67 or more, 0.68 or more, 0.69 or more, 0.70 or more, 0.71 or more, 0.72 or more, 0.73 or more, 0.74 or more, 0.75 or more, 0.76 or more, 0.77 or more, 0.78 or more, 0.79 or more, 0.80 or more, 0.81 or more, 0.82 or more, 0.83 or more, 0.84 or more, 0.85 or more, 0.86 or more, 0.87 or more, 0.88 or more, 0.89 or more or 0.90 or more.

The invention also provides a variety of assays, each comprising any 1, 2, 3, 4, 5, 6, 7, 8, 9, 10 or more (or any range derivable therein) of a variety of steps and in no particular order, including methods of the following: measuring in a sample; analyzing a sample; assessing a sample; evaluating a sample; measuring nucleic acids in a sample; assessing nucleic acids in a sample; detecting nucleic acids in a sample; measuring methylation in nucleic acids in a sample; analyzing nucleic acids in a sample; assessing nucleic acids in a sample; measuring methylation at one or more CpG dinucleotides in a sample; detecting methylation at one or more CpG dinucleotides in a sample; assaying methylation at one or more CpG dinucleotides in a sample; assessing methylation at one or more CpG dinucleotides in a sample; measuring a methylation status in a sample; assaying a methylation status in a sample; detecting methylation status in a sample; determining methylation status in a sample; identifying methylation status in a sample; measuring one or more DNA methylation markers in a sample; assessing one or more DNA methylation markers in a sample; detecting one or more DNA methylation markers in a sample; measuring the presence of methylation at one or more markers in a sample; detecting the presence of methylation at one or more markers in a sample; assessing the presence of methylation at one or more markers in a sample; assaying the presence of one of more markers in a sample; measuring one or more DNA methylation markers in a sample but excluding the measuring of one or more other DNA methylation markers in the sample; assessing one or more DNA methylation markers in a sample but excluding the assessing of one or more other DNA methylation markers in the sample; analyzing one or more DNA methylation markers in a sample but excluding the analyzing of one or more other DNA methylation markers in the sample; detecting one or more DNA methylation markers in a sample but excluding the detecting of one or more other DNA methylation markers in the sample; measuring methylation status in nucleic acids from a sample from tissue from an individual other than tissue from the individual suspected of, or at risk for, being cancerous; detecting methylation status in nucleic acids from a sample from tissue from an individual other than tissue from the individual suspected of, or at risk for, being cancerous; analyzing methylation status in nucleic acids from a sample from tissue from an individual other than tissue from the individual suspected of, or at risk for, being cancerous; assessing methylation status in nucleic acids from a sample from tissue from an individual other than tissue from the individual suspected of, or at risk for, being cancerous; measuring methylation at one or more CpG dinucleotides in a sample but excluding the measuring of methylation at one or more CpG dinucleotides in the sample; assessing methylation at one or more CpG dinucleotides in a sample but excluding the assessing of methylation at one or more CpG dinucleotides in the sample; analyzing methylation at one or more CpG dinucleotides in a sample but excluding the analyzing of methylation at one or more CpG dinucleotides in the sample; detecting methylation at one or more CpG dinucleotides in a sample but excluding the detecting of methylation at one or more CpG dinucleotides in the sample; measuring methylation at one or more CpG dinucleotides in nucleic acids from a sample from tissue from an individual other than tissue from the individual suspected of, or at risk for, being cancerous; detecting methylation at one or more CpG dinucleotides in nucleic acids from a sample from tissue from an individual other than tissue from the individual suspected of, or at risk for, being cancerous; analyzing methylation at one or more CpG dinucleotides in nucleic acids from a sample from tissue from an individual other than tissue from the individual suspected of, or at risk for, being cancerous; assessing methylation at one or more CpG dinucleotides in nucleic acids from a sample from tissue from an individual other than tissue from the individual suspected of, or at risk for, being cancerous; treating an individual for cancer when the individual has been determined to have a methylation status at one or more methylation markers; treating an individual for cancer when the individual has been determined to have methylation at one or more CpG dinucleotides;
wherein any of the aforementioned methods, or any other methods encompassed by the disclosure, may comprise any one or more of the following method steps:
measuring methylation status, wherein the measuring identifies the methylation status of one or markers from nucleic acids in a sample;
measuring methylation status, wherein the measuring identifies the presence of one or more markers from nucleic acids in a sample;
measuring the presence of one or more methylation markers from a sample;
providing DNA from a sample;
providing nucleic acids from a sample;
determining whether one or more methylation markers from nucleic acids from a sample are methylated;
measuring whether one or more methylation markers from nucleic acids from a sample are methylated;
performing a sequencing step on nucleic acids from a sample;
determining a sequence of nucleic acids from a sample;
performing bisulphite conversion on one or more markers;
performing bisulphite conversion on one or more CpG dinucleotides;
hybridizing DNA to an array comprising probes capable of determining methylated versus non-methylated markers;
hybridizing DNA to an array comprising probes capable of determining methylated versus non-methylated CpG dinucleotides;
hybridizing DNA to an array comprising probes capable of discriminating between methylated and non-methylated markers;
hybridizing DNA to an array comprising probes capable of discriminating between methylated and non-methylated CpG dinucleotides;
performing an amplification step on sequence from nucleic acids from a sample;
performing an amplification step on sequence from nucleic acids using methylation-specific primers;
performing amplification of sequence comprising one or more regions suspected of having methylation or in need of determination of a methylation status;
performing PCR on sequence comprising one or more regions suspected of having methylation or in need of determination of a methylation status;
performing a capturing step;
performing a binding step;
performing a purification step;
performing a capturing step comprising binding of polynucleotides comprising one or more methylation markers to binding molecules specific to the one or more methylation markers and collecting complexes thereof,
stratifying the grade of a cancer;
determining the risk of cancer;
determining the risk of recurrence of cancer;
obtaining a sample from an individual;
obtaining DNA from a sample from an individual;
administering a treatment to an individual;
providing DNA from a sample;
determining whether one or more methylation markers from a panel of methylation markers comprises a specific sequence; and/or
obtaining data that identifies whether each one of a group of methylation markers from a panel comprises a specific sequence.

Moreover, in some aspects of the invention, an individual who is administered a therapy or treatment has been subjected to any of the methods and steps described herein.

Described herein are assays that utilise a statistically robust panel of one or more CpGs whose methylation status can be determined to provide a reliable prediction of the presence or development of cancer in an individual. By determining the methylation status of each CpG within the panel of one or more CpGs, a cancer index value may be derived thus enabling stratification of individuals according to their risk of developing cancer or of having cancer, particularly ovarian and/or endometrial cancer, with statistically robust sensitivity and specificity. The skilled person would understand that the methylation status of each CpG within a panel of one or more CpGs can be determined by any suitable means in order to thereby derive the cancer index value. Any one method, or a combination of methods, may be used to determine the methylation status of each CpG within a panel of one or more CpGs.

Various exemplary methods for determining the methylation status of each CpG within a panel of one or more CpGs are described herein. For example, in one method a percent methylated reference (PMR) value of a CpG may be determined. In another method the methylation β-values of a CpG may be determined. Different mechanisms may be employed to determine specific values depending on the circumstances, such as PCR-based mechanisms or array-based mechanisms.

### Cancer Index Values as Diagnostic and Risk Assessment Tools

In any of the assays described herein, the assessment of the presence, absence or development of cancer in an individual is based on the cancer index value of the individual at the time of testing.

As explained herein, using panels of the specific CpGs disclosed herein, cancer index values can be established which correspond with cancer negative samples, because they are based on values derived from individuals known to be cancer negative and obtained from samples from an anatomical site other than the ovary or endometrium, such as from the cervix, vagina, buccal area, blood and/or urine, particularly from a liquid-based cytology sample, and more preferably a cervical smear sample. Similarly, using panels of the specific CpGs disclosed herein, cancer index values can be established which correspond with cancer positive samples, because they are based on values derived from an anatomical site other than the ovary or endometrium, as noted above, from tissue samples from individuals known to be cancer positive. A user can then apply these cancer index values to assess the presence, absence or development of cancer in any test individual whose cancer status is required to be tested. As also explained herein, the assays of the invention are capable of being performed with a high degree of statistical accuracy.

As explained herein, the described assays particularly relate to the assessment of the presence, absence or development of ovarian cancer and/or endometrial cancer.

A skilled person will readily appreciate that a cancer index value provides a value that indicates a “likelihood” or “risk” or “prediction” of any of the assays of the invention correctly assessing the presence, absence or development of cancer in an individual. This is because the assessment is based upon a correlation between DNA methylation profiles of tissue samples and individual disease status. Nevertheless, as demonstrated by data set out in the Examples and elsewhere herein, the assays of the invention provide such correlations with high statistical accuracy, thus providing the skilled person with a high degree of confidence that the cancer index value which is determined for any test individual whose cancer status is required to be tested will provide an accurate correlation with actual disease status for the individual.

In the context of the present invention, “likelihood”, “risk” and “prediction” may be used synonymously with each other.

Any references herein to sequences, genomic sequences and/or genomic coordinates are derived based upon Homo sapiens (human) genome assembly GRCh37 (hg19). The skilled person would understand variations in the nucleotide sequences of any given sequence, may exist due to sequencing errors and/or variation between individuals.

The assay of the invention represents a ‘prediction’ because any cancer index value (WID-OC-Index) derived in accordance with the invention is unlikely to be capable of diagnosing every individual as having or not having cancer with 100% specificity and 100% sensitivity. Rather, depending on the cancer index cutpoint threshold applied by the user for positively predicting the presence of cancer in an individual, the false positive and false negative rate will vary. In other words, the inventors have discovered that the assays of the invention can achieve variable levels of sensitivity and specificity for predicting the presence, absence or development of cancer, as defined by receiver operating characteristics, depending on the cancer index cutpoint threshold chosen and applied by the user. Such sensitivity and specificity can be seen from the data disclosed herein to be achievable at high proportions, demonstrating accurate and statistically-significant discriminatory capability.

Similarly, cancer index values which have been pre-determined to correlate with specific cancer phenotypes, such as the presence or absence of cancer, have been defined with a high level of statistical accuracy as explained further herein.

Assessing the ‘development’ of cancer in the context of the invention may refer to assessing whether an individual is likely or unlikely to develop cancer. The inventors have shown that the CpGs assayed in order to derive the cancer index value of the assays of the invention are representative of the cells within normal tissue from an anatomical site other than the ovary or endometrium, such as from the cervix, vagina, buccal area, blood and/or urine, particularly from a liquid-based cytology sample, and more preferably a cervical smear sample. As such, cells from these tissues can act as a surrogate for ovarian and/or endometrial cells that may transform to cancer. In more detail, the cancer index value derived in accordance with the present invention has been show to progressively increase in samples from normal epithelial cells derived from the cervix, vagina, buccal area, blood and/or urine, preferably a cervical liquid-based cytology sample, and more preferably a cervical smear sample, in healthy women, to samples from corresponding tissue in women with a low grade ovarian cancer lesion such as a mucinous ovarian cancer, to samples from corresponding tissue in women with a high grade ovarian cancer lesion such as a serous ovarian cancer. Thus, assessing the development of cancer in accordance with the assays of the invention may refer to assessing an increased or decreased likelihood of cancer development, particularly ovarian cancer and endometrial cancer, preferably ovarian cancer. Assessing of the development of cancer in accordance with the assays of the invention may refer to assessing progression or worsening of a pre-existing cancer lesion in an individual. Assessment of the development of cancer in accordance with the assays of the invention may refer to predicting the likelihood of recurrence of cancer.

In any of the assays described herein, the step of assessing the presence or development of cancer in an individual based on a cancer index value may involve the application of a threshold value. Threshold values can provide a risk-based indication of an individual's cancer status, whether that is cancer positive, or cancer negative. Threshold values can also provide a means for identifying whether the cancer index value is intermediate between a cancer positive value and a cancer negative value. As explained herein, the cancer index value may be dynamic and subject to change depending upon genetic and/or environmental factors. Accordingly, the cancer index value may provide a means for assessing and monitoring cancer development. Cancer index values may therefore indicate at least a low risk or a high risk that the individual has a cancer positive status or has a status that is indicative of the development of cancer. If the cancer index value of an individual is determined by the assays of the invention at two or more time points, an increase or decrease in the individual's cancer index value may indicate an increased or decreased risk of the individual having or developing cancer, particularly ovarian and/or endometrial cancer, most preferably ovarian cancer.

Throughout the disclosure herein the terms “threshold value”, “cutpoint”, and “cutpoint threshold” are to be considered synonymous and interchangeable.

As explained further herein any assay of the invention is an assay for assessing the presence, absence or development of cancer in an individual. The types of cancer are set out further herein. As explained further herein, the assays of the invention provide means for assessing whether an individual is at risk of having or developing cancer based on specific cutpoint thresholds. Such risk assessments can be provided with a high degree of confidence based on the statistical parameters which characterise the assay. Thus in any of the assays described herein involving cancer index cutpoint thresholds, the cutpoint threshold may be used for risk assessment purposes. Equally, in any of the assays described herein involving cancer index cutpoint thresholds, the cutpoint threshold value may be used to specify whether or not an individual has cancer as a pure diagnostic test. Again, such diagnostic tests can be provided with a high degree of confidence based on the statistical parameters which characterise the assay. Accordingly, in any assay described herein which specifies that a cancer index value for the individual is a specific value or more, or is “about” a specific value or more, the individual may be assessed as having cancer. In any assay described herein which specifies that a cancer index value for the individual is less than a specific value, or is less than “about” a specific value, the individual may be assessed as not having cancer. The term “about” is to be understood as providing a range of +/−5% of the value.

Accordingly, any assay of the invention is an assay for assessing the presence, absence or development of cancer in an individual, the assay comprising:
e. providing a sample which has been taken from the individual, the sample comprising a population of DNA molecules;
f. determining in the population of DNA molecules in the sample the methylation status of a panel of one or more CpGs selected from a panel of CpGs identified at nucleotide positions 61 to 62 in SEQ ID NOs 1 to 14,000;
g. deriving a cancer index value based on the methylation status of the one or more CpGs in the panel; and
h. assessing the presence, absence or development of cancer in the individual based on the cancer index value;
wherein the assay is characterised as having an area under the curve (AUC) of 0.60 or more as determined by receiver operating characteristics (ROC).

Such an assay may be performed in accordance with any of the methods disclosed and defined herein.

As explained further herein, any assay of the invention for assessing the presence, absence or development of cancer in an individual may alternatively be referred to as an assay for stratifying an individual in accordance with their cancer status.

Accordingly, any assay of the invention is an assay for stratifying an individual for the presence, absence or development of cancer in an individual, the assay comprising:
a. providing a sample which has been taken from the individual, the sample comprising a population of DNA molecules;
b. determining in the population of DNA molecules in the sample the methylation status of a panel of one or more CpGs selected from a panel of CpGs identified at nucleotide positions 61 to 62 in SEQ ID NOs 1 to 14,000;
c. deriving a cancer index value based on the methylation status of the one or more CpGs in the panel; and
d. stratifying the individual for the presence, absence or development of cancer based on the cancer index value;
wherein the assay is characterised as having an area under the curve (AUC) of 0.60 or more as determined by receiver operating characteristics (ROC).

Such an assay may be performed in accordance with any of the methods disclosed and defined herein.

Accordingly, any assay of the invention is an assay for stratifying an individual for cancer, the assay comprising:
a. providing a sample which has been taken from the individual, the sample comprising a population of DNA molecules;
b. determining in the population of DNA molecules in the sample the methylation status of a panel of one or more CpGs selected from a panel of CpGs identified at nucleotide positions 61 to 62 in SEQ ID NOs 1 to 14,000;
c. deriving a cancer index value based on the methylation status of the one or more CpGs in the panel; and
d. stratifying the individual for cancer based on the cancer index value;
wherein the assay is characterised as having an area under the curve (AUC) of 0.60 or more as determined by receiver operating characteristics (ROC).

Such an assay may be performed in accordance with any of the methods disclosed and defined herein.

The cancer index value may be derived by any suitable means. Preferably, the cancer index value may be derived by assessing the methylation status of the one or more CpGs in the panel selected from a panel of CpGs identified at nucleotide positions 61 to 62 in SEQ ID NOs 1 to 14,000, in a sample provided from an individual. The methylation status of the CpGs may be determined by any suitable means. For example, in any of the assays described herein the step of determining the methylation status of each CpG in the panel of one or more CpGs may comprise:
a. performing a sequencing step to determine the sequence of each CpG;
b. hybridising DNA to an array comprising probes capable of discriminating between methylated and non-methylated forms of the CpGs and applying a detection system to the array so as to determine the methylation status of each CpG; and/or
c. performing a PCR step using methylation-specific primers, wherein the methylation status of the CpG is determined by the presence or absence of a PCR product.

Preferably, the the step of determining in the population of DNA molecules in the sample the methylation status of a panel of one or more CpGs further comprises determining a β value of each CpG. Deriving the cancer index value may involve providing a methylation β-value data set comprising the methylation β-values for each CpG in the panel of one or more CpGs. Optionally deriving the cancer index value may also involve estimating the fraction of contaminating DNA within the DNA provided from a sample.

DNA may be DNA originating from a particular source organism, tissue or cell type. Preferably the contaminating DNA originates from one or more different cell types to one or more cell types of interest. A cell type of interest may particularly be an epithelial cell. In some aspects of the invention, it may be preferable to estimate the fraction of contaminating DNA after the step of providing a sample which has been take from an individual. The assays described herein may optionally involve estimating a contaminating DNA fraction within DNA in the sample by any suitable means. Preferably, the contaminating DNA fraction for the sample is estimated via any suitable bioinformatics analysis tool. A bioinformatics analysis tool that may be used to estimate a contaminating DNA fraction may be EpIDISH. As described herein, it may be desirable to estimate the fraction of contaminating DNA from the one or more cell types that are different to the one or more cell types of interest because the cancer index value used for predicting the presence or development of cancer in an individual may, in some instances, only be reliably derived from determining the methylation status of a set of CpGs from DNA of a particular cell type of interest. Particularly, methylation status beta-values may differ in the one or more cell types of interest within a sample relative to methylation status beta-values in contaminating DNA from different cell types within the same sample. Thus, the derived cancer index value may in some instances have a decreased predictive power without estimating and controlling for the contaminating DNA fraction within the DNA provided from the sample. In assays of the invention that involve estimating the fraction of contaminating DNA and accordingly controlling for said contaminating DNA, it is preferable to estimate an immune cell DNA fraction within the DNA provided from the sample. In particular assays of the invention, wherein the individual has an immune cell contamination of over 50% (i.e. wherein more than 50% of the DNA in the sample is deemed to be derived from immune cells), the assay may preferably involve controlling for the immune cell contamination by deriving the cancer index, in accordance with the invention, solely from the DNA molecules derived from epithelial cells.

Any of the assays described herein comprising a step of deriving a cancer index value based on the methylation status of the one or more CpGs in the panel may further comprise applying an algorithm to the methylation beta-value dataset to obtain the cancer index value. Preferably, in any of the assays described herein, the step of deriving the cancer index value based on the methylation status of the panel of CpGs comprises providing a methylation beta-value data set comprising the methylation beta-values for each CpG in the panel and applying an algorithm to the methylation beta-value data set to obtain the cancer index value.

In any of the assays described herein, the step of deriving the cancer index value based on the methylation status of the one or more CpGs in the panel comprises:
a. providing a methylation j-value data set comprising the methylation β-values for each CpG in the panel;
b. providing a mathematical model capable of generating the cancer index from the methylation β-value data set; and
c. applying the mathematical model to the methylation β-value data set, thereby generating the cancer index.

In any of the assays described herein, the cancer index value may be calculated by any suitable mathematical model such as an algorithm or formula. Preferably, the cancer index value is termed Women's risk Identification for Ovarian Cancer Index (WID-OC-index) and wherein the mathematical model which is applied to the methylation β-value data set to generate the cancer index is calculated by an algorithm according to the following formula:

$$\\ WID - OC - index = {\sum }_{i = 1}^{n} \frac{{w}_{i} {β}_{i} - μ}{σ}$$

wherein:
a. β₁, . . . , βₙ are methylation beta-values (between 0 and 1);
b. w₁, . . . , w_{14,000} are real valued coefficients;
c. p and a are real valued parameters used to scale the index; and
d. n refers to the number of CpGs in the set of test CpGs;
preferably wherein the cancer is ovarian cancer.

In any of the assays described herein, the WID-OC-index algorithm applies real value coefficients inferred by initially training a dataset (this dataset in the exemplary embodiments of the invention described in the Examples consisted of 159 ovarian cancer cases and 572 controls) to fit a ridge classifier using the β package glmnet with a mixing parameter value of alpha=0 (ridge penalty) and binomial response type. Ten-fold cross-validation was used internally by the cv.glmnet function in order to determine the optimal value of the regularisation parameter lambda. The beta values from n CpGs for individual v, β₁^{v}, . . . , βₙ^{v}, are used as inputs to the ridge classifier. The coefficients w₁, . . . , wₙ are obtained from the fitted model. The following quantity was computed for each individual v in the training set:

$$\\ {x}_{υ} = {\sum }_{i = 1}^{n} {w}_{i} {β}_{i}^{{}^{} υ}$$

Any suitable real valued coefficients may be applied to the WID-OC-Index in any of the assays described herein.

The value of the parameters μ and σ are given by the mean and standard deviation of xᵥ in the training dataset respectively.

Thus, any suitable μ and σ real valued parameters may be applied to the WID-OC-index in any of the assays described herein. Any suitable training data set may be applied to the assays described herein in order to infer real value parameters and coefficients that can subsequently be applied to the WID-OC-index formula according to the present invention. Exemplary ways of utilising a training dataset in accordance with the present invention are further described in the ‘Statistical Analyses for Classifier Development’ section of the Materials and Methods section of the Examples.

Exemplary μ and σ real valued parameters are provided in Table 1 for CpG subsets identified in SEQ ID NOs 1 to 14,000. These real valued parameters may be applied to any of the assays described herein wherein the real parameters correspond to any one of the sets of CpGs identified in SEQ ID NOs 1 to 14,000 and set out in the left hand column of Table 1.

TABLE 1
Exemplary μ and σ real valued parameters are provided in
Table 1 for CpG subsets identified in SEQ ID NOs 1 to 14,000

| SEQ ID | | |
|---|---|---|
| NOs: | mu | sigma |
| 1-100 | 13.19 | 2.32 |
| 1-500 | 18.79 | 3.05 |
| 1-1000 | 16.83 | 3.36 |
| 1-2000 | 17.62 | 3.50 |
| 1-3000 | 18.00 | 3.60 |
| 1-4000 | 17.70 | 3.66 |
| 1-5000 | 17.22 | 3.69 |
| 1-6000 | 16.89 | 3.72 |
| 1-7000 | 16.99 | 3.74 |
| 1-8000 | 17.10 | 3.75 |
| 1-9000 | 17.08 | 3.76 |
| 1-10000 | 16.98 | 3.76 |
| 1-11000 | 16.92 | 3.76 |
| 1-12000 | 16.89 | 3.76 |
| 1-13000 | 16.10 | 3.47 |
| 1-14000 | 17.02 | 3.76 |

Exemplary w₁, . . . , wₙ real value coefficients are provided below for the CpGs identified at positions 61 to 62 in SEQ ID NOs 1 to 14,000. These real value coefficients may be applied to any of the assays described herein wherein the real parameters correspond to any one of the sets of CpGs identified in SEQ ID NOs 1 to 14,000 wherein the 14,000 real value coefficients below in turn correspond to the CpGs in turn identified at nucleotide positions 61 to 62 in SEQ ID NOs 1 to 14,000. Accordingly, the listed coefficients are presented below in numerical order corresponding respectively to the CpGs identified in SEQ ID NOs 1 to 5000. Thus the first number below corresponds to SEQ ID NO 1, the second number corresponds to SEQ ID NO 2 etc. The exemplary real value coefficients are as follows:
1.11358, 1.07458, 1.03297, 0.95999, 0.90279, 0.87819, 0.87606, 0.87087, −0.86625, −0.85785, 0.85576, 0.78465, 0.7758, −0.77138, 0.76446, 0.75844, 0.74197, 0.74118, −0.70987, 0.70788, 0.70184, 0.68543, −0.68471, −0.68402, 0.67336, −0.67212, 0.66314, −0.66142, 0.66036, −0.65917, −0.64945, −0.64766, 0.64617, 0.63244, 0.63003, −0.62856, 0.62619, 0.62603, 0.61734, −0.61715, 0.61582, 0.61499, 0.61464, 0.61439, −0.61281, 0.61238, −0.60732, −0.60145, 0.60015, −0.59847, 0.5983, 0.59759, −0.59604, 0.59282, 0.59057, 0.58952, 0.58018, 0.57892, −0.5778, 0.57756, −0.56965, −0.56939, 0.56892, 0.56797, −0.56796, 0.56761, 0.56156, −0.55768, −0.55698, 0.55592, −0.55558, 0.55256, 0.55255, 0.55153, −0.54636, 0.54581, 0.54321, −0.54253, 0.54104, −0.53898, −0.53862, −0.53708, −0.53575, 0.53563, −0.53367, −0.52811, 0.5232, 0.52242, 0.51389, −0.51381, −0.51326, 0.51295, 0.50999, 0.50932, −0.50911, 0.50801, 0.50588, −0.50394, 0.50036, −0.49945, 0.49405, 0.49372, 0.49289, −0.49271, 0.49091, 0.48662, 0.48628, 0.48564, 0.48377, 0.48332, −0.48219, 0.48163, 0.48016, 0.47989, 0.47846, 0.47758, −0.47611, 0.47577, −0.47535, −0.47507, −0.47477, 0.47376, −0.4734, 0.47327, 0.4727, 0.4714, 0.47093, −0.47032, 0.46987, 0.46983, −0.46731, 0.46587, 0.46452, 0.46432, 0.46402, −0.46314, −0.4628, 0.46266, −0.46021, 0.45991, 0.45917, −0.45776, 0.45706, 0.45649, 0.45412, −0.45278, 0.44972, 0.44839, 0.44418, −0.44209, 0.4339, 0.43259, 0.43211, −0.43148, −0.42815, 0.42351, −0.42143, 0.42068, −0.42007, 0.41858, 0.41746, 0.41718, 0.41638, 0.41418, −0.41389, 0.41223, −0.41083, −0.40834, 0.40796, 0.40742, −0.40706, −0.40535, 0.40509, −0.40465, 0.40428, −0.40269, −0.40201, 0.40137, −0.4007, 0.40002, 0.39975, 0.39971, 0.39951, 0.39941, −0.39938, 0.39703, 0.39667, 0.39592, 0.39529, −0.39519, 0.3948, 0.39226, −0.39102, −0.39085, −0.3904, 0.38989, 0.38964, 0.38842, 0.38829, 0.38829, 0.38797, 0.38767, 0.38686, 0.3866, −0.38633, 0.38624, −0.3861, −0.38442, −0.38436, 0.38326, −0.3831, −0.38285, −0.3823, −0.38147, −0.38112, 0.37996, 0.3789, −0.3788, 0.37855, 0.37784, 0.37618, 0.37494, 0.3744, 0.37374, 0.37278, −0.37235, 0.37233, 0.37177, 0.37164, 0.36982, −0.36967, −0.36964, −0.36893, 0.36887, 0.36731, 0.3673, 0.36639, 0.36617, 0.3659, −0.36529, 0.36406, −0.36398, −0.36374, −0.3637, −0.36363, −0.36337, −0.36287, 0.36167, −0.36068, −0.35993, 0.35848, 0.35843, 0.35837, 0.3579, 0.35782, 0.35753, −0.35719, −0.35446, 0.35365, 0.35349, 0.35326, 0.35251, −0.35222, 0.35195, −0.35146, 0.35075, 0.35058, −0.35029, 0.34991, 0.34921, −0.34908, 0.34822, −0.34781, 0.34749, −0.34673, 0.34633, −0.34611, 0.3454, 0.34406, 0.34381, −0.34297, 0.34157, −0.34072, −0.34004, 0.33943, −0.33921, −0.3382, 0.33785, 0.33746, 0.33722, −0.3371, −0.33686, −0.3361, −0.33598, 0.33564, 0.33534, 0.33501, 0.33496, −0.3346, 0.33458, 0.33416, −0.33387, −0.33386, −0.33349, 0.33311, −0.33285, 0.33269, −0.33238, 0.33202, 0.33198, −0.33115, −0.33097, −0.3309, −0.3306, 0.32959, −0.3294, 0.32869, −0.32848, −0.32793, −0.32792, −0.32701, −0.32665, −0.32629, 0.32602, −0.32594, −0.32544, −0.32541, 0.32503, −0.32459, 0.32431, −0.32406, 0.32353, 0.32283, 0.32249, 0.32144, 0.32112, 0.32105, 0.32053, 0.31983, −0.31942, 0.31919, −0.31884, 0.3183, 0.31794, −0.31791, 0.31754, 0.31739, 0.31644, −0.31517, 0.31501, −0.31499, 0.31438, 0.31413, 0.31339, −0.31187, 0.31169, −0.31134, 0.31085, −0.31055, 0.31027, −0.31025, −0.31023, −0.30976, 0.30972, 0.30914, −0.30898, −0.30869, 0.30853, −0.30848, −0.30821, −0.30814, 0.30789, 0.3078, 0.30757, −0.30747, 0.30731, −0.30639, −0.30635, −0.30621, −0.30583, 0.30574, 0.30574, −0.30573, 0.30571, 0.30524, 0.30519, 0.3042, −0.30361, −0.30351, −0.30274, 0.30266, 0.30255, 0.30249, 0.30226, −0.30223, 0.30218, −0.30217, −0.30198, 0.30191, 0.30148, 0.30148, 0.3006, −0.30051, 0.30029, 0.30016, −0.29972, 0.29957, 0.29947, 0.29933, −0.29887, −0.29873, 0.29848, 0.2982, 0.29794, 0.29768, 0.29759, −0.29754, −0.29749, 0.29741, 0.29733, −0.29683, 0.29663, −0.29646, 0.29637, 0.29621, 0.29608, 0.29575, −0.29522, 0.2951, −0.29501, 0.29469, −0.29454, 0.2944, −0.29381, 0.29332, 0.2933, −0.29316, −0.29292, 0.29289, −0.29279, 0.29271, 0.29183, −0.29154, 0.29132, 0.2913, 0.29124, −0.29104, 0.29026, 0.28995, 0.28958, −0.28932, −0.28931, 0.28928, 0.28924, 0.28901, −0.28897, −0.28882, 0.28878, 0.28875, 0.28872, 0.28863, −0.28829, 0.28801, 0.28754, 0.28663, −0.28631, 0.28623, −0.28617, −0.28498, −0.28489, −0.28404, 0.28389, −0.28381, 0.28343, 0.28327, 0.28288, −0.28205, 0.28199, −0.28189, 0.28187, −0.28166, −0.28162, 0.28158, −0.28158, −0.28121, −0.28109, 0.28101, 0.28085, 0.28082, 0.28051, 0.28047, 0.28046, 0.28034, −0.27934, −0.27902, −0.27899, 0.27878, −0.27835, 0.27824, 0.27797, −0.27782, 0.27776, −0.27762, 0.2773, 0.27685, 0.27683, −0.27681, 0.2763, 0.27596, 0.27551, 0.27525, 0.27524, 0.27521, −0.27512, −0.27473, −0.27459, −0.27443, −0.27402, −0.27394, 0.2739, 0.27373, −0.27366, −0.27339, −0.27333, −0.27314, −0.27293, −0.27268, −0.27266, 0.27264, 0.27248, 0.27242, −0.2723, 0.27227, 0.27226, 0.27225, 0.27223, 0.27145, 0.27099, 0.27085, 0.27055, 0.27024, −0.27013, 0.26987, −0.26967, 0.26955, −0.26887, 0.26871, −0.26852, 0.26839, −0.26786, 0.26752, −0.26738, 0.26738, 0.26732, 0.26675, −0.26673, 0.26664, 0.26652, −0.26616, 0.26606, 0.26546, 0.2653, 0.26446, 0.26439, −0.26439, 0.26439, 0.26434, 0.26423, 0.26409, −0.26389, −0.26363, 0.26355, 0.26331, −0.26312, 0.26306, 0.26269, −0.26265, 0.26235, −0.26233, −0.26231, −0.26216, 0.2621, −0.26175, −0.26166, −0.26162, 0.26162, −0.26158, 0.26156, 0.26156, 0.26145, 0.26123, −0.26115, 0.26109, −0.26096, 0.26049, 0.26044, −0.26043, −0.26017, −0.25996, 0.25968, 0.25952, −0.2593, −0.25919, −0.25919, 0.25906, 0.25878, 0.25875, −0.2585, −0.25812, 0.25804, 0.2577, −0.25758, −0.25751, 0.25749, 0.25737, 0.25731, −0.25724, 0.25698, 0.25691, −0.25669, −0.25657, −0.25649, 0.25632, −0.2563, 0.25611, −0.25582, 0.25572, −0.25562, 0.2554, 0.25539, 0.25536, −0.25535, 0.25533, −0.25505, −0.25479, −0.25476, −0.25473, −0.25465, 0.2546, −0.25458, 0.25412, 0.25403, 0.25365, 0.25361, 0.2536, 0.25341, −0.25336, 0.25331, 0.25316, 0.25296, 0.25259, 0.25246, −0.25181, 0.25167, 0.25152, 0.25116, −0.25073, 0.25053, 0.25034, 0.25031, −0.25022, 0.25017, −0.25009, −0.25003, 0.24985, 0.24984, −0.24971, 0.24967, 0.24952, −0.24951, 0.24928, −0.24924, −0.24916, −0.24862, 0.24844, −0.24843, 0.2484, 0.2478, −0.24772, −0.24772, −0.24761, −0.24758, −0.2475, −0.24737, 0.24718, 0.24706, −0.24672, −0.2467, −0.24664, −0.24663, −0.2463, 0.2462, 0.24598, −0.24571, 0.24512, 0.24484, 0.24473, 0.24472, 0.24442, 0.24442, 0.2444, 0.24435, −0.24423, −0.24412, 0.24364, 0.24356, −0.24354, −0.24325, −0.24285, −0.24251, 0.24225, 0.24218, −0.24208, 0.24122, 0.24122, −0.24115, −0.24115, 0.24108, 0.24094, 0.24084, −0.24027, −0.24023, −0.24, 0.23985, −0.23978, −0.23977, 0.23975, 0.23958, 0.23952, −0.23938, 0.23932, −0.23924, 0.23922, −0.23894, 0.23863, −0.23841, −0.23834, 0.23826, −0.23776, 0.23743, 0.23735, −0.23711, −0.23688, 0.23673, −0.23662, 0.23657, 0.23649, 0.23648, 0.2364, 0.23616, 0.23558, 0.23549, 0.23544, −0.23543, −0.23527, 0.2349, 0.23483, −0.23478, −0.23476, −0.23456, 0.23425, −0.2342, −0.23416, −0.23394, −0.23388, 0.23355, −0.23351, 0.23328, 0.2329, 0.23289, 0.23279, 0.23269, 0.23243, −0.23238, −0.23232, −0.23223, −0.23192, −0.23183, 0.23171, 0.23158, −0.23145, −0.23121, −0.231, 0.23097, 0.23077, 0.23063, 0.2306, −0.23055, 0.23053, 0.23053, −0.23047, −0.23034, −0.23033, −0.23029, −0.23025, 0.22989, 0.22988, 0.2297, 0.22964, 0.22961, 0.22956, −0.22915, −0.22911, 0.22906, −0.22897, 0.2287, −0.22855, −0.22837, 0.22823, −0.22809, −0.228, 0.22795, 0.22794, −0.22755, 0.22749, −0.2273, −0.22722, −0.22719, 0.22709, 0.22694, −0.22682, −0.22679, 0.22674, −0.22663, 0.22616, 0.22575, 0.22561, −0.22552, −0.22551, 0.22551, 0.22541, −0.2254, 0.22538, −0.22506, −0.22498, 0.22475, −0.22453, 0.22448, −0.22435, 0.22416, −0.22402, 0.22375, −0.22352, −0.22333, 0.22327, −0.2232, −0.22316, 0.22306, 0.22276, −0.2227, 0.22265, 0.22263, −0.22237, 0.22233, −0.22218, 0.22208, 0.22195, −0.22192, 0.22191, −0.22184, 0.22163, −0.22153, 0.22147, 0.22117, 0.22116, 0.22097, 0.22087, −0.22068, −0.22042, 0.22019, −0.22017, −0.22012, 0.22011, −0.22006, 0.22, 0.21983, −0.21968, −0.21949, −0.21908, 0.21908, −0.21877, −0.21873, 0.21868, 0.21863, −0.2186, 0.21857, 0.21856, 0.21852, 0.21851, 0.2185, −0.21849, −0.2184, 0.21831, −0.21793, −0.21785, −0.2178, −0.21777, 0.21742, −0.21737, −0.21733, −0.21716, −0.2169, −0.21685, 0.2168, −0.21625, 0.21622, −0.21612, 0.21602, −0.21601, 0.2159, 0.21574, −0.21566, 0.21564, −0.21554, −0.21551, −0.21549, −0.21548, −0.21547, −0.21526, −0.2152, −0.21502, −0.21497, 0.21496, −0.21493, −0.21487, −0.21464, −0.21453, −0.21453, 0.21453, 0.21451, −0.2145, 0.21446, −0.21431, 0.21428, −0.21416, 0.21411, −0.21407, −0.21407, −0.21405, −0.21385, 0.21375, −0.21348, −0.21348, 0.21345, 0.21344, −0.21342, 0.21312, −0.21309, −0.2129, −0.21285, 0.2128, 0.2128, −0.2128, 0.2127, 0.21246, −0.2123, 0.21228, −0.21206, −0.21203, 0.21201, 0.21191, −0.21189, 0.21177, −0.21171, 0.21157, 0.21157, 0.21157, −0.21152, −0.2114, −0.21121, 0.21101, 0.21095, 0.21076, 0.21075, 0.2102, 0.20972, −0.20972, 0.20969, −0.20957, 0.20927, −0.20926, −0.20909, 0.2089, 0.2088, 0.20857, −0.20857, 0.20849, −0.20841, −0.20838, 0.20827, 0.20801, 0.20795, 0.20768, 0.20761, 0.20744, −0.20738, 0.20733, 0.20723, −0.20721, 0.20706, 0.20706, 0.20704, 0.20691, 0.20675, −0.20667, −0.20662, 0.20654, −0.20639, 0.20619, 0.20609, 0.20604, −0.20604, −0.20602, −0.20595, −0.20583, 0.20581, 0.2057, 0.2056, −0.2056, 0.2055, 0.2054, −0.20498, −0.20492, −0.20482, −0.20477, 0.20462, 0.20459, 0.20454, −0.20393, 0.20385, −0.20362, −0.2036, −0.20356, 0.20343, 0.20338, −0.20325, −0.20324, −0.2032, −0.20304, −0.20301, 0.20274, −0.20272, −0.20253, −0.20241, −0.20235, −0.20217, −0.20161, 0.20158, −0.20151, 0.20145, 0.20136, 0.20134, −0.20129, 0.20127, 0.20117, −0.2011, −0.20098, 0.20096, 0.20092, 0.20082, 0.2007, −0.20062, 0.20042, −0.20039, 0.20033, 0.20024, 0.20015, 0.20011, −0.20007, 0.19984, −0.19981, −0.19957, 0.19949, −0.19919, −0.19918, −0.19905, −0.19896, 0.19884, −0.19868, 0.19866, −0.19856, 0.19844, −0.19826, −0.19816, 0.19809, 0.19796, 0.1979, 0.19782, 0.19759, −0.19752, 0.1975, 0.19745, −0.19745, 0.19738, −0.19736, −0.19733, −0.19726, −0.1972, −0.19719, 0.19713, 0.19711, −0.19708, −0.1969, 0.19681, 0.19681, 0.19669, −0.19661, −0.19661, 0.19656, 0.19634, 0.19614, −0.19603, −0.19594, 0.19591, 0.19578, 0.19541, −0.19521, 0.19508, 0.19495, −0.19494, 0.19476, 0.19472, −0.19463, 0.19456, −0.19456, −0.19446, 0.19446, 0.19426, 0.19414, 0.19407, 0.19398, 0.19396, −0.1939, 0.19377, −0.19364, −0.19363, −0.19318, −0.1931, 0.19308, −0.19306, 0.19287, −0.1928, −0.19275, −0.19267, 0.19261, 0.1924, 0.19207, 0.19201, −0.19197, 0.19196, −0.1919, 0.19185, −0.19184, −0.19178, −0.19171, 0.19167, −0.19159, −0.19156, 0.19085, −0.19079, −0.19075, −0.19062, 0.1906, 0.19051, 0.19041, −0.1902, −0.19017, −0.19011, 0.19002, 0.18996, −0.18981, 0.18979, 0.18976, −0.18956, 0.18956, −0.18944, −0.18935, 0.18931, 0.18925, −0.18915, 0.18914, −0.18913, 0.18911, 0.18907, 0.18895, −0.18891, 0.18881, −0.1888, −0.18862, 0.1886, −0.18844, 0.18839, −0.18833, 0.18806, −0.1878, 0.18776, 0.18761, 0.18755, −0.18748, 0.18743, 0.18742, 0.18729, 0.18725, 0.18715, 0.18683, 0.18681, 0.1868, 0.18677, 0.1867, 0.18639, −0.18637, −0.18629, −0.18623, −0.18577, 0.18573, 0.1856, 0.18549, 0.18544, 0.18522, 0.18508, 0.18502, 0.18494, −0.18486, 0.18452, −0.18448, −0.1843, −0.18423, 0.18419, 0.18406, 0.18404, −0.1838, 0.18379, 0.18366, 0.1836, 0.18359, −0.18354, −0.18331, −0.18328, 0.18312, −0.183, 0.18296, 0.18294, −0.1828, 0.18278, −0.18276, −0.18261, −0.18258, 0.18256, 0.1823, 0.18221, 0.18206, −0.18199, −0.18196, 0.18171, 0.18165, −0.18154, 0.1815, −0.18149, 0.18142, 0.18135, 0.18128, −0.18126, −0.18125, −0.18107, −0.18106, −0.18102, −0.1809, −0.18081, 0.18076, −0.18066, 0.18056, 0.18055, −0.18047, 0.18044, 0.18037, −0.18036, 0.18013, −0.18008, 0.18005, −0.18004, 0.17988, −0.17986, 0.17983, 0.17981, 0.17942, 0.17929, −0.1792, 0.17908, 0.17903, −0.17895, 0.17894, 0.17892, −0.17891, 0.17884, −0.17873, −0.1787, −0.17864, −0.17864, −0.17849, −0.17844, −0.17824, 0.17819, 0.17813, −0.17807, −0.17794, −0.17792, −0.17791, −0.17789, 0.17785, 0.17759, 0.17758, 0.1775, 0.17746, 0.17745, 0.17735, −0.17725, −0.17725, −0.17714, −0.17704, −0.177, −0.17685, −0.17683, 0.17683, 0.17677, 0.17674, −0.17672, 0.1767, −0.17657, −0.17653, −0.17641, −0.17634, −0.17633, −0.17612, −0.17603, −0.17593, −0.17581, −0.17578, 0.17566, 0.17553, 0.17552, 0.17542, 0.17538, −0.17521, −0.1752, 0.17512, −0.17512, 0.17485, 0.17468, −0.17465, 0.17464, −0.17455, 0.17452, 0.17448, −0.17444, −0.17437, 0.17436, −0.17433, −0.17423, 0.17417, −0.17386, −0.17386, −0.17384, 0.1737, 0.1736, −0.17353, 0.1735, −0.17335, 0.17323, 0.17322, −0.17314, 0.17311, 0.17306, −0.17302, 0.17292, −0.17286, −0.17277, 0.17249, 0.17235, 0.1723, 0.17205, 0.17202, 0.17187, −0.17175, −0.17171, −0.1717, 0.17159, −0.17154, −0.1714, −0.17139, −0.17114, 0.1711, −0.17109, −0.17109, 0.17087, 0.17087, −0.17058, 0.17054, −0.17052, 0.1705, −0.17038, −0.17036, 0.17031, 0.17013, 0.17013, 0.17013, −0.17012, −0.17007, 0.16991, 0.16986, −0.16976, −0.16969, 0.16959, 0.16953, 0.1695, −0.16947, −0.16943, 0.16924, 0.16921, 0.16921, 0.1692, −0.16912, 0.1691, −0.16903, 0.16901, −0.16895, 0.16881, −0.16877, −0.16875, −0.16867, −0.16862, 0.16862, −0.16856, 0.16839, −0.16839, 0.16838, −0.16837, 0.16835, 0.16829, −0.16815, 0.16812, 0.1681, 0.16768, −0.16767, −0.16757, 0.16756, −0.16755, −0.16727, 0.16721, −0.16716, −0.16715, 0.16715, 0.16714, 0.16712, 0.16707, −0.16704, −0.16689, 0.16688, −0.16685, −0.16685, −0.16677, −0.16674, −0.16666, −0.16662, −0.16659, −0.16648, 0.16646, 0.16615, 0.16606, 0.16605, −0.166, 0.16598, 0.16596, 0.16594, 0.16587, 0.16579, 0.16575, −0.16563, −0.16554, 0.16551, −0.16543, −0.16539, −0.16535, −0.1653, 0.16529, −0.16529, −0.16513, 0.16508, −0.16497, −0.16494, 0.16492, −0.16489, 0.16487, −0.16487, 0.16474, 0.16473, 0.16453, 0.16452, −0.16446, −0.16445, −0.16431, −0.16428, −0.16428, 0.16405, 0.16402, −0.16401, −0.164, −0.16389, −0.16385, 0.16379, 0.16378, 0.16368, −0.16361, −0.16357, 0.1635, 0.1635, −0.16329, 0.16327, 0.1632, 0.1632, 0.163, 0.16287, 0.16282, −0.16281, −0.16274, 0.16258, −0.16246, 0.16244, −0.16224, −0.16221, 0.16211, −0.16207, 0.16202, 0.16195, 0.1619, 0.16183, 0.16183, 0.1617, 0.16167, 0.16164, −0.16162, −0.1615, 0.16149, −0.16141, −0.1614, −0.16136, 0.16134, 0.16133, −0.16129, 0.16129, −0.1611, 0.16102, 0.16097, −0.16095, 0.16089, 0.16089, −0.16074, −0.16054, −0.16051, 0.1605, 0.16049, 0.16041, 0.16038, 0.15998, −0.15994, 0.15991, −0.15961, 0.15935, 0.15923, 0.15921, −0.15919, −0.15911, −0.159, 0.15897, 0.15896, 0.15893, −0.1588, −0.15877, −0.15871, 0.15868, 0.1586, −0.15859, 0.15858, 0.15845, −0.15838, 0.15836, −0.15835, −0.15819, 0.15819, 0.15819, −0.15813, −0.15803, 0.15797, −0.15789, −0.15789, −0.15787, 0.15785, 0.15782, 0.15775, 0.15772, 0.15764, 0.15748, 0.15745, −0.15736, 0.15734, −0.15731, −0.15721, −0.15714, 0.15708, −0.15704, −0.15704, 0.15699, −0.15693, −0.15692, 0.15691, −0.15682, 0.15679, −0.15677, 0.15676, 0.15673, 0.15663, −0.15661, −0.1566, −0.15646, 0.15624, −0.15623, −0.1562, −0.15596, 0.15591, 0.1559, 0.15584, 0.15581, −0.15579, −0.15575, −0.15575, 0.15568, −0.15563, 0.15559, 0.15558, −0.15555, 0.15551, −0.15549, −0.15533, 0.15524, −0.15521, −0.1551, −0.15488, 0.15477, −0.15474, −0.15473, −0.15472, 0.15472, −0.15465, −0.15464, −0.15453, 0.15451, −0.15451, 0.15447, −0.15445, 0.15442, −0.15441, −0.15436, 0.15434, 0.15433, 0.1543, −0.15429, −0.15423, −0.15411, 0.15403, −0.15401, 0.15382, 0.15382, 0.1538, 0.15375, −0.15372, 0.15371, 0.1537, 0.15363, 0.15362, 0.15362, 0.15358, −0.15344, −0.15341, 0.15332, −0.15312, 0.15309, −0.15307, 0.15304, −0.153, −0.15294, −0.1529, 0.15281, −0.15274, 0.15269, −0.15253, 0.15252, 0.15251, −0.15239, 0.15231, −0.15229, −0.15228, −0.15218, −0.15217, −0.15217, −0.15216, −0.152, 0.15195, −0.15191, −0.15186, 0.15183, −0.1517, −0.15166, 0.15153, 0.15149, 0.15136, 0.15123, 0.15113, −0.1511, 0.15109, −0.15108, 0.15099, 0.15098, −0.15094, −0.1509, 0.15089, 0.15087, −0.15083, −0.15083, 0.15083, 0.1508, 0.15068, −0.15066, 0.15062, 0.1506, 0.1506, 0.15059, −0.15053, −0.15051, 0.15047, −0.15045, −0.15042, 0.1504, 0.15032, 0.15029, −0.15028, 0.15026, 0.15019, 0.15014, −0.15012, −0.1499, −0.14988, −0.14983, 0.14974, 0.14962, −0.14961, −0.14952, −0.14938, −0.14936, 0.14936, 0.1493, −0.14921, −0.14916, 0.1491, −0.14908, −0.14904, 0.14901, −0.149, 0.1489, 0.14887, 0.14885, 0.14877, −0.14877, 0.14872, −0.14872, 0.14869, 0.14869, 0.14868, −0.14866, 0.14862, 0.14859, −0.14846, 0.14834, 0.14831, −0.14827, 0.14822, −0.14822, 0.1482, 0.14798, 0.14796, −0.14796, −0.14793, 0.14792, −0.14788, 0.14786, 0.14784, 0.14777, −0.14776, −0.14775, 0.14768, −0.14747, −0.14745, −0.14744, 0.14741, −0.14741, −0.1473, 0.147, −0.14698, −0.14698, 0.14692, 0.14691, 0.1469, −0.14685, 0.14678, 0.14676, 0.14667, 0.14667, −0.14659, 0.14655, 0.14654, 0.14647, −0.14647, −0.14629, −0.14625, −0.14623, 0.14602, −0.14602, 0.14598, −0.14594, −0.14594, 0.14589, −0.14582, −0.14557, 0.14557, 0.14557, 0.14553, 0.14544, −0.14543, −0.1452, −0.14518, 0.14516, −0.14513, −0.14508, 0.14506, 0.14486, 0.14485, 0.14481, 0.14479, −0.14479, −0.14479, 0.14464, −0.1446, −0.1446, 0.14457, 0.14456, −0.14455, 0.14448, 0.14446, −0.14432, −0.14431, −0.1443, −0.14427, −0.14427, −0.14424, 0.14423, 0.14422, −0.14421, −0.14417, −0.14411, −0.14404, 0.14393, 0.14392, −0.14387, 0.14384, −0.14384, 0.14382, 0.14381, 0.14371, 0.14361, 0.1436, 0.14359, −0.14355, 0.14353, −0.14352, −0.14348, 0.14343, 0.14336, −0.14333, −0.14316, 0.14314, −0.14311, 0.14302, 0.14301, 0.14295, −0.14294, −0.14292, −0.14291, 0.14289, −0.14289, 0.14285, −0.14285, 0.14277, −0.14275, −0.14252, −0.14245, 0.1424, −0.14237, 0.14233, −0.14233, −0.1423, −0.14228, −0.14205, −0.14201, −0.14183, 0.14179, −0.14168, −0.14167, −0.14161, 0.14152, −0.1415, −0.14136, 0.14131, 0.14131, −0.14128, −0.14123, −0.1412, 0.14119, −0.14119, −0.14095, 0.14094, 0.14087, 0.14081, 0.14073, −0.14061, −0.1406, 0.14057, 0.14056, 0.14054, 0.14052, 0.1405, 0.14041, −0.14035, −0.14032, 0.14032, −0.1402, 0.14009, 0.14005, 0.14005, 0.14001, −0.14001, 0.13997, −0.13989, −0.13988, −0.13984, 0.13981, 0.13978, 0.13973, 0.1397, 0.1397, 0.13969, 0.13965, −0.13961, −0.13956, 0.13955, 0.13955, −0.13954, −0.13953, 0.1395, 0.13946, −0.13943, −0.13939, 0.13938, 0.13938, 0.13937, −0.13935, 0.13927, −0.13924, 0.13924, 0.13918, −0.13917, 0.13916, −0.13909, −0.13909, 0.13909, −0.13907, 0.13895, 0.13885, 0.1388, 0.13874, −0.13865, 0.13865, −0.13857, 0.13856, 0.13855, 0.13848, −0.13843, 0.1384, −0.1384, −0.13834, −0.13831, 0.13824, 0.13823, −0.13819, −0.13811, 0.13797, −0.13795, 0.13784, 0.13783, 0.13779, 0.13775, 0.13773, 0.13765, −0.13765, 0.13762, −0.13754, −0.13738, 0.13736, −0.13731, 0.1373, 0.13729, −0.13727, 0.13725, −0.13722, 0.13717, 0.13714, 0.13714, −0.13696, −0.13691, 0.13682, −0.13668, 0.13666, 0.13661, −0.13653, 0.13651, −0.13642, 0.13635, −0.13635, −0.13634, −0.13633, −0.1363, 0.13629, −0.1362, −0.1362, −0.13618, 0.13617, −0.13611, −0.1361, −0.13607, −0.13607, 0.13606, −0.136, 0.136, −0.13586, 0.13578, 0.13575, −0.13571, 0.13558, 0.13556, 0.13552, −0.13546, −0.13538, −0.13536, 0.13532, −0.13522, 0.13519, 0.13519, −0.13518, −0.13512, 0.13507, 0.13499, 0.13493, 0.1349, 0.13483, 0.1348, 0.13478, −0.13477, 0.13474, −0.13471, −0.13463, −0.13455, 0.13455, −0.13454, −0.13452, −0.13449, −0.13445, 0.13441, 0.1344, −0.13438, −0.13415, 0.13398, 0.13394, −0.13393, −0.1339, −0.1339, 0.13384, 0.1338, 0.13379, −0.13378, 0.13376, 0.13376, −0.13374, 0.13357, −0.13348, 0.13346, 0.13344, 0.13341, −0.13341, 0.13337, −0.13334, 0.13332, 0.13327, −0.13319, 0.13312, −0.13306, 0.13306, −0.13301, 0.1329, −0.13283, 0.13281, −0.13276, −0.13275, 0.13273, 0.13269, 0.13264, 0.13255, 0.13251, 0.1325, 0.13248, −0.13247, −0.13243, 0.13239, 0.13233, 0.13231, −0.1323, 0.13222, −0.13219, 0.13216, 0.13215, 0.13211, −0.13209, 0.13203, −0.132, −0.13199, 0.13197, 0.13191, −0.13189, −0.13174, −0.13174, −0.13169, −0.13163, 0.1315, −0.1314, 0.13138, −0.13119, 0.13115, 0.13112, −0.13108, 0.13104, 0.13093, −0.13092, −0.13086, −0.13083, 0.13083, 0.1308, −0.13073, −0.1307, −0.13066, 0.13065, −0.1306, 0.13055, 0.13054, −0.13051, −0.13046, 0.13044, 0.13043, −0.13033, −0.13029, −0.13027, 0.13025, −0.13019, −0.13019, 0.13016, −0.13012, −0.13007, −0.12999, −0.12997, 0.12997, 0.1299, 0.12988, −0.12988, −0.12981, 0.12977, 0.12971, −0.12965, −0.12965, −0.12964, −0.12954, −0.12953, −0.12951, −0.1295, −0.1295, −0.12948, 0.12944, −0.12944, 0.12944, −0.1294, −0.1294, 0.12939, −0.12932, 0.12932, −0.12931, 0.12926, −0.12923, 0.1292, 0.12919, 0.12917, −0.1291, 0.12909, 0.12907, 0.12896, 0.12888, 0.12885, −0.12885, 0.12881, 0.12881, −0.1288, −0.12879, −0.12875, −0.12875, −0.1287, 0.1287, −0.12862, 0.12861, 0.12855, −0.12853, 0.12842, 0.12838, −0.12834, 0.12833, 0.12831, −0.12829, −0.12827, −0.12824, −0.1282, −0.12813, −0.12806, 0.12799, −0.12799, −0.12798, 0.12798, −0.12793, −0.12787, −0.12786, −0.12783, −0.12779, −0.12771, −0.1277, −0.12765, 0.12764, −0.12763, −0.12757, 0.12747, 0.12737, −0.12736, −0.12735, −0.12729, −0.12727, −0.12725, −0.12723, 0.12717, 0.12716, −0.12713, −0.12706, −0.12703, −0.12703, −0.127, 0.12698, −0.12697, −0.12696, 0.12693, 0.12689, −0.12687, −0.12678, 0.12672, −0.12671, −0.1267, 0.12667, −0.12667, 0.12663, −0.12659, 0.12657, 0.12655, −0.12653, −0.12653, 0.12653, −0.12644, −0.12641, −0.12639, −0.12638, 0.12637, 0.12635, 0.12635, −0.12614, 0.12603, −0.12602, 0.12594, 0.12586, 0.12586, 0.12569, −0.12568, 0.12566, 0.12564, 0.12562, −0.1256, −0.12559, −0.12559, 0.12553, −0.12547, −0.12547, 0.12545, −0.12537, −0.12536, 0.12531, 0.12529, 0.12528, 0.12514, 0.12513, −0.1251, 0.12501, −0.125, 0.12499, −0.12497, −0.12494, −0.12494, −0.12493, 0.12482, −0.12482, 0.12464, −0.12463, −0.12455, 0.12454, 0.12447, 0.12443, 0.1244, 0.1243, −0.12425, −0.12406, 0.12406, −0.12404, 0.12402, 0.12393, −0.12383, −0.12379, 0.12378, 0.12367, −0.12355, 0.12347, −0.1234, 0.12339, 0.12338, −0.12336, 0.12332, 0.12314, −0.12312, 0.12302, −0.12297, −0.12296, −0.12293, 0.12285, −0.12274, 0.12272, 0.1227, 0.12268, 0.12264, −0.12264, −0.1225, 0.12247, −0.12245, 0.12238, 0.12237, 0.12237, −0.12226, −0.12224, −0.12223, 0.1222, −0.12219, −0.12193, 0.12193, 0.12187, −0.12181, −0.1218, −0.12177, −0.12174, −0.12171, 0.12162, 0.12161, 0.12161, 0.12148, −0.12144, 0.12132, 0.1213, 0.12128, 0.12124, 0.12123, 0.1212, −0.1212, 0.12119, −0.12113, −0.12108, 0.12107, −0.12098, 0.12092, −0.12089, 0.12088, 0.12086, 0.12086, −0.12078, −0.12077, 0.12077, −0.12074, 0.12062, −0.12054, 0.12053, −0.12045, 0.12044, −0.12044, −0.12043, −0.12043, 0.12042, 0.12036, −0.12036, 0.12031, −0.12025, 0.12022, 0.12021, 0.1202, 0.12016, −0.12012, −0.12008, 0.11997, −0.11991, 0.11979, −0.11975, 0.11968, 0.11967, 0.1196, 0.11959, −0.11956, −0.11945, 0.11944, −0.11941, −0.1194, −0.11937, 0.11935, −0.11931, 0.11929, 0.11927, −0.11924, 0.11923, 0.11913, 0.11908, −0.11907, −0.11906, 0.11904, 0.11892, −0.11891, 0.11886, −0.11883, 0.11879, 0.11874, −0.1187, 0.1187, 0.11869, 0.11866, 0.11865, 0.11863, −0.11862, −0.1186, 0.11857, −0.11856, −0.11856, −0.11852, 0.11852, −0.11852, −0.11851, −0.11849, 0.11847, −0.1184, −0.11836, −0.11832, 0.11818, 0.11815, 0.11814, −0.11812, −0.11811, 0.11807, 0.11806, −0.11805, −0.11802, 0.11802, 0.11798, 0.11798, −0.11797, −0.11794, −0.11794, 0.11792, 0.11788, −0.11787, −0.11787, 0.11786, −0.11778, 0.11773, −0.11773, 0.11773, 0.1177, −0.1177, 0.11759, −0.1175, −0.11744, 0.11743, 0.11729, 0.11726, 0.11723, 0.11715, 0.11708, 0.11704, −0.11698, 0.11696, −0.11691, −0.1169, −0.11688, −0.11687, 0.11685, 0.11682, −0.11676, 0.11676, 0.11674, −0.11671, 0.11668, −0.11663, −0.11662, 0.11662, 0.11655, −0.1165, −0.11647, −0.11645, −0.11643, 0.11638, −0.11631, 0.11627, −0.11621, 0.11618, −0.11608, −0.11608, 0.11607, −0.11607, 0.11603, 0.11597, −0.11596, −0.11594, −0.1159, −0.11586, 0.11586, 0.11585, −0.11585, −0.11581, 0.11579, 0.11576, 0.11575, −0.11573, −0.11572, −0.1157, −0.1157, −0.11562, 0.11558, −0.11558, −0.11557, −0.11557, 0.11543, 0.11543, −0.11543, −0.11542, −0.11538, 0.11537, −0.11535, −0.11532, −0.11531, 0.11523, 0.11523, 0.11513, −0.11503, 0.11501, 0.115, −0.11497, 0.11493, −0.11478, −0.11474, −0.11472, −0.11471, 0.11466, 0.11458, 0.11458, −0.11457, 0.11455, 0.11452, −0.11451, −0.11451, 0.11449, 0.11445, −0.11439, 0.11438, 0.11435, −0.11434, −0.11429, −0.11429, −0.11427, 0.11424, 0.11422, 0.11413, −0.1141, −0.11408, −0.11406, −0.11404, 0.11402, −0.11401, −0.11399, −0.11392, 0.11388, −0.11387, 0.11382, 0.1138, −0.1138, 0.11377, −0.11374, −0.11373, 0.11372, −0.11371, −0.11366, 0.11364, 0.11353, −0.11349, −0.11348, −0.11345, 0.11341, −0.1134, −0.1134, 0.11339, 0.11336, −0.11336, −0.11334, 0.11334, −0.11332, 0.11325, −0.1132, −0.11319, 0.11314, 0.11311, 0.1131, 0.11305, 0.11304, −0.11302, −0.11292, −0.11291, −0.11284, −0.1128, 0.11276, −0.11275, 0.11274, −0.11273, −0.11271, 0.11269, −0.11266, 0.11263, −0.11256, −0.11255, −0.11253, 0.11253, 0.11248, 0.11247, 0.11242, 0.11237, 0.11236, 0.11233, −0.11231, 0.11229, 0.11228, −0.11227, 0.11225, 0.11225, −0.11213, 0.11208, −0.11206, −0.11206, −0.11203, −0.11203, −0.11199, 0.11198, −0.11197, −0.11194, 0.11194, −0.11186, 0.11186, 0.11183, −0.11183, 0.11181, 0.1118, −0.11177, −0.11173, 0.11161, −0.11157, −0.11155, −0.11151, −0.11151, −0.11149, −0.11147, 0.11146, 0.11144, 0.11143, 0.11141, −0.11139, −0.11138, 0.11135, −0.11129, −0.11128, −0.11128, −0.11124, −0.11122, 0.11119, 0.11118, −0.11114, −0.11112, −0.11111, 0.11107, −0.11101, 0.11101, −0.11097, 0.11096, −0.11094, 0.11093, 0.11087, 0.11086, −0.11085, 0.11081, −0.1108, −0.11077, 0.11076, −0.11076, −0.11075, −0.11073, −0.11072, −0.11071, 0.11062, −0.1106, 0.11056, 0.11046, −0.11044, 0.11042, 0.11041, −0.1104, 0.11039, 0.11038, −0.11037, −0.11033, −0.11033, −0.1103, 0.1103, −0.11026, 0.11024, −0.11023, 0.11023, −0.11021, −0.11017, 0.11016, −0.1101, −0.11009, −0.11009, 0.11007, −0.11004, −0.11001, 0.11, 0.11, 0.10998, −0.10998, 0.10994, −0.1099, −0.10989, 0.10985, −0.10985, 0.1098, 0.1098, 0.10978, −0.1097, −0.1097, −0.1097, 0.10969, −0.10967, −0.10966, 0.10959, −0.10952, −0.10948, −0.10947, 0.10947, 0.10944, 0.10944, 0.10938, 0.10937, 0.10925, 0.10923, 0.1092, 0.10916, −0.10913, 0.10909, 0.10905, 0.10905, 0.10902, −0.10901, −0.109, −0.10898, 0.10898, −0.10895, 0.10894, 0.10885, 0.10884, −0.10884, 0.10878, 0.10878, 0.10875, −0.1087, 0.1087, 0.10867, 0.10866, 0.10864, −0.10864, 0.10861, −0.10858, −0.10851, 0.10847, −0.10843, −0.10839, −0.10837, 0.10833, −0.10819, 0.10818, −0.10815, −0.10805, −0.10804, −0.10802, −0.10798, −0.10797, −0.10797, −0.10796, −0.1079, −0.10785, −0.10784, −0.10784, 0.10784, −0.10783, −0.10778, 0.10776, −0.10775, −0.10774, 0.10771, 0.1077, −0.10768, −0.10767, −0.10764, −0.1076, 0.10759, −0.10755, 0.10753, 0.10752, 0.1075, 0.1075, −0.10741, −0.10737, −0.10732, −0.10731, 0.10731, −0.1073, −0.10727, 0.10726, −0.10723, 0.10718, 0.10714, 0.10713, −0.1071, −0.10707, 0.10704, 0.10696, 0.10694, 0.10693, 0.10693, −0.10691, −0.10689, −0.10685, 0.10681, −0.10677, 0.10669, 0.10668, 0.10667, −0.10663, 0.10658, −0.10658, 0.10654, 0.10646, 0.10641, 0.1064, 0.10638, 0.10632, −0.10628, 0.10628, 0.10627, −0.10626, −0.10626, 0.10625, −0.10625, 0.10623, −0.10621, −0.10613, −0.10613, −0.10613, −0.10604, 0.10602, −0.10595, −0.10594, −0.10592, −0.10586, 0.10573, −0.10569, −0.10567, −0.10561, 0.1056, 0.10558, −0.10557, 0.10555, −0.10552, −0.10551, 0.10549, −0.10548, −0.10542, 0.1054, 0.10538, −0.1053, 0.10529, 0.10526, 0.10518, −0.10518, 0.10515, −0.10508, 0.10503, −0.105, 0.10499, 0.10489, −0.10489, 0.10488, −0.10487, −0.10483, 0.10481, 0.10474, −0.10474, −0.10474, 0.10472, −0.10471, −0.10466, 0.10459, 0.10454, −0.10453, 0.10449, 0.10445, −0.10445, −0.10433, −0.10432, 0.10428, 0.10422, 0.10419, −0.10418, 0.10413, 0.10413, −0.10409, 0.10408, −0.10405, 0.10403, 0.10396, −0.10393, −0.1039, −0.10389, 0.10388, −0.10385, −0.10378, −0.10377, −0.10374, −0.10373, −0.10369, 0.10369, 0.10367, 0.10365, −0.10363, 0.10362, −0.10362, −0.10361, −0.1035, 0.10349, 0.10347, 0.10347, −0.10346, 0.10343, −0.10341, 0.10334, −0.10333, 0.10332, 0.10327, −0.10326, 0.10322, 0.10321, 0.10318, −0.10318, −0.10315, −0.10315, 0.10313, −0.10312, −0.10307, 0.10306, −0.10306, 0.10303, 0.10301, 0.10293, 0.10293, −0.10293, 0.10293, 0.10292, −0.10291, 0.10291, 0.10289, 0.10288, −0.10287, −0.10286, 0.10286, −0.10284, −0.10279, −0.10279, −0.10276, 0.10275, −0.1027, 0.1027, 0.10269, −0.10263, 0.1026, 0.10258, −0.10256, −0.10254, −0.10253, 0.1025, 0.10241, −0.1024, −0.10238, 0.10238, 0.10223, 0.10223, −0.1022, −0.10215, 0.10215, 0.10213, 0.1021, 0.10207, −0.10207, 0.10205, −0.10204, −0.10204, −0.10202, 0.10197, 0.10195, 0.10187, 0.10186, 0.10178, 0.10176, −0.10176, −0.10175, 0.10174, 0.10174, 0.10174, −0.10174, −0.10171, 0.10169, 0.10166, 0.10165, −0.10162, −0.10161, −0.1016, −0.1016, 0.1016, 0.1016, 0.10159, −0.10155, −0.10154, 0.1015, 0.10145, 0.10144, −0.10139, 0.10138, −0.10138, −0.10136, −0.10134, 0.10133, 0.10133, −0.1013, −0.10129, 0.10126, 0.10125, −0.10125, −0.10125, −0.10118, 0.10111, −0.10111, −0.1011, −0.1011, 0.1011, 0.10108, −0.10108, −0.10105, −0.10102, 0.10102, 0.10101, −0.10095, 0.10093, 0.10089, −0.10089, 0.10087, −0.10086, 0.10084, −0.1008, −0.10079, 0.10076, 0.10075, 0.10074, 0.10073, 0.10069, 0.10068, 0.10068, 0.10067, 0.10066, 0.1006, −0.10058, 0.10056, 0.10053, −0.10049, −0.10046, 0.1004, 0.10039, −0.10038, 0.10033, −0.10032, −0.10028, −0.10022, 0.1002, −0.10017, 0.10016, −0.10012, −0.10009, −0.10008, −0.10006, 0.1, −0.09997, 0.09992, 0.09991, 0.0999, 0.09988, −0.09985, −0.09979, 0.09973, 0.09971, −0.0997, −0.09966, −0.09965, −0.09962, −0.0996, 0.09957, −0.09957, −0.09947, −0.09946, −0.0994, 0.09937, 0.09935, −0.09934, 0.09933, −0.09933, −0.09931, −0.0993, −0.09928, −0.09925, −0.09925, 0.09924, −0.09923, −0.0992, 0.09917, 0.09913, 0.09909, −0.09905, 0.09903, 0.09897, −0.09896, 0.09895, −0.09893, 0.09886, −0.09886, −0.09884, −0.09883, −0.09883, 0.0988, 0.09879, −0.09877, 0.09871, 0.09865, 0.0986, −0.09857, −0.09857, 0.09857, −0.09851, −0.09849, 0.09846, 0.09844, −0.09844, −0.09844, 0.09843, −0.0984, 0.09839, −0.09836, −0.09835, −0.09835, 0.09833, −0.09831, 0.09828, 0.09824, 0.09821, 0.09818, 0.09817, 0.09817, −0.09816, 0.09813, 0.09812, −0.09811, 0.09801, 0.09798, −0.09794, −0.0979, −0.09789, −0.09785, −0.09783, 0.09781, 0.09779, 0.09777, 0.0977, −0.0977, −0.09767, 0.09763, 0.09757, 0.09745, 0.09738, 0.09737, −0.09736, −0.09734, −0.09728, 0.09722, 0.09719, −0.09718, 0.09714, −0.09714, −0.09714, −0.09713, −0.09713, −0.09713, −0.0971, 0.09709, −0.09702, −0.097, −0.09696, −0.09696, 0.09696, −0.09693, −0.09692, 0.0969, 0.09685, 0.09685, −0.09684, −0.09682, 0.09682, −0.09679, −0.09677, 0.09674, 0.09674, 0.09672, 0.09672, −0.09669, 0.09669, 0.09662, 0.09661, −0.09659, 0.09659, 0.09658, 0.09658, −0.09658, −0.09655, 0.09647, 0.09645, 0.09644, −0.09639, 0.09635, 0.09633, 0.09627, −0.09625, −0.09625, −0.09623, −0.09617, 0.09616, −0.09612, 0.09608, −0.09605, 0.09604, 0.09604, 0.09594, 0.09587, −0.09582, 0.0958, −0.0958, 0.09579, −0.09579, 0.09579, −0.09577, 0.0957, −0.09566, 0.09561, 0.0956, 0.09559, −0.09556, −0.09555, −0.0955, 0.09548, 0.09546, 0.09541, −0.09541, 0.09539, 0.09535, −0.0953, 0.09525, 0.09521, −0.0952, −0.09519, 0.09517, −0.09516, −0.09512, −0.09507, −0.09505, 0.09503, −0.09501, 0.09501, 0.095, 0.095, −0.095, 0.09498, −0.09495, 0.09494, −0.09492, −0.0949, −0.09488, 0.09488, −0.09487, −0.09485, −0.09484, 0.09483, −0.09482, 0.09481, −0.0948, −0.09477, −0.09477, −0.09475, 0.09471, 0.09471, −0.09466, −0.09464, −0.09462, −0.0946, 0.09456, −0.09451, 0.09449, 0.09448, −0.09441, −0.0944, −0.09439, −0.09438, −0.09434, 0.09427, 0.09426, 0.09419, −0.09418, −0.09417, 0.09415, −0.09412, 0.0941, −0.09407, −0.09407, 0.09407, 0.09404, 0.09404, 0.094, 0.09399, −0.09394, 0.09392, −0.09392, 0.09389, 0.09389, −0.09386, 0.09384, −0.09373, −0.09372, −0.09371, 0.09371, −0.09368, −0.09368, −0.09362, −0.09362, −0.09354, 0.09353, −0.09351, −0.09349, −0.09347, 0.09343, −0.0934, 0.09337, −0.09336, −0.09334, −0.09334, −0.0933, 0.09328, −0.09324, 0.09323, −0.09322, −0.09319, −0.09319, 0.09312, −0.09308, −0.09307, 0.09306, −0.09305, −0.09305, −0.09302, 0.09298, 0.09294, 0.09294, 0.09292, 0.09288, −0.09287, 0.09284, −0.09284, 0.09278, −0.09274, 0.09272, −0.09271, 0.0927, −0.09266, −0.09265, 0.09261, −0.09257, 0.09252, 0.09252, −0.09251, −0.09249, −0.09249, −0.09249, 0.09247, −0.09247, −0.09242, −0.09239, 0.09239, 0.09237, 0.09236, 0.09235, 0.09234, 0.09233, −0.09228, 0.09228, −0.09227, 0.09227, −0.09224, −0.09223, −0.09222, −0.09211, −0.09211, −0.09207, 0.09206, 0.09204, 0.09203, 0.09203, −0.09203, 0.09194, 0.09191, 0.0919, −0.09189, −0.09188, 0.09187, −0.09184, 0.09179, 0.09175, −0.09174, 0.09173, 0.09171, −0.0917, 0.09167, 0.09165, −0.09165, −0.09165, −0.09163, 0.09162, −0.09158, −0.09158, −0.09152, −0.09148, −0.09148, 0.09143, −0.09142, 0.09139, 0.09135, 0.09134, −0.09132, −0.09131, −0.09126, 0.09125, 0.09125, 0.09125, −0.09121, 0.0912, 0.09119, −0.09118, 0.09117, −0.09117, 0.09115, 0.0911, 0.09109, −0.09106, −0.09105, −0.09104, 0.09102, −0.091, 0.09098, 0.09094, −0.09093, 0.09088, −0.09082, −0.09078, −0.09075, 0.09067, 0.09067, 0.09065, −0.09058, −0.09057, −0.09051, −0.0905, 0.0905, 0.09049, −0.09048, 0.09039, 0.09035, 0.09033, 0.09031, 0.0903, 0.09023, −0.09022, −0.09021, −0.09019, 0.09019, 0.09016, 0.09014, −0.09013, 0.0901, 0.09006, −0.09002, −0.09002, −0.08994, −0.08994, −0.08991, 0.08987, −0.08986, −0.08983, 0.08976, 0.08975, −0.08973, 0.08971, −0.08967, 0.08967, 0.08964, −0.08958, −0.08957, −0.08955, 0.08954, 0.08948, −0.08945, −0.08945, −0.08944, −0.08937, 0.08937, −0.08935, 0.08935, 0.08927, 0.08918, −0.08913, −0.08912, 0.0891, −0.08908, 0.08904, −0.08902, 0.08902, −0.08901, −0.08896, 0.08894, 0.08894, −0.08894, 0.08892, −0.08891, 0.08886, 0.08881, 0.08877, 0.08876, 0.08873, 0.08866, −0.08865, 0.08863, −0.08863, 0.08863, 0.08861, 0.0886, −0.08859, 0.08858, −0.08852, 0.08851, 0.08849, −0.08849, −0.08846, 0.08845, 0.08845, 0.08845, −0.08845, 0.08843, 0.08841, 0.08839, −0.08837, −0.08832, 0.08831, 0.08831, −0.08828, −0.08826, −0.08819, −0.08819, −0.08818, 0.08816, −0.08812, 0.08812, −0.08812, 0.08811, 0.08811, −0.08808, −0.08806, 0.08805, 0.08798, −0.08792, 0.08789, −0.08786, −0.08781, −0.08779, 0.08777, −0.08777, 0.08772, −0.0877, 0.08767, −0.08765, −0.08764, 0.08763, 0.08761, −0.08761, −0.08759, −0.08758, −0.08757, −0.08755, −0.08754, −0.08753, −0.08751, 0.08749, 0.08749, −0.08747, 0.08745, 0.0874, −0.08735, 0.08735, 0.08731, 0.08725, −0.08725, −0.08724, −0.08724, −0.0872, −0.08717, −0.08717, −0.08717, −0.08716, 0.0871, 0.08709, 0.08706, −0.08704, −0.08704, −0.08701, −0.08698, −0.08697, −0.08695, −0.08689, 0.08688, 0.08686, 0.08684, 0.08683, 0.08682, 0.08673, −0.08671, 0.08669, −0.08668, −0.08668, −0.08665, −0.08665, 0.08665, −0.08664, 0.08664, 0.08663, −0.08662, 0.08655, 0.08655, 0.08653, −0.08652, −0.08652, −0.0865, 0.08646, 0.08642, −0.08641, −0.08638, 0.08638, 0.08637, 0.08635, 0.08634, 0.08633, −0.08631, −0.0863, −0.08629, 0.08628, −0.08628, 0.08627, −0.08626, −0.08623, −0.08622, 0.08622, −0.08619, 0.08618, −0.08617, 0.08616, 0.08615, 0.08607, −0.08606, −0.08602, −0.08602, 0.08601, −0.08598, −0.08597, 0.08595, −0.08594, −0.08592, 0.08591, −0.0859, −0.08585, −0.08581, 0.08575, −0.08568, 0.08567, 0.08563, −0.08561, −0.0856, 0.08559, 0.08557, −0.08555, 0.08554, −0.08552, −0.08547, −0.08544, −0.08542, −0.08539, −0.08536, 0.08534, 0.08529, −0.08527, −0.08524, −0.08523, −0.08523, −0.08523, −0.08522, 0.08519, 0.08515, 0.08515, −0.08514, 0.08514, −0.08508, −0.08508, 0.08508, 0.08506, −0.08504, 0.08502, −0.08501, 0.085, −0.08497, −0.08495, 0.08493, −0.08492, 0.08489, 0.08483, 0.08482, −0.08476, 0.08475, 0.0847, 0.08468, −0.08466, −0.08462, 0.08459, 0.08458, −0.08457, 0.08456, −0.08452, 0.08452, 0.08447, −0.08442, −0.0844, −0.08436, −0.08436, −0.08436, 0.08436, −0.08435, −0.08434, −0.0843, 0.08428, 0.08426, 0.08426, 0.08426, −0.08424, 0.08414, 0.08411, −0.08408, −0.08407, 0.08406, −0.08406, 0.08405, 0.08404, −0.08403, 0.08403, 0.08403, 0.08403, 0.08399, 0.08397, −0.08396, −0.0839, −0.0839, −0.0839, −0.08385, −0.08385, −0.08383, −0.08383, 0.08382, −0.08379, 0.08375, 0.08372, −0.08371, 0.0837, −0.08367, 0.08365, −0.08363, 0.08362, −0.08359, 0.08356, −0.08355, −0.08353, −0.08346, 0.08346, 0.08344, 0.08343, −0.08343, −0.08342, −0.08341, 0.08341, −0.0834, −0.0834, 0.08336, 0.08334, −0.08332, 0.08332, 0.08326, −0.08326, −0.08325, 0.08325, −0.08324, −0.08323, 0.08323, −0.08321, −0.0832, 0.08317, −0.08316, −0.08315, −0.08313, −0.08307, 0.08305, −0.08302, −0.08302, −0.083, −0.08299, 0.08298, 0.08297, 0.08297, 0.08296, 0.08295, −0.08292, 0.08291, −0.08289, 0.08289, 0.08284, 0.08284, −0.08284, 0.0828, 0.0828, 0.08279, 0.08279, 0.08278, −0.08273, 0.08272, 0.08272, −0.08272, 0.08268, −0.08266, −0.08265, 0.08264, −0.08262, −0.08258, −0.08251, −0.08249, −0.08246, 0.08243, −0.0824, −0.08239, 0.08239, 0.08237, 0.08233, −0.0823, −0.08229, −0.08228, −0.08228, 0.08227, −0.08225, −0.08224, 0.08219, 0.08218, −0.08216, 0.08214, 0.08212, 0.08212, −0.08209, −0.08209, 0.08207, 0.08206, 0.08205, 0.08202, −0.082, −0.08196, −0.08193, −0.08192, −0.0819, 0.08185, 0.08183, −0.08183, 0.08182, −0.0818, −0.08179, −0.08179, 0.08179, 0.08178, 0.08175, −0.08172, −0.08172, −0.08171, −0.08171, 0.08169, −0.08168, −0.08168, −0.08167, 0.08166, 0.08163, −0.08158, 0.08156, −0.08154, −0.08144, 0.08141, 0.08141, 0.08139, 0.08138, 0.08136, 0.08136, 0.08127, 0.08127, −0.08126, 0.08125, 0.08125, −0.08121, 0.0812, 0.08119, 0.08117, −0.08117, 0.08114, −0.08112, 0.08111, −0.08109, −0.08109, 0.08107, −0.08106, −0.08105, −0.08102, −0.08101, 0.08098, −0.08097, 0.08096, −0.08094, 0.08094, 0.08093, −0.08093, 0.08092, −0.0809, −0.08088, 0.08087, 0.08086, 0.08085, 0.08084, −0.08084, −0.08082, 0.08081, 0.08081, 0.08079, 0.08078, −0.08078, −0.08074, 0.08068, −0.08067, −0.08066, −0.08065, −0.08064, −0.08063, −0.08062, −0.08061, 0.08061, 0.08059, 0.08057, 0.08055, −0.08055, −0.08054, −0.08052, 0.08052, 0.08051, 0.08051, 0.08048, −0.08048, −0.08047, 0.08044, 0.08036, 0.08035, −0.08031, −0.08027, −0.08023, 0.08022, −0.08022, −0.08021, −0.08021, −0.0802, 0.08019, 0.08017, 0.08014, 0.0801, −0.0801, 0.08009, −0.08009, 0.08009, −0.08008, 0.08006, −0.08001, −0.08001, −0.08001, 0.08001, 0.07994, 0.07991, −0.0798, −0.07978, 0.07976, −0.07971, −0.07971, 0.0797, 0.07962, 0.07962, −0.0796, −0.07957, −0.07954, −0.07954, 0.07952, 0.07952, 0.07944, −0.07943, −0.07941, 0.07938, −0.07935, −0.07935, −0.07932, 0.07931, 0.0793, 0.07929, 0.07928, 0.07928, 0.07928, −0.07927, −0.07927, −0.07925, −0.07925, −0.0792, 0.07917, 0.07916, 0.07916, −0.07913, 0.07912, −0.07912, −0.07911, −0.07911, −0.07909, 0.07909, 0.07908, −0.07907, −0.07904, −0.07904, 0.07904, −0.07904, 0.079, −0.07898, 0.07894, −0.0789, 0.07885, 0.07885, −0.07885, −0.07883, 0.07883, 0.0788, −0.0788, −0.0788, −0.07878, −0.07873, 0.07871, 0.07871, 0.0787, 0.07868, −0.07866, −0.07866, 0.07862, 0.07861, 0.07858, −0.07858, −0.07855, 0.07855, −0.07854, −0.07853, −0.07845, −0.07845, 0.07844, 0.07844, −0.07842, −0.0784, −0.07837, −0.07837, −0.07835, −0.07831, 0.0783, −0.07827, −0.07827, −0.07827, 0.07825, −0.07821, −0.0782, 0.07818, −0.07816, 0.07815, 0.07812, −0.07811, −0.07811, 0.07811, −0.0781, −0.07809, 0.07808, −0.07807, −0.07806, 0.07804, −0.07798, −0.07798, 0.07797, −0.07795, −0.07789, −0.07788, −0.07788, 0.07788, −0.07785, −0.07781, −0.0778, −0.07777, 0.07775, 0.07774, 0.07774, 0.07773, 0.07773, −0.07772, 0.07768, −0.07768, 0.07767, 0.07765, 0.07761, −0.0776, 0.0776, −0.07759, 0.07758, 0.07754, −0.07754, 0.07752, −0.07752, −0.07751, −0.0775, 0.0775, 0.07747, −0.07745, −0.0774, −0.07739, −0.07738, −0.07735, 0.07731, −0.07731, 0.07731, −0.07729, −0.07729, 0.07727, 0.07725, −0.07722, 0.07722, −0.07721, −0.07719, 0.07717, 0.07716, −0.07716, −0.07706, 0.07706, 0.07703, −0.07701, −0.07699, −0.07698, −0.07695, 0.07694, 0.07692, 0.07691, −0.0769, −0.07689, −0.07686, 0.07685, 0.07683, −0.07681, −0.07677, −0.07676, 0.07675, 0.07673, 0.07672, 0.0767, −0.07667, −0.07665, 0.07663, −0.07658, 0.07657, 0.07657, −0.07655, −0.07654, −0.07652, 0.07645, 0.07643, −0.07643, 0.07643, −0.07639, −0.07638, 0.07638, −0.07636, −0.07635, 0.07629, 0.07629, −0.07623, −0.07623, 0.07623, 0.07621, 0.07621, −0.0762, 0.07618, 0.07616, −0.07615, 0.0761, 0.07608, −0.07605, −0.07605, 0.07604, 0.07604, −0.07603, −0.07597, 0.07596, 0.07595, −0.07593, −0.07591, 0.07585, −0.07582, −0.07582, 0.07581, −0.07581, −0.0758, −0.07578, 0.07578, 0.07576, 0.07574, 0.07573, −0.0757, 0.07566, 0.07566, 0.07558, 0.07555, −0.07554, 0.07547, −0.07546, −0.07544, −0.07544, −0.07541, 0.07541, 0.07537, −0.07534, 0.07532, −0.07532, −0.07531, 0.0753, 0.0753, −0.07529, −0.07527, 0.07527, 0.07517, 0.07517, 0.07516, 0.07513, −0.07513, −0.07509, 0.07509, 0.07507, 0.07503, −0.07501, −0.075, −0.07499, −0.07498, 0.07498, −0.07496, −0.07493, −0.07493, −0.07492, −0.07491, 0.07489, 0.07488, 0.07488, −0.07487, 0.07484, 0.07482, −0.07478, 0.07477, −0.07477, 0.07474, −0.07473, 0.07467, 0.07465, −0.07461, 0.07458, 0.07455, −0.07455, 0.07453, 0.07451, 0.0745, −0.07449, −0.07447, −0.07447, 0.07439, 0.07435, −0.0743, 0.07428, 0.07426, 0.07422, −0.07421, −0.0742, −0.0742, −0.0742, −0.07419, −0.07418, −0.07417, 0.07417, 0.07415, 0.07415, 0.07414, 0.07413, −0.07411, 0.0741, −0.07409, 0.07407, 0.07406, 0.074, 0.07399, −0.07399, −0.07399, 0.07398, 0.07396, 0.07395, −0.07394, 0.07394, 0.07391, −0.0739, −0.0739, 0.07388, −0.07388, −0.07384, −0.07382, −0.07378, 0.07374, −0.07374, −0.07373, −0.07372, −0.07371, 0.0737, 0.07369, −0.07369, 0.07366, −0.0736, −0.0736, −0.07358, 0.07355, 0.07353, −0.07352, −0.07352, 0.07347, 0.07347, −0.07346, 0.07344, −0.0734, −0.07338, −0.07338, −0.07337, 0.07336, 0.07333, −0.07332, −0.0733, −0.0733, −0.07329, −0.07328, 0.07327, 0.07327, −0.07324, −0.07322, −0.0732, −0.07318, −0.07318, −0.07317, −0.07316, −0.07315, −0.07315, −0.07315, 0.07315, 0.07313, 0.07309, 0.07307, 0.07306, 0.07306, 0.07305, −0.07304, −0.07303, −0.07301, −0.07301, 0.073, 0.07297, −0.07297, −0.07296, −0.07296, −0.07293, −0.07292, 0.0729, −0.07286, −0.07286, −0.07281, 0.07279, −0.07278, 0.07277, −0.07276, 0.07275, −0.07271, −0.0727, −0.0727, 0.0727, −0.07269, −0.07269, 0.07267, −0.07266, −0.07263, 0.0726, −0.0726, 0.07258, −0.07256, 0.07255, 0.07255, −0.07255, −0.07252, −0.07252, −0.07251, −0.0725, −0.07249, 0.07247, 0.07247, 0.07246, −0.07245, 0.07239, −0.07235, −0.07232, 0.07231, 0.0723, −0.07229, 0.07226, 0.07226, 0.07225, −0.07222, 0.07221, −0.0722, 0.07218, 0.07218, 0.07217, 0.07216, −0.07216, 0.07214, −0.07213, 0.07212, −0.07212, −0.07211, −0.0721, 0.07208, −0.07204, −0.07202, −0.07202, 0.07196, −0.07195, 0.07193, −0.07191, −0.07191, 0.07186, −0.07183, 0.07181, 0.07179, 0.07177, −0.07172, −0.07168, 0.07166, −0.07162, −0.07161, −0.07157, −0.07157, 0.07156, −0.07156, −0.07155, −0.07154, −0.07152, 0.07152, −0.07152, 0.07151, 0.07145, −0.07142, 0.0714, −0.0714, 0.07135, 0.07133, 0.07133, 0.07132, −0.07128, −0.07127, −0.07121, 0.07121, −0.07118, 0.07114, −0.07111, −0.07111, −0.07109, −0.07106, 0.07103, 0.07098, −0.07098, 0.07098, −0.07098, −0.07094, 0.07092, 0.07087, 0.07086, −0.07084, 0.07079, −0.07077, −0.07075, −0.07074, 0.07072, −0.07071, 0.07071, −0.07069, 0.07069, −0.07066, 0.07063, −0.07063, 0.07062, 0.07062, −0.07058, 0.07057, −0.07056, 0.07056, 0.07054, −0.07053, −0.07051, 0.0705, −0.07048, −0.07048, −0.07048, 0.07044, 0.07043, −0.07043, −0.07041, −0.07041, −0.0704, 0.0704, −0.07039, −0.07039, 0.07037, 0.07037, −0.07036, 0.07034, 0.07032, −0.07032, −0.07031, −0.07031, 0.0703, 0.07028, −0.07025, −0.07024, 0.07023, 0.07023, 0.07022, −0.07019, 0.07019, −0.07019, −0.07016, −0.07015, 0.07014, −0.07013, 0.07013, 0.07012, −0.07009, −0.07008, −0.07008, 0.07008, 0.07007, −0.07006, 0.07006, −0.07006, −0.07005, 0.07001, 0.07001, −0.07, −0.06998, 0.06998, 0.06996, 0.06993, −0.06993, 0.06992, −0.06984, −0.06984, −0.06983, 0.0698, 0.06977, −0.06974, −0.06972, 0.06971, 0.06971, −0.0697, 0.06968, 0.06967, −0.06966, −0.06965, 0.06964, −0.06961, −0.06961, 0.0696, 0.06959, −0.06957, −0.06954, −0.06954, 0.06952, −0.0695, −0.0695, 0.06949, 0.06949, 0.06948, 0.06947, −0.06945, −0.06937, −0.06935, 0.06934, 0.06932, −0.06931, 0.06929, 0.06929, −0.06928, 0.06927, −0.06925, −0.06924, 0.06924, 0.06924, 0.06919, 0.06918, 0.06916, −0.06916, −0.06916, −0.06916, −0.06913, 0.06912, −0.06911, 0.06911, 0.0691, −0.06909, −0.06906, −0.06905, −0.06901, 0.06899, 0.06898, −0.06897, 0.06896, 0.06895, 0.06895, 0.06893, −0.06892, 0.06892, 0.06891, 0.06889, 0.06887, 0.06882, 0.06882, −0.0688, −0.06877, 0.06875, 0.06875, 0.06873, 0.06873, −0.06872, 0.06872, 0.06871, 0.0687, 0.06869, −0.06863, 0.06862, 0.0686, 0.06859, 0.06858, −0.06856, 0.06856, −0.06855, −0.06853, 0.06853, −0.06852, 0.06852, 0.06848, −0.06846, 0.06845, −0.06844, 0.06842, −0.06842, 0.06842, 0.06842, −0.0684, 0.06839, −0.06835, −0.06831, 0.06831, −0.06829, 0.06827, 0.06827, 0.06826, 0.06825, −0.06823, 0.06823, −0.06821, −0.06821, −0.0682, −0.06817, −0.06816, −0.06813, 0.06812, 0.06811, −0.0681, 0.0681, 0.06808, −0.06805, 0.06803, 0.06803, 0.068, 0.06799, −0.06799, 0.06798, −0.06798, 0.06795, −0.06794, 0.06793, −0.06791, 0.06791, −0.06788, −0.06786, −0.06786, 0.06786, −0.06785, −0.06781, 0.06777, 0.06776, −0.06773, 0.06771, 0.0677, −0.0677, −0.0677, −0.06764, 0.06763, 0.06762, 0.06761, −0.06761, −0.06758, 0.06758, 0.06755, −0.06755, 0.06755, −0.06754, −0.06754, −0.06753, −0.06752, −0.06752, −0.06748, −0.06748, −0.06745, 0.06744, 0.06742, 0.06742, 0.06738, −0.06738, −0.06737, −0.06737, −0.06736, 0.06735, 0.06734, −0.06734, −0.06732, 0.06729, 0.06729, 0.06728, 0.06727, 0.06726, −0.06725, 0.06724, −0.06722, −0.06721, 0.06718, 0.06718, 0.06718, 0.06712, 0.06712, 0.06712, 0.06711, −0.06711, 0.0671, −0.0671, −0.06708, −0.06707, −0.06705, 0.06703, −0.06699, 0.06699, 0.06699, −0.06698, 0.06698, −0.06697, 0.06694, 0.06693, 0.0669, −0.06687, 0.06685, −0.06684, −0.06678, 0.06675, −0.06674, −0.06672, 0.06671, 0.06669, −0.06669, 0.06668, −0.06667, −0.06666, −0.06665, −0.06663, −0.0666, −0.0666, −0.0666, −0.06655, −0.0665, 0.06649, 0.06648, −0.06645, −0.06644, −0.06643, 0.06642, −0.06642, −0.06641, 0.06637, 0.06636, −0.06636, −0.06635, 0.06634, −0.06634, −0.06634, −0.06634, 0.06634, 0.06634, −0.06631, −0.06631, −0.06624, 0.06622, 0.06621, 0.06621, 0.0662, −0.06619, −0.06619, −0.06618, −0.06617, −0.06613, 0.06605, −0.06605, −0.06601, 0.06601, −0.06598, −0.06595, 0.06593, 0.06593, 0.06591, 0.06589, −0.06586, −0.06584, −0.06581, −0.0658, 0.06579, −0.06579, 0.06576, −0.06576, −0.06573, 0.06573, 0.06568, −0.06567, −0.06564, −0.06562, 0.0656, 0.06559, 0.06558, 0.06558, 0.06557, 0.06554, −0.06553, −0.06553, −0.0655, 0.0655, 0.06549, −0.06547, −0.06545, −0.06538, 0.06536, −0.06536, 0.06535, 0.06535, 0.06535, 0.06534, −0.06533, −0.06532, 0.0653, −0.06528, 0.06527, 0.06525, 0.06521, −0.0652, −0.06518, −0.06516, 0.06513, −0.06512, 0.06511, 0.0651, −0.06509, 0.06509, 0.06507, 0.06506, 0.06503, −0.06502, −0.06501, 0.065, −0.065, 0.06499, 0.06497, −0.06495, −0.06495, −0.06494, −0.06493, 0.06492, −0.06492, −0.0649, −0.0649, 0.06489, 0.06483, −0.06483, 0.06482, 0.06481, −0.0648, −0.06479, −0.06479, −0.06478, 0.06478, 0.06478, 0.06478, 0.06477, 0.06475, −0.06474, 0.06474, −0.0647, −0.0647, 0.06465, −0.06465, 0.06464, 0.06463, −0.06463, 0.06462, 0.0646, −0.06459, 0.06458, −0.06457, 0.06454, 0.06452, 0.0645, 0.06449, 0.06449, −0.06448, −0.06448, −0.06447, −0.06445, −0.06445, 0.06444, −0.06444, −0.06443, 0.06443, −0.06442, 0.06441, −0.06441, 0.06438, 0.06437, 0.06437, −0.06434, −0.06434, −0.06432, −0.06432, −0.06432, −0.0643, 0.0643, −0.0643, −0.06428, −0.06427, −0.06425, −0.06423, −0.06421, 0.06418, −0.06415, −0.06414, −0.06414, −0.06413, 0.06411, −0.06407, −0.06407, 0.06406, 0.06401, 0.06398, 0.06397, 0.06394, −0.06394, −0.06393, 0.06391, 0.06391, 0.06387, 0.06387, −0.06385, −0.06384, 0.06384, −0.0638, −0.06379, −0.06379, 0.06373, 0.06371, −0.0637, 0.06369, −0.06369, 0.06369, −0.06367, 0.06366, 0.06366, −0.06365, −0.06364, 0.06363, −0.06362, −0.06361, 0.0636, −0.0636, −0.06358, 0.06355, −0.06354, 0.06353, 0.06353, 0.06352, 0.06352, −0.06352, 0.06351, −0.0635, 0.06349, −0.06348, 0.06348, 0.06346, −0.06341, −0.06339, −0.06339, 0.06339, −0.06336, −0.06334, −0.06333, −0.06332, −0.06328, 0.06327, 0.06324, 0.06323, 0.06323, −0.06323, 0.06322, 0.06321, −0.0632, −0.0632, 0.06318, 0.06318, −0.06318, −0.06317, 0.06317, −0.06316, 0.06314, −0.06313, 0.06312, −0.06312, 0.06312, −0.06312, −0.06311, −0.0631, 0.06309, −0.06309, 0.06308, 0.06308, 0.06306, −0.06305, −0.06305, −0.06301, 0.063, −0.06295, 0.06294, −0.06293, −0.06289, −0.06289, −0.06289, −0.06288, 0.06285, 0.0628, 0.06278, −0.06276, 0.06274, −0.06274, −0.06273, −0.06273, 0.06272, 0.06271, 0.06271, 0.06269, 0.06268, −0.06267, −0.06266, −0.06265, 0.06263, −0.06262, −0.06262, −0.06261, 0.06261, −0.06259, 0.06259, −0.06259, −0.06258, 0.06258, −0.06257, −0.06255, 0.06255, −0.06255, −0.06254, 0.06253, 0.06253, −0.06251, 0.06249, −0.06249, 0.06245, −0.06245, −0.06245, −0.06245, 0.06244, −0.06242, −0.06242, 0.06241, 0.0624, −0.06239, −0.06238, −0.06236, −0.06235, −0.06231, 0.06231, 0.06229, 0.06228, −0.06227, 0.06227, −0.06226, 0.06226, 0.06224, −0.06224, 0.06224, −0.06223, −0.06222, −0.06221, −0.06221, −0.06221, −0.06218, 0.06216, −0.06215, −0.06213, 0.06212, −0.0621, 0.06209, −0.06207, −0.06205, −0.06203, −0.062, −0.062, 0.06196, 0.06195, 0.06194, 0.06192, 0.0619, 0.0619, 0.06187, 0.06186, −0.0618, −0.06179, 0.06178, 0.06178, −0.06176, −0.06176, 0.06174, 0.06169, 0.06167, −0.06167, −0.06166, 0.06165, 0.06161, −0.06161, 0.0616, 0.06156, −0.06155, 0.06152, −0.06152, 0.06151, 0.06151, −0.0615, −0.06148, 0.06146, 0.06146, 0.06145, −0.06145, −0.06145, −0.06143, −0.0614, −0.06138, −0.06138, 0.06137, 0.06135, −0.06135, −0.06134, −0.06132, −0.06132, −0.06132, 0.06129, 0.06129, 0.06129, 0.06128, 0.06128, 0.06128, 0.06125, 0.06124, −0.06124, −0.06121, 0.0612, −0.0612, 0.06119, −0.06119, −0.06118, 0.06114, −0.06113, 0.06112, 0.0611, −0.0611, −0.06109, 0.06108, −0.06107, 0.06106, −0.06106, 0.06105, 0.06101, 0.06099, 0.06099, 0.06098, 0.06098, 0.06096, 0.06096, −0.06095, 0.06093, −0.06093, 0.06093, 0.06093, 0.06093, −0.06093, 0.0609, 0.0609, −0.06087, −0.06085, −0.06085, −0.06084, 0.06084, −0.06082, −0.06081, 0.0608, 0.06078, 0.06077, 0.06075, 0.06075, −0.06075, 0.06074, 0.06073, 0.06071, −0.06069, 0.06069, 0.06068, −0.06067, −0.06066, 0.06063, 0.06063, 0.06059, 0.06059, −0.06058, −0.06056, 0.06052, 0.06051, 0.0605, 0.06048, −0.06045, 0.06042, −0.06039, 0.06038, 0.06036, −0.06033, 0.06032, −0.06031, −0.0603, 0.0603, −0.06028, 0.06027, 0.06026, −0.06024, 0.06021, 0.06021, −0.06019, −0.06019, −0.06017, −0.06016, −0.06016, −0.06016, −0.06015, 0.06014, 0.06013, 0.06013, 0.06012, −0.06012, 0.0601, −0.06008, −0.06007, 0.06006, 0.06006, 0.06005, 0.06003, −0.06001, 0.06001, 0.06, −0.06, −0.06, −0.05999, −0.05998, −0.05998, −0.05996, −0.05996, 0.05995, −0.05995, −0.05993, 0.05989, −0.05988, −0.05988, 0.05986, 0.05986, −0.05985, −0.05984, 0.05984, 0.05983, −0.05983, −0.05983, 0.05982, −0.05981, −0.0598, 0.05978, 0.05977, 0.05977, −0.05976, −0.05973, 0.05971, 0.05967, −0.05967, −0.05966, 0.05966, −0.05965, −0.05964, −0.05964, −0.05963, 0.05963, −0.05963, 0.05962, −0.05961, 0.0596, 0.0596, 0.0596, 0.05958, 0.05957, 0.05956, −0.05956, −0.05953, 0.05953, −0.05952, −0.05951, −0.0595, 0.05947, 0.05946, 0.05945, −0.05944, −0.05942, −0.0594, −0.0594, −0.05939, −0.05937, −0.05936, −0.05935, −0.05934, −0.05934, 0.05933, −0.05932, −0.05932, −0.05931, −0.05931, −0.0593, 0.0593, 0.05929, 0.05929, 0.05928, −0.05924, 0.05923, −0.05923, 0.05921, −0.05919, −0.05918, 0.05917, 0.05912, 0.05909, 0.05909, 0.05908, −0.05903, −0.059, −0.05898, 0.05898, −0.05897, −0.05897, 0.05897, −0.05897, 0.05896, −0.05892, 0.05891, 0.05891, 0.0589, −0.05889, −0.05886, −0.05883, 0.05881, −0.05881, 0.05881, −0.05881, −0.0588, 0.0588, 0.05879, 0.05879, −0.05879, −0.05878, −0.05878, 0.05877, −0.05875, 0.05871, −0.05869, −0.05868, 0.05868, −0.05867, −0.05866, −0.05865, −0.05862, 0.05862, 0.05859, 0.05858, 0.05856, −0.05855, −0.05855, 0.05855, −0.05854, 0.05853, 0.05851, −0.05849, −0.05846, −0.05845, −0.05843, 0.05843, −0.05841, 0.05841, −0.05841, 0.05837, −0.05834, 0.05832, 0.05831, 0.0583, −0.05829, −0.05828, −0.05826, −0.05821, 0.05821, −0.05819, 0.05819, 0.05819, −0.05819, −0.05819, −0.05816, −0.05816, 0.05814, −0.05813, 0.05812, −0.05811, −0.05809, 0.05809, −0.05808, −0.05808, −0.05806, −0.05805, −0.05805, 0.05804, 0.05804, −0.05803, 0.05798, −0.05797, −0.05797, 0.05795, 0.05791, 0.0579, 0.05789, −0.05787, 0.05785, −0.05785, 0.05784, 0.05782, −0.05782, −0.05781, −0.05781, −0.05778, −0.05775, −0.05775, 0.05773, 0.05773, −0.05772, −0.05771, 0.05768, 0.05768, 0.05764, 0.05763, −0.05762, 0.05761, 0.05758, −0.05758, −0.05758, 0.05757, −0.05753, −0.05753, 0.05752, 0.0575, −0.05748, 0.05744, −0.05744, −0.05743, −0.05742, 0.05737, −0.05734, −0.05734, 0.0573, −0.05729, −0.05727, 0.05726, −0.05724, 0.05723, −0.05723, −0.05721, −0.05719, −0.05719, 0.05718, −0.05717, 0.05716, 0.05716, −0.05715, 0.05715, −0.05713, −0.05712, −0.05712, 0.05712, 0.05711, −0.0571, 0.0571, −0.05709, 0.05709, −0.05708, 0.05704, 0.05701, −0.057, 0.05698, −0.05698, 0.05697, 0.05696, 0.05695, −0.05695, −0.05694, −0.05694, 0.05694, −0.05693, 0.05692, 0.05691, −0.05689, 0.05688, 0.05686, 0.05684, 0.05683, −0.05681, 0.05679, −0.05677, 0.05676, 0.05674, 0.05673, −0.05671, −0.0567, −0.0567, 0.05668, 0.05668, 0.05664, −0.05663, 0.05663, −0.05656, −0.05654, 0.05653, 0.05653, −0.05652, −0.05649, 0.05647, 0.05646, −0.05646, 0.05646, 0.05638, −0.05635, −0.05634, −0.05631, 0.0563, 0.05629, 0.05629, 0.05627, 0.05623, −0.05622, −0.05622, 0.05621, −0.05619, 0.05617, −0.05617, −0.05616, −0.05615, 0.05615, −0.05614, −0.05613, −0.05611, 0.05609, 0.05609, −0.05608, −0.05606, −0.05604, −0.05604, −0.05603, 0.05602, −0.05598, −0.05596, −0.05594, −0.05593, −0.05592, 0.05591, 0.0559, 0.05589, 0.05588, −0.05588, 0.05588, −0.05587, 0.05586, −0.05586, −0.05584, −0.05584, 0.05582, −0.05581, −0.05581, 0.05577, 0.05577, −0.05576, 0.05576, 0.05573, −0.0557, 0.05567, 0.05564, 0.05564, 0.05564, 0.05558, 0.05557, 0.05557, 0.05555, −0.05554, −0.05554, 0.05554, −0.05548, 0.05547, −0.05546, −0.05546, 0.05544, 0.05543, −0.05542, −0.0554, −0.05537, −0.05535, 0.05535, 0.05534, 0.05532, 0.05531, −0.0553, −0.0553, 0.0553, −0.05528, −0.05528, −0.05526, −0.05525, −0.05524, −0.05523, 0.05521, −0.05521, −0.0552, 0.05519, −0.05519, 0.05519, 0.05518, 0.05517, −0.05515, 0.05515, −0.05511, −0.0551, 0.05509, 0.05509, −0.05507, 0.05506, −0.05506, 0.05502, 0.05502, −0.055, 0.05495, −0.05495, 0.05494, 0.05493, −0.05493, −0.0549, −0.05489, −0.05488, −0.05487, −0.05486, 0.05485, −0.05484, −0.05484, 0.05484, −0.05483, −0.05483, 0.05482, −0.05482, 0.05481, −0.05481, 0.05481, 0.0548, 0.05479, −0.05478, 0.05473, 0.05473, 0.05471, 0.05469, −0.05469, −0.05469, 0.05468, 0.05467, 0.05466, 0.05466, −0.05465, −0.05463, −0.05462, −0.05462, 0.05461, −0.05461, 0.05461, 0.0546, 0.05458, −0.05457, 0.05456, −0.05455, −0.05454, −0.05453, 0.05453, −0.05452, −0.05452, −0.05451, 0.0545, 0.0545, 0.05449, −0.05449, −0.05448, −0.05445, −0.05445, −0.05444, 0.05444, −0.05444, −0.05442, 0.05442, −0.05442, 0.05442, 0.05441, 0.05439, 0.05438, −0.05436, 0.05436, −0.05434, 0.05433, 0.05433, −0.0543, −0.05429, 0.05427, 0.05427, 0.05425, −0.05425, 0.05424, −0.05421, −0.0542, −0.05419, −0.05419, 0.05416, 0.05414, −0.05413, 0.05413, 0.05413, −0.05412, 0.05411, 0.05411, −0.0541, −0.0541, −0.05408, 0.05405, −0.05404, −0.05403, −0.05402, −0.05401, 0.054, 0.05398, 0.05396, −0.05394, −0.05389, 0.05385, 0.05383, 0.05383, 0.05383, 0.05382, 0.05381, 0.0538, 0.05379, −0.05378, 0.05377, −0.05377, 0.05375, 0.05374, 0.05374, −0.05373, −0.05373, 0.05372, −0.05372, −0.05371, 0.05368, 0.05367, −0.05366, 0.05365, 0.05364, −0.05361, −0.0536, −0.0536, 0.05357, −0.05355, −0.05353, −0.05352, 0.05352, −0.05351, −0.0535, 0.05349, −0.05349, −0.05346, −0.05345, 0.05345, 0.05345, −0.05344, −0.05343, −0.05343, −0.05343, 0.05342, 0.05341, 0.0534, −0.0534, 0.05337, 0.05337, −0.05335, −0.05335, 0.05334, 0.05334, −0.05333, −0.05329, −0.05329, 0.05326, 0.05326, 0.05326, 0.05324, −0.05323, −0.05322, −0.05321, 0.05321, −0.0532, −0.0532, −0.05318, −0.05318, 0.05315, 0.05312, 0.05312, −0.05311, −0.0531, −0.05308, −0.05307, −0.05307, 0.05306, −0.05305, 0.05303, 0.05302, −0.053, −0.05298, 0.05297, −0.05294, 0.05294, −0.05291, −0.05289, 0.05288, −0.05287, −0.05286, −0.05286, 0.05286, 0.05284, −0.05284, −0.05283, 0.05282, −0.05281, 0.0528, −0.0528, −0.05279, −0.05279, 0.05278, 0.05278, −0.05276, 0.05276, 0.05273, −0.0527, 0.05269, 0.05268, 0.05267, −0.05267, 0.05265, 0.05265, 0.05265, −0.05264, 0.05264, −0.05264, −0.05264, 0.05262, −0.05262, −0.05261, 0.0526, 0.05258, −0.05255, −0.05252, −0.05251, 0.05249, −0.05247, 0.05247, 0.05245, 0.05243, −0.05241, 0.05239, −0.05239, 0.05239, 0.05238, −0.05238, 0.05237, −0.05236, −0.05235, −0.05234, −0.05234, −0.05233, 0.05232, −0.05232, 0.0523, 0.05228, −0.05226, −0.05226, −0.05225, 0.05224, 0.05223, 0.05223, −0.05222, 0.05221, 0.0522, 0.05217, −0.05217, 0.05216, 0.05214, −0.05213, 0.0521, 0.05209, 0.05207, 0.05206, 0.05204, −0.05204, −0.05201, −0.052, −0.05198, 0.05197, 0.05195, −0.05195, −0.05194, −0.05193, 0.05193, −0.05192, −0.05191, −0.0519, −0.0519, −0.05188, −0.05187, −0.05186, −0.05183, 0.05181, −0.05181, −0.05181, 0.05181, −0.05179, 0.05178, −0.05177, −0.05177, −0.05176, 0.05176, 0.05175, 0.05175, −0.05172, 0.05172, 0.05171, 0.05171, −0.0517, 0.05169, −0.05169, −0.05168, −0.05168, 0.05168, −0.05166, 0.05165, −0.05165, −0.05164, −0.05164, −0.05163, 0.05162, 0.05161, 0.05161, −0.0516, 0.0516, 0.05159, −0.05158, −0.05158, 0.05157, −0.05157, 0.05154, 0.05153, 0.0515, 0.0515, −0.05149, −0.05149, 0.05148, 0.05141, 0.05141, 0.05141, −0.05141, −0.0514, 0.05137, −0.05137, 0.05137, −0.05136, −0.05136, −0.05134, 0.05133, −0.05133, −0.05132, −0.05132, −0.0513, −0.05127, −0.05127, 0.05126, −0.05124, 0.05123, −0.05123, 0.05123, −0.05122, 0.05121, 0.05117, −0.05116, 0.05116, 0.05115, 0.05115, −0.05113, 0.05113, −0.05112, −0.05112, −0.05111, 0.0511, −0.0511, 0.0511, 0.05107, 0.05107, −0.05107, 0.05106, 0.05105, 0.05105, −0.05104, −0.05103, −0.05102, 0.051, −0.05099, −0.05098, 0.05098, −0.05096, 0.05095, 0.05093, 0.05091, 0.05085, 0.05085, 0.05085, −0.05083, −0.05083, 0.05083, 0.05083, 0.05082, 0.05082, −0.05082, −0.0508, −0.05077, 0.05074, −0.05073, 0.05073, −0.05071, −0.05069, −0.05068, 0.05067, 0.05066, −0.05066, −0.05062, −0.05061, −0.0506, −0.0506, −0.0506, −0.05059, −0.05058, 0.05057, 0.05054, 0.05052, 0.05049, −0.05048, 0.05047, −0.05044, −0.05044, 0.05043, −0.05041, 0.05041, −0.05041, 0.05039, −0.05038, −0.05037, 0.05037, −0.05036, 0.05036, −0.05035, 0.05035, −0.05035, −0.05035, −0.05034, −0.05033, −0.05031, −0.05031, −0.05031, −0.0503, 0.0503, −0.0503, −0.05029, −0.05029, −0.05026, 0.05024, −0.05023, −0.05021, 0.0502, 0.0502, −0.05019, −0.05018, 0.05018, 0.05017, −0.05016, −0.05015, −0.05013, 0.05012, −0.05011, −0.0501, −0.05009, −0.05008, 0.05007, 0.05006, 0.05006, −0.05005, −0.05005, −0.05004, −0.05003, 0.05002, 0.05001, −0.05, −0.04997, −0.04997, 0.04997, 0.04996, 0.04996, −0.04995, 0.04995, 0.04992, −0.04991, −0.04991, 0.04991, 0.04989, −0.04989, 0.04988, −0.04987, 0.04987, 0.04984, 0.04984, 0.04982, −0.04982, −0.04981, 0.04981, −0.04976, −0.04976, 0.04973, −0.04973, 0.04972, 0.04972, −0.04971, 0.04968, −0.04967, −0.04966, 0.04964, −0.04961, 0.0496, −0.04958, 0.04958, −0.04957, 0.04955, −0.04955, −0.04955, 0.04953, 0.04953, 0.04952, 0.04951, −0.0495, 0.04948, −0.04947, 0.04946, −0.04945, −0.04945, −0.04943, 0.04941, −0.04941, 0.04941, −0.04939, 0.04938, −0.04936, 0.04934, −0.04934, −0.04933, −0.04932, 0.04931, −0.0493, −0.0493, 0.04929, 0.04928, −0.04928, −0.04928, −0.04926, −0.04925, −0.04925, −0.04925, 0.04922, 0.04921, 0.04918, −0.04916, −0.04916, −0.04912, −0.04907, 0.04906, −0.04906, −0.04906, 0.04904, 0.04903, −0.04902, −0.04902, −0.049, 0.049, 0.04897, −0.04897, −0.04897, −0.04897, −0.04895, 0.04894, −0.04894, −0.04892, 0.0489, 0.04888, 0.04885, 0.04883, −0.04883, 0.04883, −0.04883, 0.0488, 0.04877, −0.04875, 0.04875, 0.04874, −0.04871, 0.0487, 0.04868, 0.04866, 0.04861, −0.04861, −0.0486, 0.04859, −0.04856, −0.04856, 0.04854, 0.04854, 0.04853, −0.04852, 0.04852, −0.04851, 0.04851, −0.0485, −0.04849, −0.04848, −0.04847, 0.04846, 0.04845, −0.04845, 0.04843, −0.04843, −0.04842, 0.04842, −0.04842, −0.04841, 0.04841, 0.04841, 0.0484, 0.0484, −0.04838, −0.04836, 0.04832, −0.04831, 0.0483, 0.04829, −0.04829, 0.04828, 0.04827, −0.04827, 0.04826, 0.04826, 0.04826, −0.04825, 0.04825, 0.04824, −0.04824, −0.04824, 0.04821, −0.04821, 0.04818, 0.04817, −0.04817, −0.04816, 0.04813, 0.04813, 0.04812, −0.04811, 0.04811, −0.04811, −0.0481, 0.04808, −0.04806, −0.04806, −0.04806, 0.04805, 0.04805, 0.04804, 0.04802, 0.048, −0.048, 0.04799, 0.04797, 0.04795, −0.04794, 0.04793, 0.04792, −0.04792, −0.04792, 0.04791, 0.04791, 0.04791, 0.04789, −0.04788, −0.04787, 0.04784, −0.04782, 0.04781, 0.04781, 0.04781, −0.04779, 0.04778, −0.04776, −0.04776, −0.04776, −0.04775, 0.04774, −0.04774, 0.04773, −0.04773, 0.04772, −0.0477, 0.0477, −0.0477, 0.04769, 0.04769, 0.04768, −0.04767, −0.04766, 0.04765, 0.04764, 0.04764, −0.04762, 0.04762, 0.04762, −0.04759, −0.04756, −0.04755, 0.04755, −0.04753, 0.04753, −0.04752, 0.04752, 0.0475, −0.0475, 0.04749, −0.04749, 0.04748, 0.04748, −0.04747, 0.04747, −0.04746, 0.04745, −0.04744, 0.04744, −0.04743, −0.04741, 0.04741, 0.0474, −0.04738, −0.04737, 0.04736, 0.04736, −0.04734, 0.04733, 0.04732, 0.04732, 0.04731, −0.0473, 0.0473, 0.0473, 0.04729, −0.04728, −0.04727, 0.04725, −0.04725, −0.04724, −0.04723, 0.04723, 0.04723, −0.04723, −0.04722, 0.04722, 0.04721, 0.04719, −0.04718, 0.04718, 0.04718, 0.04713, −0.04713, −0.04711, 0.04711, −0.04711, −0.04709, −0.04708, 0.04707, −0.04704, −0.04704, −0.04703, −0.04703, −0.04701, −0.04697, −0.04695, 0.04695, −0.04695, 0.04694, −0.04694, 0.04692, 0.04691, 0.04691, 0.04691, −0.04691, 0.0469, −0.04688, 0.04687, 0.04687, 0.04686, −0.04685, −0.04685, −0.04685, −0.04684, 0.04684, 0.04683, −0.04683, −0.04682, −0.04682, 0.0468, −0.0468, −0.0468, 0.04679, −0.04678, 0.04676, −0.04676, 0.04676, −0.04673, 0.04673, 0.04669, −0.04669, −0.04668, 0.04668, −0.04668, 0.04668, 0.04667, 0.04666, −0.04665, 0.04665, 0.04663, 0.04663, −0.04663, −0.04662, 0.04662, 0.04662, −0.04661, 0.0466, 0.04658, 0.04657, 0.04656, −0.04656, −0.04655, −0.04654, 0.04654, 0.04651, 0.04651, 0.04651, −0.04649, 0.04648, 0.04647, −0.04647, 0.04646, 0.04645, −0.04641, −0.04639, −0.04639, −0.04638, 0.04637, 0.04636, −0.04633, 0.04632, −0.04632, −0.0463, 0.04629, 0.04629, 0.04628, 0.04627, −0.04627, −0.04626, 0.04626, −0.04626, −0.04625, 0.04624, 0.04623, −0.04621, −0.04621, 0.04619, 0.04619, −0.04618, −0.04617, −0.04617, −0.04615, −0.04615, 0.04614, −0.04613, −0.04612, 0.04612, 0.04611, −0.04611, 0.0461, −0.04609, 0.04608, −0.04606, 0.04605, −0.04603, 0.04603, 0.04603, −0.04603, −0.04601, −0.04599, −0.04599, 0.04597, 0.04597, 0.04597, −0.04597, −0.04596, −0.04596, 0.04592, 0.04591, −0.04591, 0.0459, −0.04589, 0.04588, −0.04586, −0.04586, −0.04586, −0.04585, −0.04585, 0.04584, 0.04583, −0.04583, 0.04582, 0.04581, 0.0458, 0.04579, 0.04579, 0.04579, 0.04578, −0.04578, −0.04578, 0.04578, 0.04576, −0.04576, 0.04575, 0.04575, −0.04575, −0.04574, 0.04574, −0.04572, 0.04572, −0.04572, −0.04572, 0.04569, −0.04568, 0.04567, −0.04566, −0.04566, −0.04565, −0.04565, 0.04565, −0.04564, −0.04563, −0.04563, 0.04562, 0.04562, −0.04562, 0.04561, −0.04559, −0.04559, −0.04559, 0.04557, 0.04555, 0.04555, −0.04553, −0.04552, 0.04551, −0.0455, −0.04546, 0.04545, 0.04544, −0.04543, −0.0454, −0.04539, 0.04539, 0.04538, 0.04537, 0.04537, 0.04537, −0.04537, 0.04536, −0.04536, −0.04535, 0.04535, −0.04534, −0.04534, −0.04533, 0.04533, 0.04531, −0.0453, 0.04528, 0.04526, −0.04526, 0.04524, 0.04523, −0.04523, −0.04523, 0.04522, 0.04521, 0.04521, −0.0452, −0.04519, 0.04518, 0.04516, −0.04515, −0.04514, 0.04514, −0.04508, −0.04507, 0.04505, −0.04505, 0.04504, 0.04504, 0.04504, 0.04504, −0.04503, 0.04503, 0.04502, 0.04502, −0.045, −0.045, 0.045, −0.045, −0.04499, −0.04496, −0.04496, 0.04494, −0.04494, −0.04493, 0.04493, −0.04493, 0.04493, −0.0449, −0.0449, −0.04489, −0.04488, −0.04488, −0.04488, −0.04486, 0.04483, 0.04483, −0.04482, −0.04482, −0.04481, 0.04481, −0.0448, −0.0448, −0.04479, 0.04478, 0.04476, 0.04475, −0.04474, −0.04473, 0.04471, 0.0447, 0.04469, 0.04469, −0.04469, −0.04468, −0.04468, −0.04466, 0.04465, −0.04464, −0.04464, −0.04464, −0.04462, 0.04461, 0.04461, 0.04459, −0.04459, −0.04457, −0.04456, 0.04456, −0.04454, 0.04454, 0.04453, −0.04453, −0.04453, −0.04452, 0.04452, 0.04451, 0.04451, −0.0445, 0.0445, 0.04449, −0.04449, −0.04448, −0.04448, 0.04446, −0.04446, −0.04445, 0.04445, 0.04445, 0.04442, 0.04442, 0.0444, 0.04436, 0.04436, −0.04436, 0.04435, 0.04432, −0.04431, 0.04429, −0.04429, 0.04428, −0.04428, −0.04427, 0.04427, 0.04427, 0.04427, 0.04427, −0.04424, −0.04424, 0.04424, −0.04423, −0.04422, −0.04418, 0.04418, −0.04417, −0.04417, 0.04417, 0.04414, −0.04414, −0.04413, 0.04412, 0.04411, 0.04411, −0.0441, 0.04409, −0.04408, 0.04408, 0.04408, −0.04408, 0.04407, 0.04407, 0.04407, −0.04406, 0.04406, −0.04403, −0.04402, −0.04401, 0.04401, 0.044, 0.04398, 0.04397, 0.04397, −0.04396, −0.04396, −0.04395, 0.04393, 0.04393, 0.04393, −0.04389, −0.04389, −0.04389, −0.04388, −0.04388, 0.04387, 0.04387, 0.04386, −0.04385, −0.04384, 0.04384, −0.04384, 0.04383, 0.04382, −0.04382, 0.04381, −0.04378, 0.04376, 0.04375, −0.04374, −0.04374, 0.04373, −0.04373, 0.04373, 0.0437, −0.0437, −0.04369, 0.04367, −0.04367, 0.04366, −0.04366, 0.04366, 0.04364, 0.04363, −0.04363, 0.04363, −0.04362, 0.0436, −0.04359, −0.04353, −0.04352, −0.04352, −0.04351, −0.0435, −0.04349, −0.04348, 0.04348, 0.04345, −0.04344, −0.04342, −0.04342, 0.04341, 0.0434, 0.0434, −0.0434, −0.04338, 0.04337, 0.04337, −0.04337, 0.04336, −0.04336, −0.04336, 0.04335, 0.04334, −0.04333, −0.04332, 0.04332, −0.04331, −0.04331, 0.0433, −0.04329, 0.04326, −0.04326, 0.04326, 0.04325, −0.04325, −0.04325, −0.04324, −0.04324, 0.04324, −0.04324, 0.04323, −0.04323, 0.04323, −0.04321, −0.0432, −0.04319, −0.04319, −0.04317, 0.04316, −0.04316, −0.04316, −0.04315, −0.04314, 0.0431, 0.0431, 0.04309, −0.04306, −0.04305, −0.04304, 0.04304, −0.04302, −0.04301, −0.043, 0.043, 0.043, −0.04298, 0.04297, −0.04296, −0.04296, −0.04293, 0.04293, −0.04293, 0.04293, −0.04292, 0.04286, −0.04286, 0.04285, −0.04284, 0.04284, −0.04282, 0.04281, −0.04279, 0.04277, −0.04277, −0.04275, −0.04274, 0.04273, −0.04273, 0.04273, 0.04272, −0.04271, −0.04271, −0.04271, 0.0427, 0.04267, 0.04267, 0.04266, 0.04264, −0.04263, 0.04263, 0.04262, −0.04262, −0.0426, 0.04258, 0.04257, −0.04256, 0.04254, 0.04254, 0.04254, −0.04254, −0.04253, −0.04252, 0.04252, 0.04251, 0.0425, 0.0425, 0.04249, 0.04248, 0.04247, −0.04246, −0.04245, −0.04245, 0.04244, 0.04244, 0.04243, 0.04242, 0.04242, −0.04241, 0.04239, −0.04238, 0.04237, −0.04237, −0.04236, 0.04236, −0.04234, −0.04233, −0.04232, 0.04232, 0.04229, −0.04229, −0.04228, 0.04228, −0.04227, −0.04227, −0.04227, −0.04227, −0.04226, 0.04225, 0.04224, −0.04223, −0.04221, 0.0422, 0.04218, −0.04217, −0.04217, −0.04216, 0.04216, −0.04216, 0.04212, −0.04212, −0.04211, 0.0421, −0.04209, −0.04206, −0.04206, −0.04205, −0.04205, −0.04205, 0.04204, 0.04203, −0.04203, −0.04203, 0.04202, −0.04201, −0.04198, −0.04198, −0.04198, 0.04196, 0.04196, 0.04195, −0.04194, −0.04193, −0.04193, −0.04193, 0.04193, −0.04192, −0.04192, 0.0419, 0.0419, 0.0419, −0.0419, −0.04189, −0.04189, 0.04188, 0.04188, −0.04188, 0.04188, 0.04184, −0.04182, 0.04181, −0.04179, 0.04179, 0.04178, 0.04178, 0.04177, 0.04177, −0.04176, −0.04176, −0.04174, −0.04174, −0.04171, 0.04169, 0.04168, 0.04167, −0.04166, −0.04165, −0.04165, −0.04164, 0.04161, −0.04161, 0.0416, 0.0416, −0.04158, 0.04157, −0.04157, 0.04155, −0.04154, 0.04154, 0.04153, 0.04153, 0.04152, 0.04152, −0.04151, 0.04151, −0.04151, −0.04148, 0.04147, −0.04146, 0.04146, 0.04146, −0.04145, 0.04145, 0.04144, −0.04144, −0.04144, −0.04144, −0.04143, −0.04142, −0.04142, 0.04142, −0.04142, −0.04141, −0.04139, 0.04139, −0.04137, −0.04134, 0.04133, −0.04132, 0.04132, −0.04131, −0.0413, −0.04129, 0.04129, 0.04129, 0.04129, 0.04128, 0.04128, 0.04127, 0.04125, −0.04125, −0.04125, 0.04124, −0.04124, 0.04123, −0.04123, −0.04122, 0.04122, −0.04121, 0.0412, −0.0412, −0.04119, 0.04118, 0.04118, −0.04118, −0.04118, 0.04116, −0.04116, −0.04111, −0.0411, 0.0411, −0.04108, 0.04107, 0.04105, 0.04104, 0.04104, 0.04103, −0.04103, 0.04102, −0.041, 0.04099, 0.04098, 0.04098, 0.04097, −0.04093, −0.04092, 0.04092, 0.0409, 0.04089, −0.04088, 0.04086, −0.04085, 0.04084, 0.04084, 0.04083, 0.04083, −0.04082, −0.04082, 0.04081, 0.0408, −0.04079, −0.04075, 0.04073, −0.04073, −0.04073, −0.04073, −0.04072, −0.04071, 0.0407, 0.04069, 0.04069, 0.04068, −0.04068, −0.04067, −0.04066, −0.04065, 0.04065, −0.04065, 0.04063, −0.04062, −0.04062, 0.04061, 0.04061, 0.0406, −0.04059, 0.04058, 0.04058, 0.04058, 0.04056, 0.04056, −0.04055, 0.04053, −0.04053, −0.04051, −0.04051, −0.04051, −0.0405, −0.04049, −0.04049, 0.04048, −0.04047, 0.04046, 0.04044, −0.04044, −0.04043, −0.04043, −0.04043, 0.04043, 0.04042, 0.0404, −0.0404, 0.04039, −0.04039, 0.04038, −0.04037, −0.04036, 0.04034, 0.04034, −0.04034, −0.04033, 0.04032, 0.04032, −0.04031, 0.0403, −0.0403, 0.0403, −0.04029, 0.04029, 0.04028, 0.04026, −0.04025, 0.04024, −0.04024, 0.04021, −0.04021, 0.0402, 0.0402, −0.04019, 0.04018, −0.04018, −0.04018, 0.04017, −0.04016, −0.04016, 0.04013, 0.04013, 0.0401, 0.0401, 0.0401, 0.0401, 0.04007, −0.04007, 0.04005, −0.04004, 0.04001, 0.03999, 0.03997, −0.03997, −0.03995, −0.03995, −0.03994, 0.03993, 0.03993, 0.03992, 0.03992, −0.03992, 0.03991, −0.0399, 0.0399, 0.03988, −0.03988, 0.03988, −0.03986, 0.03985, 0.03985, −0.03983, −0.03983, 0.03982, −0.03982, −0.03981, −0.03979, 0.03979, −0.03978, 0.03978, −0.03976, 0.03976, 0.03975, 0.03974, 0.03974, 0.03973, −0.03973, −0.03972, 0.03972, 0.03972, 0.03972, −0.03971, 0.0397, 0.0397, 0.03969, −0.03968, 0.03968, −0.03967, −0.03966, −0.03966, 0.03965, 0.03964, 0.03963, −0.03962, 0.03961, 0.03961, 0.03961, 0.0396, 0.0396, 0.03959, 0.03959, 0.03958, −0.03956, 0.03956, 0.03954, −0.03953, −0.03953, −0.03952, −0.03951, −0.03949, −0.03949, 0.03949, 0.03949, 0.03948, 0.03946, 0.03944, 0.03943, 0.03942, 0.03941, −0.03941, 0.03941, −0.03941, −0.0394, −0.03939, −0.03938, 0.03937, 0.03935, −0.03935, 0.03935, −0.03934, 0.03933, −0.03933, 0.03932, −0.03931, −0.0393, 0.03929, −0.03929, 0.03926, −0.03924, 0.03922, −0.03921, −0.0392, −0.0392, −0.03918, −0.03917, −0.03917, −0.03915, −0.03915, −0.03915, −0.03913, 0.03913, 0.03912, −0.03911, −0.03911, 0.0391, −0.0391, −0.03909, −0.03906, 0.03905, 0.03903, 0.03903, 0.039, 0.03899, 0.03899, −0.03899, 0.03897, 0.03897, −0.03897, −0.03897, 0.03897, 0.03896, −0.03896, 0.03896, −0.03895, −0.03894, 0.03894, −0.03893, −0.03893, −0.03892, 0.03891, 0.03891, 0.0389, −0.0389, 0.03889, 0.03889, −0.03888, 0.03887, −0.03885, −0.03885, 0.03884, 0.03883, 0.03882, −0.03882, 0.03881, 0.03881, 0.0388, 0.03879, −0.03879, 0.03879, 0.03879, 0.03877, −0.03877, −0.03877, 0.03877, −0.03877, −0.03877, 0.03876, 0.03875, 0.03875, 0.03875, 0.03874, −0.03873, 0.03872, −0.03872, −0.03871, 0.03871, −0.0387, −0.03868, 0.03868, −0.03867, 0.03864, 0.03864, 0.03863, 0.03862, −0.03861, −0.03861, 0.0386, 0.03857, −0.03856, −0.03856, −0.03856, 0.03855, −0.03855, −0.03855, 0.03855, 0.03853, −0.03853, −0.03852, 0.0385, 0.0385, 0.03848, −0.03848, 0.03847, 0.03846, 0.03846, 0.03841, −0.03839, 0.03838, −0.03838, −0.03837, 0.03836, −0.03835, −0.03835, 0.03835, −0.03834, −0.03834, −0.03834, −0.03833, 0.03832, −0.03831, 0.0383, 0.0383, −0.03828, −0.03828, 0.03828, 0.03826, −0.03825, −0.03824, 0.03822, −0.03821, 0.03819, 0.03817, −0.03817, −0.03817, 0.03817, 0.03816, 0.03816, 0.03815, −0.03815, 0.03814, 0.03814, 0.03814, 0.03812, −0.0381, −0.0381, 0.0381, 0.03808, −0.03807, −0.03807, −0.03807, −0.03805, −0.03805, 0.03805, 0.03804, 0.03804, −0.03803, −0.03802, −0.03799, 0.03799, 0.03797, −0.03796, −0.03795, −0.03794, −0.03792, 0.03792, 0.03791, 0.03789, −0.03788, 0.03786, −0.03785, −0.03784, −0.03783, −0.03782, 0.03782, 0.03781, −0.03781, 0.03781, −0.03781, −0.0378, 0.03779, 0.03779, 0.03779, −0.03778, 0.03778, 0.03777, −0.03777, 0.03776, 0.03775, −0.03775, 0.03775, −0.03773, −0.03771, −0.0377, −0.03769, −0.03769, −0.03769, 0.03766, 0.03764, 0.03763, −0.03763, 0.03761, −0.03761, 0.0376, 0.0376, −0.0376, 0.0376, −0.03759, 0.03759, −0.03758, 0.03758, −0.03757, −0.03757, −0.03757, −0.03756, 0.03756, −0.03752, 0.03752, 0.03751, −0.03751, −0.03751, −0.0375, −0.03749, 0.03747, 0.03747, −0.03746, 0.03746, −0.03743, −0.03743, 0.03743, 0.03742, −0.03742, −0.03742, 0.03741, 0.03741, 0.0374, 0.0374, −0.03739, −0.03738, 0.03737, −0.03737, 0.03736, −0.03733, −0.03731, −0.03729, 0.03728, −0.03728, 0.03726, 0.03725, 0.03724, −0.03723, −0.03723, 0.03722, 0.03721, −0.03721, −0.0372, 0.03719, 0.03719, 0.03718, 0.03717, −0.03716, 0.03715, −0.03715, −0.03714, 0.03713, 0.03713, −0.03712, −0.03711, 0.03708, −0.03708, −0.03706, −0.03706, −0.03706, 0.03705, −0.03704, −0.03703, −0.03701, 0.03701, 0.037, −0.037, −0.03699, 0.03698, 0.03696, −0.03696, −0.03695, 0.03695, 0.03695, −0.03694, 0.03692, −0.03692, −0.03692, −0.03691, 0.0369, 0.03689, 0.03688, 0.03685, −0.03681, 0.03681, −0.0368, 0.03677, −0.03675, −0.03675, 0.03673, 0.03672, −0.03672, −0.03671, 0.0367, 0.03669, −0.03669, 0.03668, 0.03667, 0.03666, 0.03666, 0.03664, 0.03661, −0.03661, −0.03661, 0.0366, −0.0366, −0.03658, −0.03658, 0.03655, −0.03654, 0.03653, 0.03653, −0.03653, −0.03651, 0.03651, −0.03651, −0.03651, 0.0365, 0.03649, −0.03649, −0.03648, −0.03648, −0.03647, 0.03646, −0.03644, −0.03644, −0.03644, 0.03644, 0.03643, −0.03643, 0.03643, 0.03643, −0.03642, −0.03642, −0.03642, −0.03642, 0.03639, 0.03638, 0.03637, −0.03636, −0.03634, 0.03634, 0.03633, −0.03632, −0.03632, 0.03632, 0.0363, −0.0363, −0.03629, −0.03629, −0.03629, −0.03627, −0.03627, 0.03626, −0.03626, 0.03625, −0.03625, 0.03624, 0.03624, −0.03623, 0.03623, −0.03623, −0.03621, 0.03621, 0.03618, −0.03618, −0.03617, 0.03617, 0.03616, 0.03615, −0.03615, 0.03615, 0.03615, −0.03613, −0.03612, 0.03611, −0.03611, 0.03611, 0.03611, −0.03608, −0.03607, −0.03606, 0.03605, −0.03601, −0.036, −0.036, 0.036, −0.036, −0.03598, 0.03598, −0.03597, 0.03596, 0.03596, 0.03595, −0.03595, 0.03595, 0.03595, 0.03594, −0.03594, 0.03589, −0.03589, −0.03589, −0.03589, −0.03587, −0.03586, 0.03586, −0.03584, −0.03583, −0.03582, −0.03582, −0.0358, −0.0358, −0.03579, 0.03579, −0.03579, 0.03578, −0.03578, −0.03577, 0.03575, −0.03574, 0.03573, −0.03573, 0.03573, −0.03572, −0.03572, 0.03572, 0.03572, 0.03571, 0.03571, −0.03571, −0.03571, 0.0357, 0.0357, 0.03569, 0.03569, −0.03569, 0.03567, 0.03567, −0.03567, −0.03565, 0.03565, 0.03565, −0.03564, 0.03563, 0.03563, −0.03563, 0.03562, 0.03562, 0.0356, −0.03559, −0.03559, −0.03559, −0.03558, −0.03558, −0.03558, −0.03557, 0.03556, 0.03556, 0.03556, −0.03556, 0.03555, 0.03554, −0.03554, −0.03554, 0.03554, −0.03553, 0.03552, −0.03552, −0.03552, −0.03551, −0.03551, −0.03551, −0.0355, −0.0355, −0.03549, 0.03547, 0.03546, 0.03545, 0.03544, −0.03542, 0.03541, −0.03541, −0.0354, 0.0354, 0.0354, 0.03539, 0.03539, 0.03539, −0.03539, 0.03538, 0.03537, −0.03536, −0.03535, 0.03535, 0.03535, 0.03534, 0.03534, −0.03534, 0.03533, 0.03532, 0.03531, 0.0353, 0.03529, −0.03527, −0.03526, −0.03526, 0.03526, −0.03525, −0.03524, 0.03523, 0.03523, −0.03522, −0.03522, 0.03522, −0.0352, −0.03519, 0.03519, 0.03518, 0.03517, −0.03517, −0.03517, 0.03516, −0.03515, −0.03514, −0.03513, 0.03513, −0.03513, 0.03512, −0.03511, −0.03511, 0.0351, −0.0351, −0.03509, 0.03508, 0.03507, 0.03505, −0.03505, 0.03505, −0.03504, −0.03503, 0.03503, −0.03503, −0.03502, −0.03501, −0.03501, 0.03498, 0.03497, 0.03497, −0.03495, −0.03495, −0.03495, −0.03494, −0.03493, −0.03492, −0.03491, 0.0349, −0.03489, −0.03488, −0.03487, 0.03486, 0.03485, 0.03484, 0.03483, −0.03483, −0.03481, 0.03481, −0.03481, 0.0348, 0.03479, −0.03478, −0.03477, −0.03477, −0.03476, −0.03475, −0.03475, 0.03475, 0.03474, −0.03474, −0.03473, 0.03472, −0.03472, 0.0347, 0.03468, 0.03468, 0.03468, 0.03468, 0.03468, −0.03467, 0.03467, −0.03467, 0.03467, 0.03467, −0.03466, 0.03466, −0.03465, −0.03464, −0.03464, −0.03463, 0.03462, 0.0346, 0.03458, −0.03458, 0.03458, 0.03458, 0.03457, −0.03456, −0.03456, 0.03456, −0.03453, 0.03451, 0.03449, −0.03449, −0.03449, −0.03447, −0.03446, −0.03446, −0.03445, −0.03445, −0.03444, 0.03443, −0.03443, −0.03442, 0.03442, 0.03441, 0.03441, −0.03441, 0.0344, −0.03439, −0.03437, −0.03434, −0.03434, 0.03432, −0.03432, −0.03432, 0.03431, −0.03431, 0.0343, −0.03429, −0.03429, 0.03429, −0.03428, −0.03428, −0.03428, −0.03428, −0.03427, 0.03426, −0.03426, −0.03425, 0.03425, −0.03425, −0.03425, 0.03423, −0.03423, 0.03422, −0.03419, −0.03419, −0.03417, −0.03417, 0.03417, 0.03417, −0.03417, −0.03415, −0.03415, 0.03415, −0.03415, 0.03414, −0.03414, 0.03412, −0.03412, −0.03412, 0.03412, −0.03412, −0.03411, −0.03411, 0.03411, 0.03408, 0.03407, 0.03407, −0.03407, −0.03406, 0.03405, 0.03405, 0.03402, 0.034, 0.03399, −0.03396, −0.03395, −0.03394, −0.03393, 0.03392, −0.03391, 0.03391, −0.0339, −0.0339, −0.03388, 0.03388, 0.03387, 0.03387, −0.03387, −0.03386, −0.03385, 0.03385, −0.03384, −0.03384, −0.03383, −0.03383, −0.03383, −0.03382, −0.03379, −0.03379, 0.03377, −0.03376, 0.03376, 0.03375, 0.03375, −0.03375, 0.03374, 0.03374, 0.03373, 0.03372, 0.03372, 0.03372, −0.03372, −0.03372, −0.03369, 0.03368, 0.03366, −0.03366, −0.03366, −0.03365, 0.03364, 0.03364, 0.03362, −0.03361, −0.03361, −0.0336, −0.0336, −0.0336, −0.03359, −0.03358, 0.03358, 0.03358, 0.03357, −0.03357, −0.03356, 0.03356, −0.03354, −0.03353, −0.03353, 0.03353, 0.03353, −0.03352, 0.03352, 0.03351, 0.03351, 0.03351, 0.0335, 0.03349, 0.03349, −0.03348, 0.03347, 0.03347, −0.03347, 0.03343, 0.0334, −0.03339, −0.03338, −0.03338, 0.03338, 0.03337, 0.03336, 0.03336, −0.03335, −0.03335, −0.03334, 0.03333, −0.03333, 0.03333, 0.03332, −0.03332, −0.03331, 0.0333, 0.0333, −0.0333, 0.0333, 0.03329, −0.03329, 0.03329, −0.03329, 0.03329, 0.03328, −0.03326, −0.03326, −0.03325, 0.03325, 0.03325, 0.03324, −0.03323, −0.03323, −0.03321, 0.03319, 0.03319, 0.03318, −0.03317, 0.03317, 0.03317, −0.03316, 0.03316, 0.03316, 0.03315, −0.03313, −0.03313, 0.03313, −0.03312, −0.03312, 0.03312, −0.03311, 0.03311, 0.03309, 0.03308, 0.03307, −0.03307, 0.03306, −0.03305, 0.03305, 0.03305, 0.03304, 0.03302, −0.03301, 0.03301, 0.033, −0.033, 0.03299, 0.03298, −0.03297, −0.03297, −0.03295, −0.03295, −0.03294, 0.03292, −0.03292, −0.03291, 0.0329, −0.03287, 0.03287, 0.03287, 0.03286, −0.03285, 0.03285, 0.03285, 0.03284, 0.03282, −0.03282, −0.03282, −0.03282, −0.03281, −0.03281, −0.03279, −0.03279, 0.03278, −0.03277, 0.03277, −0.03276, 0.03276, −0.03276, −0.03276, −0.03275, 0.03274, 0.03274, −0.03273, 0.03271, −0.03271, −0.0327, −0.03269, −0.03267, −0.03266, −0.03265, −0.03263, 0.03263, −0.03262, 0.03262, 0.03262, 0.03261, −0.0326, 0.03259, 0.03259, −0.03259, 0.03258, −0.03258, −0.03257, −0.03257, −0.03256, −0.03256, −0.03256, −0.03255, −0.03255, 0.03254, −0.03253, −0.03253, 0.03253, −0.03253, 0.03252, −0.03251, 0.03251, 0.0325, −0.0325, −0.03249, 0.03248, −0.03247, 0.03245, 0.03245, 0.03244, −0.03243, 0.03243, −0.03243, 0.03241, 0.03241, −0.03239, 0.03239, 0.03237, 0.03237, 0.03237, 0.03237, −0.03236, −0.03235, 0.03234, 0.03233, 0.03233, −0.03233, −0.03233, −0.03231, −0.0323, −0.03229, 0.03229, 0.03228, 0.03227, 0.03227, −0.03227, 0.03226, 0.03225, 0.03225, −0.03225, −0.03222, −0.03222, −0.03221, −0.03219, −0.03219, 0.03219, 0.03218, 0.03218, −0.03217, −0.03217, 0.03216, 0.03215, −0.03215, −0.03215, 0.03214, −0.03214, −0.03214, 0.03214, 0.03212, −0.03212, 0.03212, −0.03212, −0.03211, 0.03209, −0.03208, −0.03207, 0.03207, −0.03207, 0.03206, 0.03206, −0.03205, −0.03205, 0.03205, 0.03205, 0.03205, 0.03204, −0.03204, 0.03203, 0.03202, 0.03202, −0.03202, 0.03201, 0.03201, −0.03199, 0.03199, 0.03196, 0.03194, −0.03193, 0.03192, 0.03191, −0.03189, 0.03188, 0.03187, −0.03186, 0.03185, 0.03185, −0.03185, −0.03183, −0.03183, 0.03182, 0.03181, 0.03178, −0.03178, 0.03175, 0.03175, −0.03174, 0.03173, 0.03173, −0.03173, −0.03172, 0.03172, 0.03171, −0.0317, −0.0317, −0.0317, −0.03169, 0.03169, 0.03166, 0.03165, −0.03165, 0.03164, 0.03163, 0.03162, 0.03161, −0.0316, −0.0316, 0.03159, 0.03159, 0.03158, 0.03156, 0.03155, −0.03154, −0.03153, 0.03152, −0.0315, −0.0315, −0.0315, −0.03149, 0.03148, −0.03147, −0.03147, 0.03146, −0.03146, −0.03144, −0.03143, 0.03143, 0.03139, −0.03139, 0.03139, −0.03139, 0.03138, −0.03138, −0.03137, −0.03137, 0.03136, 0.03136, −0.03135, 0.03135, 0.03133, −0.03133, −0.03133, −0.03132, −0.0313, −0.0313, −0.03129, −0.03128, 0.03128, 0.03128, 0.03127, −0.03127, −0.03125, −0.03125, 0.03124, 0.03124, 0.03124, 0.03123, −0.03122, 0.03122, −0.03121, −0.03121, 0.03121, −0.03117, 0.03117, −0.03116, −0.03116, −0.03115, −0.03115, 0.03114, 0.03113, 0.03113, 0.03112, 0.03112, −0.03111, 0.0311, −0.03109, 0.03109, −0.03108, 0.03108, 0.03108, 0.03107, −0.03106, 0.03105, 0.03105, −0.03103, 0.03101, −0.031, −0.031, −0.031, 0.031, 0.03099, 0.03099, 0.03099, −0.03099, −0.03098, −0.03097, 0.03096, −0.03096, −0.03093, −0.03093, −0.03092, −0.03092, −0.0309, −0.03089, −0.03089, −0.03088, −0.03088, −0.03088, −0.03087, 0.03087, 0.03085, 0.03084, −0.03084, −0.03083, 0.03082, −0.03082, 0.03082, −0.03082, −0.03081, −0.03081, −0.0308, −0.0308, −0.0308, −0.03077, −0.03076, −0.03075, 0.03074, 0.03072, 0.03071, 0.03071, 0.0307, −0.03069, 0.03069, −0.03068, 0.03068, 0.03068, −0.03067, 0.03064, −0.03064, −0.03062, −0.03062, −0.03062, 0.0306, −0.0306, 0.03059, −0.03059, −0.03059, −0.03058, −0.03057, 0.03057, 0.03057, −0.03056, 0.03056, 0.03056, 0.03055, −0.03054, −0.03053, −0.03052, 0.0305, −0.0305, 0.0305, 0.03049, 0.03048, −0.03048, −0.03047, −0.03047, −0.03047, −0.03044, 0.03043, 0.03042, 0.03041, 0.03041, 0.03039, 0.03036, −0.03036, 0.03036, 0.03035, 0.03032, −0.03032, 0.03031, 0.03031, −0.03031, 0.03031, 0.0303, 0.03029, 0.03029, −0.03028, −0.03028, 0.03027, 0.03027, 0.03025, 0.03025, −0.03024, −0.03023, 0.03021, −0.0302, −0.0302, −0.0302, 0.0302, 0.03019, 0.03018, 0.03018, −0.03018, −0.03017, −0.03017, −0.03016, 0.03015, 0.03015, −0.03015, −0.03014, 0.03013, 0.03012, 0.0301, 0.03009, −0.03009, 0.03008, 0.03008, −0.03008, 0.03006, −0.03005, 0.03005, 0.03004, 0.03002, 0.03002, 0.03002, 0.03001, 0.03001, −0.03001, 0.02999, 0.02998, −0.02998, −0.02998, −0.02998, −0.02997, 0.02997, 0.02994, −0.02992, −0.02992, −0.02992, 0.02991, −0.0299, −0.02989, 0.02988, −0.02988, 0.02988, 0.02986, 0.02986, −0.02986, −0.02985, −0.02985, −0.02984, 0.02984, −0.02982, 0.02982, −0.02981, 0.0298, 0.0298, −0.0298, −0.0298, −0.02979, 0.02979, 0.02979, −0.02979, −0.02977, −0.02977, −0.02976, −0.02976, −0.02976, 0.02975, −0.02974, −0.02973, 0.02973, 0.02971, −0.0297, 0.0297, −0.02969, 0.02968, −0.02968, 0.02967, −0.02966, −0.02965, −0.02964, 0.02964, −0.02963, 0.02963, −0.02963, −0.02963, −0.02961, 0.02961, 0.02961, 0.02957, 0.02956, −0.02956, −0.02955, −0.02955, −0.02952, −0.02952, 0.02951, −0.02951, 0.02951, −0.0295, −0.02949, 0.02949, −0.02947, 0.02947, −0.02947, 0.02945, 0.02945, 0.02945, −0.02945, 0.02944, −0.02944, −0.02943, −0.02942, −0.02941, 0.02941, 0.0294, −0.02937, 0.02937, 0.02937, −0.02936, −0.02936, −0.02935, −0.02935, −0.02934, 0.02934, 0.02934, −0.02933, −0.02933, −0.02932, −0.02932, 0.02932, 0.0293, −0.02929, 0.02929, 0.02928, 0.02927, −0.02927, −0.02926, 0.02926, −0.02922, −0.02921, −0.02921, −0.0292, −0.0292, 0.02919, −0.02919, 0.02918, −0.02918, −0.02918, −0.02917, −0.02917, 0.02917, −0.02917, 0.02915, −0.02914, −0.02913, −0.02913, 0.02913, −0.02912, 0.02912, 0.0291, 0.0291, −0.02909, −0.02909, −0.02908, 0.02906, 0.02904, 0.02903, 0.02901, 0.02901, 0.029, 0.02899, −0.02897, −0.02897, −0.02896, 0.02896, −0.02896, 0.02895, −0.02895, −0.02894, −0.02893, 0.02893, −0.02892, 0.02892, 0.02891, −0.02891, −0.0289, −0.0289, 0.0289, 0.02889, 0.02889, −0.02888, −0.02887, 0.02887, −0.02887, −0.02886, 0.02886, 0.02885, −0.02885, −0.02884, 0.02883, −0.02883, 0.02882, 0.02881, 0.02881, −0.02881, 0.0288, 0.0288, −0.02879, −0.02879, −0.02879, −0.02878, −0.02878, 0.02876, −0.02875, 0.02875, 0.02874, 0.02873, −0.02872, 0.02871, 0.02871, −0.02871, −0.0287, 0.02869, −0.02868, −0.02867, −0.02866, 0.02862, −0.02862, 0.02862, −0.02862, −0.02861, −0.02861, 0.02861, −0.02861, −0.02861, −0.0286, −0.02858, −0.02857, 0.02857, 0.02856, 0.02852, 0.02852, 0.02851, −0.0285, −0.0285, −0.0285, −0.02849, 0.02848, 0.02848, −0.02847, −0.02847, −0.02847, 0.02847, −0.02846, 0.02845, 0.02845, 0.02845, 0.02844, 0.02843, 0.02842, −0.02842, 0.0284, −0.02839, −0.02838, −0.02838, −0.02837, 0.02837, −0.02837, −0.02836, 0.02836, −0.02835, 0.02833, −0.02833, −0.02833, 0.02832, 0.02832, 0.02832, 0.02832, 0.02832, −0.02832, 0.02832, 0.02831, −0.02831, −0.02828, −0.02828, 0.02826, 0.02826, 0.02825, −0.02825, 0.02824, −0.02823, −0.02823, −0.02822, −0.02822, 0.02822, −0.02821, 0.0282, 0.0282, −0.02819, 0.02819, −0.02819, 0.02818, 0.02818, −0.02817, 0.02817, 0.02817, −0.02816, −0.02815, 0.02815, 0.02815, 0.02815, 0.02815, −0.02815, −0.02813, 0.02813, −0.02811, −0.02811, 0.0281, 0.0281, 0.02809, 0.02809, −0.02809, −0.02809, 0.02809, −0.02808, −0.02807, 0.02807, 0.02807, −0.02807, 0.02806, 0.02806, −0.02805, 0.02805, −0.02805, −0.02803, 0.02802, 0.02802, 0.02802, −0.02801, −0.02801, 0.028, −0.028, 0.028, 0.028, 0.02799, −0.02799, 0.02797, −0.02797, −0.02797, 0.02795, 0.02795, −0.02794, −0.02791, 0.02791, −0.02789, −0.02789, −0.02789, 0.02788, −0.02784, −0.02783, 0.02783, −0.02782, 0.02782, −0.02781, 0.02781, −0.0278, 0.02779, 0.02778, −0.02776, 0.02776, −0.02775, 0.02775, 0.02774, 0.02774, −0.02774, −0.02773, −0.02773, −0.02772, 0.02772, 0.02772, −0.02772, 0.02771, −0.0277, −0.0277, −0.02769, −0.02769, 0.02768, −0.02768, 0.02768, −0.02767, 0.02767, −0.02765, 0.02765, −0.02764, 0.02764, 0.02764, −0.02762, −0.02762, 0.02762, −0.02759, 0.02759, 0.02757, 0.02754, 0.02754, 0.02754, −0.02752, 0.02751, 0.0275, −0.0275, 0.0275, 0.02749, 0.02748, −0.02747, −0.02747, 0.02747, −0.02746, 0.02746, −0.02746, −0.02745, −0.02744, 0.02743, −0.02742, −0.02741, 0.02739, 0.02739, −0.02739, −0.02737, −0.02737, 0.02735, 0.02735, −0.02735, 0.02733, −0.02732, 0.02732, −0.02731, 0.0273, −0.0273, 0.0273, 0.02729, 0.02729, 0.02728, −0.02728, −0.02727, −0.02727, 0.02727, 0.02727, 0.02727, 0.02726, −0.02725, −0.02725, −0.02725, 0.02724, 0.02724, 0.02723, −0.02721, −0.02721, −0.02721, −0.0272, 0.0272, −0.02719, 0.02719, −0.02718, 0.02718, −0.02717, −0.02717, 0.02715, 0.02715, 0.02714, −0.02713, 0.02712, −0.02712, −0.02711, −0.02711, 0.02711, 0.0271, 0.0271, −0.02709, −0.02709, 0.02708, 0.02708, −0.02706, −0.02705, 0.02704, 0.02703, 0.02703, 0.02702, 0.02701, −0.02701, 0.02699, −0.02699, −0.02699, −0.02696, −0.02694, −0.02694, −0.02694, −0.02694, 0.02693, 0.02691, −0.02691, 0.0269, 0.0269, −0.02689, −0.02689, −0.02689, −0.02689, −0.02689, 0.02688, −0.02687, −0.02687, −0.02686, −0.02686, 0.02685, 0.02685, 0.02684, 0.02684, −0.02684, −0.02684, 0.02684, 0.02682, 0.02681, 0.02681, 0.02681, −0.02681, 0.0268, 0.02679, −0.02679, −0.02679, −0.02678, 0.02678, 0.02678, −0.02676, −0.02676, 0.02676, 0.02675, 0.02675, −0.02675, 0.02674, −0.02674, −0.02674, −0.02674, 0.02673, 0.02672, −0.02672, 0.02672, −0.02671, −0.0267, −0.02669, −0.02668, 0.02667, −0.02667, −0.02666, −0.02666, 0.02665, −0.02664, 0.02663, −0.02662, −0.0266, −0.0266, −0.0266, −0.02658, −0.02658, 0.02656, 0.02656, 0.02655, −0.02655, −0.02655, 0.02655, −0.02655, 0.02655, −0.02654, −0.02653, −0.02651, −0.02651, 0.0265, 0.0265, 0.02649, 0.02648, −0.02647, 0.02647, −0.02646, −0.02644, 0.02644, 0.02643, −0.02643, 0.02643, −0.02643, −0.02642, 0.02642, 0.0264, −0.0264, −0.02638, 0.02637, −0.02636, −0.02634, 0.02634, −0.02634, −0.02634, 0.02632, −0.02631, −0.02629, −0.02628, −0.02628, 0.02628, 0.02628, 0.02627, −0.02627, 0.02627, −0.02626, 0.02625, 0.02623, −0.02623, 0.02623, 0.02623, −0.02623, 0.02621, 0.02621, −0.0262, 0.0262, −0.02617, 0.02617, 0.02617, −0.02615, −0.02615, −0.02615, −0.02614, 0.02614, −0.02614, 0.02614, −0.02614, 0.02612, 0.02612, −0.02611, −0.02611, −0.0261, −0.0261, −0.0261, −0.02609, −0.02609, 0.02609, 0.02608, 0.02607, −0.02607, −0.02607, −0.02606, −0.02606, −0.02604, −0.02603, −0.02603, 0.02603, −0.02602, −0.02602, 0.02602, 0.02602, −0.02602, −0.02601, 0.02601, 0.02601, −0.026, 0.02598, 0.02597, −0.02596, 0.02596, 0.02596, −0.02595, 0.02595, −0.02594, −0.02594, −0.02594, 0.02593, −0.02593, −0.02593, 0.02593, 0.02593, 0.02592, −0.02592, −0.02592, 0.02591, 0.02591, 0.02591, −0.0259, 0.02588, 0.02588, −0.02587, −0.02586, −0.02586, −0.02585, −0.02585, 0.02584, 0.02584, −0.02584, 0.02583, 0.02583, 0.02581, 0.0258, 0.0258, 0.0258, −0.0258, 0.02579, 0.02579, 0.02578, −0.02577, −0.02576, 0.02576, −0.02575, −0.02575, −0.02574, 0.02572, 0.02571, 0.02569, 0.02568, −0.02568, 0.02568, −0.02565, 0.02565, −0.02564, 0.02564, −0.02564, 0.02564, −0.02563, −0.02562, 0.02562, −0.02561, 0.02561, 0.0256, 0.02558, −0.02558, −0.02556, 0.02555, −0.02555, 0.02553, −0.02552, 0.02552, −0.02552, 0.02551, −0.0255, 0.0255, −0.02549, −0.02549, −0.02549, −0.02547, −0.02547, 0.02546, −0.02545, 0.02544, 0.02542, 0.02542, 0.02541, −0.0254, 0.02539, −0.02538, −0.02538, −0.02536, −0.02536, 0.02536, 0.02534, 0.02533, −0.02533, 0.02532, −0.02531, −0.0253, −0.0253, −0.0253, −0.0253, −0.0253, 0.0253, −0.02529, 0.02528, −0.02527, 0.02525, 0.02525, −0.02524, 0.02524, −0.02524, −0.02522, 0.02522, 0.02522, 0.02522, 0.02521, −0.02521, 0.02521, −0.02521, −0.02521, 0.0252, 0.0252, −0.02519, 0.02518, −0.02517, −0.02517, −0.02517, −0.02517, 0.02516, −0.02516, 0.02515, −0.02514, −0.02514, 0.02514, −0.02513, −0.02512, 0.02511, −0.02511, −0.02511, 0.02509, −0.02508, −0.02508, −0.02508, −0.02508, 0.02507, 0.02507, 0.02507, 0.02506, −0.02505, 0.02504, −0.02504, 0.02504, 0.02503, −0.02503, 0.02503, −0.02503, −0.02501, −0.025, −0.025, 0.02499, −0.02499, 0.02498, −0.02497, −0.02497, 0.02497, 0.02496, 0.02495, 0.02495, −0.02495, −0.02494, −0.02492, 0.02491, 0.02491, −0.02491, 0.0249, 0.0249, 0.02489, 0.02489, −0.02489, 0.02488, 0.02487, −0.02486, −0.02485, −0.02483, 0.02483, −0.02481, −0.0248, −0.0248, −0.02479, 0.02479, −0.02477, 0.02475, −0.02474, −0.02474, −0.02473, 0.02473, −0.02472, 0.02471, 0.0247, 0.02469, 0.02469, 0.02469, −0.02468, −0.02468, 0.02468, −0.02467, −0.02467, −0.02467, −0.02466, −0.02466, −0.02466, −0.02465, −0.02465, 0.02465, −0.02464, −0.02463, −0.02463, 0.02462, −0.02462, −0.02461, −0.0246, 0.0246, −0.0246, −0.02459, −0.02459, −0.02459, 0.02457, 0.02456, 0.02455, 0.02455, −0.02455, −0.02455, −0.02454, −0.02454, −0.02454, 0.02453, −0.02452, −0.02452, 0.02452, −0.02451, −0.02451, −0.0245, 0.0245, −0.02449, 0.02448, −0.02447, 0.02447, −0.02446, −0.02445, 0.02445, 0.02445, −0.02444, −0.02444, −0.02444, 0.02443, 0.02443, −0.02442, −0.02442, 0.02442, 0.02442, 0.02442, 0.02441, −0.02441, −0.0244, 0.0244, 0.02439, 0.02437, −0.02437, 0.02437, −0.02437, 0.02437, −0.02435, 0.02434, 0.02433, −0.02433, −0.02432, −0.02431, −0.02431, −0.02431, −0.02431, 0.0243, −0.0243, 0.0243, 0.02429, 0.02429, −0.02429, −0.02428, 0.02428, 0.02428, 0.02428, 0.02427, −0.02426, 0.02425, −0.02424, 0.02423, 0.02423, 0.02423, 0.02422, 0.02421, −0.02421, −0.0242, −0.02419, 0.02418, −0.02418, −0.02418, 0.02416, 0.02416, 0.02415, −0.02414, −0.02414, 0.02413, 0.02412, 0.02412, −0.02411, 0.0241, 0.0241, −0.0241, −0.0241, −0.02409, −0.02408, −0.02408, −0.02406, 0.02405, 0.02404, −0.02404, 0.02403, −0.02402, 0.02401, −0.024, 0.02399, −0.02398, 0.02398, 0.02398, −0.02398, 0.02397, −0.02396, 0.02396, 0.02396, −0.02395, 0.02395, 0.02394, 0.02394, −0.02394, 0.02394, −0.02393, 0.02393, −0.02391, 0.0239, −0.0239, −0.02389, −0.02387, 0.02386, −0.02386, −0.02385, −0.02385, 0.02384, 0.02383, −0.02383, −0.02383, −0.02382, 0.02381, 0.0238, 0.0238, 0.02379, −0.02379, 0.02378, −0.02378, −0.02378, 0.02377, 0.02377, −0.02376, −0.02375, 0.02375, 0.02374, 0.02373, 0.02373, 0.02372, 0.02372, −0.02371, −0.0237, 0.02369, 0.02369, 0.02368, −0.02368, −0.02368, −0.02368, 0.02367, 0.02366, 0.02365, −0.02365, −0.02365, −0.02365, 0.02364, 0.02363, 0.02362, −0.02362, −0.02362, 0.02362, 0.02361, 0.02361, −0.0236, 0.0236, 0.02358, 0.02358, −0.02358, 0.02358, −0.02357, −0.02356, −0.02355, 0.02354, 0.02353, 0.02353, −0.02353, −0.02352, 0.02352, 0.0235, −0.02348, −0.02348, 0.02348, −0.02348, 0.02347, −0.02347, 0.02346, −0.02346, −0.02346, −0.02346, −0.02345, 0.02345, −0.02345, 0.02344, −0.02344, −0.02344, −0.02343, 0.02342, 0.02341, 0.0234, 0.0234, 0.02339, 0.02339, 0.02338, 0.02338, 0.02338, 0.02338, 0.02336, −0.02336, 0.02336, 0.02336, −0.02334, 0.02333, −0.02333, 0.02333, 0.02332, 0.02331, 0.02331, −0.02331, −0.02331, −0.0233, −0.02329, −0.02329, 0.02328, −0.02328, 0.02328, 0.02327, 0.02326, −0.02326, −0.02325, −0.02323, −0.02322, −0.02322, 0.02321, −0.0232, 0.02319, −0.02319, −0.02319, 0.02317, 0.02317, 0.02317, −0.02317, −0.02316, 0.02316, 0.02316, −0.02314, −0.02312, −0.02312, 0.02311, −0.0231, 0.0231, −0.02309, −0.02309, −0.02309, 0.02309, −0.02309, −0.02309, −0.02309, −0.02307, −0.02307, −0.02307, −0.02306, −0.02306, 0.02306, 0.02305, −0.02302, −0.02301, 0.023, −0.023, −0.023, −0.023, 0.023, 0.02299, 0.02299, −0.02298, 0.02298, −0.02298, 0.02297, 0.02297, −0.02297, 0.02297, 0.02297, −0.02296, 0.02296, −0.02296, −0.02294, 0.02294, −0.02293, 0.02292, −0.0229, −0.0229, −0.0229, −0.02289, −0.02289, −0.02289, 0.02288, −0.02288, 0.02288, 0.02287, −0.02287, −0.02287, −0.02287, −0.02286, 0.02286, −0.02283, 0.02283, −0.0228, 0.02279, 0.02279, 0.02278, 0.02277, −0.02276, −0.02275, 0.02275, −0.02275, 0.02275, 0.02274, 0.02274, 0.02273, −0.02273, 0.02272, 0.02271, −0.02271, 0.02271, 0.02271, −0.02271, 0.0227, 0.0227, −0.02269, −0.02269, −0.02267, −0.02267, −0.02266, −0.02266, −0.02266, −0.02265, 0.02264, 0.02264, 0.02263, −0.02261, −0.0226, 0.0226, 0.02259, −0.02259, 0.02258, −0.02258, 0.02257, 0.02257, 0.02257, 0.02255, 0.02254, 0.02254, −0.02253, 0.02252, −0.02251, 0.0225, −0.02249, −0.02249, 0.02249, 0.02248, −0.02248, 0.02248, 0.02248, 0.02247, −0.02247, −0.02247, 0.02246, −0.02246, −0.02245, 0.02243, 0.02242, 0.02242, −0.02242, 0.02242, −0.02241, 0.02241, −0.02241, 0.0224, 0.0224, 0.0224, −0.0224, −0.02237, −0.02237, 0.02237, 0.02237, 0.02235, −0.02234, 0.02234, −0.02233, 0.02233, −0.02233, 0.02232, −0.02232, −0.02231, 0.02231, 0.0223, 0.0223, −0.02229, −0.02229, 0.02229, 0.02228, 0.02228, −0.02227, 0.02226, 0.02226, −0.02225, −0.02225, 0.02223, −0.02222, −0.02222, −0.02222, 0.0222, 0.02219, 0.02219, 0.02219, 0.02218, −0.02218, 0.02218, −0.02217, 0.02217, 0.02217, 0.02216, 0.02216, 0.02215, 0.02215, 0.02214, −0.02214, −0.02214, 0.02214, −0.02212, 0.02212, −0.02209, 0.02207, 0.02207, 0.02207, 0.02207, −0.02206, −0.02206, 0.02206, −0.02206, 0.02205, −0.02205, −0.02205, −0.02204, −0.02204, 0.02204, 0.02201, 0.022, 0.022, −0.02199, 0.02199, −0.02198, 0.02198, 0.02197, −0.02197, −0.02197, −0.02196, 0.02196, 0.02196, −0.02196, 0.02196, 0.02196, −0.02193, 0.02193, −0.02193, −0.02193, −0.02192, −0.02192, 0.02192, −0.02192, −0.02191, 0.02191, −0.0219, 0.02189, −0.02189, 0.02188, 0.02188, −0.02187, 0.02186, −0.02186, 0.02186, 0.02186, 0.02186, −0.02185, −0.02185, 0.02185, 0.02185, −0.02185, 0.02185, −0.02184, −0.02184, −0.02184, 0.02183, 0.02183, 0.02183, −0.02182, −0.0218, −0.0218, −0.0218, 0.02178, 0.02177, 0.02176, −0.02176, 0.02175, 0.02175, −0.02173, −0.02173, −0.02171, −0.02171, 0.02169, −0.02169, 0.02167, −0.02167, 0.02167, −0.02166, 0.02166, −0.02164, 0.02164, 0.02164, 0.02161, 0.02161, 0.02161, 0.0216, −0.0216, −0.02159, 0.02159, 0.02159, −0.02158, −0.02158, −0.02156, −0.02156, 0.02155, 0.02155, −0.02154, −0.02154, 0.02154, 0.02153, 0.02153, −0.02153, 0.02152, −0.02152, −0.02151, 0.02151, 0.02151, 0.02148, −0.02148, −0.02147, −0.02147, −0.02147, 0.02146, −0.02146, −0.02146, −0.02145, 0.02144, −0.02144, −0.02143, −0.02141, 0.02141, −0.02141, 0.0214, −0.0214, 0.02139, 0.02139, −0.02138, −0.02138, 0.02137, −0.02137, 0.02136, 0.02135, 0.02134, −0.02133, 0.02133, −0.02132, 0.02132, 0.02132, 0.02131, −0.02131, −0.02131, −0.02129, 0.02128, −0.02127, 0.02127, 0.02127, 0.02127, −0.02125, −0.02124, 0.02124, 0.02124, −0.02123, 0.02122, 0.02122, 0.02122, −0.02121, −0.02121, 0.02118, 0.02118, −0.02115, −0.02115, 0.02115, −0.02115, 0.02113, −0.02112, −0.02112, −0.02111, −0.0211, −0.02109, 0.02108, 0.02108, 0.02107, 0.02107, −0.02106, 0.02106, 0.02105, 0.02104, −0.02101, −0.021, 0.02099, −0.02099, −0.02099, 0.02098, 0.02098, −0.02098, 0.02097, −0.02097, −0.02093, 0.02093, 0.02092, 0.02092, −0.02092, 0.02092, −0.02091, 0.0209, −0.0209, −0.0209, 0.02089, −0.02089, −0.02088, −0.02086, 0.02086, 0.02085, −0.02085, −0.02084, −0.02084, 0.02084, 0.02084, 0.02083, −0.02083, −0.02083, −0.02083, 0.02082, 0.02081, 0.02081, −0.02081, −0.0208, 0.02079, 0.02078, 0.02078, 0.02077, −0.02076, 0.02076, −0.02075, −0.02074, −0.02073, 0.02073, −0.02072, 0.02072, 0.02072, 0.02071, 0.02071, 0.02071, −0.0207, −0.0207, −0.0207, −0.0207, −0.02068, −0.02068, −0.02067, −0.02066, 0.02066, 0.02066, −0.02066, −0.02065, −0.02065, −0.02064, 0.02064, −0.02064, −0.02064, −0.02063, 0.02063, −0.02062, −0.02062, −0.02062, 0.02061, −0.02061, 0.02061, −0.02061, −0.0206, 0.02058, −0.02057, −0.02057, 0.02056, −0.02055, −0.02055, −0.02055, 0.02055, −0.02054, −0.02054, 0.02051, −0.02051, 0.02048, 0.02048, 0.02048, −0.02047, −0.02047, −0.02046, −0.02046, 0.02045, 0.02044, 0.02043, 0.02042, −0.02042, 0.0204, −0.02039, 0.02038, −0.02038, −0.02038, −0.02038, 0.02037, −0.02037, −0.02036, 0.02036, −0.02034, −0.02034, 0.02034, −0.02034, −0.02032, 0.02032, 0.02032, −0.02031, 0.02031, 0.02031, −0.0203, 0.0203, 0.0203, −0.02029, 0.02029, 0.02029, 0.02028, 0.02028, 0.02027, −0.02025, −0.02024, −0.02024, 0.02024, −0.02023, −0.02023, −0.02022, 0.02022, −0.02021, −0.02021, −0.0202, −0.02018, 0.02018, −0.02018, −0.02018, 0.02015, 0.02013, −0.02012, −0.02011, −0.02009, 0.02007, −0.02007, 0.02007, −0.02006, −0.02005, 0.02004, −0.02004, −0.02003, −0.02002, 0.02002, 0.02001, −0.02001, 0.02001, 0.02001, 0.02, 0.02, 0.01998, −0.01997, −0.01997, −0.01996, −0.01996, −0.01996, 0.01996, 0.01996, −0.01995, −0.01994, −0.01994, −0.01993, −0.01993, 0.01991, −0.01991, −0.0199, −0.0199, 0.01989, −0.01988, −0.01987, −0.01987, 0.01985, 0.01985, 0.01985, 0.01985, 0.01985, 0.01984, 0.01984, 0.01984, 0.01984, 0.01983, 0.01983, −0.01983, 0.01982, 0.01982, −0.01982, 0.01981, 0.01981, 0.01981, 0.0198, −0.01979, 0.01979, 0.01979, 0.01978, −0.01976, −0.01976, −0.01975, −0.01974, −0.01974, −0.01974, 0.01973, 0.01972, −0.01972, 0.01971, −0.01971, −0.0197, 0.01969, −0.01969, 0.01969, −0.01969, −0.01968, −0.01968, −0.01968, 0.01967, 0.01967, 0.01966, −0.01965, 0.01964, 0.01964, 0.01964, 0.01964, 0.01962, −0.01962, −0.0196, 0.01959, −0.01959, 0.01959, −0.01958, 0.01957, 0.01957, −0.01956, −0.01955, −0.01955, −0.01955, −0.01953, −0.01953, 0.01953, 0.01953, −0.01953, 0.01953, 0.01952, −0.01951, −0.01951, −0.01951, −0.01951, −0.0195, 0.0195, −0.01948, 0.01948, −0.01946, −0.01945, 0.01944, −0.01943, −0.01943, 0.01941, 0.01941, 0.0194, −0.01939, −0.01939, −0.01939, −0.01938, 0.01937, −0.01936, 0.01936, −0.01935, −0.01935, −0.01935, −0.01935, −0.01933, −0.01933, 0.01932, −0.01931, 0.01931, −0.01931, 0.0193, −0.01929, 0.01929, 0.01928, −0.01927, −0.01927, 0.01926, 0.01926, −0.01925, 0.01923, 0.01923, −0.01923, −0.01922, 0.01922, −0.0192, −0.01919, −0.01918, −0.01918, 0.01917, 0.01917, 0.01917, 0.01917, −0.01915, −0.01915, 0.01915, −0.01915, 0.01913, 0.01913, −0.01913, −0.01912, 0.01911, −0.01911, −0.01911, −0.01911, 0.0191, 0.0191, −0.0191, −0.0191, −0.01909, −0.01909, 0.01906, −0.01906, −0.01904, 0.01903, −0.01903, −0.01903, −0.01902, −0.01902, 0.01902, 0.01902, 0.01901, −0.01901, −0.01901, −0.01901, −0.01899, −0.01899, −0.01899, 0.01899, 0.01898, 0.01897, −0.01896, 0.01895, 0.01895, −0.01894, −0.01892, 0.01889, 0.01888, −0.01887, −0.01886, −0.01886, 0.01886, 0.01884, 0.01884, 0.01883, −0.01883, −0.01883, 0.01882, 0.01882, −0.01882, 0.01882, −0.01881, 0.01879, 0.01879, 0.01878, −0.01878, −0.01878, −0.01878, −0.01877, −0.01877, 0.01876, −0.01876, 0.01875, 0.01875, 0.01874, −0.01873, 0.01872, 0.01871, 0.01871, −0.0187, −0.01869, 0.01867, −0.01867, 0.01867, 0.01866, −0.01866, −0.01866, 0.01865, 0.01864, −0.01864, 0.01863, −0.01862, 0.01861, −0.0186, −0.01859, 0.01859, −0.01858, 0.01858, 0.01858, −0.01858, 0.01857, 0.01855, −0.01854, 0.01853, 0.01853, −0.01852, 0.01852, 0.01851, −0.0185, 0.01849, −0.01849, −0.01848, −0.01848, 0.01847, 0.01847, 0.01847, −0.01847, −0.01846, 0.01845, 0.01845, −0.01845, 0.01844, 0.01844, 0.01844, −0.01844, −0.01843, −0.01842, −0.01842, −0.0184, −0.01839, −0.01839, −0.01838, −0.01838, −0.01838, −0.01834, 0.01834, −0.01833, −0.01833, 0.01833, −0.01832, 0.01832, 0.01831, 0.01831, 0.01831, 0.01829, −0.01829, 0.01828, 0.01828, 0.01827, −0.01825, −0.01825, 0.01824, 0.01822, −0.01822, −0.01821, 0.01821, 0.0182, 0.0182, 0.01818, 0.01817, 0.01816, −0.01816, 0.01815, −0.01815, −0.01814, −0.01813, −0.01812, 0.01812, 0.01812, 0.01811, 0.01811, −0.0181, 0.0181, 0.01809, −0.01808, −0.01807, −0.01806, 0.01805, 0.01805, −0.01805, −0.01804, −0.01803, 0.01802, 0.01801, 0.018, −0.01799, −0.01799, 0.01799, 0.01798, 0.01798, −0.01798, −0.01798, −0.01797, 0.01797, 0.01796, 0.01796, 0.01794, 0.01793, 0.01792, 0.01792, 0.01792, 0.0179, −0.0179, 0.0179, −0.0179, 0.01787, 0.01787, 0.01786, 0.01786, −0.01784, −0.01783, −0.01783, −0.01783, 0.01782, 0.01782, −0.0178, 0.0178, 0.0178, −0.0178, 0.01779, −0.01778, 0.01777, −0.01776, 0.01776, −0.01775, 0.01774, 0.01774, −0.01774, −0.01774, 0.01773, −0.01772, −0.01772, −0.0177, −0.0177, 0.01769, −0.01769, 0.01769, 0.01768, 0.01768, 0.01767, 0.01767, −0.01766, −0.01765, −0.01765, −0.01765, −0.01764, −0.01764, 0.01763, 0.01763, 0.01763, 0.01762, −0.01761, 0.01761, −0.0176, 0.01759, 0.01757, 0.01757, 0.01756, 0.01756, 0.01756, −0.01755, 0.01754, 0.01754, −0.01753, 0.01753, 0.01752, 0.0175, 0.01749, 0.01748, −0.01747, 0.01745, −0.01744, 0.01743, −0.01743, 0.01742, 0.01742, 0.01741, −0.0174, −0.01739, −0.01739, −0.01738, −0.01737, 0.01737, 0.01737, 0.01736, 0.01736, 0.01735, −0.01732, −0.01732, −0.01731, 0.01729, −0.01729, 0.01729, 0.01728, 0.01727, 0.01727, 0.01727, 0.01727, 0.01726, −0.01725, 0.01724, −0.01723, −0.01723, −0.01723, −0.01722, −0.01722, −0.01722, 0.01722, −0.01721, 0.0172, −0.0172, −0.01719, −0.01718, 0.01718, 0.01717, 0.01717, 0.01717, −0.01717, 0.01715, 0.01715, −0.01714, 0.01714, 0.01713, −0.01713, −0.01713, 0.01713, −0.01711, 0.01708, −0.01708, −0.01707, −0.01706, −0.01706, −0.01705, 0.01705, 0.01704, 0.01703, −0.01702, 0.017, −0.017, 0.017, 0.017, −0.01699, −0.01697, −0.01695, −0.01695, 0.01694, −0.01694, 0.01693, 0.01693, 0.01693, −0.01693, −0.01692, 0.01691, 0.01691, 0.0169, −0.01689, 0.01688, 0.01688, 0.01687, −0.01687, 0.01687, 0.01685, −0.01685, 0.01685, 0.01685, −0.01685, 0.01685, 0.01684, −0.01684, −0.01683, 0.01682, −0.01682, 0.01682, 0.01681, −0.0168, −0.0168, −0.0168, 0.01679, 0.01678, −0.01678, −0.01677, 0.01676, 0.01676, 0.01674, −0.01673, 0.01673, 0.01673, 0.01673, 0.01672, −0.01672, 0.01671, −0.0167, −0.0167, −0.0167, 0.0167, −0.0167, −0.01669, −0.01669, 0.01668, −0.01668, 0.01668, 0.01667, 0.01667, −0.01667, 0.01666, −0.01664, −0.01663, −0.01662, 0.01662, −0.01662, 0.01661, 0.01661, 0.01661, −0.0166, 0.0166, 0.01659, −0.01658, −0.01657, −0.01656, 0.01656, −0.01656, −0.01655, −0.01655, 0.01655, −0.01653, −0.01653, 0.01653, −0.01652, −0.01652, 0.01652, 0.01651, −0.01651, −0.0165, 0.0165, 0.01649, −0.01649, −0.01647, 0.01646, −0.01646, −0.01646, 0.01644, 0.01644, 0.01644, −0.01643, 0.01642, −0.01642, 0.01641, 0.0164, −0.0164, −0.01639, 0.01636, −0.01634, −0.01634, 0.01633, −0.01632, 0.01632, 0.01632, 0.0163, −0.01629, 0.01629, 0.01629, 0.01628, −0.01627, 0.01626, −0.01625, −0.01624, 0.01624, 0.01623, −0.01623, −0.01622, 0.01622, 0.01621, −0.0162, −0.01619, −0.01619, 0.01619, 0.01619, 0.01613, 0.01613, 0.01612, −0.01612, −0.01611, −0.0161, −0.0161, −0.01609, −0.01609, 0.01608, −0.01608, −0.01608, 0.01605, 0.01604, 0.01604, 0.01604, 0.01603, −0.01602, −0.01602, 0.01602, 0.01601, 0.01601, −0.01601, −0.016, 0.016, −0.01598, 0.01598, −0.01598, 0.01597, −0.01597, −0.01597, −0.01596, 0.01595, 0.01595, −0.01594, 0.01594, −0.01594, 0.01593, −0.01592, −0.01592, −0.01591, 0.0159, 0.01589, 0.01589, −0.01588, −0.01587, 0.01586, 0.01586, 0.01585, 0.01585, −0.01584, −0.01584, 0.01583, −0.01582, −0.01582, 0.01582, −0.01582, 0.01581, 0.01581, −0.0158, −0.0158, −0.0158, −0.0158, −0.0158, −0.0158, 0.01579, 0.01579, −0.01579, 0.01578, 0.01578, 0.01578, −0.01577, 0.01575, 0.01575, −0.01575, 0.01575, −0.01574, 0.01574, −0.01574, 0.01573, −0.01573, 0.01573, −0.01573, 0.01572, 0.01572, 0.01571, −0.01571, −0.01568, −0.01567, 0.01566, 0.01565, 0.01564, 0.01563, −0.01563, 0.01563, 0.01563, −0.01563, −0.01562, −0.01562, −0.01562, 0.01561, −0.01561, 0.0156, 0.0156, 0.01559, 0.01559, −0.01559, 0.01557, −0.01556, −0.01556, 0.01556, 0.01556, 0.01556, −0.01555, −0.01553, 0.01552, −0.01552, −0.01551, 0.0155, 0.01548, 0.01547, −0.01547, 0.01546, −0.01546, −0.01545, −0.01545, −0.01545, −0.01544, 0.01543, 0.01542, 0.01541, −0.01541, −0.01541, 0.0154, 0.0154, 0.01539, 0.01539, 0.01539, 0.01538, −0.01538, −0.01538, −0.01536, 0.01536, −0.01535, 0.01535, 0.01534, 0.01534, 0.01533, −0.01532, 0.01532, −0.01531, 0.01531, −0.01531, 0.01531, 0.01531, −0.0153, −0.0153, 0.0153, −0.0153, −0.0153, −0.01529, 0.01529, 0.01529, −0.01529, 0.01529, −0.01529, −0.01528, 0.01528, 0.01528, −0.01526, 0.01525, −0.01524, 0.01524, −0.01523, 0.01522, 0.01522, 0.01521, 0.01521, −0.01521, −0.01521, −0.01521, 0.0152, −0.01519, 0.01516, −0.01516, −0.01516, −0.01516, −0.01515, −0.01514, 0.01514, −0.01514, 0.01514, −0.01513, −0.0151, 0.0151, 0.0151, 0.01509, 0.01509, −0.01509, 0.01508, 0.01508, 0.01507, −0.01507, −0.01506, 0.01505, −0.01505, 0.01504, 0.01501, 0.01499, 0.01499, −0.01498, 0.01497, −0.01497, −0.01497, −0.01496, 0.01496, 0.01495, −0.01495, 0.01495, 0.01494, −0.01494, 0.01494, 0.01493, 0.01493, −0.01492, −0.01492, −0.01492, 0.01491, −0.01491, 0.01491, −0.0149, −0.0149, 0.0149, −0.0149, −0.01489, −0.01488, −0.01488, −0.01487, 0.01486, 0.01486, 0.01485, 0.01485, −0.01484, −0.01484, −0.01483, 0.01483, 0.01483, 0.01483, −0.01481, 0.01481, −0.01481, 0.0148, 0.01479, −0.01479, 0.01478, 0.01478, −0.01475, 0.01475, −0.01474, 0.01473, 0.01473, 0.01472, 0.01471, −0.01471, −0.0147, 0.0147, 0.0147, 0.0147, 0.0147, −0.0147, 0.01469, −0.01469, 0.01468, 0.01468, −0.01467, 0.01467, 0.01466, −0.01466, −0.01466, 0.01466, −0.01465, −0.01464, 0.01464, −0.01463, 0.01463, 0.01463, −0.01463, 0.01462, −0.01461, 0.01461, −0.01461, −0.01461, −0.01459, −0.01459, −0.01459, 0.01459, −0.01458, 0.01457, 0.01457, −0.01456, −0.01456, 0.01456, −0.01454, 0.01454, 0.01452, −0.01452, −0.01451, 0.01451, 0.01451, 0.01451, 0.01451, −0.01449, −0.01449, 0.01449, −0.01448, 0.01448, −0.01447, 0.01446, −0.01446, 0.01446, 0.01445, −0.01444, −0.01443, −0.01443, −0.01442, −0.01441, 0.01441, −0.0144, 0.0144, −0.01439, 0.01439, −0.01439, −0.01438, −0.01438, 0.01438, −0.01437, −0.01437, −0.01436, 0.01435, 0.01435, 0.01434, −0.01434, 0.01433, 0.01432, −0.01431, −0.01429, 0.01429, −0.01429, 0.01428, −0.01428, 0.01427, −0.01426, −0.01425, 0.01425, 0.01424, −0.01423, 0.01423, −0.01423, −0.01423, −0.01423, 0.01421, −0.01421, 0.0142, 0.01419, −0.01419, −0.01419, −0.01419, −0.01417, −0.01416, 0.01416, 0.01415, 0.01415, −0.01414, −0.01414, 0.01412, 0.01412, −0.01412, −0.01412, 0.01412, −0.01411, −0.01411, 0.0141, 0.01408, −0.01408, 0.01407, 0.01407, 0.01407, −0.01407, 0.01406, 0.01406, 0.01405, −0.01405, 0.01404, 0.01404, −0.01403, −0.01403, −0.01402, −0.01402, 0.01401, −0.014, 0.01399, −0.01398, −0.01398, 0.01398, 0.01397, 0.01396, 0.01396, −0.01395, 0.01395, −0.01395, 0.01395, 0.01394, 0.01394, −0.01393, 0.01393, −0.01392, −0.01391, −0.01391, −0.01391, −0.01391, −0.01391, −0.0139, −0.01389, 0.01389, −0.01388, −0.01387, −0.01387, 0.01386, 0.01385, −0.01385, −0.01384, −0.01384, 0.01384, −0.01384, −0.01383, 0.0138, −0.0138, −0.0138, −0.01379, −0.01378, −0.01377, −0.01377, 0.01377, −0.01377, −0.01377, 0.01377, −0.01377, 0.01376, 0.01376, 0.01376, 0.01375, −0.01374, 0.01374, 0.01374, 0.01373, 0.01372, 0.01371, −0.01371, −0.0137, 0.0137, −0.0137, −0.01369, −0.01368, −0.01367, −0.01365, −0.01365, −0.01363, −0.01362, −0.01362, −0.01362, −0.01362, −0.01362, −0.0136, −0.01359, −0.01358, −0.01356, −0.01356, 0.01355, 0.01355, −0.01355, 0.01355, −0.01354, −0.01352, 0.01351, 0.01351, 0.01351, −0.0135, −0.01349, −0.01347, −0.01347, 0.01345, −0.01345, −0.01344, −0.01343, −0.01342, 0.01342, 0.01341, −0.01341, 0.0134, −0.01339, −0.01338, −0.01338, 0.01338, 0.01338, 0.01337, −0.01337, 0.01337, 0.01337, −0.01336, 0.01336, 0.01335, 0.01334, −0.01334, 0.01334, −0.01333, −0.01333, 0.01332, 0.0133, −0.0133, 0.0133, 0.0133, −0.01329, −0.01329, −0.01329, 0.01328, 0.01328, 0.01328, −0.01327, −0.01326, −0.01324, −0.01323, 0.01323, 0.01323, −0.01322, −0.01321, 0.0132, 0.01319, −0.01319, 0.01319, −0.01317, 0.01315, 0.01315, −0.01315, −0.01314, 0.01314, −0.01314, −0.01314, 0.01314, −0.01313, 0.01313, 0.01313, −0.01312, 0.01312, 0.01311, 0.01311, 0.01311, 0.01311, 0.01311, −0.0131, 0.0131, −0.01309, −0.01309, −0.01309, 0.01309, 0.01308, 0.01307, −0.01305, 0.01304, 0.01304, 0.01304, −0.01299, 0.01297, −0.01297, 0.01297, 0.01296, −0.01296, 0.01295, −0.01295, −0.01294, 0.01293, 0.01293, 0.01293, −0.01293, −0.01293, −0.01292, −0.01292, 0.01292, −0.01292, −0.01292, −0.01291, 0.01291, −0.0129, −0.0129, −0.0129, −0.01289, 0.01289, 0.01288, 0.01288, 0.01288, 0.01287, −0.01287, −0.01287, −0.01286, −0.01286, 0.01285, −0.01285, −0.01285, 0.01285, −0.01284, −0.01283, −0.01283, 0.01282, −0.01282, 0.01282, −0.01282, −0.01281, −0.0128, 0.0128, 0.01279, 0.01279, −0.01278, −0.01278, −0.01278, −0.01278, −0.01277, −0.01277, 0.01277, 0.01276, 0.01275, 0.01274, 0.01274, −0.01274, 0.01274, −0.01274, 0.01273, 0.01271, 0.0127, 0.01269, 0.01268, 0.01268, −0.01267, 0.01267, −0.01266, −0.01265, 0.01265, 0.01265, 0.01265, 0.01264, −0.01264, −0.01264, −0.01263, −0.01263, −0.01262, 0.0126, −0.0126, 0.0126, 0.01259, −0.01258, 0.01257, −0.01257, 0.01257, −0.01256, −0.01255, −0.01255, −0.01255, 0.01254, −0.01253, −0.01253, 0.01253, 0.01252, 0.01252, −0.01251, 0.01249, 0.01249, −0.01249, 0.01249, 0.01248, −0.01248, −0.01248, −0.01248, −0.01247, −0.01247, −0.01246, −0.01245, −0.01245, −0.01245, −0.01243, 0.01243, 0.01243, −0.01243, −0.01242, −0.01242, −0.01242, −0.01242, 0.01241, 0.01241, −0.01241, −0.0124, 0.0124, 0.0124, 0.0124, −0.01239, −0.01239, −0.01237, 0.01237, 0.01237, 0.01236, 0.01236, −0.01235, −0.01235, 0.01234, 0.01234, 0.01234, 0.01234, −0.01233, 0.01233, −0.01233, −0.01232, −0.01232, 0.01232, −0.01232, −0.01232, 0.01232, 0.01231, 0.01231, 0.0123, −0.0123, −0.0123, 0.01228, 0.01225, −0.01224, −0.01224, 0.01223, 0.01223, −0.01222, −0.01221, 0.0122, 0.01218, 0.01217, 0.01216, −0.01216, 0.01216, −0.01215, 0.01215, −0.01215, 0.01214, 0.01213, 0.01213, −0.0121, 0.01209, −0.01208, −0.01208, 0.01208, −0.01207, −0.01207, −0.01207, 0.01206, −0.01206, −0.01205, 0.01204, −0.01203, 0.01202, −0.01201, −0.01201, 0.01197, −0.01195, 0.01193, 0.01191, −0.01191, −0.01191, 0.0119, 0.0119, −0.0119, −0.01189, −0.01189, −0.01188, 0.01188, −0.01188, 0.01188, −0.01187, 0.01187, −0.01186, 0.01186, −0.01184, 0.01184, 0.01184, 0.01182, 0.01182, −0.01182, 0.01182, 0.01181, −0.0118, 0.0118, 0.0118, −0.0118, 0.01179, −0.01179, 0.01179, −0.01178, 0.01177, −0.01177, 0.01176, 0.01176, 0.01174, 0.01174, −0.01173, −0.01171, −0.01171, −0.0117, −0.0117, 0.01167, 0.01167, −0.01166, 0.01165, 0.01165, −0.01165, −0.01164, 0.01163, −0.01163, −0.01163, 0.01162, 0.01162, 0.0116, 0.01159, −0.01159, −0.01158, 0.01158, −0.01157, −0.01156, −0.01156, −0.01155, −0.01154, −0.01154, −0.01153, −0.01153, −0.01152, 0.01152, 0.01151, 0.01151, −0.0115, 0.0115, 0.0115, −0.0115, 0.01149, −0.01148, −0.01148, 0.01147, 0.01147, 0.01146, 0.01146, 0.01145, 0.01145, −0.01145, 0.01145, −0.01145, −0.01144, −0.01142, 0.01142, −0.01142, 0.01142, 0.01141, −0.01141, −0.0114, −0.01139, 0.01139, −0.01139, −0.01139, 0.01138, −0.01137, 0.01137, 0.01137, −0.01134, 0.01134, 0.01133, −0.01132, 0.01131, −0.01131, 0.01131, −0.01131, −0.01131, −0.0113, −0.0113, −0.0113, −0.0113, 0.01129, 0.01129, 0.01127, −0.01127, 0.01125, −0.01123, 0.01123, 0.01122, −0.01121, −0.01121, 0.01121, −0.0112, 0.01119, −0.01118, 0.01118, 0.01117, −0.01116, −0.01114, 0.01114, 0.01112, 0.01112, 0.01112, −0.01111, −0.01111, 0.0111, −0.0111, −0.01108, −0.01108, −0.01107, −0.01107, −0.01107, −0.01107, −0.01106, −0.01104, 0.01104, 0.01104, −0.01104, −0.01101, −0.01101, 0.01101, −0.01101, −0.011, 0.01099, 0.01098, 0.01098, 0.01098, −0.01097, −0.01096, 0.01096, −0.01095, −0.01094, −0.01094, 0.01093, 0.01093, 0.01092, −0.0109, 0.01089, 0.01088, 0.01088, −0.01088, 0.01088, −0.01087, −0.01087, −0.01086, −0.01086, 0.01085, −0.01084, −0.01083, −0.01083, 0.01082, −0.01081, 0.01079, 0.01079, 0.01079, 0.01078, −0.01078, 0.01078, 0.01077, −0.01077, 0.01075, −0.01074, 0.01074, 0.01073, −0.01073, −0.01073, 0.01073, 0.01072, −0.01072, 0.01071, −0.01071, 0.01071, −0.0107, −0.0107, −0.0107, −0.0107, 0.01069, 0.01069, −0.01068, −0.01067, 0.01066, 0.01066, 0.01066, 0.01064, −0.01064, 0.01063, −0.01063, 0.01061, −0.01061, 0.01061, 0.0106, −0.0106, −0.0106, 0.0106, −0.01059, −0.01059, −0.01058, 0.01058, −0.01058, −0.01057, 0.01057, 0.01057, −0.01056, −0.01056, −0.01056, −0.01054, −0.01053, 0.01053, −0.01052, −0.01052, 0.01052, 0.01051, 0.01051, −0.0105, −0.0105, −0.01049, 0.01048, 0.01047, −0.01047, 0.01047, −0.01047, −0.01046, 0.01046, 0.01046, −0.01046, 0.01045, 0.01045, −0.01045, −0.01045, −0.01045, −0.01043, −0.01043, −0.01043, −0.01042, 0.01041, 0.0104, −0.0104, −0.0104, 0.0104, −0.0104, 0.0104, 0.01039, 0.01039, 0.01038, −0.01038, −0.01038, −0.01037, −0.01037, −0.01036, 0.01035, −0.01035, −0.01035, 0.01035, 0.01034, 0.01034, 0.01034, −0.01031, 0.01031, 0.01031, 0.01031, 0.01031, −0.01029, −0.01029, −0.01028, 0.01027, 0.01027, 0.01027, 0.01026, −0.01025, 0.01025, 0.01024, −0.01024, −0.01023, −0.01023, 0.01023, −0.01022, −0.01022, −0.01022, −0.0102, 0.0102, 0.0102, −0.0102, 0.01019, 0.01019, −0.01018, 0.01018, 0.01018, 0.01017, −0.01015, 0.01015, −0.01015, 0.01015, 0.01014, −0.01013, −0.01013, −0.01012, 0.01012, −0.01012, −0.01011, −0.0101, −0.0101, −0.01008, −0.01008, −0.01008, −0.01007, −0.01007, −0.01006, 0.01005, 0.01005, 0.01004, −0.01003, −0.01002, 0.01001, −0.01001, −0.01001, 0.01001, 0.01, 0.01, 0.01, 0.00999, 0.00999, −0.00999, 0.00998, −0.00998, −0.00998, −0.00997, 0.00997, −0.00997, 0.00996, 0.00996, −0.00996, −0.00995, 0.00994, 0.00993, 0.00993, 0.00993, −0.00993, −0.00992, 0.00992, −0.00991, 0.00991, −0.00991, 0.0099, 0.00989, −0.00989, 0.00988, 0.00988, 0.00987, −0.00986, −0.00984, 0.00984, 0.00984, 0.00984, 0.00983, −0.00982, 0.00982, −0.00981, −0.0098, −0.00979, −0.00979, −0.00977, −0.00976, −0.00976, 0.00974, −0.00973, −0.00973, −0.00972, −0.00972, 0.00972, 0.00971, 0.00971, −0.0097, 0.00969, 0.00968, 0.00968, 0.00967, −0.00967, −0.00967, 0.00967, 0.00965, −0.00965, 0.00965, −0.00964, 0.00964, 0.00962, −0.00962, 0.00962, −0.00961, −0.00961, −0.00961, −0.0096, 0.0096, −0.00959, 0.00959, 0.00959, 0.00958, −0.00958, 0.00957, 0.00956, 0.00956, −0.00955, 0.00955, 0.00955, −0.00955, −0.00955, 0.00953, 0.00952, −0.00951, −0.00951, 0.00951, −0.00951, 0.00951, 0.0095, −0.0095, −0.00949, −0.00949, −0.00949, 0.00949, −0.00948, −0.00948, 0.00948, −0.00947, 0.00947, 0.00947, −0.00946, 0.00946, −0.00946, 0.00945, 0.00945, −0.00944, 0.00944, 0.00944, −0.00944, −0.00944, −0.00943, 0.00943, 0.00943, −0.00942, −0.00942, 0.00942, 0.00942, 0.00942, 0.00941, −0.00939, −0.00938, −0.00937, 0.00937, −0.00937, 0.00937, 0.00935, −0.00935, −0.00934, −0.00934, 0.00934, −0.00933, −0.00933, 0.00933, −0.00933, −0.00933, 0.00931, 0.0093, 0.0093, 0.00929, −0.00929, 0.00928, 0.00928, 0.00927, 0.00927, 0.00926, −0.00926, 0.00925, 0.00924, −0.00924, 0.00923, −0.00923, 0.00923, −0.00923, 0.00923, −0.00922, 0.00921, −0.00921, 0.00921, −0.00919, 0.00919, 0.00919, −0.00919, 0.00918, −0.00918, −0.00917, 0.00917, 0.00917, −0.00916, −0.00916, 0.00916, −0.00915, 0.00915, 0.00915, 0.00915, 0.00915, −0.00915, 0.00914, −0.00914, 0.00912, 0.00911, −0.0091, −0.00909, 0.00909, −0.00907, −0.00906, −0.00906, −0.00906, −0.00906, −0.00905, 0.00905, −0.00905, 0.00903, −0.00903, −0.00902, 0.00902, −0.00901, 0.00901, 0.009, 0.009, 0.009, 0.009, −0.009, −0.00899, 0.00898, 0.00898, 0.00898, 0.00897, 0.00896, 0.00895, 0.00895, −0.00895, 0.00893, 0.00892, 0.00892, 0.00891, −0.00891, −0.00891, −0.0089, 0.0089, −0.0089, −0.00889, −0.00889, −0.00889, −0.00888, −0.00888, −0.00886, 0.00886, −0.00885, 0.00885, 0.00885, −0.00884, −0.00884, 0.00883, −0.00882, −0.00882, 0.00881, −0.00881, 0.00879, −0.00879, 0.00879, 0.00879, 0.00879, 0.00877, 0.00877, −0.00876, −0.00876, 0.00875, 0.00875, −0.00874, −0.00874, 0.00874, 0.00873, 0.00872, 0.0087, −0.0087, −0.00869, −0.00869, 0.00869, 0.00868, −0.00868, 0.00867, 0.00867, 0.00867, −0.00867, 0.00867, −0.00867, 0.00867, 0.00866, −0.00864, 0.00863, −0.00862, 0.00861, 0.00861, 0.0086, −0.00859, −0.00858, −0.00858, 0.00857, 0.00857, −0.00855, 0.00854, −0.00853, −0.00851, −0.00851, −0.0085, −0.0085, −0.0085, 0.00849, 0.00849, 0.00848, 0.00848, −0.00847, −0.00847, −0.00846, 0.00846, −0.00845, 0.00845, 0.00844, −0.00844, 0.00844, −0.00843, −0.00843, 0.00842, 0.00841, −0.0084, 0.0084, −0.0084, −0.00839, 0.00838, −0.00838, −0.00837, 0.00837, −0.00836, 0.00835, −0.00835, 0.00835, −0.00833, −0.00833, 0.00833, 0.00833, 0.00833, −0.00832, −0.00831, 0.00831, −0.00831, −0.00831, 0.00829, 0.00828, 0.00828, −0.00828, −0.00827, 0.00827, −0.00826, −0.00826, −0.00826, −0.00825, 0.00825, −0.00824, 0.00824, 0.00824, 0.00823, −0.00823, 0.00822, −0.00822, −0.00821, −0.00821, 0.00821, 0.0082, −0.0082, −0.00819, −0.00819, 0.00819, −0.00818, −0.00818, 0.00818, 0.00817, −0.00817, −0.00816, −0.00816, −0.00816, 0.00816, −0.00816, 0.00816, 0.00815, 0.00815, 0.00815, −0.00815, 0.00814, −0.00814, 0.00813, 0.00813, −0.00813, 0.00813, −0.00812, 0.0081, 0.0081, −0.00809, 0.00808, −0.00807, −0.00806, 0.00806, 0.00806, −0.00804, 0.00804, −0.00804, 0.00802, −0.00802, −0.008, 0.008, 0.008, −0.00799, −0.00798, −0.00795, −0.00795, 0.00795, −0.00794, −0.00793, 0.00793, 0.00793, −0.00792, −0.00791, 0.00791, 0.00791, −0.0079, −0.00789, 0.00789, 0.00789, 0.00789, −0.00789, 0.00788, −0.00788, −0.00788, −0.00787, −0.00785, −0.00785, −0.00781, 0.00781, −0.0078, −0.0078, 0.0078, −0.00778, 0.00778, −0.00778, −0.00777, −0.00777, 0.00777, −0.00776, 0.00773, −0.00773, 0.00772, −0.00772, −0.0077, 0.0077, 0.0077, −0.0077, 0.00769, −0.00769, −0.00769, −0.00768, −0.00767, 0.00766, 0.00765, 0.00765, −0.00765, 0.00764, −0.00764, −0.00764, −0.00764, −0.00762, −0.00762, −0.00761, −0.00761, −0.0076, −0.00759, 0.00759, 0.00759, 0.00759, 0.00759, 0.00758, −0.00758, 0.00757, −0.00757, 0.00757, 0.00756, −0.00755, −0.00755, −0.00755, 0.00754, −0.00754, −0.00753, 0.00752, 0.00752, 0.00752, 0.0075, 0.00748, −0.00747, −0.00746, −0.00746, 0.00746, 0.00745, 0.00745, 0.00745, 0.00745, 0.00744, −0.00744, −0.00742, 0.00742, −0.00742, −0.00741, 0.00741, −0.00738, −0.00737, −0.00737, −0.00736, −0.00736, −0.00735, 0.00735, 0.00734, 0.00734, 0.00734, −0.00734, −0.00734, 0.00734, 0.00733, −0.00733, −0.00733, −0.00733, 0.00731, −0.00731, 0.0073, −0.00729, 0.00728, −0.00727, 0.00726, 0.00725, 0.00725, 0.00725, 0.00725, 0.00724, 0.00723, 0.00722, −0.0072, 0.0072, −0.0072, −0.00719, 0.00719, −0.00718, 0.00717, 0.00717, −0.00717, 0.00716, −0.00715, 0.00712, 0.00711, −0.00711, −0.0071, 0.0071, 0.00709, 0.00708, 0.00708, −0.00706, −0.00705, 0.00705, 0.00705, −0.00704, 0.00704, 0.00704, −0.00702, 0.007, −0.007, 0.00699, 0.00698, −0.00698, −0.00698, 0.00697, −0.00696, 0.00696, −0.00696, −0.00696, −0.00695, −0.00695, 0.00694, −0.00692, 0.00692, −0.00692, 0.00691, −0.00691, −0.00691, −0.0069, −0.00689, 0.00689, 0.00689, 0.00687, 0.00687, −0.00687, 0.00686, −0.00686, 0.00684, −0.00684, −0.00683, −0.00683, 0.00683, 0.00683, −0.00682, 0.00681, −0.00681, −0.00681, 0.0068, 0.0068, 0.0068, 0.0068, −0.0068, −0.00679, −0.00679, −0.00679, 0.00677, −0.00677, 0.00677, 0.00676, 0.00676, 0.00676, −0.00676, −0.00675, 0.00675, 0.00675, −0.00675, 0.00674, 0.00673, −0.00673, −0.00672, 0.00672, 0.00672, 0.00671, −0.00671, 0.00671, −0.00671, 0.0067, 0.0067, −0.00669, 0.00669, −0.00668, 0.00667, −0.00667, 0.00666, −0.00666, −0.00666, −0.00665, 0.00663, −0.00662, 0.0066, −0.00659, −0.00659, −0.00659, 0.00658, 0.00657, 0.00657, 0.00657, −0.00655, 0.00655, −0.00653, −0.00653, −0.00652, 0.00651, −0.00649, 0.00649, 0.00648, −0.00648, −0.00647, 0.00646, 0.00646, 0.00644, 0.00643, −0.00642, 0.00639, −0.00639, −0.00638, 0.00637, 0.00637, 0.00636, −0.00635, −0.00635, 0.00634, −0.00634, −0.00634, −0.00633, −0.00632, 0.00631, −0.00631, −0.00631, −0.0063, −0.0063, −0.00629, 0.00629, 0.00629, 0.00629, 0.00629, 0.00628, −0.00627, 0.00627, 0.00627, 0.00626, −0.00626, 0.00625, −0.00624, −0.00624, 0.00624, 0.00623, 0.00623, 0.00622, 0.00622, −0.00622, 0.00622, −0.00622, 0.0062, −0.0062, 0.0062, 0.00619, −0.00618, 0.00618, 0.00617, −0.00615, −0.00615, 0.00613, 0.00612, 0.00612, 0.00612, 0.00612, −0.00612, −0.00612, −0.00611, −0.00611, −0.0061, 0.0061, −0.00609, 0.00608, 0.00608, 0.00608, 0.00607, 0.00605, −0.00604, 0.00603, −0.00602, 0.00602, −0.006, −0.006, 0.00599, 0.00599, 0.00598, 0.00597, −0.00596, −0.00596, 0.00595, 0.00595, 0.00594, −0.00593, −0.00593, 0.00592, 0.00592, 0.00591, 0.00591, 0.00591, 0.00591, −0.0059, 0.0059, 0.00589, 0.00587, −0.00586, 0.00586, 0.00585, −0.00584, −0.00583, −0.00582, 0.00581, 0.00581, −0.00581, −0.0058, 0.0058, 0.0058, −0.0058, −0.00579, −0.00579, 0.00579, 0.00579, 0.00577, −0.00576, 0.00575, 0.00575, −0.00575, 0.00572, 0.00572, −0.00572, −0.00572, 0.00572, −0.00572, −0.0057, −0.0057, −0.00569, −0.00568, 0.00567, −0.00567, 0.00566, 0.00566, −0.00566, −0.00566, −0.00566, 0.00565, 0.00565, 0.00565, −0.00565, 0.00565, 0.00564, 0.00564, −0.00564, −0.00562, −0.00562, 0.00562, 0.00562, 0.00562, 0.00561, −0.00561, −0.00561, 0.0056, −0.00559, 0.00559, −0.00559, 0.00559, 0.00558, 0.00558, 0.00558, 0.00558, 0.00556, 0.00556, −0.00556, −0.00553, −0.00551, −0.00551, 0.0055, 0.0055, 0.00549, 0.00548, −0.00547, 0.00547, 0.00546, 0.00545, −0.00545, 0.00545, −0.00545, −0.00545, 0.00544, 0.00544, 0.00543, −0.00543, 0.00542, 0.00542, 0.00542, −0.00542, 0.00541, −0.0054, 0.0054, 0.0054, 0.00539, 0.00539, 0.00539, −0.00539, 0.00539, −0.00538, 0.00538, −0.00536, −0.00535, −0.00535, −0.00534, −0.00534, −0.00534, −0.00534, −0.00533, 0.00532, −0.00532, 0.00532, 0.00532, −0.00531, −0.00531, 0.00531, 0.00531, −0.00531, 0.0053, 0.0053, 0.00529, 0.00529, 0.00528, 0.00528, −0.00527, −0.00527, 0.00527, 0.00526, −0.00525, −0.00525, −0.00525, −0.00524, 0.00524, 0.00523, −0.00523, −0.00523, −0.00523, −0.00522, 0.00521, −0.00521, −0.0052, −0.0052, −0.0052, −0.0052, −0.00519, 0.00519, −0.00519, −0.00519, −0.00518, 0.00517, −0.00517, 0.00516, −0.00516, −0.00516, 0.00515, 0.00514, 0.00513, 0.00513, −0.00511, −0.0051, 0.0051, 0.00509, −0.00509, 0.00508, 0.00505, 0.00504, 0.00504, −0.00504, 0.00504, −0.00504, 0.00503, 0.00503, −0.00502, −0.00502, −0.00501, 0.00501, −0.005, 0.00499, −0.00499, −0.00499, −0.00498, −0.00498, −0.00498, 0.00497, −0.00497, 0.00497, 0.00496, 0.00496, −0.00496, −0.00496, −0.00494, −0.00491, 0.00491, −0.0049, 0.00489, 0.00489, 0.00488, 0.00488, −0.00487, 0.00487, −0.00486, 0.00486, −0.00485, −0.00483, 0.00482, 0.00482, −0.00482, 0.00481, −0.00478, 0.00478, 0.00478, 0.00478, −0.00477, −0.00476, −0.00476, 0.00475, 0.00474, −0.00474, −0.00474, −0.00474, −0.00473, 0.00472, −0.00472, −0.00472, −0.00471, −0.00471, 0.00469, −0.00468, −0.00467, 0.00467, 0.00467, 0.00467, 0.00466, −0.00465, 0.00464, −0.00464, −0.00463, −0.00463, 0.00462, −0.00462, −0.0046, 0.0046, −0.00459, −0.00459, 0.00459, 0.00458, 0.00457, −0.00456, −0.00455, 0.00455, 0.00455, 0.00454, −0.00453, 0.00453, 0.00452, 0.00451, 0.00451, −0.00451, −0.00451, −0.00451, 0.00451, −0.00451, 0.0045, 0.00449, 0.00449, −0.00449, 0.00449, −0.00448, −0.00448, −0.00447, 0.00446, −0.00446, 0.00445, 0.00444, 0.00443, 0.00443, −0.00443, 0.00443, 0.00442, −0.00442, −0.00442, 0.0044, 0.0044, 0.0044, 0.00439, −0.00439, −0.00438, −0.00438, 0.00438, −0.00438, 0.00437, 0.00437, −0.00436, 0.00436, −0.00435, −0.00435, 0.00432, −0.0043, −0.0043, 0.00429, −0.00428, −0.00428, 0.00428, −0.00428, −0.00426, −0.00426, 0.00426, −0.00426, −0.00425, −0.00425, −0.00424, −0.00424, 0.00423, 0.00421, −0.00421, 0.0042, 0.0042, 0.00418, −0.00418, 0.00418, −0.00418, 0.00417, 0.00417, −0.00417, 0.00416, −0.00414, −0.00414, −0.00414, −0.00413, −0.00412, 0.00412, −0.00411, 0.0041, 0.0041, −0.00407, 0.00407, 0.00406, 0.00406, −0.00406, −0.00406, 0.00405, 0.00405, −0.00405, −0.00404, −0.00402, −0.00402, 0.00402, 0.00402, 0.00401, −0.00401, −0.00401, 0.004, −0.00399, −0.00399, −0.00399, −0.00398, −0.00398, 0.00398, −0.00397, −0.00397, 0.00397, −0.00397, −0.00397, −0.00396, 0.00396, −0.00395, 0.00395, −0.00395, −0.00394, 0.00394, −0.00393, −0.00392, 0.00392, 0.00391, 0.0039, −0.0039, 0.00389, 0.00389, −0.00389, 0.00388, −0.00387, 0.00387, −0.00385, 0.00385, −0.00384, −0.00383, −0.00382, 0.00381, −0.00381, 0.00381, 0.00379, 0.00379, 0.00379, 0.00379, −0.00379, −0.00379, −0.00379, −0.00378, 0.00377, 0.00377, −0.00376, −0.00374, 0.00372, 0.00372, 0.00371, 0.00371, 0.00371, 0.0037, 0.0037, 0.00369, 0.00367, −0.00367, 0.00367, 0.00366, 0.00365, 0.00365, −0.00365, 0.00363, −0.0036, 0.0036, 0.0036, 0.0036, −0.00359, −0.00359, 0.00358, −0.00357, 0.00357, 0.00357, 0.00354, 0.00354, 0.00354, −0.00353, −0.00352, −0.00351, −0.00351, 0.0035, 0.00349, −0.00349, −0.00348, −0.00348, −0.00347, −0.00347, 0.00345, 0.00344, 0.00344, 0.00344, 0.00344, 0.00343, 0.00341, −0.00341, 0.00341, −0.00341, 0.00341, −0.0034, 0.0034, 0.0034, 0.00339, −0.00339, −0.00338, 0.00338, −0.00338, 0.00338, 0.00337, −0.00334, 0.00334, −0.00333, −0.00333, 0.00333, 0.00332, −0.00332, −0.00332, 0.00332, −0.00331, −0.0033, −0.0033, −0.0033, −0.00329, −0.00329, −0.00329, 0.00328, 0.00328, −0.00327, 0.00327, −0.00327, 0.00326, 0.00326, −0.00326, 0.00323, 0.00323, 0.00322, −0.00322, −0.00321, −0.0032, 0.00319, −0.00319, −0.00318, −0.00318, −0.00318, −0.00316, −0.00315, 0.00314, 0.00314, 0.00314, −0.00313, 0.00313, 0.00312, −0.00312, 0.00312, 0.00311, 0.00311, −0.00311, 0.0031, 0.0031, −0.00309, −0.00309, −0.00308, 0.00307, −0.00307, 0.00307, 0.00306, −0.00306, −0.00306, −0.00306, 0.00306, 0.00305, −0.00305, −0.00304, −0.00304, −0.00303, 0.00302, −0.00302, −0.00302, −0.00301, 0.003, 0.003, 0.00298, −0.00298, −0.00298, −0.00298, 0.00297, 0.00297, 0.00296, 0.00296, 0.00296, 0.00296, 0.00295, 0.00295, −0.00295, 0.00295, −0.00294, −0.00294, −0.00294, 0.00294, −0.00294, 0.00293, −0.00293, −0.00292, 0.0029, 0.00289, 0.00289, 0.00288, −0.00288, 0.00287, 0.00287, 0.00286, 0.00286, −0.00285, 0.00285, 0.00284, 0.00284, −0.00281, 0.00281, 0.0028, −0.0028, 0.0028, 0.00279, 0.00279, 0.00277, 0.00277, 0.00276, 0.00276, −0.00275, −0.00274, 0.00274, 0.00274, 0.00273, −0.00273, 0.00272, −0.00272, −0.00272, −0.0027, −0.0027, 0.00269, 0.00269, 0.00269, 0.00269, 0.00267, 0.00267, −0.00267, −0.00265, 0.00265, −0.00264, 0.00264, −0.00264, −0.00264, 0.00263, −0.00262, −0.00262, −0.00261, 0.00261, −0.00261, 0.00261, −0.0026, −0.00258, −0.00258, 0.00257, 0.00257, 0.00256, 0.00256, −0.00255, −0.00255, −0.00254, −0.00253, −0.00253, −0.00252, −0.00251, 0.00249, 0.00249, −0.00249, 0.00249, 0.00248, −0.00247, 0.00247, −0.00247, −0.00245, 0.00245, −0.00245, −0.00243, 0.00243, −0.00243, −0.00241, 0.0024, 0.0024, −0.0024, 0.00239, 0.00238, −0.00238, 0.00236, 0.00236, 0.00236, 0.00235, −0.00235, 0.00234, −0.00234, 0.00232, −0.00232, 0.00231, 0.00231, −0.0023, 0.00229, −0.00229, 0.00229, −0.00227, −0.00227, 0.00227, 0.00226, 0.00226, −0.00225, 0.00225, −0.00225, 0.00225, −0.00224, 0.00223, −0.00222, 0.00222, −0.0022, 0.0022, 0.0022, −0.00219, −0.00219, 0.00219, −0.00219, 0.00218, −0.00218, −0.00218, −0.00218, −0.00218, −0.00217, −0.00217, 0.00216, 0.00216, 0.00216, 0.00215, 0.00213, 0.00213, −0.00213, −0.00213, −0.00212, −0.00211, −0.00211, −0.0021, −0.00209, 0.00208, −0.00208, −0.00208, 0.00205, −0.00205, 0.00205, −0.00204, 0.00203, −0.00203, −0.00202, −0.002, 0.002, 0.002, −0.002, −0.002, 0.00199, −0.00198, −0.00198, 0.00198, 0.00198, −0.00198, −0.00198, −0.00197, −0.00195, −0.00195, 0.00195, −0.00195, 0.00194, −0.00193, 0.00193, 0.00193, −0.00193, 0.00193, 0.00192, 0.00192, −0.00192, −0.00192, 0.00192, −0.00192, 0.00191, −0.0019, −0.0019, 0.0019, 0.00189, −0.00189, 0.00188, 0.00188, −0.00188, −0.00187, −0.00187, −0.00187, 0.00186, −0.00186, −0.00185, −0.00183, 0.00181, −0.00181, −0.00181, 0.0018, −0.00179, −0.00178, −0.00178, 0.00177, 0.00177, −0.00175, −0.00175, −0.00175, 0.00174, 0.00174, 0.00174, −0.00174, 0.00174, −0.00173, 0.00173, −0.00173, −0.00172, 0.00171, 0.0017, 0.00169, −0.00169, 0.00169, −0.00169, 0.00167, 0.00167, 0.00166, 0.00166, −0.00165, −0.00165, −0.00165, −0.00164, 0.00164, −0.00163, 0.00163, 0.00163, −0.00162, −0.00161, 0.0016, −0.0016, −0.0016, −0.0016, −0.0016, 0.00159, 0.00158, 0.00158, 0.00157, 0.00157, 0.00157, −0.00156, 0.00156, −0.00156, −0.00153, 0.00153, 0.00152, −0.00151, −0.00151, −0.0015, −0.0015, 0.00149, 0.00148, −0.00148, −0.00147, 0.00147, −0.00147, 0.00147, −0.00146, −0.00142, −0.00142, 0.00141, −0.00141, −0.0014, −0.00139, 0.00139, −0.00139, −0.00138, −0.00138, −0.00138, −0.00137, −0.00137, −0.00137, 0.00136, 0.00135, 0.00135, −0.00134, 0.00131, −0.00131, 0.0013, −0.00129, 0.00128, −0.00127, −0.00125, 0.00125, 0.00125, 0.00124, 0.00123, −0.00123, −0.00123, 0.00123, −0.00123, 0.00122, 0.0012, 0.0012, −0.00119, −0.00119, −0.00118, 0.00118, −0.00117, 0.00117, −0.00116, 0.00114, 0.00114, 0.00114, −0.00113, −0.00113, 0.00112, −0.00112, 0.00111, −0.00111, 0.00111, 0.0011, −0.0011, 0.00109, −0.00109, 0.00108, −0.00108, −0.00108, 0.00107, −0.00107, 0.00104, −0.00104, −0.00104, −0.00104, 0.00103, −0.00103, −0.00103, 0.00103, −0.00102, −0.00102, −0.00101, −0.00101, 0.00101, −0.001, 0.001, −0.001, 0.00099, 0.00098, −0.00097, 0.00096, 0.00096, −0.00094, 0.00093, −0.00093, −0.00093, −0.00093, 0.00091, −0.00091, −0.00091, 9.00E-04, −0.00089, −0.00087, −0.00087, −0.00086, 0.00086, 0.00085, 0.00085, −0.00084, 0.00083, 0.00083, −0.00083, 0.00082, −0.00082, −0.00082, 0.00082, 0.00081, −8.00E-04, 0.00079, −0.00079, −0.00078, 0.00077, −0.00077, −0.00076, −0.00076, 0.00074, 0.00074, −0.00074, −0.00072, 0.00072, −0.00069, −0.00068, 0.00068, 0.00068, 0.00068, −0.00068, −0.00068, −0.00068, −0.00067, 0.00067, −0.00066, −0.00063, −0.00063, −0.00062, −0.00061, −6.00E-04, −6.00E-04, 6.00E-04, −6.00E-04, 0.00059, 0.00058, −0.00058, 0.00058, 0.00058, −0.00056, 0.00055, 0.00055, −0.00054, 0.00054, −0.00054, 0.00052, −5.00E-04, 5.00E-04, −0.00049, −0.00049, 0.00048, 0.00048, 0.00048, −0.00048, −0.00047, 0.00047, −0.00046, −0.00046, 0.00045, 0.00043, −0.00043, −0.00042, −0.00042, −0.00042, 0.00041, 0.00041, 0.00041, 4.00E-04, 0.00039, −0.00038, 0.00038, 0.00038, −0.00038, −0.00038, −0.00038, 0.00038, −0.00037, −0.00037, −0.00037, −0.00035, 0.00034, −0.00033, 0.00033, −0.00032, −0.00031, −3.00E-04, −0.00029, −0.00029, 0.00029, 0.00029, 0.00027, 0.00027, 0.00026, −0.00026, −0.00025, 0.00024, 0.00023, −0.00023, 0.00023, −0.00022, −0.00022, −0.00022, 0.00021, 0.00021, 0.00021, −0.00019, −0.00019, −0.00018, −0.00018, −0.00017, −0.00017, −0.00017, 0.00017, −0.00017, −0.00017, 0.00016, −0.00016, 0.00016, 0.00015, −0.00014, −0.00013, −0.00013, 0.00012, −0.00012, −0.00011, 0.00011, −0.00011, −1.00E-04, −1.00E-04, 9.00E-05, 9.00E-05, −9.00E-05, −8.00E-05, −8.00E-05, −8.00E-05, 6.00E-05, 6.00E-05, −6.00E-05, −2.00E-05, −1.00E-05, 1.00E-05, 1.00E-05, −1.00E-05, 1.00E-05, −1.00E-05, −1.00E-05, and 0.

The predicting the presence, absence or development of cancer in an individual may particularly involve a threshold cancer index value being applied in order to assess or stratify an individual has having or not having cancer or of having a high or low risk of cancer development.

In any of the assays described herein, wherein when the cancer index value for the individual is about −0.056 or more, the individual is assessed as having cancer or as having a high risk of cancer development, or wherein when the cancer index value for the individual is less than about 0.056, the individual is assessed as not having cancer or as having a low risk of cancer development, preferably wherein the assay comprises determining methylation β-values for each CpG in the panel of one or more CpGs, and more preferably wherein the assessing the presence, absence or development of cancer in an individual is based on the WID-OC-Index. The panel of one or more CpGs used to derive the cancer index value may particularly comprise:
a. at least 500 of the CpGs identified at nucleotide positions 61 to 62 in SEQ ID NOs 1 to 14,000, and wherein the sensitivity is at least 64% and the specificity is at least 63%;
b. at least the CpGs defined by SEQ ID NOs 1 to 500 and identified at nucleotide positions 61 to 62, and wherein the sensitivity is at least 72% and specificity is at least 62%;
c. at least the CpGs defined by SEQ ID NOs 1 to 1000 and identified at nucleotide positions 61 to 62, and wherein the sensitivity is at least 80% and specificity is at least 61%; or
d. at least the CpGs defined by SEQ ID NOs 1 to 2000 and identified at nucleotide positions 61 to 62, and wherein the sensitivity is at least 76% and specificity is at least 61%.

In any of the assays described herein, wherein when the cancer index value for the individual is about −0.056 or more, the individual is assessed as having cancer or as having a high risk of cancer development, or wherein when the cancer index value for the individual is less than about 0.056, the individual is assessed as not having cancer or as having a low risk of cancer development, preferably wherein the assay comprises determining methylation β-values for each CpG in the panel of one or more CpGs, and more preferably wherein the assessing the presence, absence or development of cancer in an individual is based on the WID-OC-Index. The panel of one or more CpGs used to derive the cancer index value may particularly comprise:
1. at least the CpGs defined by SEQ ID NOs 1 to 500 and identified at nucleotide positions 61 to 62, and wherein the sensitivity is at least 72% and specificity is at least 62%;
2. at least the CpGs defined by SEQ ID NOs 501 to 100 and identified at nucleotide positions 61 to 62, and wherein the sensitivity is at least 77% and specificity is at least 58%; or
3. at least the CpGs defined by SEQ ID NOs 1001 to 1500 and identified at nucleotide positions 61 to 62, and wherein the sensitivity is at least 76% and specificity is at least 53%;
4. at least the CpGs defined by SEQ ID NOs 1501 to 2000 and identified at nucleotide positions 61 to 62, and wherein the sensitivity is at least 71% and specificity is at least 58%;
5. at least the CpGs defined by SEQ ID NOs 2001 to 2500 and identified at nucleotide positions 61 to 62, and wherein the sensitivity is at least 70% and specificity is at least 64%;
6. at least the CpGs defined by SEQ ID NOs 2501 to 3000 and identified at nucleotide positions 61 to 62, and wherein the sensitivity is at least 77% and specificity is at least 60%;
7. at least the CpGs defined by SEQ ID NOs 3001 to 3500 and identified at nucleotide positions 61 to 62, and wherein the sensitivity is at least 77% and specificity is at least 56%;
8. at least the CpGs defined by SEQ ID NOs 3501 to 4000 and identified at nucleotide positions 61 to 62, and wherein the sensitivity is at least 72% and specificity is at least 62%;
9. at least the CpGs defined by SEQ ID NOs 4001 to 4500 and identified at nucleotide positions 61 to 62, and wherein the sensitivity is at least 75% and specificity is at least 58%;
10. at least the CpGs defined by SEQ ID NOs 4501 to 5000 and identified at nucleotide positions 61 to 62, and wherein the sensitivity is at least 75% and specificity is at least 57%;
11. at least the CpGs defined by SEQ ID NOs 5001 to 5500 and identified at nucleotide positions 61 to 62, and wherein the sensitivity is at least 73% and specificity is at least 57%;
12. at least the CpGs defined by SEQ ID NOs 5501 to 6000 and identified at nucleotide positions 61 to 62, and wherein the sensitivity is at least 72% and specificity is at least 58%;
13. at least the CpGs defined by SEQ ID NOs 6001 to 6500 and identified at nucleotide positions 61 to 62, and wherein the sensitivity is at least 73% and specificity is at least 56%;
14. at least the CpGs defined by SEQ ID NOs 6501 to 7000 and identified at nucleotide positions 61 to 62, and wherein the sensitivity is at least 67% and specificity is at least 62%;
15. at least the CpGs defined by SEQ ID NOs 7001 to 7500 and identified at nucleotide positions 61 to 62, and wherein the sensitivity is at least 72% and specificity is at least 58%;
16. at least the CpGs defined by SEQ ID NOs 7501 to 8000 and identified at nucleotide positions 61 to 62, and wherein the sensitivity is at least 65% and specificity is at least 62%;
17. at least the CpGs defined by SEQ ID NOs 8001 to 8500 and identified at nucleotide positions 61 to 62, and wherein the sensitivity is at least 64% and specificity is at least 60%;
18. at least the CpGs defined by SEQ ID NOs 8501 to 9000 and identified at nucleotide positions 61 to 62, and wherein the sensitivity is at least 66% and specificity is at least 61%;
19. at least the CpGs defined by SEQ ID NOs 9001 to 9500 and identified at nucleotide positions 61 to 62, and wherein the sensitivity is at least 65% and specificity is at least 60%;
20. at least the CpGs defined by SEQ ID NOs 9501 to 10000 and identified at nucleotide positions 61 to 62, and wherein the sensitivity is at least 64% and specificity is at least 61%;
21. at least the CpGs defined by SEQ ID NOs 10001 to 10500 and identified at nucleotide positions 61 to 62, and wherein the sensitivity is at least 60% and specificity is at least 60%;
22. at least the CpGs defined by SEQ ID NOs 10501 to 11000 and identified at nucleotide positions 61 to 62, and wherein the sensitivity is at least 66% and specificity is at least 58%;
23. at least the CpGs defined by SEQ ID NOs 11001 to 11500 and identified at nucleotide positions 61 to 62, and wherein the sensitivity is at least 61% and specificity is at least 62%;
24. at least the CpGs defined by SEQ ID NOs 11501 to 12000 and identified at nucleotide positions 61 to 62, and wherein the sensitivity is at least 65% and specificity is at least 62%;
25. at least the CpGs defined by SEQ ID NOs 12001 to 12500 and identified at nucleotide positions 61 to 62, and wherein the sensitivity is at least 63% and specificity is at least 63%;
26. at least the CpGs defined by SEQ ID NOs 12501 to 13000 and identified at nucleotide positions 61 to 62, and wherein the sensitivity is at least 60% and specificity is at least 64%;
27. at least the CpGs defined by SEQ ID NOs 13001 to 13500 and identified at nucleotide positions 61 to 62, and wherein the sensitivity is at least 61% and specificity is at least 63%;
28. at least the CpGs defined by SEQ ID NOs 13501 to 14000 and identified at nucleotide positions 61 to 62, and wherein the sensitivity is at least 64% and specificity is at least 63%;
29. at least the CpGs defined by SEQ ID NOs 1 to 100 and identified at nucleotide positions 61 to 62, and wherein the sensitivity is at least 65% and specificity is at least 58%;
30. at least the CpGs defined by SEQ ID NOs 1 to 500 and identified at nucleotide positions 61 to 62, and wherein the sensitivity is at least 72% and specificity is at least 62%;
31. at least the CpGs defined by SEQ ID NOs 1 to 1000 and identified at nucleotide positions 61 to 62, and wherein the sensitivity is at least 80% and specificity is at least 61%;
32. at least the CpGs defined by SEQ ID NOs 1 to 2000 and identified at nucleotide positions 61 to 62, and wherein the sensitivity is at least 76% and specificity is at least 61%;
33. at least the CpGs defined by SEQ ID NOs 1 to 3000 and identified at nucleotide positions 61 to 62, and wherein the sensitivity is at least 73% and specificity is at least 63%;
34. at least the CpGs defined by SEQ ID NOs 1 to 4000 and identified at nucleotide positions 61 to 62, and wherein the sensitivity is at least 76% and specificity is at least 63%;
35. at least the CpGs defined by SEQ ID NOs 1 to 5000 and identified at nucleotide positions 61 to 62, and wherein the sensitivity is at least 77% and specificity is at least 61%;
36. at least the CpGs defined by SEQ ID NOs 1 to 6000 and identified at nucleotide positions 61 to 62, and wherein the sensitivity is at least 78% and specificity is at least 62%;
37. at least the CpGs defined by SEQ ID NOs 1 to 7000 and identified at nucleotide positions 61 to 62, and wherein the sensitivity is at least 78% and specificity is at least 61%;
38. at least the CpGs defined by SEQ ID NOs 1 to 8000 and identified at nucleotide positions 61 to 62, and wherein the sensitivity is at least 78% and specificity is at least 61%;
39. at least the CpGs defined by SEQ ID NOs 1 to 9000 and identified at nucleotide positions 61 to 62, and wherein the sensitivity is at least 78% and specificity is at least 60%;
40. at least the CpGs defined by SEQ ID NOs 1 to 10000 and identified at nucleotide positions 61 to 62, and wherein the sensitivity is at least 78% and specificity is at least 60%;
41. at least the CpGs defined by SEQ ID NOs 1 to 11000 and identified at nucleotide positions 61 to 62, and wherein the sensitivity is at least 78% and specificity is at least 60%;
42. at least the CpGs defined by SEQ ID NOs 1 to 12000 and identified at nucleotide positions 61 to 62, and wherein the sensitivity is at least 78% and specificity is at least 60%;
43. at least the CpGs defined by SEQ ID NOs 1 to 13000 and identified at nucleotide positions 61 to 62, and wherein the sensitivity is at least 78% and specificity is at least 60%;
44. at least the CpGs defined by SEQ ID NOs 1 to 14000 and identified at nucleotide positions 61 to 62, and wherein the sensitivity is at least 78% and specificity is at least 60%;
45. at least the CpGs defined by SEQ ID NOs 13,001 to 14,000 and identified at nucleotide positions 61 to 62, and wherein the sensitivity is at least 61% and specificity is at least 64%;
46. at least the CpGs defined by SEQ ID NOs 12,001 to 14,000 and identified at nucleotide positions 61 to 62, and wherein the sensitivity is at least 64% and specificity is at least 64%;
47. at least the CpGs defined by SEQ ID NOs 11,001 to 14,000 and identified at nucleotide positions 61 to 62, and wherein the sensitivity is at least 60% and specificity is at least 64%;
48. at least the CpGs defined by SEQ ID NOs 10,001 to 14,000 and identified at nucleotide positions 61 to 62, and wherein the sensitivity is at least 60% and specificity is at least 62%;
49. at least the CpGs defined by SEQ ID NOs 9,001 to 14,000 and identified at nucleotide positions 61 to 62, and wherein the sensitivity is at least 60% and specificity is at least 62%;
50. at least the CpGs defined by SEQ ID NOs 8,001 to 14,000 and identified at nucleotide positions 61 to 62, and wherein the sensitivity is at least 61% and specificity is at least 62%;
51. at least the CpGs defined by SEQ ID NOs 7,001 to 14,000 and identified at nucleotide positions 61 to 62, and wherein the sensitivity is at least 64% and specificity is at least 62%;
52. at least the CpGs defined by SEQ ID NOs 6,001 to 14,000 and identified at nucleotide positions 61 to 62, and wherein the sensitivity is at least 65% and specificity is at least 60%;
53. at least the CpGs defined by SEQ ID NOs 5,001 to 14,000 and identified at nucleotide positions 61 to 62, and wherein the sensitivity is at least 67% and specificity is at least 60%;
54. at least the CpGs defined by SEQ ID NOs 4,001 to 14,000 and identified at nucleotide positions 61 to 62, and wherein the sensitivity is at least 69% and specificity is at least 59%;
55. at least the CpGs defined by SEQ ID NOs 3,001 to 14,000 and identified at nucleotide positions 61 to 62, and wherein the sensitivity is at least 72% and specificity is at least 60%;
56. at least the CpGs defined by SEQ ID NOs 2,001 to 14,000 and identified at nucleotide positions 61 to 62, and wherein the sensitivity is at least 76% and specificity is at least 61%;
57. at least the CpGs defined by SEQ ID NOs 1,001 to 14,000 and identified at nucleotide positions 61 to 62, and wherein the sensitivity is at least 76% and specificity is at least 62%;
58. at least the CpGs defined by SEQ ID NOs 501 to 14,000 and identified at nucleotide positions 61 to 62, and wherein the sensitivity is at least 76% and specificity is at least 60%;
59. at least the CpGs defined by SEQ ID NOs 101 to 14,000 and identified at nucleotide positions 61 to 62, and wherein the sensitivity is at least 77% and specificity is at least 60%; or
60. at least the CpGs defined by SEQ ID NOs 1 to 14,000 and identified at nucleotide positions 61 to 62, and wherein the sensitivity is at least 78% and specificity is at least 60%.

In any of the described assays, the methylation status of the one or more CpGs in the panel is preferably determined by a p-value analysis, and the cancer is ovarian cancer or endometrial cancer. Preferably, the cancer is ovarian cancer.

In any of the assays described herein, wherein when the cancer index value for the individual is about 0.485 or more, the individual is assessed as having cancer or as having a high risk of cancer development, or wherein when the cancer index value for the individual is less than about 0.485, the individual is assessed as not having cancer or as having a low risk of cancer development, preferably wherein the assay comprises determining methylation β-values for each CpG in the panel of one or more CpGs, and more preferably wherein the assessing the presence, absence or development of cancer in an individual is based on the WID-OC-Index. The panel of one or more CpGs used to derive the cancer index value may particularly comprise:
a. at least 500 of the CpGs identified at nucleotide positions 61 to 62 in SEQ ID NOs 1 to 14,000, and wherein the sensitivity is at least 43% and the specificity is at least 80%;
b. at least the CpGs defined by SEQ ID NOs 1 to 500 and identified at nucleotide positions 61 to 62, and wherein the sensitivity is at least 51% and specificity is at least 87%;
c. at least the CpGs defined by SEQ ID NOs 1 to 1000 and identified at nucleotide positions 61 to 62, and wherein the sensitivity is at least 45% and specificity is at least 87%;
d. at least the CpGs defined by SEQ ID NOs 1 to 2000 and identified at nucleotide positions 61 to 62, and wherein the sensitivity is at least 49% and specificity is at least 89%.

In any of the assays described herein, wherein when the cancer index value for the individual is about 0.485 or more, the individual is assessed as having cancer or as having a high risk of cancer development, or wherein when the cancer index value for the individual is less than about 0.485, the individual is assessed as not having cancer or as having a low risk of cancer development, preferably wherein the assay comprises determining methylation β-values for each CpG in the panel of one or more CpGs, and more preferably wherein the assessing the presence, absence or development of cancer in an individual is based on the WID-OC-Index. The panel of one or more CpGs used to derive the cancer index value may particularly comprise:
1. at least the CpGs defined by SEQ ID NOs 1 to 500 and identified at nucleotide positions 61 to 62, and wherein the sensitivity is at least 51% and specificity is at least 87%;
2. at least the CpGs defined by SEQ ID NOs 501 to 100 and identified at nucleotide positions 61 to 62, and wherein the sensitivity is at least 51% and specificity is at least 82%; or
3. at least the CpGs defined by SEQ ID NOs 1001 to 1500 and identified at nucleotide positions 61 to 62, and wherein the sensitivity is at least 53% and specificity is at least 82%;
4. at least the CpGs defined by SEQ ID NOs 1501 to 2000 and identified at nucleotide positions 61 to 62, and wherein the sensitivity is at least 51% and specificity is at least 81%;
5. at least the CpGs defined by SEQ ID NOs 2001 to 2500 and identified at nucleotide positions 61 to 62, and wherein the sensitivity is at least 48% and specificity is at least 81%;
6. at least the CpGs defined by SEQ ID NOs 2501 to 3000 and identified at nucleotide positions 61 to 62, and wherein the sensitivity is at least 53% and specificity is at least 84%;
7. at least the CpGs defined by SEQ ID NOs 3001 to 3500 and identified at nucleotide positions 61 to 62, and wherein the sensitivity is at least 54% and specificity is at least 80%;
8. at least the CpGs defined by SEQ ID NOs 3501 to 4000 and identified at nucleotide positions 61 to 62, and wherein the sensitivity is at least 53% and specificity is at least 82%;
9. at least the CpGs defined by SEQ ID NOs 4001 to 4500 and identified at nucleotide positions 61 to 62, and wherein the sensitivity is at least 52% and specificity is at least 80%;
10. at least the CpGs defined by SEQ ID NOs 4501 to 5000 and identified at nucleotide positions 61 to 62, and wherein the sensitivity is at least 47% and specificity is at least 82%;
11. at least the CpGs defined by SEQ ID NOs 5001 to 5500 and identified at nucleotide positions 61 to 62, and wherein the sensitivity is at least 52% and specificity is at least 80%;
12. at least the CpGs defined by SEQ ID NOs 5501 to 6000 and identified at nucleotide positions 61 to 62, and wherein the sensitivity is at least 48% and specificity is at least 78%;
13. at least the CpGs defined by SEQ ID NOs 6001 to 6500 and identified at nucleotide positions 61 to 62, and wherein the sensitivity is at least 48% and specificity is at least 78%;
14. at least the CpGs defined by SEQ ID NOs 6501 to 7000 and identified at nucleotide positions 61 to 62, and wherein the sensitivity is at least 49% and specificity is at least 77%;
15. at least the CpGs defined by SEQ ID NOs 7001 to 7500 and identified at nucleotide positions 61 to 62, and wherein the sensitivity is at least 47% and specificity is at least 80%;
16. at least the CpGs defined by SEQ ID NOs 7501 to 8000 and identified at nucleotide positions 61 to 62, and wherein the sensitivity is at least 51% and specificity is at least 77%;
17. at least the CpGs defined by SEQ ID NOs 8001 to 8500 and identified at nucleotide positions 61 to 62, and wherein the sensitivity is at least 49% and specificity is at least 77%;
18. at least the CpGs defined by SEQ ID NOs 8501 to 9000 and identified at nucleotide positions 61 to 62, and wherein the sensitivity is at least 47% and specificity is at least 78%;
19. at least the CpGs defined by SEQ ID NOs 9001 to 9500 and identified at nucleotide positions 61 to 62, and wherein the sensitivity is at least 46% and specificity is at least 76%;
20. at least the CpGs defined by SEQ ID NOs 9501 to 10000 and identified at nucleotide positions 61 to 62, and wherein the sensitivity is at least 46% and specificity is at least 78%;
21. at least the CpGs defined by SEQ ID NOs 10001 to 10500 and identified at nucleotide positions 61 to 62, and wherein the sensitivity is at least 47% and specificity is at least 76%;
22. at least the CpGs defined by SEQ ID NOs 10501 to 11000 and identified at nucleotide positions 61 to 62, and wherein the sensitivity is at least 49% and specificity is at least 79%;
23. at least the CpGs defined by SEQ ID NOs 11001 to 11500 and identified at nucleotide positions 61 to 62, and wherein the sensitivity is at least 43% and specificity is at least 78%;
24. at least the CpGs defined by SEQ ID NOs 11501 to 12000 and identified at nucleotide positions 61 to 62, and wherein the sensitivity is at least 47% and specificity is at least 79%;
25. at least the CpGs defined by SEQ ID NOs 12001 to 12500 and identified at nucleotide positions 61 to 62, and wherein the sensitivity is at least 43% and specificity is at least 77%;
26. at least the CpGs defined by SEQ ID NOs 12501 to 13000 and identified at nucleotide positions 61 to 62, and wherein the sensitivity is at least 45% and specificity is at least 78%;
27. at least the CpGs defined by SEQ ID NOs 13001 to 13500 and identified at nucleotide positions 61 to 62, and wherein the sensitivity is at least 41% and specificity is at least 80%;
28. at least the CpGs defined by SEQ ID NOs 13501 to 14000 and identified at nucleotide positions 61 to 62, and wherein the sensitivity is at least 43% and specificity is at least 80%;
29. least the CpGs defined by SEQ ID NOs 1 to 100 and identified at nucleotide positions 61 to 62, and wherein the sensitivity is at least 41% and specificity is at least 84%;
30. at least the CpGs defined by SEQ ID NOs 1 to 500 and identified at nucleotide positions 61 to 62, and wherein the sensitivity is at least 51% and specificity is at least 87%;
31. at least the CpGs defined by SEQ ID NOs 1 to 1000 and identified at nucleotide positions 61 to 62, and wherein the sensitivity is at least 45% and specificity is at least 87%;
32. at least the CpGs defined by SEQ ID NOs 1 to 2000 and identified at nucleotide positions 61 to 62, and wherein the sensitivity is at least 49% and specificity is at least 89%;
33. at least the CpGs defined by SEQ ID NOs 1 to 3000 and identified at nucleotide positions 61 to 62, and wherein the sensitivity is at least 48% and specificity is at least 89%;
34. at least the CpGs defined by SEQ ID NOs 1 to 4000 and identified at nucleotide positions 61 to 62, and wherein the sensitivity is at least 47% and specificity is at least 89%;
35. at least the CpGs defined by SEQ ID NOs 1 to 5000 and identified at nucleotide positions 61 to 62, and wherein the sensitivity is at least 49% and specificity is at least 88%;
36. at least the CpGs defined by SEQ ID NOs 1 to 6000 and identified at nucleotide positions 61 to 62, and wherein the sensitivity is at least 51% and specificity is at least 88%;
37. at least the CpGs defined by SEQ ID NOs 1 to 7000 and identified at nucleotide positions 61 to 62, and wherein the sensitivity is at least 51% and specificity is at least 89%;
38. at least the CpGs defined by SEQ ID NOs 1 to 8000 and identified at nucleotide positions 61 to 62, and wherein the sensitivity is at least 48% and specificity is at least 89%;
39. at least the CpGs defined by SEQ ID NOs 1 to 9000 and identified at nucleotide positions 61 to 62, and wherein the sensitivity is at least 48% and specificity is at least 89%;
40. at least the CpGs defined by SEQ ID NOs 1 to 10000 and identified at nucleotide positions 61 to 62, and wherein the sensitivity is at least 48% and specificity is at least 88%;
41. at least the CpGs defined by SEQ ID NOs 1 to 11000 and identified at nucleotide positions 61 to 62, and wherein the sensitivity is at least 48% and specificity is at least 88%;
42. at least the CpGs defined by SEQ ID NOs 1 to 12000 and identified at nucleotide positions 61 to 62, and wherein the sensitivity is at least 48% and specificity is at least 88%;
43. at least the CpGs defined by SEQ ID NOs 1 to 13000 and identified at nucleotide positions 61 to 62, and wherein the sensitivity is at least 47% and specificity is at least 88%;
44. at least the CpGs defined by SEQ ID NOs 1 to 14000 and identified at nucleotide positions 61 to 62, and wherein the sensitivity is at least 48% and specificity is at least 88%;
45. at least the CpGs defined by SEQ ID NOs 13,001 to 14,000 and identified at nucleotide positions 61 to 62, and wherein the sensitivity is at least 45% and specificity is at least 80%;
46. at least the CpGs defined by SEQ ID NOs 12,001 to 14,000 and identified at nucleotide positions 61 to 62, and wherein the sensitivity is at least 45% and specificity is at least 79%;
47. at least the CpGs defined by SEQ ID NOs 11,001 to 14,000 and identified at nucleotide positions 61 to 62, and wherein the sensitivity is at least 40% and specificity is at least 78%;
48. at least the CpGs defined by SEQ ID NOs 10,001 to 14,000 and identified at nucleotide positions 61 to 62, and wherein the sensitivity is at least 45% and specificity is at least 78%;
49. at least the CpGs defined by SEQ ID NOs 9,001 to 14,000 and identified at nucleotide positions 61 to 62, and wherein the sensitivity is at least 45% and specificity is at least 78%;
50. at least the CpGs defined by SEQ ID NOs 8,001 to 14,000 and identified at nucleotide positions 61 to 62, and wherein the sensitivity is at least 46% and specificity is at least 78%;
51. at least the CpGs defined by SEQ ID NOs 7,001 to 14,000 and identified at nucleotide positions 61 to 62, and wherein the sensitivity is at least 46% and specificity is at least 78%;
52. at least the CpGs defined by SEQ ID NOs 6,001 to 14,000 and identified at nucleotide positions 61 to 62, and wherein the sensitivity is at least 47% and specificity is at least 78%;
53. at least the CpGs defined by SEQ ID NOs 5,001 to 14,000 and identified at nucleotide positions 61 to 62, and wherein the sensitivity is at least 49% and specificity is at least 77%;
54. at least the CpGs defined by SEQ ID NOs 4,001 to 14,000 and identified at nucleotide positions 61 to 62, and wherein the sensitivity is at least 48% and specificity is at least 78%;
55. at least the CpGs defined by SEQ ID NOs 3,001 to 14,000 and identified at nucleotide positions 61 to 62, and wherein the sensitivity is at least 47% and specificity is at least 80%;
56. at least the CpGs defined by SEQ ID NOs 2,001 to 14,000 and identified at nucleotide positions 61 to 62, and wherein the sensitivity is at least 47% and specificity is at least 80%;
57. at least the CpGs defined by SEQ ID NOs 1,001 to 14,000 and identified at nucleotide positions 61 to 62, and wherein the sensitivity is at least 51% and specificity is at least 84%;
58. at least the CpGs defined by SEQ ID NOs 501 to 14,000 and identified at nucleotide positions 61 to 62, and wherein the sensitivity is at least 48% and specificity is at least 84%;
59. at least the CpGs defined by SEQ ID NOs 101 to 14,000 and identified at nucleotide positions 61 to 62, and wherein the sensitivity is at least 48% and specificity is at least 86%; or
60. at least the CpGs defined by SEQ ID NOs 1 to 14,000 and identified at nucleotide positions 61 to 62, and wherein the sensitivity is at least 48% and specificity is at least 88%;

In any of the described assays, the methylation status of the one or more CpGs in the panel is preferably determined by a p-value analysis, and the cancer is ovarian cancer or endometrial cancer. Preferably, the cancer is ovarian cancer.

In any of the assays described herein, wherein when the cancer index value for the individual is about 1.006 or more, the individual is assessed as having cancer or as having a high risk of cancer development, or wherein when the cancer index value for the individual is less than about 1.006, the individual is assessed as not having cancer or as having a low risk of cancer development, preferably wherein the assay comprises determining methylation β-values for each CpG in the panel of one or more CpGs, and more preferably wherein the assessing the presence, absence or development of cancer in an individual is based on the WID-OC-Index. The panel of one or more CpGs used to derive the cancer index value may particularly comprise:
a. at least 500 of the CpGs identified at nucleotide positions 61 to 62 in SEQ ID NOs 1 to 14,000, and wherein the sensitivity is at least 30% and the specificity is at least 87%;
b. at least the CpGs defined by SEQ ID NOs 1 to 500 and identified at nucleotide positions 61 to 62, and wherein the sensitivity is at least 24% and specificity is at least 95%;
c. at least the CpGs defined by SEQ ID NOs 1 to 1000 and identified at nucleotide positions 61 to 62, and wherein the sensitivity is at least 22% and specificity is at least 95%;
d. at least the CpGs defined by SEQ ID NOs 1 to 2000 and identified at nucleotide positions 61 to 62, and wherein the sensitivity is at least 25% and specificity is at least 96%.

In any of the assays described herein, wherein when the cancer index value for the individual is about 1.006 or more, the individual is assessed as having cancer or as having a high risk of cancer development, or wherein when the cancer index value for the individual is less than about 1.006, the individual is assessed as not having cancer or as having a low risk of cancer development, preferably wherein the assay comprises determining methylation β-values for each CpG in the panel of one or more CpGs, and more preferably wherein the assessing the presence, absence or development of cancer in an individual is based on the WID-OC-Index. The panel of one or more CpGs used to derive the cancer index value may particularly comprise:
1. at least the CpGs defined by SEQ ID NOs 1 to 500 and identified at nucleotide positions 61 to 62, and wherein the sensitivity is at least 24% and specificity is at least 95%;
2. at least the CpGs defined by SEQ ID NOs 501 to 100 and identified at nucleotide positions 61 to 62, and wherein the sensitivity is at least 29% and specificity is at least 94%; or
3. at least the CpGs defined by SEQ ID NOs 1001 to 1500 and identified at nucleotide positions 61 to 62, and wherein the sensitivity is at least 30% and specificity is at least 93%;
4. at least the CpGs defined by SEQ ID NOs 1501 to 2000 and identified at nucleotide positions 61 to 62, and wherein the sensitivity is at least 27% and specificity is at least 93%;
5. at least the CpGs defined by SEQ ID NOs 2001 to 2500 and identified at nucleotide positions 61 to 62, and wherein the sensitivity is at least 29% and specificity is at least 93%;
6. at least the CpGs defined by SEQ ID NOs 2501 to 3000 and identified at nucleotide positions 61 to 62, and wherein the sensitivity is at least 36% and specificity is at least 94%;
7. at least the CpGs defined by SEQ ID NOs 3001 to 3500 and identified at nucleotide positions 61 to 62, and wherein the sensitivity is at least 33% and specificity is at least 93%;
8. at least the CpGs defined by SEQ ID NOs 3501 to 4000 and identified at nucleotide positions 61 to 62, and wherein the sensitivity is at least 33% and specificity is at least 91%;
9. at least the CpGs defined by SEQ ID NOs 4001 to 4500 and identified at nucleotide positions 61 to 62, and wherein the sensitivity is at least 31% and specificity is at least 92%;
10. at least the CpGs defined by SEQ ID NOs 4501 to 5000 and identified at nucleotide positions 61 to 62, and wherein the sensitivity is at least 31% and specificity is at least 92%;
11. at least the CpGs defined by SEQ ID NOs 5001 to 5500 and identified at nucleotide positions 61 to 62, and wherein the sensitivity is at least 35% and specificity is at least 92%;
12. at least the CpGs defined by SEQ ID NOs 5501 to 6000 and identified at nucleotide positions 61 to 62, and wherein the sensitivity is at least 31% and specificity is at least 90%;
13. at least the CpGs defined by SEQ ID NOs 6001 to 6500 and identified at nucleotide positions 61 to 62, and wherein the sensitivity is at least 31% and specificity is at least 89%;
14. at least the CpGs defined by SEQ ID NOs 6501 to 7000 and identified at nucleotide positions 61 to 62, and wherein the sensitivity is at least 30% and specificity is at least 87%;
15. at least the CpGs defined by SEQ ID NOs 7001 to 7500 and identified at nucleotide positions 61 to 62, and wherein the sensitivity is at least 36% and specificity is at least 92%;
16. at least the CpGs defined by SEQ ID NOs 7501 to 8000 and identified at nucleotide positions 61 to 62, and wherein the sensitivity is at least 31% and specificity is at least 87%;
17. at least the CpGs defined by SEQ ID NOs 8001 to 8500 and identified at nucleotide positions 61 to 62, and wherein the sensitivity is at least 34% and specificity is at least 86%;
18. at least the CpGs defined by SEQ ID NOs 8501 to 9000 and identified at nucleotide positions 61 to 62, and wherein the sensitivity is at least 30% and specificity is at least 87%;
19. at least the CpGs defined by SEQ ID NOs 9001 to 9500 and identified at nucleotide positions 61 to 62, and wherein the sensitivity is at least 34% and specificity is at least 86%;
20. at least the CpGs defined by SEQ ID NOs 9501 to 10000 and identified at nucleotide positions 61 to 62, and wherein the sensitivity is at least 27% and specificity is at least 88%;
21. at least the CpGs defined by SEQ ID NOs 10001 to 10500 and identified at nucleotide positions 61 to 62, and wherein the sensitivity is at least 31% and specificity is at least 87%;
22. at least the CpGs defined by SEQ ID NOs 10501 to 11000 and identified at nucleotide positions 61 to 62, and wherein the sensitivity is at least 30% and specificity is at least 87%;
23. at least the CpGs defined by SEQ ID NOs 11001 to 11500 and identified at nucleotide positions 61 to 62, and wherein the sensitivity is at least 31% and specificity is at least 86%;
24. at least the CpGs defined by SEQ ID NOs 11501 to 12000 and identified at nucleotide positions 61 to 62, and wherein the sensitivity is at least 30% and specificity is at least 89%;
25. at least the CpGs defined by SEQ ID NOs 12001 to 12500 and identified at nucleotide positions 61 to 62, and wherein the sensitivity is at least 33% and specificity is at least 86%;
26. at least the CpGs defined by SEQ ID NOs 12501 to 13000 and identified at nucleotide positions 61 to 62, and wherein the sensitivity is at least 30% and specificity is at least 87%;
27. at least the CpGs defined by SEQ ID NOs 13001 to 13500 and identified at nucleotide positions 61 to 62, and wherein the sensitivity is at least 30% and specificity is at least 87%;
28. at least the CpGs defined by SEQ ID NOs 13501 to 14000 and identified at nucleotide positions 61 to 62, and wherein the sensitivity is at least 30% and specificity is at least 87%;
29. at least the CpGs defined by SEQ ID NOs 1 to 100 and identified at nucleotide positions 61 to 62, and wherein the sensitivity is at least 22% and specificity is at least 95%;
30. at least the CpGs defined by SEQ ID NOs 1 to 500 and identified at nucleotide positions 61 to 62, and wherein the sensitivity is at least 24% and specificity is at least 95%;
31. at least the CpGs defined by SEQ ID NOs 1 to 1000 and identified at nucleotide positions 61 to 62, and wherein the sensitivity is at least 22% and specificity is at least 95%;
32. at least the CpGs defined by SEQ ID NOs 1 to 2000 and identified at nucleotide positions 61 to 62, and wherein the sensitivity is at least 25% and specificity is at least 96%;
33. at least the CpGs defined by SEQ ID NOs 1 to 3000 and identified at nucleotide positions 61 to 62, and wherein the sensitivity is at least 24% and specificity is at least 97%;
34. at least the CpGs defined by SEQ ID NOs 1 to 4000 and identified at nucleotide positions 61 to 62, and wherein the sensitivity is at least 24% and specificity is at least 97%;
35. at least the CpGs defined by SEQ ID NOs 1 to 5000 and identified at nucleotide positions 61 to 62, and wherein the sensitivity is at least 22% and specificity is at least 97%;
36. at least the CpGs defined by SEQ ID NOs 1 to 6000 and identified at nucleotide positions 61 to 62, and wherein the sensitivity is at least 23% and specificity is at least 97%;
37. at least the CpGs defined by SEQ ID NOs 1 to 7000 and identified at nucleotide positions 61 to 62, and wherein the sensitivity is at least 24% and specificity is at least 97%;
38. at least the CpGs defined by SEQ ID NOs 1 to 8000 and identified at nucleotide positions 61 to 62, and wherein the sensitivity is at least 25% and specificity is at least 97%;
39. at least the CpGs defined by SEQ ID NOs 1 to 9000 and identified at nucleotide positions 61 to 62, and wherein the sensitivity is at least 25% and specificity is at least 97%;
40. at least the CpGs defined by SEQ ID NOs 1 to 10000 and identified at nucleotide positions 61 to 62, and wherein the sensitivity is at least 24% and specificity is at least 97%;
41. at least the CpGs defined by SEQ ID NOs 1 to 11000 and identified at nucleotide positions 61 to 62, and wherein the sensitivity is at least 24% and specificity is at least 97%;
42. at least the CpGs defined by SEQ ID NOs 1 to 12000 and identified at nucleotide positions 61 to 62, and wherein the sensitivity is at least 25% and specificity is at least 97%;
43. at least the CpGs defined by SEQ ID NOs 1 to 13000 and identified at nucleotide positions 61 to 62, and wherein the sensitivity is at least 25% and specificity is at least 97%;
44. at least the CpGs defined by SEQ ID NOs 1 to 14000 and identified at nucleotide positions 61 to 62, and wherein the sensitivity is at least 25% and specificity is at least 97%;
45. at least the CpGs defined by SEQ ID NOs 13,001 to 14,000 and identified at nucleotide positions 61 to 62, and wherein the sensitivity is at least 30% and specificity is at least 88%;
46. at least the CpGs defined by SEQ ID NOs 12,001 to 14,000 and identified at nucleotide positions 61 to 62, and wherein the sensitivity is at least 30% and specificity is at least 87%;
47. at least the CpGs defined by SEQ ID NOs 11,001 to 14,000 and identified at nucleotide positions 61 to 62, and wherein the sensitivity is at least 31% and specificity is at least 87%;
48. at least the CpGs defined by SEQ ID NOs 10,001 to 14,000 and identified at nucleotide positions 61 to 62, and wherein the sensitivity is at least 33% and specificity is at least 87%;
49. at least the CpGs defined by SEQ ID NOs 9,001 to 14,000 and identified at nucleotide positions 61 to 62, and wherein the sensitivity is at least 33% and specificity is at least 87%;
50. at least the CpGs defined by SEQ ID NOs 8,001 to 14,000 and identified at nucleotide positions 61 to 62, and wherein the sensitivity is at least 31% and specificity is at least 87%;
51. at least the CpGs defined by SEQ ID NOs 7,001 to 14,000 and identified at nucleotide positions 61 to 62, and wherein the sensitivity is at least 31% and specificity is at least 86%;
52. at least the CpGs defined by SEQ ID NOs 6,001 to 14,000 and identified at nucleotide positions 61 to 62, and wherein the sensitivity is at least 29% and specificity is at least 87%;
53. at least the CpGs defined by SEQ ID NOs 5,001 to 14,000 and identified at nucleotide positions 61 to 62, and wherein the sensitivity is at least 30% and specificity is at least 87%;
54. at least the CpGs defined by SEQ ID NOs 4,001 to 14,000 and identified at nucleotide positions 61 to 62, and wherein the sensitivity is at least 31% and specificity is at least 88%;
55. at least the CpGs defined by SEQ ID NOs 3,001 to 14,000 and identified at nucleotide positions 61 to 62, and wherein the sensitivity is at least 34% and specificity is at least 90%;
56. at least the CpGs defined by SEQ ID NOs 2,001 to 14,000 and identified at nucleotide positions 61 to 62, and wherein the sensitivity is at least 34% and specificity is at least 92%;
57. at least the CpGs defined by SEQ ID NOs 1,001 to 14,000 and identified at nucleotide positions 61 to 62, and wherein the sensitivity is at least 30% and specificity is at least 93%;
58. at least the CpGs defined by SEQ ID NOs 501 to 14,000 and identified at nucleotide positions 61 to 62, and wherein the sensitivity is at least 30% and specificity is at least 94%;
59. at least the CpGs defined by SEQ ID NOs 101 to 14,000 and identified at nucleotide positions 61 to 62, and wherein the sensitivity is at least 29% and specificity is at least 96%; or
60. at least the CpGs defined by SEQ ID NOs 1 to 14,000 and identified at nucleotide positions 61 to 62, and wherein the sensitivity is at least 25% and specificity is at least 97%.

In any of the described assays, the methylation status of the one or more CpGs in the panel is preferably determined by a p-value analysis, and the cancer is ovarian cancer or endometrial cancer. Preferably, the cancer is ovarian cancer.

In any of the assays described herein, the sensitivity and specificity of the cancer index threshold values vary depending on the number of CpGs comprised within the set, and specifically what CpGs are comprised within the set. Tables 2, 3 and 4 exemplify this assertion.

In any of the assays of the invention described herein, the individual may be stratified according to their cancer index value, and consequently be defined according to their cancer status and/or cancer risk. In any of the assays described herein, wherein when the cancer index value for the individual is:
a. less than about −0.570 the individual is assessed as not having cancer;
b. about −0.570 or more and less than about −0.210 the individual is assessed as having a low risk of cancer;
c. about −0.210 or more and less than about 0.170 the individual is assessed as having a moderate risk of cancer;
d. about 0.170 or more the individual is assessed as having a high risk of cancer;
preferably wherein the assay comprises determining methylation β-values for each CpG in the panel of one or more CpGs, more preferably wherein the cancer is ovarian cancer.

TABLE 2

| | | Sensitivity | Specificity | Sensitivity | Specificity | Sensitivity | Specificity |
|---|---|---|---|---|---|---|---|
| | | (cancer index | (cancer index | (cancer index | (cancer index | (cancer index | (cancer index |
| CpG subset | AUC (internal | cutpoint = | cutpoint = | cutpoint = | cutpoint = | cutpoint = | cutpoint = |
| (SEQ ID NOs) | validation) | 1.006) | 1.006) | 0.485) | 0.485) | −0.056) | −0.056) |
| 1-14000 | 0.78 | 0.25 | 0.97 | 0.48 | 0.88 | 0.78 | 0.6 |
| 101-14000 | 0.77 | 0.29 | 0.96 | 0.48 | 0.86 | 0.77 | 0.6 |
| 501-14000 | 0.75 | 0.3 | 0.94 | 0.48 | 0.84 | 0.76 | 0.6 |
| 1001-14000 | 0.75 | 0.3 | 0.93 | 0.51 | 0.84 | 0.76 | 0.62 |
| 2001-14000 | 0.73 | 0.34 | 0.92 | 0.47 | 0.8 | 0.76 | 0.61 |
| 3001-14000 | 0.72 | 0.34 | 0.9 | 0.47 | 0.8 | 0.72 | 0.6 |
| 4001-14000 | 0.7 | 0.31 | 0.88 | 0.48 | 0.78 | 0.69 | 0.59 |
| 5001-14000 | 0.69 | 0.3 | 0.87 | 0.49 | 0.77 | 0.67 | 0.6 |
| 6001-14000 | 0.68 | 0.29 | 0.87 | 0.47 | 0.78 | 0.65 | 0.6 |
| 7001-14000 | 0.67 | 0.31 | 0.86 | 0.46 | 0.78 | 0.64 | 0.62 |
| 8001-14000 | 0.67 | 0.31 | 0.87 | 0.46 | 0.78 | 0.61 | 0.62 |
| 9001-14000 | 0.67 | 0.33 | 0.87 | 0.45 | 0.78 | 0.6 | 0.62 |
| 10001-14000 | 0.67 | 0.33 | 0.87 | 0.45 | 0.78 | 0.6 | 0.62 |
| 11001-14000 | 0.66 | 0.31 | 0.87 | 0.4 | 0.78 | 0.6 | 0.64 |
| 12001-14000 | 0.68 | 0.3 | 0.87 | 0.45 | 0.79 | 0.64 | 0.64 |
| 13001-14000 | 0.68 | 0.3 | 0.88 | 0.45 | 0.8 | 0.61 | 0.64 |

TABLE 3

| | | Sensitivity | Specificity | Sensitivity | Specificity | Sensitivity | Specificity |
|---|---|---|---|---|---|---|---|
| | | (cancer index | (cancer index | (cancer index | (cancer index | (cancer index | (cancer index |
| CpG subset | AUC (internal | cutpoint = | cutpoint = | cutpoint = | cutpoint = | cutpoint = | cutpoint = |
| (SEQ ID NOs) | validation) | 1.006) | 1.006) | 0.485) | 0.485) | −0.056) | −0.056) |
| 1-500 | 0.74 | 0.24 | 0.95 | 0.51 | 0.87 | 0.72 | 0.62 |
| 501-1000 | 0.74 | 0.29 | 0.94 | 0.51 | 0.82 | 0.77 | 0.58 |
| 1001-1500 | 0.74 | 0.3 | 0.93 | 0.53 | 0.82 | 0.76 | 0.53 |
| 1501-2000 | 0.7 | 0.27 | 0.93 | 0.51 | 0.81 | 0.71 | 0.58 |
| 2001-2500 | 0.72 | 0.29 | 0.93 | 0.48 | 0.81 | 0.7 | 0.64 |
| 2501-3000 | 0.75 | 0.36 | 0.94 | 0.53 | 0.84 | 0.77 | 0.6 |
| 3001-3500 | 0.74 | 0.3 | 0.93 | 0.54 | 0.8 | 0.77 | 0.56 |
| 3501-4000 | 0.73 | 0.33 | 0.91 | 0.53 | 0.82 | 0.72 | 0.62 |
| 4001-4500 | 0.73 | 0.33 | 0.92 | 0.52 | 0.8 | 0.75 | 0.58 |
| 4501-5000 | 0.75 | 0.31 | 0.92 | 0.47 | 0.82 | 0.75 | 0.57 |
| 5001-5500 | 0.73 | 0.35 | 0.92 | 0.52 | 0.8 | 0.73 | 0.57 |
| 5501-6000 | 0.7 | 0.31 | 0.9 | 0.48 | 0.78 | 0.72 | 0.58 |
| 6001-6500 | 0.7 | 0.31 | 0.89 | 0.48 | 0.78 | 0.73 | 0.56 |
| 6501-7000 | 0.68 | 0.3 | 0.87 | 0.49 | 0.77 | 0.67 | 0.62 |
| 7001-7500 | 0.73 | 0.36 | 0.92 | 0.47 | 0.8 | 0.72 | 0.58 |
| 7501-8000 | 0.69 | 0.31 | 0.87 | 0.51 | 0.77 | 0.65 | 0.62 |
| 8001-8500 | 0.69 | 0.34 | 0.86 | 0.49 | 0.77 | 0.64 | 0.6 |
| 8501-9000 | 0.68 | 0.3 | 0.87 | 0.47 | 0.78 | 0.66 | 0.61 |
| 9001-9500 | 0.66 | 0.34 | 0.86 | 0.46 | 0.76 | 0.65 | 0.6 |
| 9501-10000 | 0.68 | 0.27 | 0.88 | 0.46 | 0.78 | 0.64 | 0.61 |
| 10001-10500 | 0.67 | 0.31 | 0.87 | 0.47 | 0.76 | 0.6 | 0.6 |
| 10501-11000 | 0.68 | 0.3 | 0.87 | 0.49 | 0.79 | 0.66 | 0.58 |
| 11001-11500 | 0.65 | 0.31 | 0.86 | 0.43 | 0.78 | 0.61 | 0.62 |
| 11501-12000 | 0.68 | 0.3 | 0.89 | 0.47 | 0.79 | 0.65 | 0.62 |
| 12001-12500 | 0.67 | 0.33 | 0.86 | 0.43 | 0.77 | 0.63 | 0.63 |
| 12501-13000 | 0.67 | 0.3 | 0.87 | 0.45 | 0.78 | 0.6 | 0.64 |
| 13001-13500 | 0.66 | 0.3 | 0.87 | 0.41 | 0.8 | 0.61 | 0.63 |
| 13501-14000 | 0.67 | 0.3 | 0.87 | 0.43 | 0.8 | 0.64 | 0.63 |

TABLE 4

| | | Sensitivity | Specificity | Sensitivity | Specificity | Sensitivity | Specificity |
|---|---|---|---|---|---|---|---|
| | | (cancer index | (cancer index | (cancer index | (cancer index | (cancer index | (cancer index |
| CpG subset | AUC (internal | cutpoint = | cutpoint = | cutpoint = | cutpoint = | cutpoint = | cutpoint = |
| (SEQ ID NOs) | validation) | 1.006) | 1.006) | 0.485) | 0.485) | −0.056) | −0.056) |
| 1-100 | 0.69 | 0.22 | 0.95 | 0.41 | 0.84 | 0.65 | 0.58 |
| 1-500 | 0.74 | 0.24 | 0.95 | 0.51 | 0.87 | 0.72 | 0.62 |
| 1-1000 | 0.75 | 0.22 | 0.95 | 0.45 | 0.87 | 0.8 | 0.61 |
| 1-2000 | 0.75 | 0.25 | 0.96 | 0.49 | 0.89 | 0.76 | 0.61 |
| 1-3000 | 0.76 | 0.24 | 0.97 | 0.48 | 0.89 | 0.73 | 0.63 |
| 1-4000 | 0.77 | 0.24 | 0.97 | 0.47 | 0.89 | 0.76 | 0.63 |
| 1-5000 | 0.78 | 0.22 | 0.97 | 0.49 | 0.88 | 0.77 | 0.61 |
| 1-6000 | 0.78 | 0.23 | 0.97 | 0.51 | 0.88 | 0.78 | 0.62 |
| 1-7000 | 0.78 | 0.24 | 0.97 | 0.51 | 0.89 | 0.78 | 0.61 |
| 1-8000 | 0.78 | 0.25 | 0.97 | 0.48 | 0.89 | 0.78 | 0.61 |
| 1-9000 | 0.78 | 0.25 | 0.97 | 0.48 | 0.89 | 0.78 | 0.6 |
| 1-10000 | 0.78 | 0.24 | 0.97 | 0.48 | 0.88 | 0.78 | 0.6 |
| 1-11000 | 0.78 | 0.24 | 0.97 | 0.48 | 0.88 | 0.78 | 0.6 |
| 1-12000 | 0.78 | 0.25 | 0.97 | 0.48 | 0.88 | 0.78 | 0.6 |
| 1-13000 | 0.78 | 0.25 | 0.97 | 0.47 | 0.88 | 0.78 | 0.6 |
| 1-14000 | 0.78 | 0.25 | 0.97 | 0.48 | 0.88 | 0.78 | 0.6 |

### Relationship Between Cancer Index Value and Determining Methylation Status of CpGs

In view of the observations described herein (see Examples), the inventors derived a cancer index based on an analysis of methylation status (DNAme; as described above) for use in assays for assessing the presence or development of cancer in an individual.

As explained herein, the described assays particularly relate to the assessment of assessing the presence, absence or development of ovarian cancer and/or endometrial cancer, particularly ovarian cancer.

Any of the assays described herein involve deriving a cancer index value based on the methylation of status of a panel of one or more CpGs assayed in a sample provided from an individual, as described and defined herein.

The cancer index value may be derived by any suitable means.

The inventors have identified specific CpGs, as described and defined herein, which may be used to form a panel of CpGs whose methylation status is determined in order to establish cancer index values in accordance with the assays described and defined herein. Using these panels the inventors have demonstrated that it is possible to derive a cancer index value which correlates with and is indicative of normal tissue, i.e. tissue which is cancer negative, in particular ovarian and/or endometrial tissue which is cancer negative. Accordingly, cancer can be assessed to be absent in the individual. Using these panels the inventors have demonstrated that it is possible to derive a cancer index value which correlates with and is indicative of cancer tissue, i.e. tissue which is cancer positive, in particular ovarian and/or endometrial tissue which is cancer negative. Accordingly, cancer can be assessed to be present in the individual. As explained herein, the inventors have shown that using panels of the CpGs that have been identified it can be shown that the DNA methylation profile of epithelial cells from normal tissue such as from the cervix, vagina, buccal area, or from blood and/or urine, particularly from a liquid-based cytology sample, and more preferably a cervical smear sample, as indicated by the cancer index value, is dynamic and subject to change on a continuum from indicating cancer negative to cancer positive tissue. In particular, the cancer index value described herein acts as a surrogate for indicating whether the ovarian and/or endometrial tissue of an individual is cancer negative or cancer positive to a high degree of statistical accuracy. As such, using panels of the CpGs that have been identified it is possible to establish a cancer index value scale that can be used to assess the presence, absence or development of cancer in an individual.

As described herein, the inventors have used certain methods for determining the methylation status of specific CpGs in the population of DNA molecules in the sample. For example, in one method a percent methylated reference (PMR) value of a CpG may be determined. In another method the methylation β-values of a CpG may be determined. Different mechanisms may be employed to determine specific values depending on the circumstances, such as PCR-based mechanisms or array-based mechanisms.

As will be apparent to a skilled person, in the assays of the invention the steps of determining the methylation status of specific CpGs in the population of DNA molecules in the sample are not limited to any one specific methodology. As the skilled person will appreciate, because the cancer index value is based on the methylation status of CpGs, and since the methylation status of CpGs can be represented by values which may be specific to a specific methodology, e.g. percent methylated reference (PMR) value or methylation β-value, then the range of cancer index values which define cancer negative and cancer positive samples may be dependent upon the methodology used to determine the methylation status of CpGs. Nevertheless, a user may readily reproduce and implement the assays of the invention using any suitable methodology for determining the methylation status of CpGs, provided that the same methodology is used consistently. Moreover, the user can readily establish, de novo, cancer index values which define cancer negative and cancer positive samples by determining the methylation status of CpGs in panels constituting the specific CpGs disclosed herein from known cancer negative and cancer positive patient samples. Once such cancer index values are established using the CpGs identified herein, a user may use these values as a basis for assessing the presence, absence or development of cancer in any test individual whose cancer status is to be determined. Accordingly, cancer index values according to the present invention are not limited to specific methods of determination of methylation status of CpGs. On the contrary, the skilled person will appreciate that cancer index values can be established which reflect the intrinsic capabilities of the CpGs identified herein to correlate methylation status with cancer disease status.

Accordingly, the cancer index value may be derived by assessing the methylation status of the one or more CpGs in the panel in a sample provided from an individual by any suitable means.

The step of determining the methylation status of each CpG in the panel of one or more CpGs may be achieved by determining a percent methylated reference (PMR) value of each one of the one or more CpGs. The step of determining the methylation status of each CpG in the panel of one or more CpGs may be achieved by determining the methylation β-value of each one of the one or more CpGs.

In any of the assays described herein, the methylation status of the CpGs may be determined by any suitable means. For example, in any of the assays described herein the step of determining the methylation status of each CpG in the panel of one or more CpGs may comprise:
a. performing a sequencing step to determine the sequence of each CpG;
b. hybridising DNA to an array comprising probes capable of discriminating between methylated and non-methylated forms of the CpGs and applying a detection system to the array so as to determine the methylation status of each CpG; and/or
c. performing a PCR step using methylation-specific primers, wherein the methylation status of the CpG is determined by the presence or absence of a PCR product.

The step of determining the methylation status of each CpG in the panel of one or more CpGs may comprise a conversion step in order to distinguish methylated CpG dinucleotides relative to non-methylated CpG dinucleotides. The conversion step may comprise e.g. bisulfite conversion or TAPS (TET-assisted pyridine borane sequencing) conversion of the DNA in a sample that is to be applied to any one or more of a. to c. above. TAPS may particularly involve the steps of oxidising 5-methylcytosine bases (5mC) to 5-carboxylcytosine bases (5caC), preferably by ten-eleven translocation (TET), and/or oxidising 5-hydroxymethylcytosine bases (5hmC) to 5-carboxylcytosine bases (5caC), preferably by ten-eleven translocation (TET); followed by reducing 5-carboxylcytosine bases (5caC) to dihydrouracil bases (DHU), optionally with pyridine borane.

The step of determining the methylation status of each CpG in the panel of one or more CpGs may additionally, or alternatively, comprise the use of TempO-seq (templated Olig-sequencing). The oligonucleotides in the context of TempO-seq may or may not be designed such that they hybridise with methylated CpG dinucleotides following a prior conversion as described herein.

The step of determining the methylation status of each CpG in the panel of one or more CpGs may comprise the contacting the DNA in the sample with one or more methylation sensitive restriction endonucleases that cleave methylated and/or unmethylated forms of their restriction sites, and preferably the contacting of the DNA is prior to performing any one of a. to c. above. In assays of the invention wherein methylation sensitive restriction enzymes are used, one or more control reactions are performed. Preferably, the one or more control reactions involve interrogation of known loci that contain (i) no restriction endonuclease sites; (ii) a restriction site that is methylated; (iii) a restriction site that is unmethylated.

Using any of the methods for determining the methylation status of each CpG in the panel of one or more CpGs, the proportion of methylated and unmethylated CpGs at any given locus may be determined, thereby enabling generation of a cancer index value.

Preferably, the the step of determining in the population of DNA molecules in the sample the methylation status of a panel of one or more CpGs further comprises determining a β value of each CpG. Deriving the cancer index value may involve providing a methylation j-value data set comprising the methylation β-values for each CpG in the panel of one or more CpGs.

### Assessment of Methylation Status of CpGs

Methylation of DNA is a recognised form of epigenetic modification which has the capability of altering the expression of genes and other elements such as microRNAs. In cancer development and progression, methylation may have the effect of e.g. silencing tumor suppressor genes and/or increasing the expression of oncogenes. Other forms of dysregulation may occur as a result of methylation. Methylation of DNA occurs at discrete loci which are predominately dinucleotides consisting of a CpG motif, but may also occur at CHH motifs (where H is A, C, or T). During methylation, a methyl group is added to the fifth carbon of cytosine bases to create methylcytosine.

Methylation can occur throughout the genome and is not limited to regions with respect to an expressed sequence such as a gene. Methylation typically, but not always, occurs in a promoter or other regulatory region of an expressed sequence such as enhancer elements. Most typically, the methylation status of CpGs is clustered in CpG islands, for example CpG islands present in the regulatory regions of genes, especially in their promoter regions.

Typically, an assessment of DNA methylation status involves analysing the presence or absence of methyl groups in DNA, for example methyl groups on the 5 position of one or more cytosine nucleotides. Preferably, the methylation status of one or more cytosine nucleotides present as a CpG dinucleotide (where C stands for Cytosine, G for Guanine and p for the phosphate group linking the two) is assessed.

A variety of techniques are available for the identification and assessment of CpG methylation status, as will be outlined briefly below. The assays described herein encompass any suitable technique for the determination of CpG methylation status.

Methyl groups are lost from a starting DNA molecule during conventional in vitro handling steps such as PCR. To avoid this, techniques for the detection of methyl groups commonly involve the preliminary treatment of DNA prior to subsequent processing, in a way that preserves the methylation status information of the original DNA molecule. Such preliminary techniques involve three main categories of processing, i.e. bisulphite modification, restriction enzyme digestion and affinity-based analysis. Products of these techniques can then be coupled with sequencing or array-based platforms for subsequent identification or qualitative assessment of CpG methylation status.

Techniques involving bisulphite modification of DNA have become the most common assays for detection and assessment of methylation status of CpG dinucleotides. Treatment of DNA with bisulphite, e.g. sodium bisulphite, converts cytosine bases to uracil bases, but has no effect on 5-methylcytosines. Thus, the presence of a cytosine in bisulphite-treated DNA is indicative of the presence of a cytosine base which was previously methylated in the starting DNA molecule. Such cytosine bases can be detected by a variety of techniques. For example, primers specific for unmethylated versus methylated DNA can be generated and used for PCR-based identification of methylated CpG dinucleotides. DNA may be amplified, either before or after bisulphite conversion. A separation/capture step may be performed, e.g. using binding molecules such as complementary oligonucleotide sequences. Standard and next-generation DNA sequencing protocols can also be used.

In other approaches, methylation-sensitive enzymes can be employed which digest or cut only in the presence of methylated DNA. Analysis of resulting fragments is commonly carried out using mircroarrays.

Affinity-based techniques exploit binding interactions to capture fragments of methylated DNA for the purposes of enrichment. Binding molecules such as anti-5-methylcytosine antibodies are commonly employed prior to subsequent processing steps such as PCR and sequencing.

Olkhov-Mitsel and Bapat (2012) provide a comprehensive review of techniques available for the identification and assessment of biomarkers involving methylcytosine.

For the purposes of assessing the methylation status of the CpG-based biomarkers characterised and described herein, any suitable assay can be employed.

Assays described herein may comprise determining methylation status of CpGs by bisulphite converting the DNA. Preferred assays involve bisulphite treatment of DNA, including amplification of the identified CpG loci for methylation specific PCR and/or sequencing and/or assessment of the methylation status of target loci using methylation-discriminatory microarrays.

Amplification of CpG loci can be achieved by a variety of approaches. Preferably, CpG loci are amplified using PCR. A variety of PCR-based approaches may be used. For example, methylation-specific primers may be hybridized to DNA containing the CpG sequence of interest. Such primers may be designed to anneal to a sequence derived from either a methylated or non-methylated CpG locus. Following annealing, a PCR reaction is performed and the presence of a subsequent PCR product indicates the presence of an annealed CpG of identifiable sequence. In such assays, DNA is bisulphite converted prior to amplification. Such techniques are commonly referred to as methylation specific PCR (MSP).

In other techniques, PCR primers may anneal to the CpG sequence of interest independently of the methylation status, and further processing steps may be used to determine the status of the CpG. Assays are designed so that the CpG site(s) are located between primer annealing sites. This assay scheme is used in techniques such as bisulphite genomic sequencing, COBRA, Ms-SNuPE. In such assay, DNA can be bisulphite converted before or after amplification.

Small-scale PCR approaches may be used. Such approaches commonly involve mass partitioning of samples (e.g. digital PCR). These techniques offer robust accuracy and sensitivity in the context of a highly miniaturised system (pico-liter sized droplets), ideal for the subsequent handling of small quantities of DNA obtainable from the potentially small volume of cellular material present in biological samples, particularly urine samples. A variety of such small-scale PCR techniques are widely available. For example, microdroplet-based PCR instruments are available from a variety of suppliers, including RainDance Technologies, Inc. (Billerica, MA; http://raindancetech.com/) and Bio-Rad, Inc. (http://www.bio-rad.com/). Microarray platforms may also be used to carry out small-scale PCR. Such platforms may include microfluidic network-based arrays e.g. available from Fluidigm Corp. (www.fluidigm.com).

Following amplification of CpG loci, amplified PCR products may be coupled to subsequent analytical platforms in order to determine the methylation status of the CpGs of interest. For example, the PCR products may be directly sequenced to determine the presence or absence of a methylcytosine at the target CpG or analysed by array-based techniques.

Any suitable sequencing techniques may be employed to determine the sequence of target DNA. In the assays of the present invention the use of high-throughput, so-called “second generation”, “third generation” and “next generation” techniques to sequence bisulphite-treated DNA can be used.

In second generation techniques, large numbers of DNA molecules are sequenced in parallel. Typically, tens of thousands of molecules are anchored to a given location at high density and sequences are determined in a process dependent upon DNA synthesis. Reactions generally consist of successive reagent delivery and washing steps, e.g. to allow the incorporation of reversible labelled terminator bases, and scanning steps to determine the order of base incorporation. Array-based systems of this type are available commercially e.g. from Illumina, Inc. (San Diego, CA; http://www.illumina.com/).

Third generation techniques are typically defined by the absence of a requirement to halt the sequencing process between detection steps and can therefore be viewed as real-time systems. For example, the base-specific release of hydrogen ions, which occurs during the incorporation process, can be detected in the context of microwell systems (e.g. see the Ion Torrent system available from Life Technologies; http://www.lifetechnologies.com/). Similarly, in pyrosequencing the base-specific release of pyrophosphate (PPi) is detected and analysed. In nanopore technologies, DNA molecules are passed through or positioned next to nanopores, and the identities of individual bases are determined following movement of the DNA molecule relative to the nanopore. Systems of this type are available commercially e.g. from Oxford Nanopore (https://www.nanoporetech.com/). In an alternative assay, a DNA polymerase enzyme is confined in a “zero-mode waveguide” and the identity of incorporated bases are determined with florescence detection of gamma-labeled phosphonucleotides (see e.g. Pacific Biosciences; http://www.pacificbiosciences.com/).

In other assays sequencing steps may be omitted. For example, amplified PCR products may be applied directly to hybridization arrays based on the principle of the annealing of two complementary nucleic acid strands to form a double-stranded molecule. Hybridization arrays may be designed to include probes which are able to hybridize to amplification products of a CpG and allow discrimination between methylated and non-methylated loci. For example, probes may be designed which are able to selectively hybridize to an CpG locus containing thymine, indicating the generation of uracil following bisulphite conversion of an unmethylated cytosine in the starting template DNA. Conversely, probes may be designed which are able to selectively hybridize to a CpG locus containing cytosine, indicating the absence of uracil conversion following bisulphite treatment. This corresponds with a methylated CpG locus in the starting template DNA.

Following the application of a suitable detection system to the array, computer-based analytical techniques can be used to determine the methylation status of a CpG. Detection systems may include, e.g. the addition of fluorescent molecules following a methylation status-specific probe extension reaction. Such techniques allow CpG status determination without the specific need for the sequencing of CpG amplification products. Such array-based discriminatory probes may be termed methylation-specific probes.

Any suitable methylation-discriminatory microarrays may be employed to assess the methylation status of the CpGs described herein. One particular methylation-discriminatory microarray system is provided by Illumina, Inc. (San Diego, CA; http://www.illumina.com/). In particular, the Infinium MethylationEPIC BeadChip array and the Infinium HumanMethylation450 BeadChip array systems may be used to assess the methylation status of CpGs for predicting cancer development as described herein. Such a system exploits the chemical modifications made to DNA following bisulphite treatment of the starting DNA molecule. Briefly, the array comprises beads to which are coupled oligonucleotide probes specific for DNA sequences corresponding to the unmethylated form of a CpG, as well as separate beads to which are coupled oligonucleotide probes specific for DNA sequences corresponding to the methylated form of an CpG. Candidate DNA molecules are applied to the array and selectively hybridize, under appropriate conditions, to the oligonucleotide probe corresponding to the relevant epigenetic form. Thus, a DNA molecule derived from a CpG which was methylated in the corresponding genomic DNA will selectively attach to the bead comprising the methylation-specific oligonucleotide probe, but will fail to attach to the bead comprising the non-methylation-specific oligonucleotide probe. Single-base extension of only the hybridized probes incorporates a labeled ddNTP, which is subsequently stained with a fluorescence reagent and imaged. The methylation status of the CpG is determined by calculating the ratio of the fluorescent signal derived from the methylated and unmethylated sites.

Infinium HumanMethylation450 BeadChip array systems can be used to interrogate CpGs in the assays described herein. Alternative or customised arrays could, however, be employed to interrogate the cancer-specific CpG biomarkers defined herein, provided that they comprise means for interrogating all CpG for a given assay, as defined herein.

Techniques involving combinations of the above-described assays may also be used. For example, DNA containing CpG sequences of interest may be hybridized to microarrays and then subjected to DNA sequencing to determine the status of the CpG as described above.

In the assays described above, sequences corresponding to CpG loci may also be subjected to an enrichment process if desired. DNA containing CpG sequences of interest may be captured by binding molecules such as oligonucleotide probes complementary to the CpG target sequence of interest. Sequences corresponding to CpG loci may be captured before or after bisulphite conversion or before or after amplification. Probes may be designed to be complementary to bisulphite converted DNA. Captured DNA may then be subjected to further processing steps to determine the status of the CpG, such as DNA sequencing steps.

Capture/separation steps may be custom designed. Alternatively a variety of such techniques are available commercially, e.g. the SureSelect target enrichment system available from Agilent Technologies (http://www.agilent.com/home). In this system biotinylated “bait” or “probe” sequences (e.g. RNA) complementary to the DNA containing CpG sequences of interest are hybridized to sample nucleic acids. Streptavidin-coated magnetic beads are then used to capture sequences of interest hybridized to bait sequences. Unbound fractions are discarded. Bait sequences are then removed (e.g. by digestion of RNA) thus providing an enriched pool of CpG target sequences separated from non-CpG sequences. Template DNA may be subjected to bisulphite conversion and target loci amplified by small-scale PCR such as microdroplet PCR using primers which are independent of the methylation status of the CpG. Following amplification, samples may be subjected to a capture step to enrich for PCR products containing the target CpG, e.g. captured and purified using magnetic beads, as described above. Following capture, a standard PCR reaction is carried out to incorporate DNA sequencing barcodes into CpG-containing amplicons. PCR products are again purified and then subjected to DNA sequencing and analysis to determine the presence or absence of a methylcytosine at the target genomic CpG.

CpG biomarker loci defined herein by SEQ ID NOs 1 to 14,000 correspond to Illumina□ identifiers (IlmnID) known in the art. These CpG loci identifiers refer to individual CpG sites used in the commercially available Illumina® Infinium Methylation EPIC BeadChip kit and Illumina® Infinium Human Methylation450 BeadChip kit. The identity of each CpG site represented by each CpG loci identifier is publicly available from the Illumina, Inc. website under reference to the CpG sites used in the Infinium Methylation EPIC BeadChip kit and the Infinium Human Methylation450 BeadChip kit.

To complement evolving public databases to provide accurate CpG loci identifiers and strand orientation, Illumina® has developed a method to consistently designate CpG loci based on the actual or contextual sequence of each individual CpG locus. To unambiguously refer to CpG loci in any species, Illumina® has developed a consistent and deterministic CpG loci database to ensure uniformity in the reporting of methylation data. The Illumina® method takes advantage of sequences flanking a CpG locus to generate a unique CpG locus cluster ID. This number is based on sequence information only and is unaffected by genome version. Illumina's standardized nomenclature also parallels the TOP/BOT strand nomenclature (which indicates the strand orientation) commonly used for single nucleotide polymorphism (SNP) designation.

Illumina® Identifiers for the Infinium MethylationEPIC BeadChip and Infinium Human Methylation450 BeadChip system are also available from public repositories such as Gene Expression Omnibus (GEO) (http://www.ncbi.nlm.nih.gov/geo/).

By assessing the methylation status of a CpG it is meant that a determination is made as to whether a given CpG is methylated or unmethylated. In addition, it is meant that a determination is made as to the degree to which a given CpG site is methylated across a population of CpG loci in a sample.

CpG methylation status may be measured indirectly using a detection system such as fluorescence. A methylation-discriminatory microarray may be used. When calculating the degree of methylation of a given CpG, the Illumina® definition of beta-values may be used. The Illumina® methylation beta-value of a specific CpG site is calculated from the intensity of the methylated (M) and unmethylated (U) alleles, as the ratio of fluorescent signals β=Max(M,0)/[Max(M,0)+Max(U,0)+100]. On this scale, 0<β<1, β values of 1 or close to 1 indicate 100% methylation whereas values of 0 or close to 0 indicate 0% methylation.

The methylation status of any one or more CpGs of the 14,000 CpGs defined according to SEQ ID NOS: 1 to 14,000 may be assessed by any suitable technique. As explained in more detail in the Examples below, one particular exemplary technique which the inventors have used is a methylation discriminatory array, such as an Illumina InfiniumMethylation EPIC BeadChip. These assays utilise probes directed to methylated and unmethylated CpGs at a given locus.

Another exemplary technique which the inventors have used to determine the methylation status of any one or more CpGs is a fluorescence-based PCR technique referred to as MethyLight. These assays utilise forward and reverse PCR primers specific for sequences encompassing any one or more of the 14,000 CpGs defined according to SEQ ID NOS: 1 to 14,000. The methylation status of one or more of the 14,000 CpGs defined according to SEQ ID NOS: 1 to 14,000 may therefore be determined by MethyLight analysis. The detectable probes are typically designed such that they hybridise only to methylated forms of the one or more CpGs to be assayed.

### Bioinformatic Tools and Statistical Metrics for CpG-Based Assays

Software programs which aid in the in silico analysis of bisulphite converted DNA sequences and in primer design for the purposes of methylation-specific analyses are generally available and have been described previously.

In risk models for predicting cancer, a receiver-operating-characteristic (ROC) curve analysis is often used, in which the area under the curve (AUC) is assessed. Each point on the ROC curve shows the effect of a rule for turning a risk/likelihood estimate into a prediction of the presence, absence or development of cancer in an individual. The AUC measures how well the model discriminates between case subjects and control subjects. An ROC curve that corresponds to a random classification of case subjects and control subjects is a straight line with an AUC of 50%. An ROC curve that corresponds to perfect classification has an AUC of 100%.

In any of the methods described herein, the 95% confidence interval for the ROC AUC may be between 0.60 and 1.

In any of the methods described herein, the interval may be defined as a range having as an upper limit any number between 0.60 and 1. The upper limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99 or 1.00.

In any of the methods described herein, the interval may be defined as a range having as a lower limit any number between 0.60 and 1. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99 or 1.00.

In any of the methods described herein, the interval range may comprise any of the above lower limit numbers combined with any of the above upper limit numbers as appropriate.

Preferably, the upper limit number is 1. Thus, the 95% confidence ROC AUC interval may be defined as a range having an upper limit of 1 and as a lower limit any number between 0.60 and 1. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99 or 1.00.

The upper limit number may be 0.99. Thus, the 95% confidence ROC AUC interval may be defined as a range having an upper limit of 0.99 and as a lower limit any number between 0.60 and 0.99. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98 or 0.99.

The upper limit number may be 0.98. Thus, the 95% confidence ROC AUC interval may be defined as a range having an upper limit of 0.98 and as a lower limit any number between 0.60 and 0.98. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97 or 0.98.

The upper limit number may be 0.97. Thus, the 95% confidence ROC AUC interval may be defined as a range having an upper limit of 0.97 and as a lower limit any number between 0.60 and 0.97. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96 or 0.97.

The upper limit number may be 0.96. Thus, the 95% confidence ROC AUC interval may be defined as a range having an upper limit of 0.96 and as a lower limit any number between 0.60 and 0.96. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95 or 0.96.

The upper limit number may be 0.95. Thus, the 95% confidence ROC AUC interval may be defined as a range having an upper limit of 0.95 and as a lower limit any number between 0.60 and 0.95. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94 or 0.95.

The upper limit number may be 0.94. Thus, the 95% confidence ROC AUC interval may be defined as a range having an upper limit of 0.94 and as a lower limit any number between 0.60 and 0.94. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93 or 0.94.

The upper limit number may be 0.93. Thus, the 95% confidence ROC AUC interval may be defined as a range having an upper limit of 0.93 and as a lower limit any number between 0.60 and 0.93. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92 or 0.93.

The upper limit number may be 0.92. Thus, the 95% confidence ROC AUC interval may be defined as a range having an upper limit of 0.92 and as a lower limit any number between 0.60 and 0.92. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91 or 0.92.

The upper limit number may be 0.91. Thus, the 95% confidence ROC AUC interval may be defined as a range having an upper limit of 0.91 and as a lower limit any number between 0.60 and 0.91. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90 or 0.91.

The upper limit number may be 0.90. Thus, the 95% confidence ROC AUC interval may be defined as a range having an upper limit of 0.90 and as a lower limit any number between 0.60 and 0.90. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89 or 0.90.

The upper limit number may be 0.89. Thus, the 95% confidence ROC AUC interval may be defined as a range having an upper limit of 0.89 and as a lower limit any number between 0.60 and 0.89. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88 or 0.89.

The upper limit number may be 0.88. Thus, the 95% confidence ROC AUC interval may be defined as a range having an upper limit of 0.88 and as a lower limit any number between 0.60 and 0.88. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87 or 0.88.

The upper limit number may be 0.87. Thus, the 95% confidence ROC AUC interval may be defined as a range having an upper limit of 0.87 and as a lower limit any number between 0.60 and 0.87. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86 or 0.87.

The upper limit number may be 0.86. Thus, the 95% confidence ROC AUC interval may be defined as a range having an upper limit of 0.86 and as a lower limit any number between 0.60 and 0.86. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85 or 0.86.

The upper limit number may be 0.85. Thus, the 95% confidence ROC AUC interval may be defined as a range having an upper limit of 0.85 and as a lower limit any number between 0.60 and 0.85. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84 or 0.85.

The upper limit number may be 0.84. Thus, the 95% confidence ROC AUC interval may be defined as a range having an upper limit of 0.84 and as a lower limit any number between 0.60 and 0.84. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83 or 0.84.

The upper limit number may be 0.83. Thus, the 95% confidence ROC AUC interval may be defined as a range having an upper limit of 0.83 and as a lower limit any number between 0.60 and 0.83. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82 or 0.83.

The upper limit number may be 0.82. Thus, the 95% confidence ROC AUC interval may be defined as a range having an upper limit of 0.82 and as a lower limit any number between 0.60 and 0.82. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81 or 0.82.

The upper limit number may be 0.81. Thus, the 95% confidence ROC AUC interval may be defined as a range having an upper limit of 0.81 and as a lower limit any number between 0.60 and 0.81. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80 or 0.81.

The upper limit number may be 0.80. Thus, the 95% confidence ROC AUC interval may be defined as a range having an upper limit of 0.80 and as a lower limit any number between 0.60 and 0.80. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79 or 0.80.

The upper limit number may be 0.79. Thus, the 95% confidence ROC AUC interval may be defined as a range having an upper limit of 0.79 and as a lower limit any number between 0.60 and 0.79. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78 or 0.79.

The upper limit number may be 0.78. Thus, the 95% confidence ROC AUC interval may be defined as a range having an upper limit of 0.78 and as a lower limit any number between 0.60 and 0.78. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77 or 0.78.

The upper limit number may be 0.77. Thus, the 95% confidence ROC AUC interval may be defined as a range having an upper limit of 0.77 and as a lower limit any number between 0.60 and 0.77. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76 or 0.77.

The upper limit number may be 0.76. Thus, the 95% confidence ROC AUC interval may be defined as a range having an upper limit of 0.76 and as a lower limit any number between 0.60 and 0.76. The lower limit number may be 0.60, 0.61, 0.62, 0.63, 0.64, 0.65, 0.66, 0.67, 0.68, 0.69, 0.70, 0.71, 0.72, 0.73, 0.74, 0.75 or 0.76.

The upper limit number may be 0.75. Thus, the 95% confidence ROC AUC interval may be defined as a range having an upper limit of 0.75 and as a lower limit any number between 0.60 and 0.75. The lower limit number may be 0.60, 0.61, 0.62, 0.63, 0.64, 0.65, 0.66, 0.67, 0.68, 0.69, 0.70, 0.71, 0.72, 0.73, 0.74 or 0.75.

The upper limit number may be 0.74. Thus, the 95% confidence ROC AUC interval may be defined as a range having an upper limit of 0.74 and as a lower limit any number between 0.60 and 0.74. The lower limit number may be 0.60, 0.61, 0.62, 0.63, 0.64, 0.65, 0.66, 0.67, 0.68, 0.69, 0.70, 0.71, 0.72, 0.73 or 0.74.

The upper limit number may be 0.73. Thus, the 95% confidence ROC AUC interval may be defined as a range having an upper limit of 0.73 and as a lower limit any number between 0.60 and 0.73. The lower limit number may be 0.60, 0.61, 0.62, 0.63, 0.64, 0.65, 0.66, 0.67, 0.68, 0.69, 0.70, 0.71, 0.72 or 0.73.

The upper limit number may be 0.72. Thus, the 95% confidence ROC AUC interval may be defined as a range having an upper limit of 0.72 and as a lower limit any number between 0.60 and 0.72. The lower limit number may be 0.60, 0.61, 0.62, 0.63, 0.64, 0.65, 0.66, 0.67, 0.68, 0.69, 0.70, 0.71 or 0.72.

The upper limit number may be 0.71. Thus, the 95% confidence ROC AUC interval may be defined as a range having an upper limit of 0.71 and as a lower limit any number between 0.60 and 0.71. The lower limit number may be 0.60, 0.61, 0.62, 0.63, 0.64, 0.65, 0.66, 0.67, 0.68, 0.69, 0.70 or 0.71.

The upper limit number may be 0.70. Thus, the 95% confidence ROC AUC interval may be defined as a range having an upper limit of 0.70 and as a lower limit any number between 0.60 and 0.70. The lower limit number may be 0.60, 0.61, 0.62, 0.63, 0.64, 0.65, 0.66, 0.67, 0.68, 0.69 or 0.70.

### Methods of Treatment and Diagnosis

The term “treatment” as used herein is intended to refer to any intervention or procedure performed on an individual, including a surgical intervention or a pharmacological intervention such as the administration of a compound or drug. Any such treatment may be performed for therapeutic purposes or for preventative or prophylactic purposes.

The invention also encompasses the performance of one or more treatment steps following a positive classification of cancer, particularly ovarian and/or endometrial cancer, based on any of the methods described herein. Said treatments may be considered “therapeutic” treatments.

The invention also encompasses the performance of one or more treatment steps following a negative classification of cancer or prediction of an individual being at risk of cancer development, particularly ovarian and/or endometrial cancer, based on any of the methods described herein. Said treatments may be considered “risk prevention”, “preventative” or “prophylactic” treatments.

The invention also encompasses the performance of one or more treatment steps following a negative classification of cancer or prediction of an individual being at risk of cancer development based on any of the methods described herein, in an individual that harbours one or more mutations that predispose the individual to an increased risk of developing cancer, particularly ovarian and/or endometrial cancer, such as a BRCA1 and/or a BRCA2 mutation.

The invention thus encompasses a method of treating a cancer patient comprising administering chemotherapy, radiation, immunotherapy or any cancer therapy described herein to the patient determined to have a cancer index value which indicates that the patient has is positive for cancer based on any of the assays described herein, preferably wherein the cancer is ovarian cancer.

The invention thus encompasses a method of treating or preventing cancer in an individual, the method comprising:
a. assessing the cancer status of the individual by assessing the presence, absence or development of cancer in the individual by performing any one of the assays described herein;
b. administering one or more therapeutic or preventative treatments to the individual based on the assessment,
preferably wherein the cancer in ovarian and/or endometrial cancer, most
preferably wherein the cancer is ovarian cancer.

The invention thus encompasses a method of treating or preventing cancer in an individual, the method comprising:
a. assessing the cancer status of the individual by assessing the presence, absence or development of cancer in the individual comprising:
   i. providing a sample which has been taken from the individual, the sample comprising a population of DNA molecules;
   ii. determining in the population of DNA molecules in the sample the methylation status of a panel of one or more CpGs selected from a panel of CpGs identified at nucleotide positions 61 to 62 in SEQ ID NOs 1 to 14,000;
   iii. deriving a cancer index value based on the methylation status of the one or more CpGs in the panel; and
   iv. assessing the presence, absence or development of cancer in the individual based on the cancer index value, wherein the assay is characterised as having an area under the curve (AUC) of 0.60 or more as determined by receiver operating characteristics (ROC);
b. administering one or more therapeutic or preventative treatments to the individual based on the assessment,
preferably wherein the cancer in ovarian and/or endometrial cancer, most
preferably wherein the cancer is ovarian cancer.

In any of the methods of treatment encompassed by the invention, the step of predicting the presence or development of cancer, preferably wherein the cancer in ovarian and/or endometrial cancer, in an individual may involve deriving a cancer index value.

In any of the methods of treatment encompassed by the invention, the step of predicting the presence or development of cancer in an individual may involve the use of any one of the arrays described herein.

In any of the methods of treatment encompassed by the invention, the step of stratifying the individual may involve applying any one of the thresholds according to any one of the assays of the invention described herein.

The step of administering one or more treatments may comprise different treatment steps depending on the stratification of an individual on the basis of their risk of having cancer or on the basis of risk of cancer development, particularly ovarian and/or endometrial cancer. Particularly the amount of an invasiveness of the treatments administered may vary dependent on the stratification of an individual on the basis of their risk of having cancer or on the basis of their risk of cancer development. The treatments administered to the individual may comprise any treatments considered suitable by a person skilled in the art.

For example, wherein the individual is assessed as not having cancer or as having a low risk of cancer development, and wherein the cancer index value is about −0.570 or more and less than about −0.210, and preferably wherein the assay comprises determining methylation β-values for each CpG in the panel of one or more CpGs, the individual is subjected to one or more treatments according to their cancer index value, the one or more treatments may comprise intensified screening, preferably wherein the intensified screening comprises any of:
a. a test for a BRCA1 and/or BRCA2 germline mutation;
b. a test for CA125, preferably wherein the test is repeated annually;
c. a test for cell-free tumour DNA methylation in plasma/serum, preferably wherein the test is repeated annually;
d. a test for cell-free tumour DNA methylation in vaginal fluid, preferably wherein the test is repeated annually;
e. a repeat assay according to any one of the assays of the invention, preferably wherein the repeat assay is performed about two years after the previous assay,
   preferably wherein the individual being tested by any one or more of b. to d. is post-menopausal, preferably wherein the cancer is ovarian and/or endometrial cancer, most preferably wherein the cancer is ovarian cancer.

Wherein the individual is assessed as having cancer or as having a moderate risk of cancer development, and wherein the cancer index value is about −0.210 or more and less than about 0.170, and preferably wherein the assay comprises determining methylation β-values for each CpG in the panel of one or more CpGs, the individual is subjected to one or more treatments according to their cancer index value, the one or more treatments may comprise any of:
a. intensified screening, preferably wherein the intensified screening comprises one or more of:
   i. a test for a BRCA1 and/or BRCA2 germline mutation;
   ii. a test for CA125, preferably wherein the test is repeated annually;
   iii. a test for cell-free tumour DNA methylation in plasma/serum, preferably wherein the test is repeated annually;
   iv. a test for cell-free tumour DNA methylation in vaginal fluid, preferably wherein the test is repeated annually
   v. a pelvic MRI scan, preferably wherein the individual being subjected to the pelvic MRI scan is post-menopausal, and preferably wherein the scan is repeated annually;
   vi. a repeat assay according to any one of the assays of the invention, preferably wherein the repeat assay is performed about one year after the previous assay;
b. administration of one or more of Aspirin, oral contraceptive pill, selective estrogen receptor modulators (SERMS), and selective progesterone receptor modulators (SPRMs);
preferably wherein the cancer is ovarian and/or endometrial cancer, most preferably wherein the cancer is ovarian cancer.

Wherein the individual is assessed as having cancer or as having a high risk of cancer development, and wherein the cancer index value is about 0.170 or more, and preferably wherein the assay comprises determining methylation β-values for each CpG in the panel of one or more CpGs, the individual is subjected to one or more treatments according to their cancer index value, the one or more treatments may comprise any of:
a. intensified screening, preferably wherein the intensified screening comprises one or more of:
   i. a test for a BRCA1 and/or BRCA2 germline mutation;
   ii. a test for CA125, preferably wherein the test is repeated three-monthly;
   iii. a test for cell-free tumour DNA methylation in plasma/serum, preferably wherein the test is repeated annually;
   iv. a test for cell-free tumour DNA methylation in vaginal fluid, preferably wherein the test is repeated annually
   v. a pelvic MRI scan, preferably wherein the scan is repeated annually;
   vi. a repeat assay according to any one of the assays of the invention, preferably wherein the repeat assay is performed about one year after the previous assay;
b. administration of one or more of Aspirin, oral contraceptive pill, selective estrogen receptor modulators (SERMS), and selective progesterone receptor modulators (SPRMs); and/or
c. a total hysterectomy and bilateral salpingo-oophorectomy;
   preferably wherein the cancer is ovarian and/or endometrial cancer, most preferably wherein the cancer is ovarian cancer.

In any of the assays described herein, wherein tests are performed as part of intensified screening the tests may be repeated at any suitable interval. A test for CA125 may be performed three-monthly, six-monthly, annually or about once every two, three or four years. A test for cell-free tumour DNA methylation in plasma/serum may be performed three-monthly, six-monthly, annually or about once every two, three or four years. A test for cell-free tumour DNS methylation in vaginal fluid may be performed three-monthly, six-monthly, annually or about once every two, three or four years. A pelvic MRI scan may be performed three-monthly, six-monthly, annually or about once every two, three or four years.

Wherein the individual is assessed as having a moderate to high risk of having cancer or a moderate to high risk of cancer development, and/or if the individual has a germline tissue mutation of BRCA1 or BRCA2, preferably BRCA1, the one or more treatments may comprise administration of one or more of Aspirin, oral contraceptive pill, selective estrogen receptor modulators (SERMS), and selective progesterone receptor modulators (SPRMs). The SERMs may comprise Anordin, Bazedoxifene, Broparestrol, Broparestrol, Clomifene, Cyclofenil, Lasofoxifene, Ormeloxifene, Ospemifene, Raloxifene, Tamoxifen, preferably wherein the SERMs comprise Tamoxifen, Bazedoxifene and Raloxifene. Thee SPRMs may comprise Mifepristone, Ulipristal, Asoprisnil, Proellex, Onapristone, Asoprisnil and Lonaprisan.

Other exemplary treatments comprise one or more surgical procedures, one or more chemotherapeutic agents, one or more cytotoxic chemotherapeutic agents one or more radiotherapeutic agents, one or more immunotherapeutic agents, one or more biological therapeutics, one or more anti-hormonal treatments or any combination of the above following a positive diagnosis of cancer.

In any of the methods of treatment described herein, the individual may particularly be administered treatments recited in Table 7. Four sub-groups defined by ranges of cancer index values are specified in Table 7 as corresponding to preferred clinical actions, comprising intensified screening, administration of therapeutics and surgery.

Cancer treatments may be administered to an individual harbouring cancer or at risk of cancer development, in an amount sufficient to prevent, treat, cure, alleviate or partially arrest cancer or one or more of its symptoms. Such treatments may result in a decrease in severity, and/or decreased cancer index value, of cancer symptoms, or an increase in frequency or duration of symptom-free periods. A treatment amount adequate to accomplish this is defined as “therapeutically effective amount”. Effective amounts for a given purpose will depend on the severity of cancer and/or the individual's cancer index value as well as the weight and general state of the individual. As used herein, the term “individual” includes any human, preferably the human is a woman. As used herein, “treatment” is to be considered synonymous with “therapeutic agent”.

The following therapeutic agents may be administered to an individual based on their cancer risk alone or in combination with any other treatment described herein. The therapeutic agent may be directly attached, for example by chemical conjugation, to an antibody. Methods of conjugating agents or labels to an antibody are known in the art. For example, carbodiimide conjugation (Bauminger & Wilchek (1980) Methods Enzymol. 70, 151-159) may be used to conjugate a variety of agents, including doxorubicin, to antibodies or peptides. The water-soluble carbodiimide, 1-ethyl-3-(3-dimethylaminopropyl) carbodiimide (EDC) is particularly useful for conjugating a functional moiety to a binding moiety. Other methods for conjugating a moiety to antibodies can also be used. For example, sodium periodate oxidation followed by reductive alkylation of appropriate reactants can be used, as can glutaraldehyde cross-linking. However, it is recognised that, regardless of which method of producing a conjugate of the invention is selected, a determination must be made that the antibody maintains its targeting ability and that the functional moiety maintains its relevant function.

A cytotoxic moiety may be directly and/or indirectly cytotoxic. By “directly cytotoxic” it is meant that the moiety is one which on its own is cytotoxic. By “indirectly cytotoxic” it is meant that the moiety is one which, although is not itself cytotoxic, can induce cytotoxicity, for example by its action on a further molecule or by further action on it. The cytotoxic moiety may be cytotoxic only when intracellular and is preferably not cytotoxic when extracellular.

Cytotoxic chemotherapeutic agents are well known in the art. Cytotoxic chemotherapeutic agents, such as anticancer agents, include: alkylating agents including nitrogen mustards such as mechlorethamine (HN2), cyclophosphamide, ifosfamide, melphalan (L-sarcolysin) and chlorambucil; ethylenimines and methylmelamines such as hexamethylmelamine, thiotepa; alkyl sulphonates such as busulfan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); Antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin). Natural Products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes. Miscellaneous agents including platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MIH); and adrenocortical suppressant such as mitotane (o,p′-DDD) and aminoglutethimide; taxol and analogues/derivatives; and hormone agonists/antagonists such as flutamide and tamoxifen.

A cytotoxic chemotherapeutic agent may be a cytotoxic peptide or polypeptide moiety which leads to cell death. Cytotoxic peptide and polypeptide moieties are well known in the art and include, for example, ricin, abrin, Pseudomonas exotoxin, tissue factor and the like. Methods for linking them to targeting moieties such as antibodies are also known in the art. Other ribosome inactivating proteins are described as cytotoxic agents in WO 96/06641. Pseudomonas exotoxin may also be used as the cytotoxic polypeptide. Certain cytokines, such as TNFα and IL-2, may also be useful as cytotoxic agents.

Certain radioactive atoms may also be cytotoxic if delivered in sufficient doses. Radiotherapeutic agents may comprise a radioactive atom which, in use, delivers a sufficient quantity of radioactivity to the target site so as to be cytotoxic. Suitable radioactive atoms include phosphorus-32, iodine-125, iodine-131, indium-111, rhenium-186, rhenium-188 or yttrium-90, or any other isotope which emits enough energy to destroy neighbouring cells, organelles or nucleic acid. Preferably, the isotopes and density of radioactive atoms in the agents of the invention are such that a dose of more than 4000 cGy (preferably at least 6000, 8000 or 10000 cGy) is delivered to the target site and, preferably, to the cells at the target site and their organelles, particularly the nucleus.

The radioactive atom may be attached to an antibody, antigen-binding fragment, variant, fusion or derivative thereof in known ways. For example, EDTA or another chelating agent may be attached to the binding moiety and used to attach 111In or 90Y. Tyrosine residues may be directly labelled with 125I or 131I.

A cytotoxic chemotherapeutic agent may be a suitable indirectly-cytotoxic polypeptide. In a particularly preferred embodiment, the indirectly cytotoxic polypeptide is a polypeptide which has enzymatic activity and can convert a non-toxic and/or relatively non-toxic prodrug into a cytotoxic drug. With antibodies, this type of system is often referred to as ADEPT (Antibody-Directed Enzyme Prodrug Therapy). The system requires that the antibody locates the enzymatic portion to the desired site in the body of the patient and after allowing time for the enzyme to localise at the site, administering a prodrug which is a substrate for the enzyme, the end product of the catalysis being a cytotoxic compound. The object of the approach is to maximise the concentration of drug at the desired site and to minimise the concentration of drug in normal tissues. In a preferred embodiment, the cytotoxic moiety is capable of converting a non-cytotoxic prodrug into a cytotoxic drug.

In any of the methods of treatment described herein, the one or more treatments that the individual is subjected to may be repeated on one or more occasions. The one or more treatments may be repeated at regular intervals. The repetitive nature of the treatment administration may depend on the particular treatment being administered. Some treatments may require repetitive administration at greater frequency than others. The skilled person would be aware of the frequency of administration required for therapies known in the art. The one or more treatments may be repeated weekly, two weekly, three weekly, four weekly, monthly, three monthly, six monthly, yearly, two yearly, three yearly, four yearly, or five yearly.

In any of the methods described herein, when the individual is assessed as having a cancer index value of less than about −0.570, and preferably wherein the assay comprises determining methylation β-values for each CpG in the panel of one or more CpGs, no treatment is administered to the individual.

### Methods of Monitoring

The invention also provides methods of monitoring the risk of the presence or development of cancer in an individual.

“Monitoring” in the context of the present invention may refer to longitudinal assessment of an individual's risk of harbouring cancer or risk of cancer development. This longitudinal assessment may be carried out according to the assays of the invention described herein. This longitudinal assessment may involve performance of the assays of the invention described herein to predict the presence or development of cancer in an individual at more than one time point over the course of an undetermined time window. The time window may be any period of time whilst the individual is still living. The time window may persist for the lifetime of the individual. The time window may persist until the individual's risk of harbouring cancer or risk of cancer development falls below a certain level. The level may be a particular cancer index value.

The invention thus encompasses a method of monitoring for the presence, absence or development of cancer, particularly ovarian and/or endometrial cancer, most preferably ovarian cancer, in an individual, the method comprising:
a. assessing the presence or absence of cancer in an individual or assessing cancer development in an individual to establish a cancer status for the individual by performing any one of the assays of the invention described herein at a first time point;
b. assessing the presence or absence of cancer in the individual or assessing cancer development in the individual to establish a cancer status for the individual by performing any one of the assays of the invention described herein at one or more further time points, preferably wherein the cancer status of the individual in steps a and b are assessed using the same assay; and
c. monitoring any change in the cancer status of the individual between time points.

The invention also encompasses a method of monitoring for the presence, absence or development of cancer, particularly ovarian and/or endometrial cancer, most preferably ovarian cancer, in an individual, the method comprising:
a. assessing the presence or absence of cancer in an individual or assessing cancer development in an individual to establish a cancer status for the individual by performing an assay at a first time point, comprising:
   i. providing a sample which has been taken from the individual, the sample comprising a population of DNA molecules;
   ii. determining in the population of DNA molecules in the sample the methylation status of a panel of one or more CpGs selected from a panel of CpGs identified at nucleotide positions 61 to 62 in SEQ ID NOs 1 to 14,000;
   iii. deriving a cancer index value based on the methylation status of the one or more CpGs in the panel; and
   iv. assessing the presence, absence or development of cancer in the individual based on the cancer index value, wherein the assay is characterised as having an area under the curve (AUC) of 0.60 or more as determined by receiver operating characteristics (ROC);
b. assessing the presence or absence of cancer in the individual or assessing cancer development in the individual to establish a cancer status for the individual by performing the assay of steps a(i) to a(iv) or by performing any one of the assays of the invention described herein at one or more further time points; and
c. monitoring any change in the cancer status of the individual between time points.

In any of the methods of monitoring described herein, the steps of assessing the presence, absence or development of cancer in an individual based on a cancer index value may involve the application of threshold values. Threshold values can provide an indication of an individual's risk of having cancer or an individual's risk of cancer development. For example, cancer index values may indicate a high or low risk of harbouring or developing cancer. In any of the methods of monitoring encompassed by the invention, the step of predicting the presence, absence or development of cancer in an individual involves deriving a cancer index value.

The invention further encompasses a method of measuring methylation in a patient at multiple time points comprising (a) assessing the presence, absence or development of cancer in an individual by performing any one of the assays of the invention described herein at a first time point; (b) assessing the presence, absence or development of cancer in the individual by performing any one of the assays of the invention described herein at one or more further time points, and (c) detecting differential methylation status between (a) and (b).

In any of the methods of monitoring described herein, the individual may already harbour cancer, particularly ovarian and/or endometrial cancer. The individual may not have cancer. The individual may not harbour cancer. The individual may not harbour cancer but may harbour one or more genetic mutations that predispose the individual to an increased risk of cancer development e.g. the individual may harbour one or more mutations in a BRCA gene. Other mutations may include any mutations in the art that are considered to pre-dispose individuals to cancer. In any of the methods of monitoring described herein, the individual may not harbour cancer but may harbour one or more genetic mutations that pre-dispose the individual to cancer, and this individual may be subjected to any of the methods of monitoring described herein in order to determine their risk of having cancer or of developing cancer. For example, in any of the methods described herein, the individual does not harbour cancer and harbours one or more mutations that predispose the individual to an increased risk of developing cancer, particularly ovarian and/or endometrial cancer, and wherein one or more treatments are administered to the individual in accordance with any of the methods of treatment described herein as a method of prophylaxis. In any of the methods described herein, the individual does not harbour cancer and harbours one or more mutations that predispose the individual to an increased risk of developing cancer, and wherein one or more treatments are administered to the individual in accordance with any of the methods of treatment described herein as a method of prophylaxis, and wherein the one or more treatments administered to the individual comprises one or more doses of Aspirin, oral contraceptive pill, selective estrogen receptor modulators (SERMS), and selective progesterone receptor modulators (SPRMs). The SERMs may comprise Anordin, Bazedoxifene, Broparestrol, Broparestrol, Clomifene, Cyclofenil, Lasofoxifene, Ormeloxifene, Ospemifene, Raloxifene, Tamoxifen. The SPRMs may comprise Mifepristone, Ulipristal, Asoprisnil, Proellex, Onapristone, Asoprisnil and Lonaprisan. Preferably the one or more prophylactic treatments administered to the individual comprises Tamoxifen, Bazedoxifene and Raloxifene.

In any of the methods of monitoring described herein, depending on the risk of the presence or development of cancer in the individual, one or more treatments are administered to the individual according to any one of the methods of treatment encompassed by the invention and described herein, or wherein the cancer index value of the individual is less than about −0.570 no treatment is administered to the individual. Different treatments may be administered depending on the stratification of an individual on the basis of their risk of harbouring cancer or on the basis of their risk of cancer development. The method may further comprise administration of one or more treatments according to the methods of treatment described herein.

The cancer index value may change between any two or more time points. For this reason, longitudinal monitoring of an individual's cancer index value could be of particular benefit to the assessment of, for example, cancer progression, prevention of recurrence of cancer, cancer treatment efficacy, or cancer efficacy.

In any of the methods of monitoring described herein, the one or more further time points may be any suitable time point. Preferably the one or more further time points may of suitable distance apart for sufficiently frequent screening in order to predict any particularly early onset cases of presence or development of cancer in an individual. Preferably the one or more further time points may be of suitable distance apart for assessing the efficacy of one or more treatments. Preferably the one or more further time points may be of suitable distance apart for predicting whether an individual remains free of cancer after a successful course of treatment. The one or more further time points may be about monthly, about two monthly, about three monthly, about four monthly, about five monthly, about six monthly, about seven monthly, about eight monthly, about nine monthly, about ten monthly, about eleven monthly, about yearly, about two yearly, or more than two yearly.

In any of the methods of monitoring described herein, changes may be made to the one or more treatments wherein a positive or negative responses to the one or more treatments are observed. Treatments may be changed in accordance with the methods of treatments described herein. Treatments may particularly be changed if the individual's risk stratification, based on their cancer index value, changes.

In any of the methods of monitoring encompassed by the invention, the step of predicting the presence or development of cancer in an individual may involve the use of any one of the arrays described herein.

### Biological Samples

The assays described herein are preferably performed on samples comprising epithelial cells, particularly obtained from an anatomical site other than the ovary or endometrium. The sample may particularly be derived from the cervix, the vagina, the buccal area, blood and/or urine. The sample is preferably a cervical liquid-based cytology sample, and more preferably a cervical smear sample.

Preferably, any one of the assays described herein for assessing the presence, absence or development of cancer in an individual comprises providing a sample which has been taken from the individual. Preferably the individual is a woman.

In any of the assays described herein, the assay may or may not encompass the step of obtaining the sample from the individual. In assays which do not encompass the step of obtaining the sample from the individual, a sample which has previously been obtained from the individual is provided.

The sample may be provided directly from the individual for analysis or may be derived from stored material, e.g. frozen, preserved, fixed or cryopreserved material.

In any of the assays described herein, the sample may be self-collected or collected by any suitable medical professional.

Any of the assays described herein, the sample may comprise cells. The sample may comprise genetic material such as DNA and/or RNA.

Any of the assays described herein may involve providing a biological sample from the patient as the source of patient DNA for methylation analysis.

Any of the assays described herein may involve obtaining patient DNA from a biological sample which has previously been obtained from the patient.

Any of the assays described herein may involve obtaining a biological sample from the patient as the source of patient DNA for methylation analysis. The sample may be self-collected or collected by any suitable medical professional. Procedures for obtaining a biological sample include biopsy.

Methods for sample isolation and for the subsequent extraction and isolation of DNA from such cell or tissue samples in preparation for assessing DNA methylation, are well known to those skilled in the art. In the context of the assays or methods described herein, the entirety of a sample may be used, or alternatively cells may be concentrated or cell types may be fractionated in order to only apply subsets of one or more cell types to the present assays or methods. Any suitable methods of concentration or fractionation may be used.

In any one of the assays described and defined herein the sample from the individual, or the sample which has been taken from the individual, may derive from a tissue which is different from the tissue which harbours the tumour, if a tumour is present in the individual. Accordingly, in any one of the assays described and defined herein the sample from the individual, or the sample which has been taken from the individual, may not comprise nucleic acid, including DNA, which derives from the tumour, i.e. tumour-specific nucleic acid, including tumour-specific DNA. Thus, consistent with the data set out in the examples and the disclosures herein, methylation profiles derived from DNA molecules in the sample are used as surrogate markers for tumour-specific nucleic acid, including tumour-specific DNA, which exists at an anatomical site in the body of the individual which is remote from the anatomical site from which the sample is derived. A skilled person would able to identify the absence of tumour-specific DNA in any given population of sample-specific DNA molecules by routine means, such as determining the absence of known genetic mutations which are characteristic of the particular cancer, by e.g. sequence based screening. However, the performance of any one of the assays described and defined herein does not require any assessment to verify the absence of tumour-specific DNA in any given population of DNA molecules.

### Types of Cancers

The methods described herein may be applied to any cancer. Preferably, the methods described herein may be applied to ovarian cancer and/or endometrial cancer. The methods described herein are most preferably applied to ovarian cancer.

The cancer may be a primary cancer lesion. The cancer may be a secondary cancer lesion. The cancer may be a metastatic lesion.

In assays described herein, wherein the assays is for assessing the presence, absence or development of ovarian cancer, the ovarian cancer may preferably be serious carcinoma, mucinous carcinoma, endometrioid carcinoma, clear cell carcinoma, lop malignant potential (LMP) tumor, borderline epithelial ovarian cancer, teratoma, dysgerminoma, endodermal sinus tumor, Choriocarcinoma, granulosa-theca tumor, Sertoli-Leydig tumor, granulosa cell tumor, small cell carcinoma of the ovary or primary peritoneal carcinoma. Any of the assays described herein may additionally, or alternatively, be for assessing the presence, absence or development of endometrial cancer.

In assays described herein, wherein the assays is for assessing the presence, absence or development of endometrial cancer, the endometrial cancer may preferably be an endometriod cancer, uterine carcinosarcoma, squamous cell carcinoma, small cell carcinoma, transitional carcinoma, serous carcinoma, clear-cell carcinoma, mucinous adenocarcinoma, undifferentiated carcinoma, dedifferentiated carcinoma or serous adenocarcinoma.

### Arrays and Kits

The invention also encompasses arrays capable of discriminating between methylated and non-methylated forms of CpGs as defined herein; the arrays may comprise oligonucleotide probes specific for methylated forms of CpGs as defined herein and oligonucleotide probes specific for non-methylated forms of CpGs as defined herein.

In any of the arrays described herein, the array may comprise oligonucleotide probes specific for a methylated form of each CpG in a CpG panel and oligonucleotide probes specific for a non-methylated form of each CpG in the panel; wherein the panel consists of at least 500 CpGs selected from the CpGs identified at nucleotide position 61 to 62 in SEQ ID NOs 1 to 14000.

The panel may consist of at least 1000 CpGs selected from the CpGs identified at nucleotide position 61 to 62 in SEQ ID NOs 1 to 14000, preferably wherein the CpGs are the CpGs identified at nucleotide position 61 to 62 in SEQ ID NOs 1 to 1000.

The panel may consist of at least 2000 CpGs selected from the CpGs identified at nucleotide position 61 to 62 in SEQ ID NOs 1 to 14000, preferably wherein the CpGs are the CpGs identified at nucleotide position 61 to 62 in SEQ ID NOs 1 to 2000.

The panel may consist of at least 3000 CpGs selected from the CpGs identified at nucleotide position 61 to 62 in SEQ ID NOs 1 to 14000, preferably wherein the CpGs are the CpGs identified at nucleotide position 61 to 62 in SEQ ID NOs 1 to 3000.

The panel may consist of at least 4000 CpGs selected from the CpGs identified at nucleotide position 61 to 62 in SEQ ID NOs 1 to 14000, preferably wherein the CpGs are the CpGs identified at nucleotide position 61 to 62 in SEQ ID NOs 1 to 4000.

The panel may consist of at least 5000 CpGs selected from the CpGs identified at nucleotide position 61 to 62 in SEQ ID NOs 1 to 14000, preferably wherein the CpGs are the CpGs identified at nucleotide position 61 to 62 in SEQ ID NOs 1 to 5000.

The panel may consist of at least 6000 CpGs selected from the CpGs identified at nucleotide position 61 to 62 in SEQ ID NOs 1 to 14000, preferably wherein the CpGs are the CpGs identified at nucleotide position 61 to 62 in SEQ ID NOs 1 to 6000.

The panel may consist of at least 7000 CpGs selected from the CpGs identified at nucleotide position 61 to 62 in SEQ ID NOs 1 to 14000, preferably wherein the CpGs are the CpGs identified at nucleotide position 61 to 62 in SEQ ID NOs 1 to 7000.

The panel may consist of at least 8000 CpGs selected from the CpGs identified at nucleotide position 61 to 62 in SEQ ID NOs 1 to 14000, preferably wherein the CpGs are the CpGs identified at nucleotide position 61 to 62 in SEQ ID NOs 1 to 8000.

The panel may consist of at least 9000 CpGs selected from the CpGs identified at nucleotide position 61 to 62 in SEQ ID NOs 1 to 14000, preferably wherein the CpGs are the CpGs identified at nucleotide position 61 to 62 in SEQ ID NOs 1 to 9000.

The panel may consist of at least 10000 CpGs selected from the CpGs identified at nucleotide position 61 to 62 in SEQ ID NOs 1 to 14000, preferably wherein the CpGs are the CpGs identified at nucleotide position 61 to 62 in SEQ ID NOs 1 to 10000.

The panel may consist of at least 11000 CpGs selected from the CpGs identified at nucleotide position 61 to 62 in SEQ ID NOs 1 to 14000, preferably wherein the CpGs are the CpGs identified at nucleotide position 61 to 62 in SEQ ID NOs 1 to 11000.

The panel may consist of at least 12000 CpGs selected from the CpGs identified at nucleotide position 61 to 62 in SEQ ID NOs 1 to 14000, preferably wherein the CpGs are the CpGs identified at nucleotide position 61 to 62 in SEQ ID NOs 1 to 12000.

The panel may consist of at least 13000 CpGs selected from the CpGs identified at nucleotide position 61 to 62 in SEQ ID NOs 1 to 14000, preferably wherein the CpGs are the CpGs identified at nucleotide position 61 to 62 in SEQ ID NOs 1 to 13000.

The panel may consist of all CpGs identified in SEQ ID NOs 1 to 14000.

In some embodiments the array is not an Infinium MethylationEPIC BeadChip array or an Illumina Infinium HumanMethylation450 BeadChip array.

Separately or additionally, in some embodiments the number of CpG-specific oligonucleotide probes of the array is 482,000 or less, 480,000 or less, 450,000 or less, 440,000 or less, 430,000 or less, 420,000 or less, 410,000 or less, or 400,000 or less, 375,000 or less, 350,000 or less, 325,000 or less, 300,000 or less, 275,000 or less, 250,000 or less, 225,000 or less, 200,000 or less, 175,000 or less, 150,000 or less, 125,000 or less, 100,000 or less, 75,000 or less, 50,000 or less, 45,000 or less, 40,000 or less, 35,000 or less, 30,000 or less, 25,000 or less, 20,000 or less, 15,000 or less, 10,000 or less, 5,000 or less, 4,000 or less, 3,000 or less or 2,000 or less.

The CpG panel may comprise any set of CpGs defined in the assays of the invention described herein.

The arrays of the invention may comprise one or more oligonucleotides comprising any set of CpGs defined in the assays of the invention, wherein the one or more oligonucleotides are hybridized to corresponding oligonucleotide probes of the array.

The invention also encompasses a process for making a hybridized array described herein, comprising contacting an array according to the present invention with a group of oligonucleotides comprising any set of CpGs defined in the assays of the invention.

Any of the arrays as defined herein may be comprised in a kit. The kit may comprise any array as defined herein together with instructions for use.

The invention further encompasses the use of any of the arrays as defined herein in any of the assays for determining the methylation status of CpGs for the purposes of predicting the presence or development of cancer in an individual.

The following Examples serve to illustrate but not to limit the invention.

### EXAMPLES

In the Examples described herein, WID-OC-Index is a cancer index value wherein the index value has been determined by assaying in a population of DNA molecules derived from a given sample from an individual the methylation status of a panel of CpGs selected from the CpGs defined by SEQ ID NOs: 1 to 14,000.

In some instances within the Examples, all CpGs defined by SEQ ID NOs: 1 to 14,00 have been included in the panel which has been assayed to obtain a cancer index value. In addition, specific sub-selections of CpGs from among the 14,000 CpGs defined by SEQ ID NOs: 1 to 14,000 have been included in the panel which has been assayed to obtain a cancer index value. In these instances, the cancer index value's ability to discriminate between cancer positive and cancer negative women is described, wherein discriminatory ability of the index is characterised by AUC and received operating characteristics.

To date, DNA methylation of only a very few genes has been assessed in cervical smear samples from women with and without ovarian cancer and not one of these very small studies reached significance after multiple test adjustment (17).

Here, the inventors performed an epigenome-wide DNAme analysis in cervical smear samples from women who were subsequently diagnosed with ovarian cancer, and in matched controls, and established the WID-OC-index (Women's risk IDentification for Ovarian Cancer index) which was further validated in an independent set of cervical samples. The inventors established that the WID-OC signature is not driven by tumour DNA, is high in healthy women with a BRCA1 mutation and has a very high sensitivity/specificity that also identifies women with endometrial cancer, which also arises from Müllerian Duct epithelial cells. In addition, the inventors assessed the WID-OC-index in normal fimbrial and high grade serous tissue and cell lines and a large range of tissue samples and showed that the WID-OC index is significantly associated with the rate of BRCA-1/2 germline-mediated cancer formation.

### Materials and Methods

### Study Design and Epidemiological Data Acquisition

The study was conducted as part of a multi-centre study involving several recruitment sites in five European countries (i.e. the UK, Czech Republic, Italy, Norway and Germany). Women over the age of 18 were eligible to participate in the study who had not undergone a previous hysterectomy, had not received treatment (within 2 years of recruitment) for a non-gynaecological cancer, were not pregnant or menstruating at time of recruitment and had not undergone a cervical smear in the last 12 weeks. Prior to taking part, each prospective study volunteer was given a Participant Information Sheet as well as a Consent Form and the rationale for the study was explained. Additional resources, including an explanatory video and further online resources, were also made available. Women diagnosed with ovarian or endometrial cancer (case) or a non-malignant benign gynaecological condition (control) were approached during outpatient hospital clinics, while women recruited with a documented BRCA1 or BRCA2 mutation or as healthy volunteers from the general population (control) were approached via outreach campaigns, public engagement, and as part of cervical screening programmes. After signing an informed consent, participants completed an epidemiological questionnaire as well as a feedback form after their participation. The study itself is a sub-study of the FORECEE (4C) Programme, which has ethical approval from the UK Health Research Authority (REC 14/LO/1633).

The epidemiological survey was administered via the Qualtrics web-based survey application on dedicated iPads. The survey contained questions relating to current and historical health habits, relevant risk factors, as well as obtaining a thorough medical and obstetric history. Cervical samples were collected at appropriate clinical venues by trained staff and the cervical smears were carried out by a small group of research midwives or physicians with a view to establishing standard practice.

Biological samples and survey data were pseudo-anonymised using a participant study number. Each recruitment site maintained a securely stored file linking personal identifiers to the study number. Following sample taking, an email survey was sent to each participant, enabling them to feedback with respect to the recruitment process. Women with a current diagnosis of (a) primary malignant ovarian cancer of high grade serous, endometrioid, mucinous or clear cell morphology or (b) endometrial cancer with poor prognostic features (endometrioid, serous or clear cell morphology of Grade III and/or stage >IB) and recruited prior to receiving any systemic chemotherapy treatment or surgery or radiotherapy were eligible as ovarian or endometrial cancer cases. Cancer histological data was collected post-recruitment either by clinicians directly involved in the diagnosis/treatment of the cancer cases or by a nominated data manager with access to the in-house hospital systems.

### Cervical Smear Sample Collection

Cervical smears were taken at collaborating hospitals and recruitment centres using the ThinPrep system (Hologic Inc., cat #70098-002). Cervical cells were sampled from the cervix using a cervix brush (Rovers Medical Devices, cat #70671-001) which was rotated 5 times through 360 degrees whilst in contact with the cervix to maximise cell sampling. The brush was removed from the vagina and immersed in a ThinPrep vial containing Preserve-cyt fluid and then pushed against the bottom of the vial 10 times to facilitate release of the cells from the brush into the solution. The sample vial was sealed and stored locally at room temperature.

### Primary Fallopian Tube Secretory Epithelial Cell Culture

Patients undergoing a salpingectomy at University College London Hospitals (UCLH), were consented in order to donate Fallopian Tube tissue surplus to diagnostic requirements following UCL ethical guidelines (women provided written informed consent and samples were collected under the NRES Committee London-Surrey Borders Research Ethics Committee approval; 14/LO/1633). Fimbrial Fallopian Tube secretory epithelial cells were isolated and cultured as described previously (T. E. Bartlett et al., Epigenetic reprogramming of fallopian tube fimbriae in BRCA mutation carriers defines early ovarian cancer evolution. Nat Commun 7, 11620 (2016)) with slight modifications. Briefly, fimbrial tissues were carefully excised by an experienced pathologist, macerated and digested in a dissociation medium (0.05% collagenase and 0.01% DNase in DMEM) for 48 h at 4° C. Cells were harvested by centrifugation and resuspended in DMEM/F-12 supplemented with 2% Ultroser G (Pall Corporation, France) and 1% penicillin-streptomycin, transferred into a tissue culture flask. Cells were phenotyped: firstly by determining mRNA expression of PAX8 (Müllerian marker) and Cytokeratin 7 (CK7, epithelial marker) using quantitative PCR; and secondly by immunofluorescent staining. All experiments were performed before the cells started to senesce, and all FT cells were used without modification (such as hTERT or SV40T antigen immortalisation) in order to enhance self-renewal.

### Real-Time PCR Analysis

MethyLight, a quantitative PCR analysis specific to bisulfite converted DNA, was performed on cervical smear samples from 20 endometrial cancer patients, 20 ovarian cancer patients, and 20 controls collected as part of the FORCEE project. Primers and probes specific to ZNF154: forward primer: 5′-TTT ATT TTA GGT TTG ACG TGG GTT T-3′, reverse primer: 5′-CGT CGT CCC TCC TAC ACG AA-3′, probe sequence: 5′-6-Fam-TAG GGC GGC GTC GTT AAG GTT TAG ACG-BHQ-1-3′ were used. Ct values of the target reaction were normalised for DNA concentration using a reference gene reaction against COL2A1: forward primer: 5′-TCT AAC AAT TAT AAA CTC CAA CCA CCA A-3′, reverse primer: 5′-GGG AAG ATG GGA TAG AAG GGA ATA T-3′, probe sequence: 5′-6-Fam-CCT TCA TTC TAA CCC AAT ACC TAT CCC ACC TCT AAA-BHQ-1-3′. Specificity of the reactions for methylated DNA was confirmed separately using SssI-treated fully methylated human white blood cell DNA. The percentage of fully methylated molecules at a specific locus was calculated by dividing the ZNF154:COL2A1 ratio of a sample by the ZNF154:COL2A1 ratio of the SssI-treated human white blood cell DNA and multiplied by 100. Results are expressed as ‘PMR’ (percentage of methylated reference).

### Sample Processing and DNA Extraction

When preparing for sample storage in the laboratory, cervical smear samples were poured into 50 ml Falcon tubes and left to sediment at room temperature for 2 hours. 1 mL wide bore tips were then used to transfer the enriched cellular sediment into a 2 mL vial. The cervical sediments were washed twice with PBS, lysed, and stored temporarily at −20° C. ahead of extraction. For ovarian tissues, DNA was extracted from 30 mg of tissue using the AllPrep DNA/RNA Mini Kit (#80204, Qiagen Ltd), following the manufacturer's protocol. DNA concentration and quality absorbance ratios were measured using Nanodrop-8000, Thermoscientific Inc. Extracted DNA was stored at −80° C. until further analysis.

### DNA Methylation Array Analysis

Cervical, and Fallopian Tube and ovarian cancer cell line DNA was normalised to 25 ng/μl and 500 ng total DNA was bisulfite modified using the EZ-96 DNA Methylation-Lightning kit (Zymo Research Corp, cat #D5047) on the Hamilton Star Liquid handling platform. 8 μl of modified DNA was subjected to methylation analysis on the Illumina InfiniumMethylation EPIC BeadChip (Illumina, CA, USA) at UCL Genomics according to the manufacturer's standard protocol.

### Methylation Analysis

All methylation microarray data were processed through the same standardised pipeline. Raw data was loaded using the R package minfi. Any samples with median methylated and unmethylated intensities <9.5 were removed. Any probes with a detection p-value >0.01 were regarded as failed. Any samples with >10% failed probes, and any probes with >10% failure rate were removed from the dataset. Beta values from failed probes (approximately 0.001% of the dataset) were imputed using the impute.knn function as part of the impute R package.

Non-CpG probes (2,932), SNP-related probes as identified by Zhou et.al. (W. Zhou, P. W. Laird, H. Shen, Comprehensive characterization, annotation and innovative use of Infinium DNA methylation BeadChip probes. Nucleic Acids Res 45, e22 (2017)) (82,108), and chrY probes were removed from the dataset. An additional 6,102 previously identified probes that followed a trimodal methylation pattern characteristic of an underlying SNP were removed.

Background intensity correction and dye bias correction was performed using the minfi single sample preprocessNoob function. Probe bias correction was performed using the beta mixture quantile normalisation (BMIQ) algorithm.

The fraction of immune cell contamination, and the relative proportions of different immune cell subtypes in each sample, were estimated using the EpiDISH algorithm using the epithelial, fibroblast and immune cell reference dataset. The top 1,000 most variable probes (ranked by standard deviation) were used in a principal component analysis. Statistical tests were performed in order to identify any anomalous associations between plate, sentrix position, date of array processing, date of DNA creation, study centre, immune contamination fraction, age, type (case versus control) and the top ten principal components. Finally, two-thirds of the discovery dataset was randomly selected for use as the training dataset and the remaining third was allocated to the internal validation dataset. This split was carried out once, and the same training and validation sets were used in all subsequent analyses.

113 samples were downloaded from the ENCODE database (https://www.encodeproject.org/). The beta mixture quantile normalisation was applied to these samples after using minfi to extract beta values.

### Statistical Analyses for Classifier Development

Contamination by immune cells presented a challenge with respect to the identification of differentially methylated positions (DMPs) as differential methylation that occurred solely in epithelial cells was diminished in samples with high IC and vice versa. In order to overcome this, the inventors linearly regressed the beta values on IC for each CpG site, the linear models being fitted to cases and controls separately. The intercept points at IC=0 were used as estimates of mean beta values in cases and controls in a pure epithelial cell population. The difference between these intercept points provided a delta-beta estimate in epithelial cells. The difference between intercept points at IC=1 provided immune cell delta-beta estimates. A list of ranked CpGs was produced according to delta-beta estimates in epithelial cells.

The R package glmnet was used to train classifiers with a mixing parameter value of alpha=0 (ridge penalty) and alpha=1 (lasso penalty) with binomial response type. Data from the training dataset were used to fit the classifiers. The top n CpGs from the list of CpGs ranked by epithelial delta-beta estimates were used as inputs to the classifier. Ten-fold cross-validation was used inside the training set by the cv.glmnet function in order to determine the optimal value of the regularisation parameter lambda. The AUC was used as a metric of classifier performance which was evaluated on the internal validation dataset as a function of n, the number of CpGs used as inputs during training. The maximum value of n was 30,000.

The optimal classifier was selected based on the highest AUC obtained in the internal validation dataset. Once the optimal number of inputs was determined, the training and internal validation datasets were combined and the classifier was refitted using the entire discovery dataset with alpha and lambda fixed to their optimal values. This finalised classifier was then applied to the external validation dataset and the corresponding AUC was computed.

Denoting the top n CpGs as x₁, . . . , xₙ and the regression coefficients from the trained classifier as w₁, . . . , wₙ then WID-OC-index=Σᵢ₌₁ⁿ(wᵢxᵢ−μ)/σ where μ and σ are defined as the mean and standard deviation of the quantity Σᵢ₌₁ⁿ wᵢxᵢ in the training dataset (that is, the index is scaled to have zero mean and unit standard deviation in the training dataset).

### Enrichment Analyses

The epithelial delta-beta estimates were used to compute the top 1,000 hyper and hypo CpGs. These were used as inputs to the eFORGE 2.0 tool 20 (accessed at https://eforge.altiusinstitute.org/). Data from the “Consolidated Roadmap Epigenomics DHS” were used for the analysis. The default options of 1 kb proximity window, 1,000 background repetitions, and strict and marginal significance thresholds of 0.01 and 0.05 were used.

A gene set enrichment analysis (GSEA) 21 was carried out by first selecting for each gene TSS200 region in the CpG with the largest epithelial delta-beta estimate (both hyper- and hypo-methylated). Genes were then ranked according to the absolute value of these delta-beta estimates. The C2 curated gene set, c2.all.v6.2.symbols.gmt, was downloaded from MSigDB. The fgsea R package was used to perform the enrichment analysis with parameters minSize, maxSize, and nperm set to 15, 500, and 10,000 respectively.

### Estimation of Tumour DNA Proportion

The Epidish algorithm provides an estimate of cell type proportions within a given sample. A reference dataset consisting of CpGs that are unique to each cell type must be provided. In order to construct such a reference dataset 11 epithelial, 7 fibroblast, 48 immune, and 11 high grade serous ovarian cancer cell line samples were downloaded from GEO (Additional Data 1). Each cell type was in turn compared to the other three cell types (which were combined into one group) in order to identify CpGs that are unique to that cell type. A Wilcoxon rank sum test was used to test for differential methylation at each CpG. For epithelial cells any CpGs with a p-value >0.01 after false discovery rate (FDR) adjustment and an absolute difference in methylation >0.54 were selected (204 in total). For fibroblasts any CpGs with FDR adjusted p-values >0.01 and differential methylation >0.7 were selected (208 in total). For immune cells any CpGs with FDR adjusted p-values >0.01 and differential methylation >0.89 were selected (225 in total). For HGSOC cells any CpGs with FDR adjusted p-values >0.01 and differential methylation >0.77 were selected (203 in total). The final reference dataset therefore consisted of 840 CpGs.

It was observed that the inferred proportion of tumour DNA and epithelial cells were strongly associated in control samples. Local polynomial regression fitting (using the loess R function) was used to regress the inferred tumour DNA proportion on the epithelial proportion (in control samples only, FIG. 11A) and the residuals were used as estimates for tumour DNA proportion.

### Estimation of Epithelial and Immune Variance.

The inventors aimed to estimate how much variability across the 14,000 CpGs in the WID-OC-index could be attributed to epithelial cells or immune cells. An example of a CpG with high variability in epithelial cells and low variability in immune cells is given in FIG. 11B. For each CpG the inventors applied the following model. The inventors assumed that the epithelial beta values follow a beta distribution Beta(β|a₀, b₀) with shape parameters a₀>0 and b₀>0, and that immune beta values followed Beta(β|a₁, b₁) with shape parameters a₁>0 and b₁>0. The inventors assumed that each sample is a combination of epithelial and immune cells and that ρᵢϵ[0,1] is the proportion of immune cells in sample i, i=1, . . . , N. The quantities ρᵢ were obtained from the EpiDISH algorithm. The following log likelihood function was numerically optimised with respect to a₀, b₀, a₁, b₁:

$$\\ L ( {a}_{0} , {b}_{0} , {a}_{1} , {b}_{1} ) = - \frac{1}{N} {\sum }_{i = 1}^{N} log [ ( 1 - {ρ}_{i} ) Beta ( {β}_{i} \vert {a}_{0} , {b}_{0} ) + {ρ}_{i} Beta ( {β}_{i} \vert {a}_{1} , {b}_{1} ) ]$$

and the variance of the epithelial and immune beta distributions were used as estimates of epithelial and immune variance.

### SNP Genotyping, QC and Imputation

In total, 74 ovarian cancer case subjects and 225 controls from the methylation discovery cohort were genotyped using the Illumina 650k Infinium Global Screening Array (GSA). Whole blood DNA was normalised to 75 ng/μl and a total of 300 ng applied to the Infinium Global Screening Array—24 V2 (Illumina, CA, USA) at UCL Genomics according to the manufacturer's standard protocol.

One ovarian cancer case and one control subject from this cohort failed to genotype. Genotype calling was performed using GenomeStudio, with genetic variants found to be clustering poorly removed from further analyses. For duplicate genetic variant pairs, the variant within each pair with the lowest calling and clustering score was excluded. Autosomal SNPs were used in subsequent QC and PRS analyses (except for checks for sex mismatches, where the X chromosome was used to infer sex).

General subject and single nucleotide polymorphism (SNP) quality control (QC) was performed using PLINK version 1.9. Four ovarian cancer cases and eight controls with a call rate less than 95% were excluded. Three controls were further removed due to genetically inferred sex not being female. Genetic variants with a missing genotype rate greater than 5%, minor allele frequency (MAF) less than 1% or a significant departure from Hardy-Weinberg equilibrium (p-value <5×10⁻⁶) were excluded.

KING, a relatedness inference algorithm, was used to identify duplicate/monozygotic twin or first-degree relative pairs. One control subject pair was identified as being a duplicate/monozygotic twin pair, and nine control pairs were inferred to be first-degree relatives. The subject within each related pair with the lowest call rate was excluded. After performing QC, 225 ovarian cancer case subjects, 816 controls and 479,105 variants were retained in the SNP discovery sample.

Non-European subjects were identified by plotting the top two principal components, generated using GCTA version 1.26.0, for the SNP discovery samples and 270 HapMap phase II release 23 samples (CEU, YRI, JPT and CHB individuals) downloaded in PLINK-formatted binary files. Subjects found not to cluster around HapMap European samples were excluded from further analyses. After excluding non-European subjects, 217 ovarian cancer cases and 752 controls were retained in the SNP discovery sample.

Using the Michigan Imputation Server and Haplotype Reference Consortium (HRC) reference panel, the SNP discovery dataset went through further QC before being phased (Eagle2) and imputed. Variants where strand, allele, genetic position or allele frequencies were not concordant with the HRC reference panel were removed before phasing and imputation using Strand Tools. After imputation, variants with imputation R²<0.5 were removed. LD-based clumping was performed to retain a set of independent variants (r²>0.1). 28 SNPs, associated with ovarian cancer, were used to develop an ovarian cancer polygenic risk score (PRS; Additional Data 3). The inventors constructed an ovarian cancer PRS for each subject in the discovery cohort, such that the PRS is equal to:

$$\\ {PRS}_{j} = {\sum }_{i = 1}^{28} {\hat{β}}_{ι} {x}_{ij}$$

where, \hat{β}ₗ is the log odds ratio for the i-th SNP taken from publicly available ovarian cancer summary association results and xᵢⱼ is the number of copies of the effect allele present in each discovery cohort subject. The ovarian cancer summary results used were based on weights given in Phelan et al. (C. M. Phelan et al., Identification of 12 new susceptibility loci for different histotypes of epithelial ovarian cancer. Nat Genet 49, 680-691 (2017)), which were downloaded using GWAS Catalog (Accession number: GCST004415). Scores were generated using PLINK version 1.9.

### Example

The inventors performed an epigenome-wide DNAme analysis in cervical smear samples from women who were subsequently diagnosed with ovarian cancer, and in matched controls, and established the WID-OC-index (Women's risk IDentification for Ovarian Cancer index) which the inventors further validated in an independent set of cervical samples. The inventors established that the WID-OC signature is not driven by tumour DNA, is high in healthy women with a BRCA1 mutation and has a very high sensitivity/specificity that also identifies women with endometrial cancer, which also arises from Müllerian Duct epithelial cells. In addition, the inventors assessed the WID-OC-index in normal fimbrial and high grade serous tissue and cell lines and a large range of tissue samples and showed that the WID-OC index is significantly associated with the rate of BRCA-1/2 germline-mediated cancer formation.

For the Discovery Set (FIG. 5), the inventors collected samples from 242 women with ovarian cancers from 15 European centres before a definitive histological diagnosis was undertaken (either during surgery or via a percutaneous biopsy) and 869 women without a cancer (593 from the general population and 276 from women attending hospital for benign women-specific conditions) (Table 6; samples from a greater proportion of younger women were deliberately used in the discovery set in order to develop a risk predictor applicable also to younger women; the external validation set was composed of age-matched cases and controls). Epigenome-wide DNAme was analysed using an Illumina Infinium EPIC bead chip array that encompasses over 850,000 CpG sites.

### Sample Heterogeneity and Differential Methylation

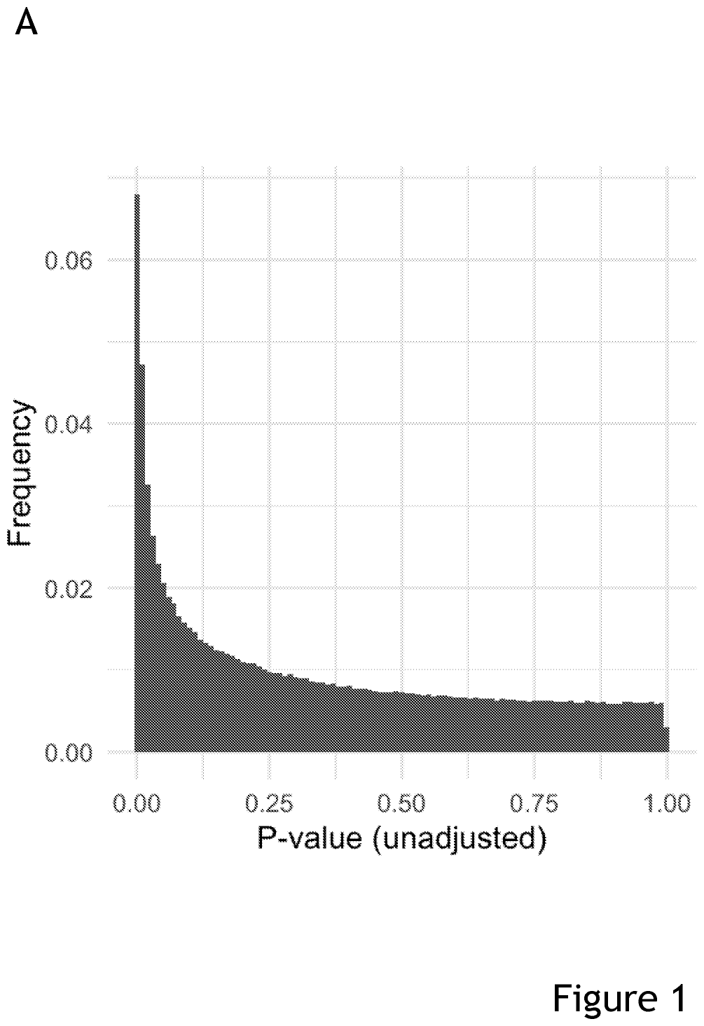

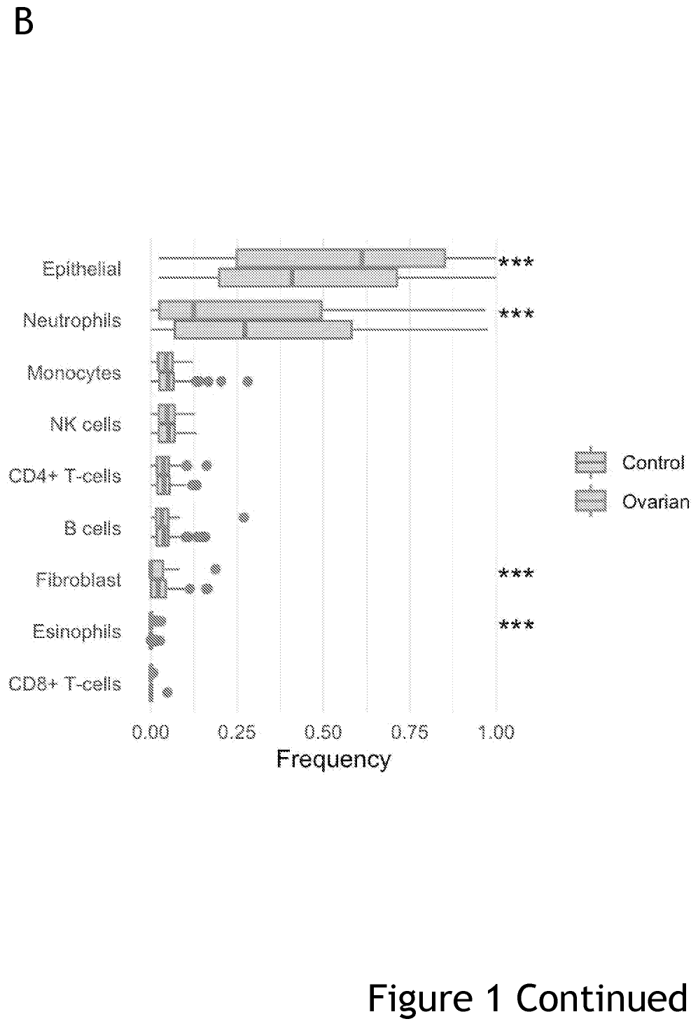

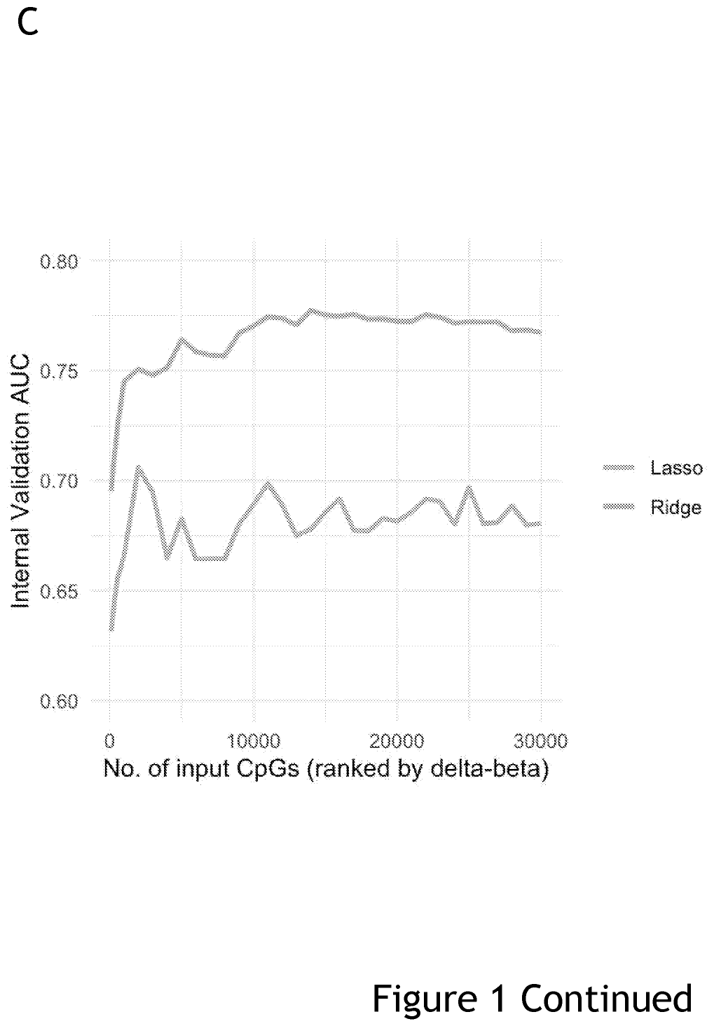

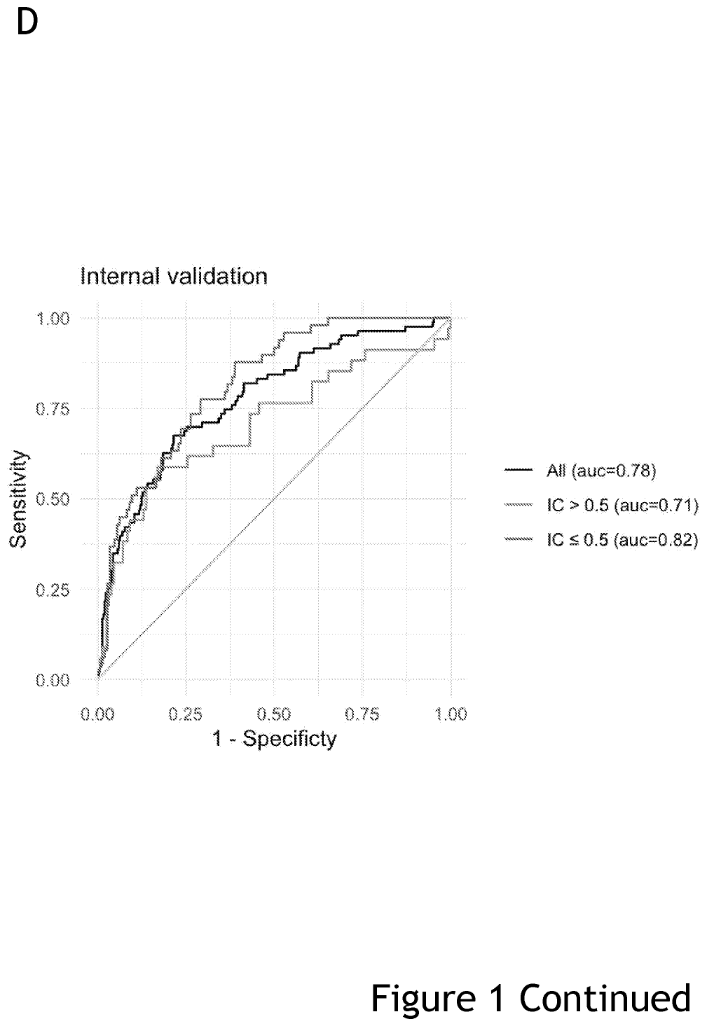

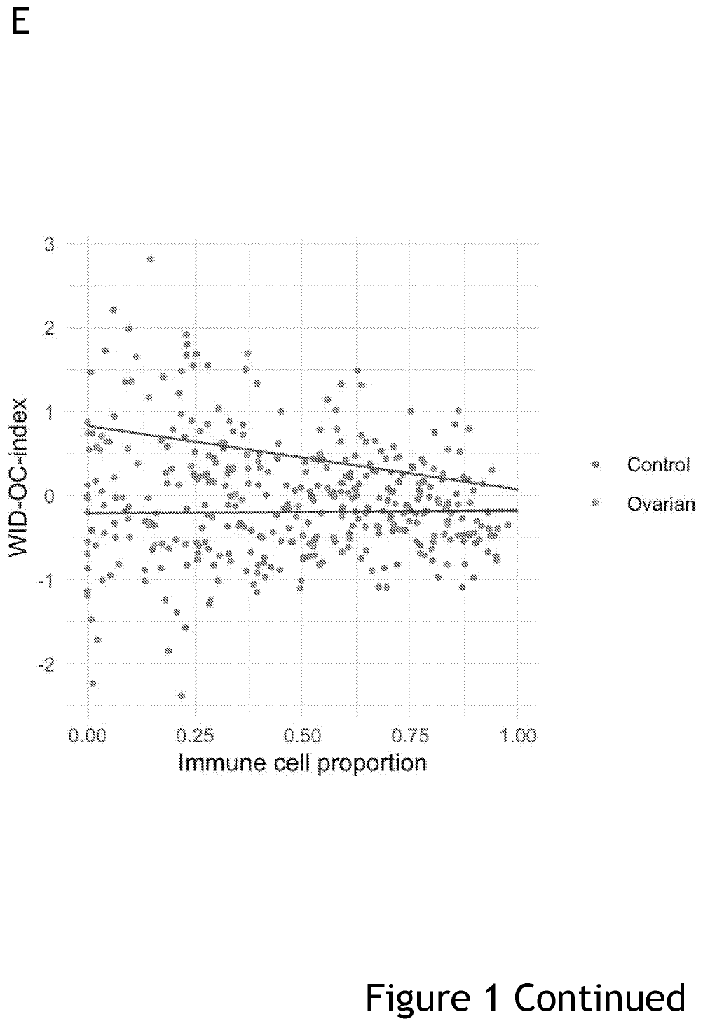

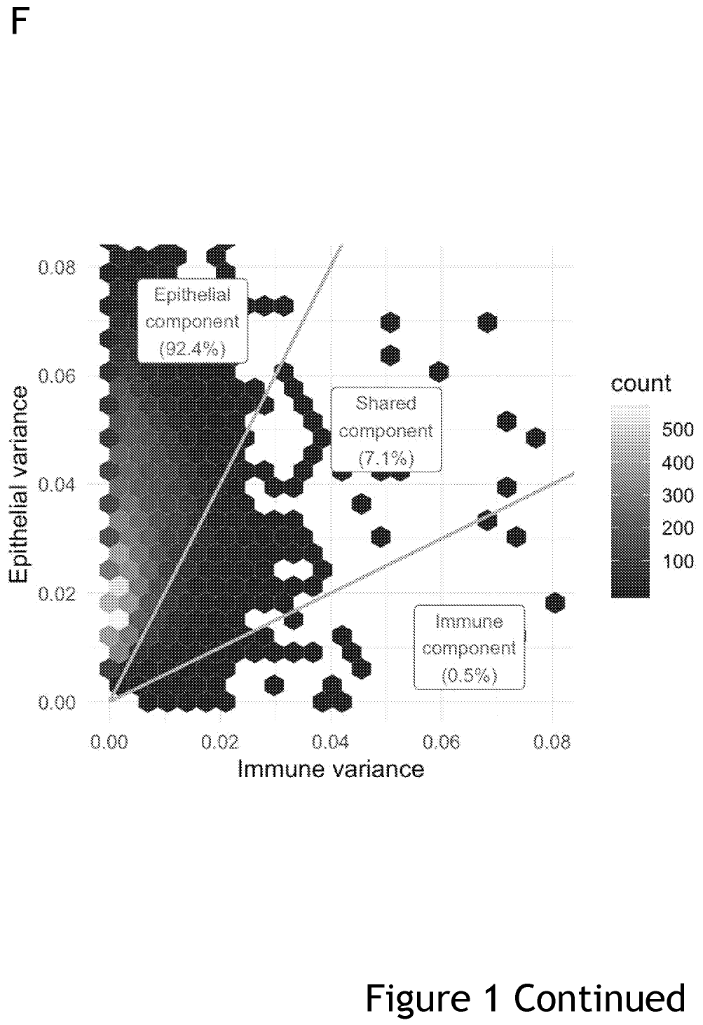

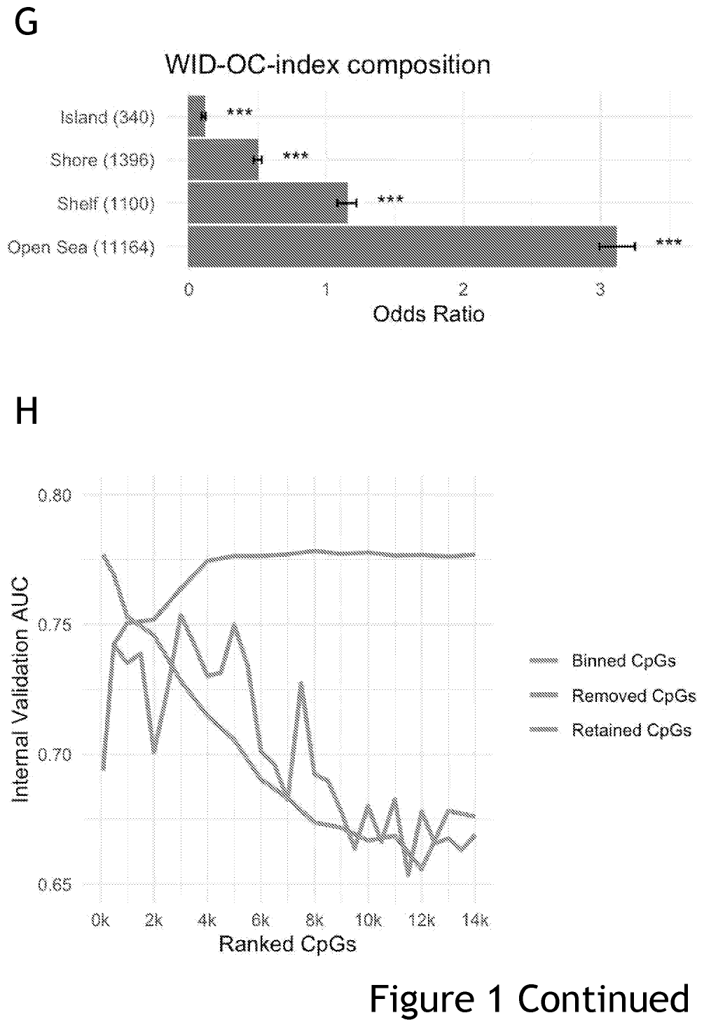

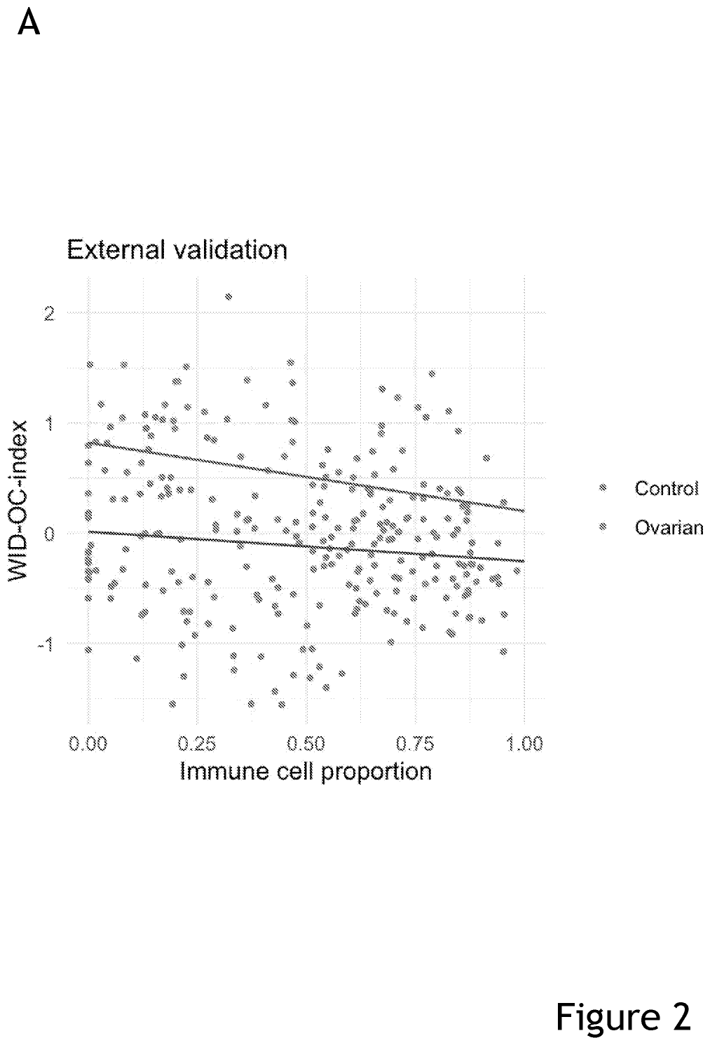

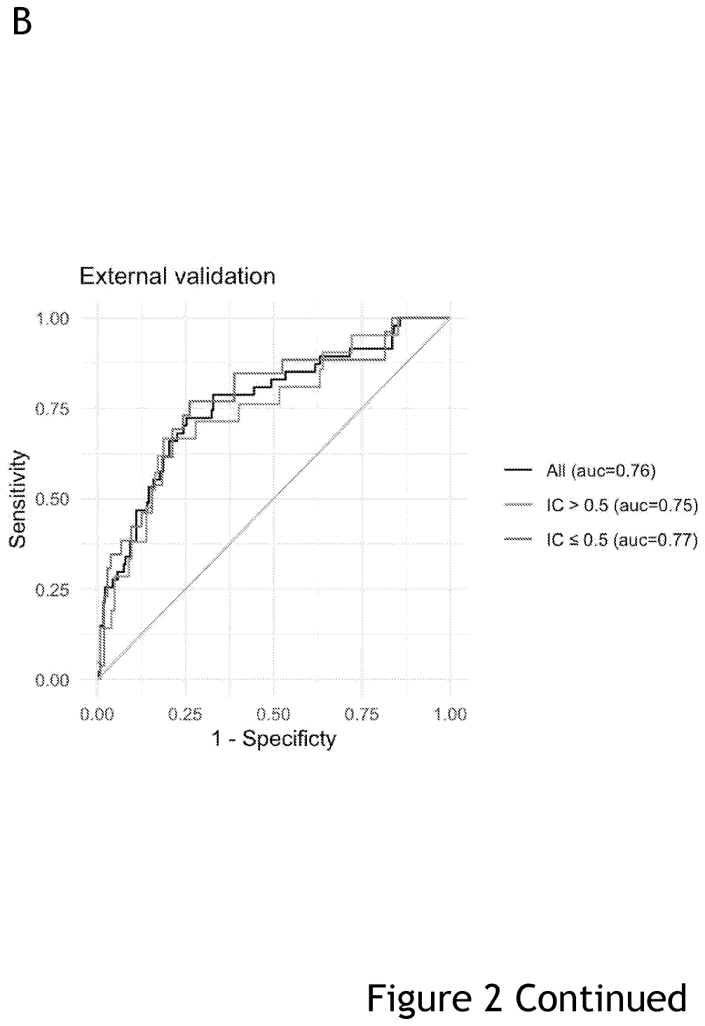

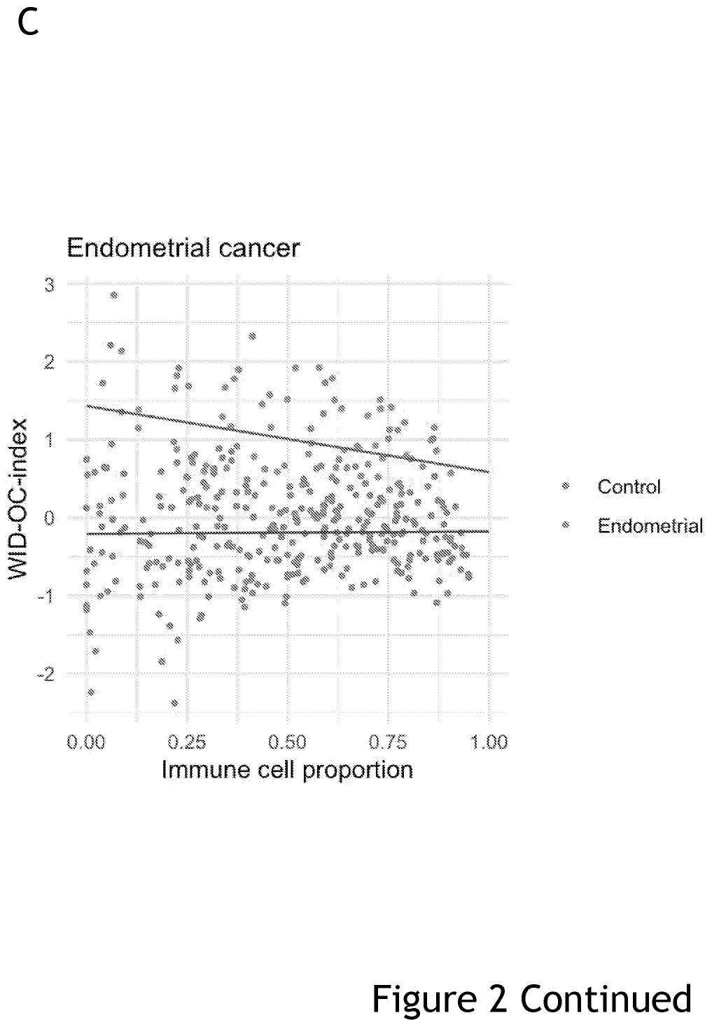

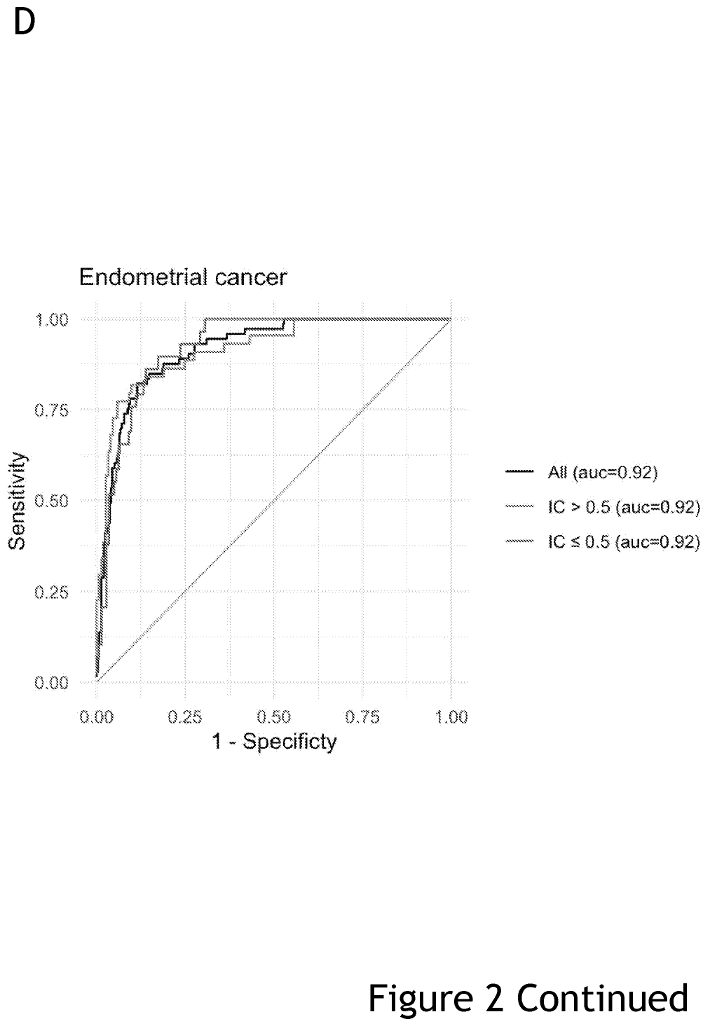

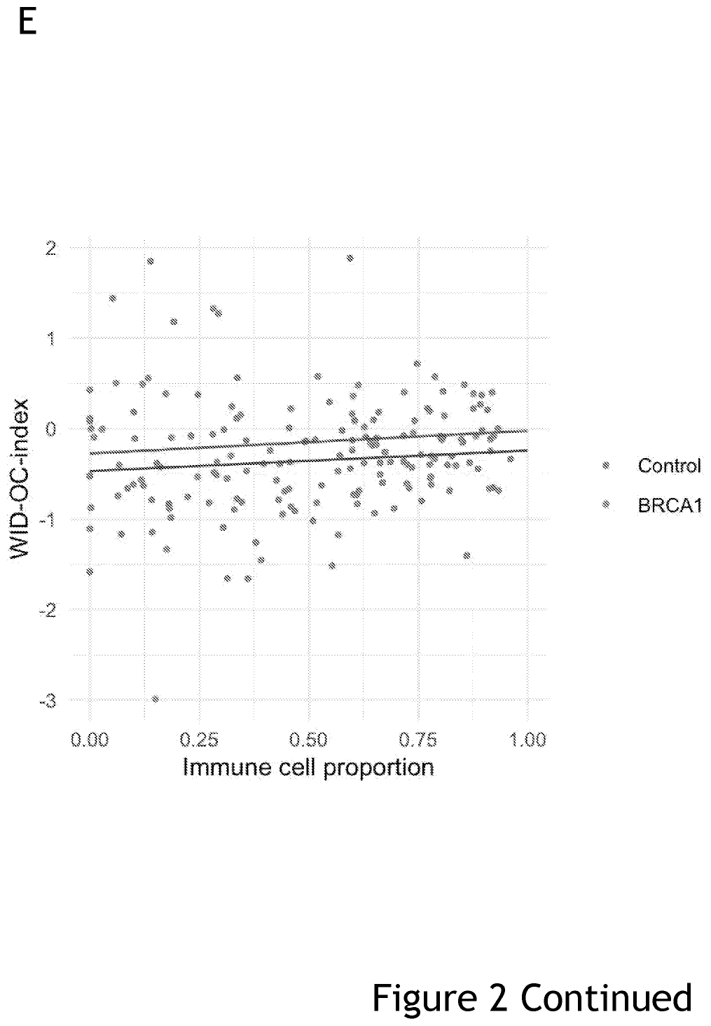

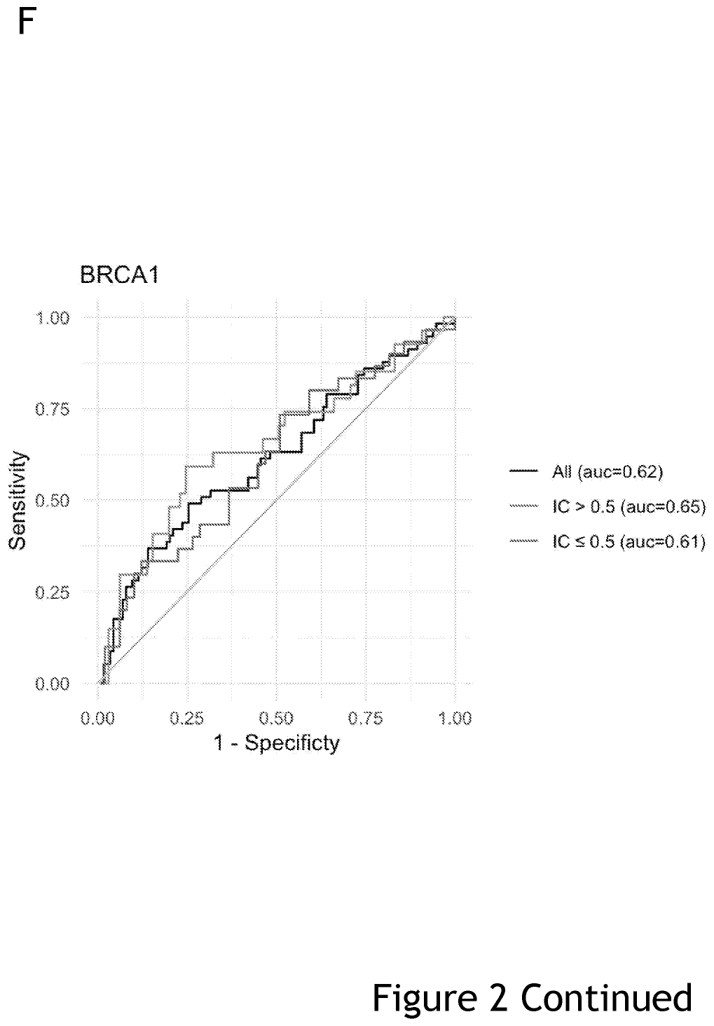

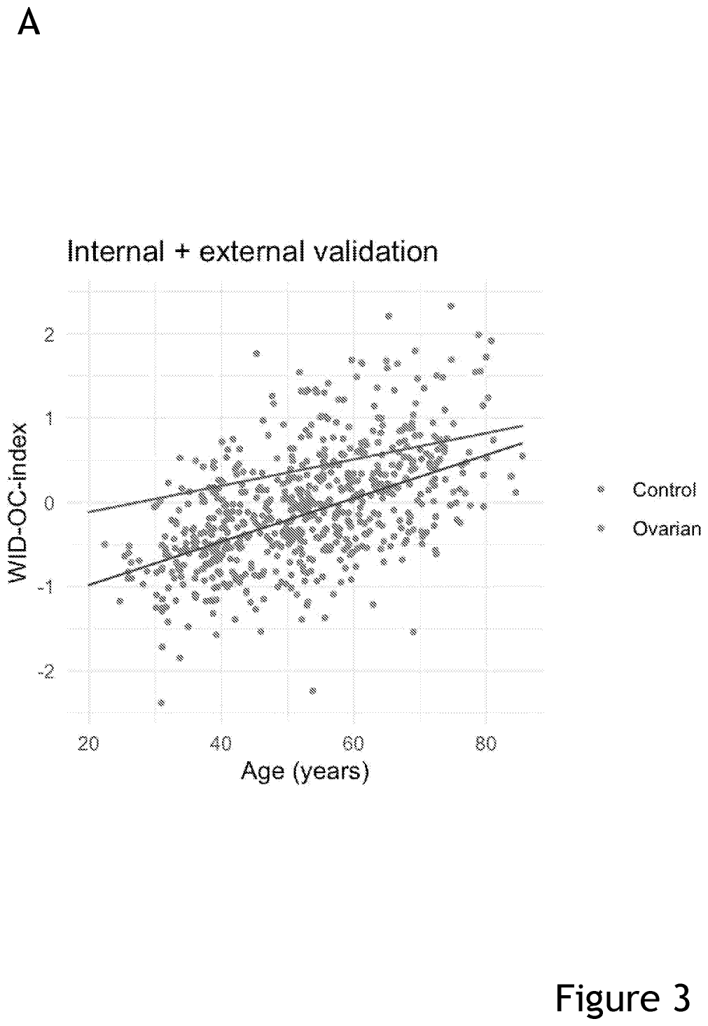

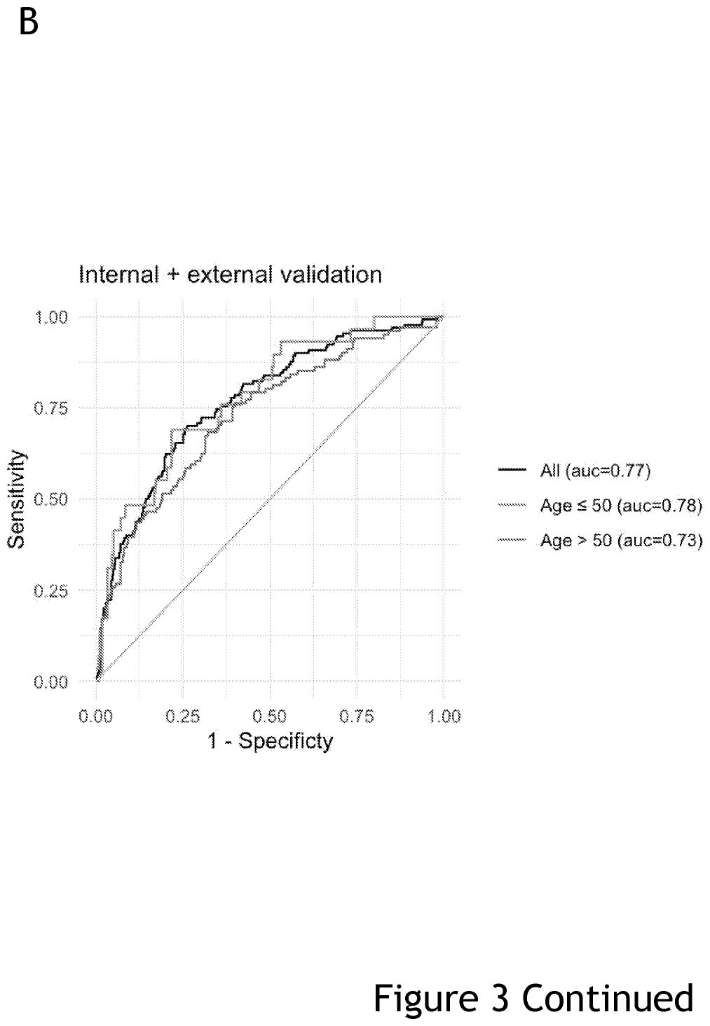

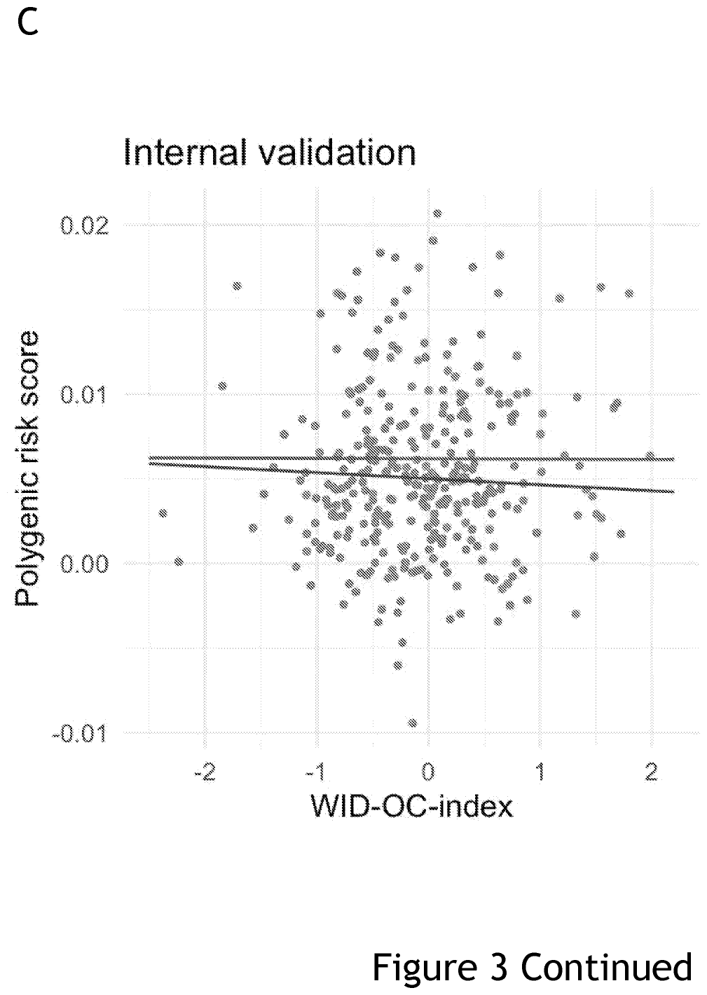

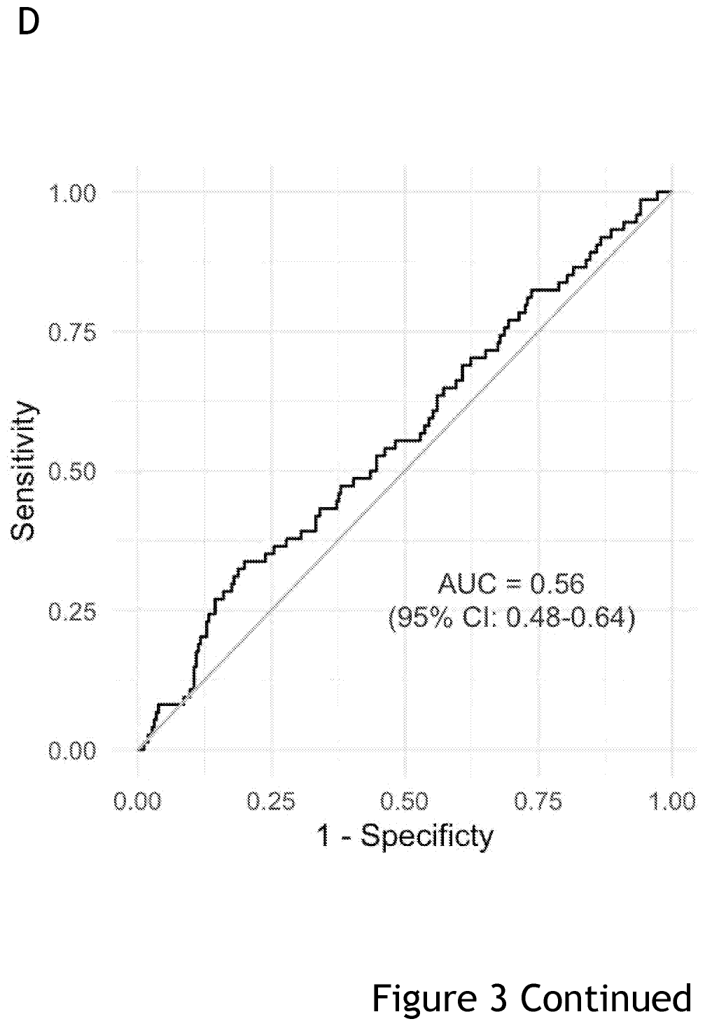

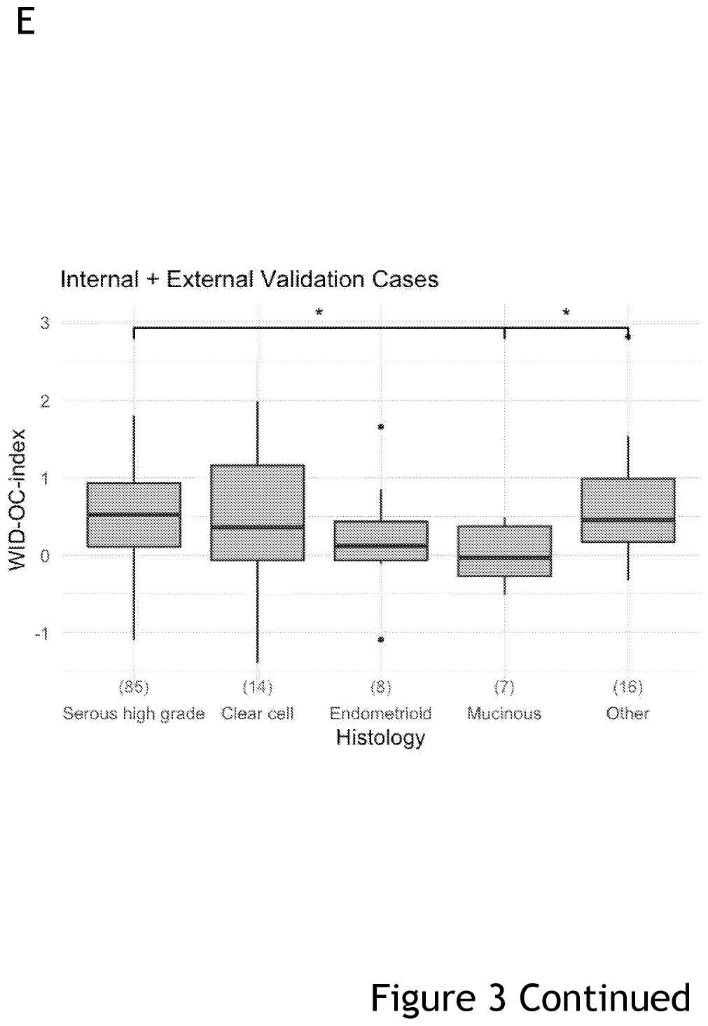

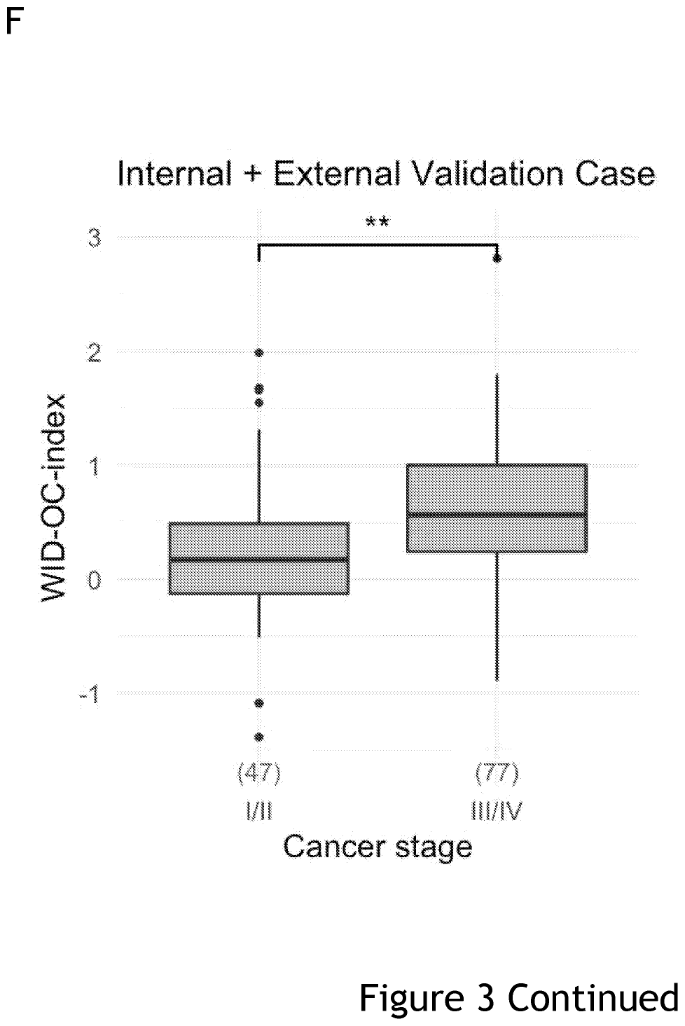

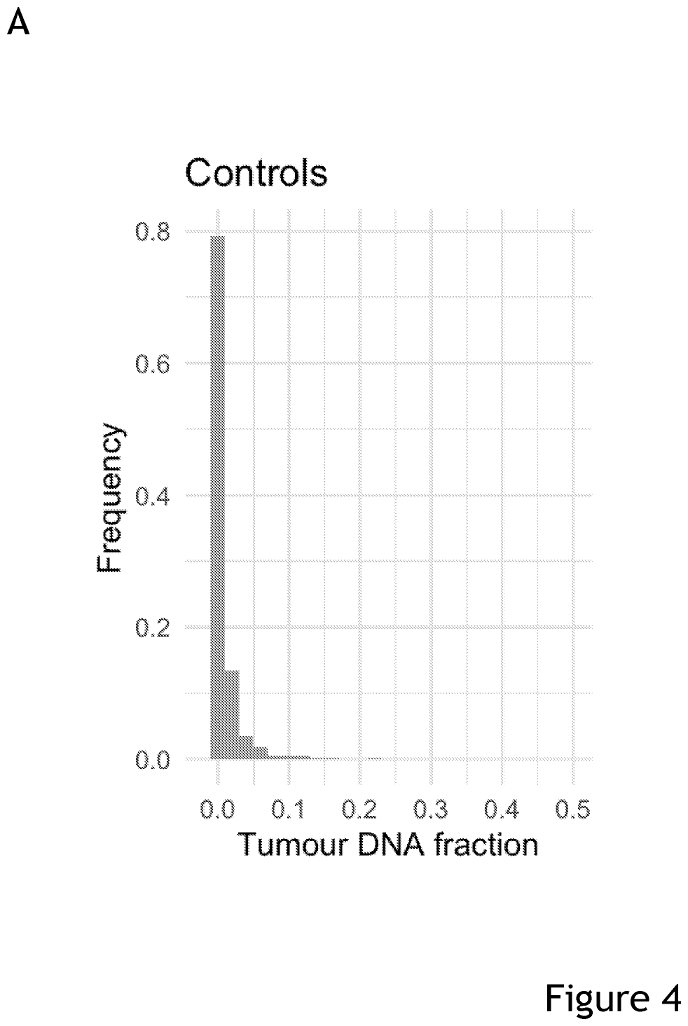

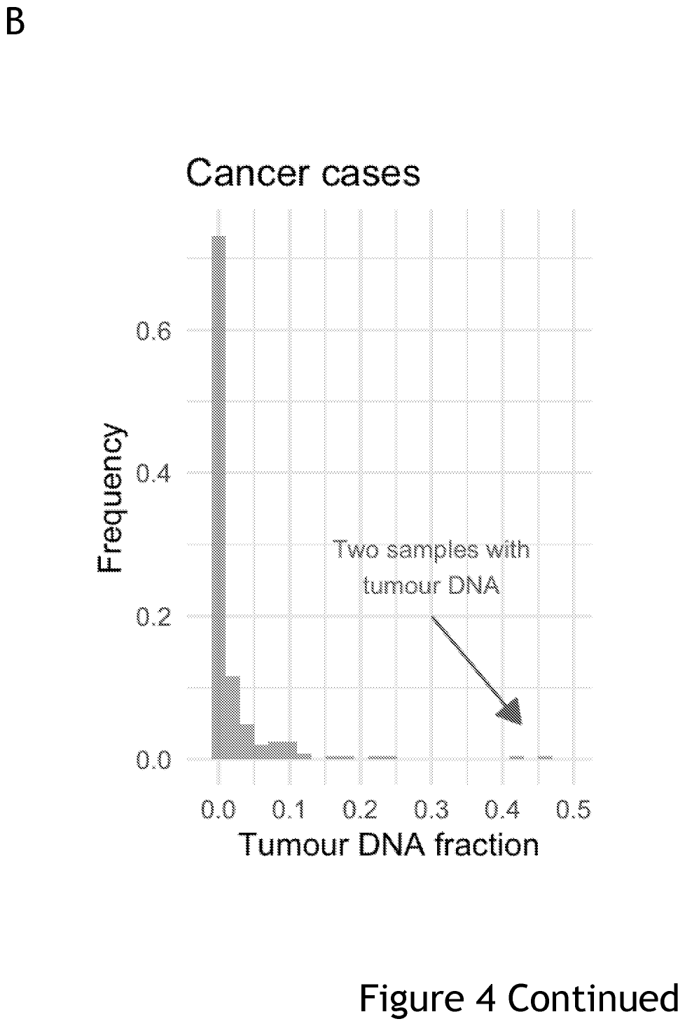

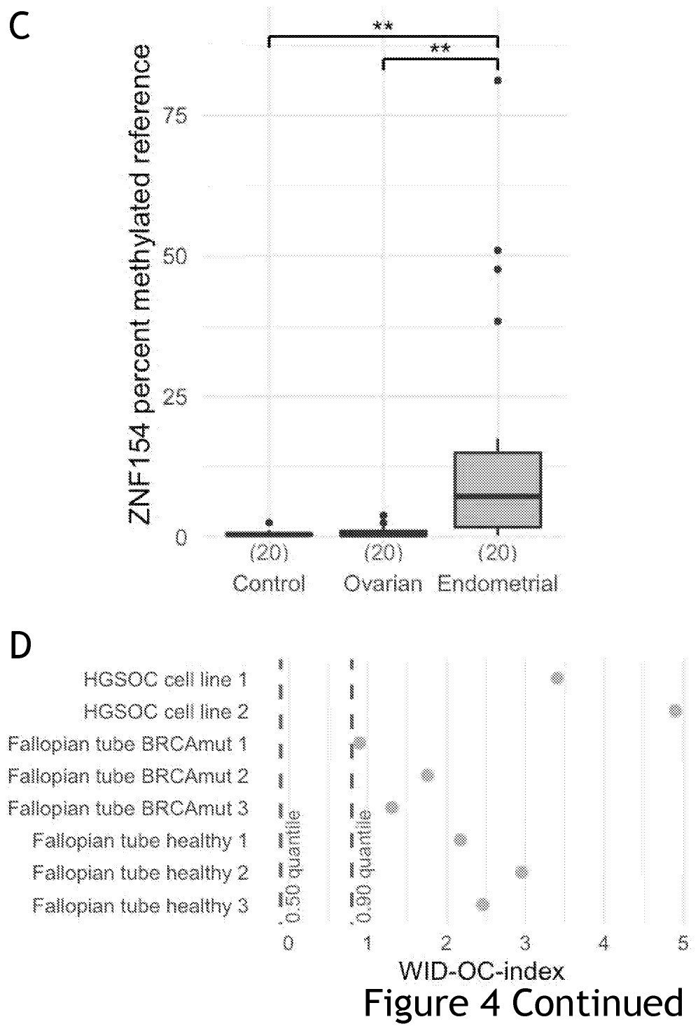

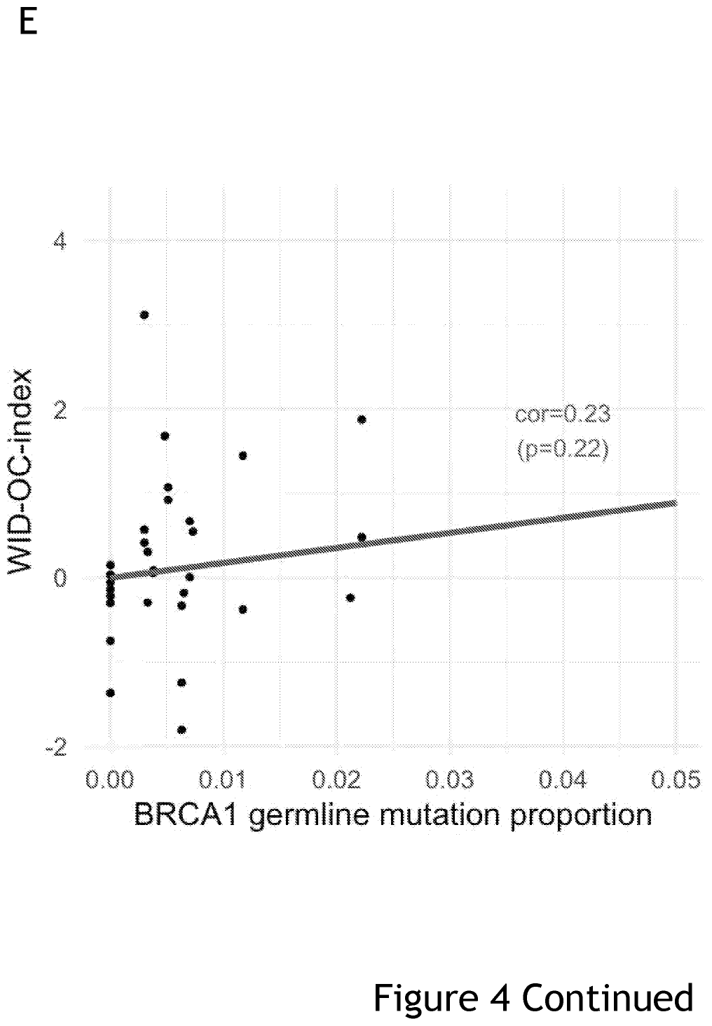

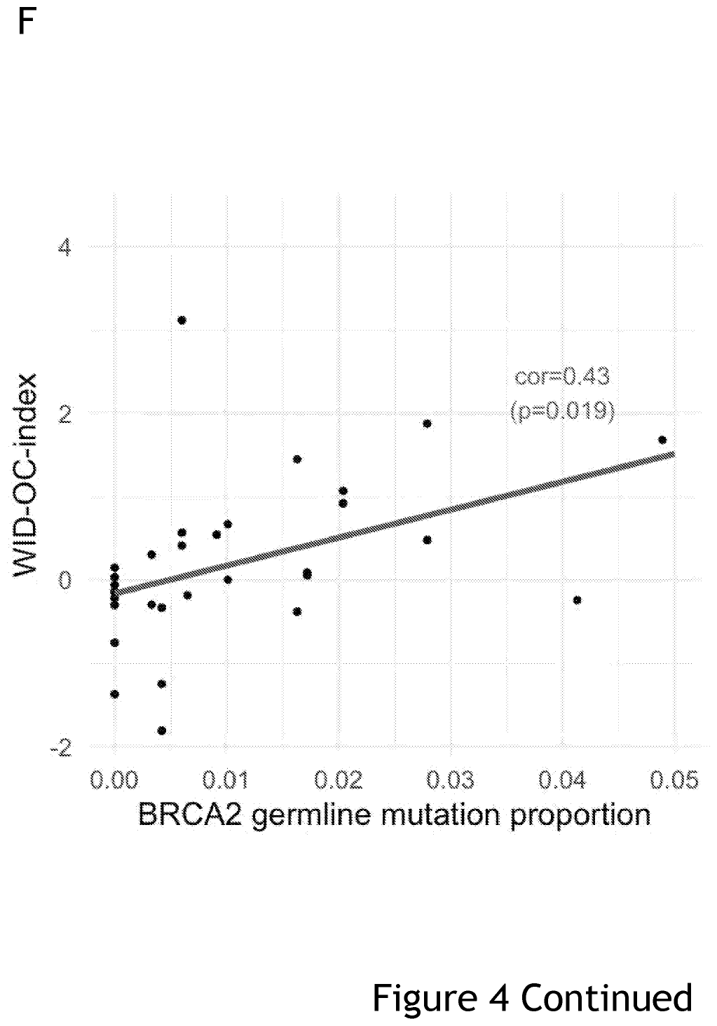

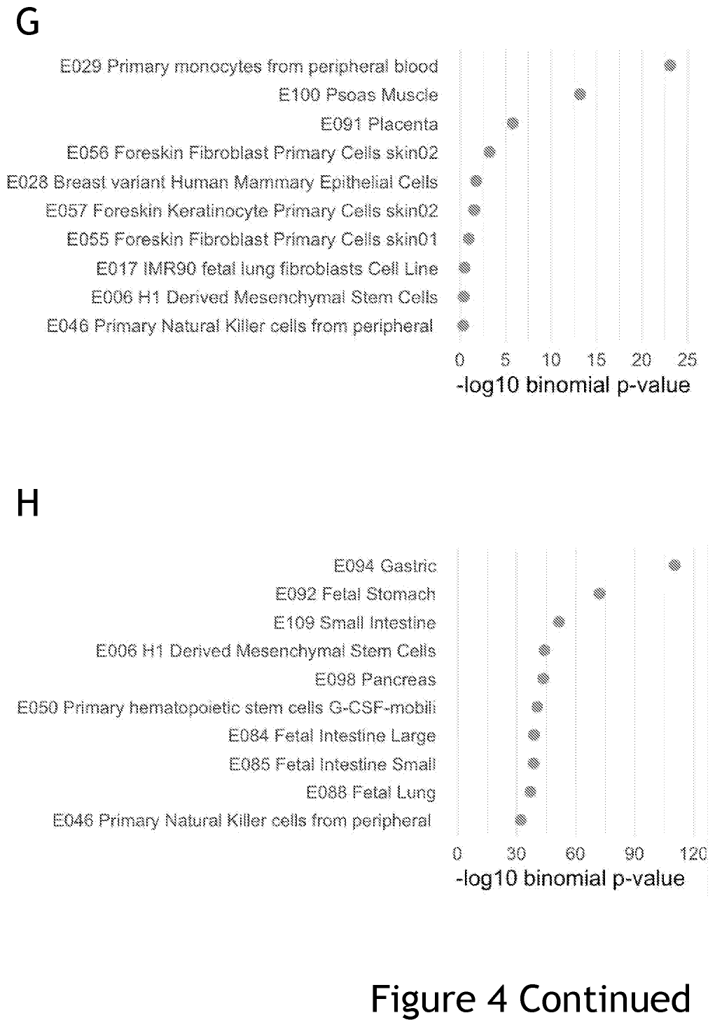

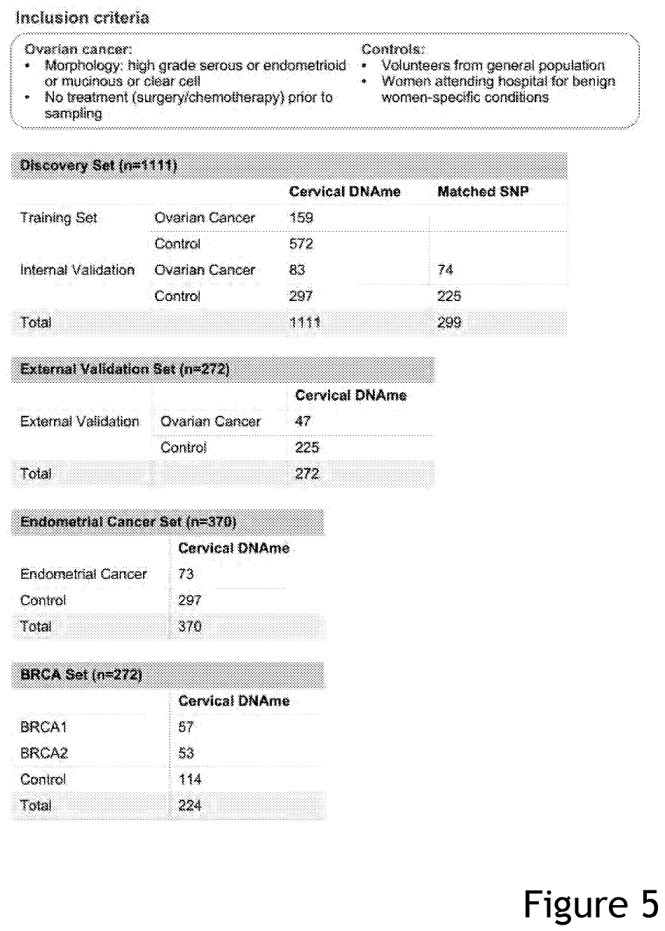

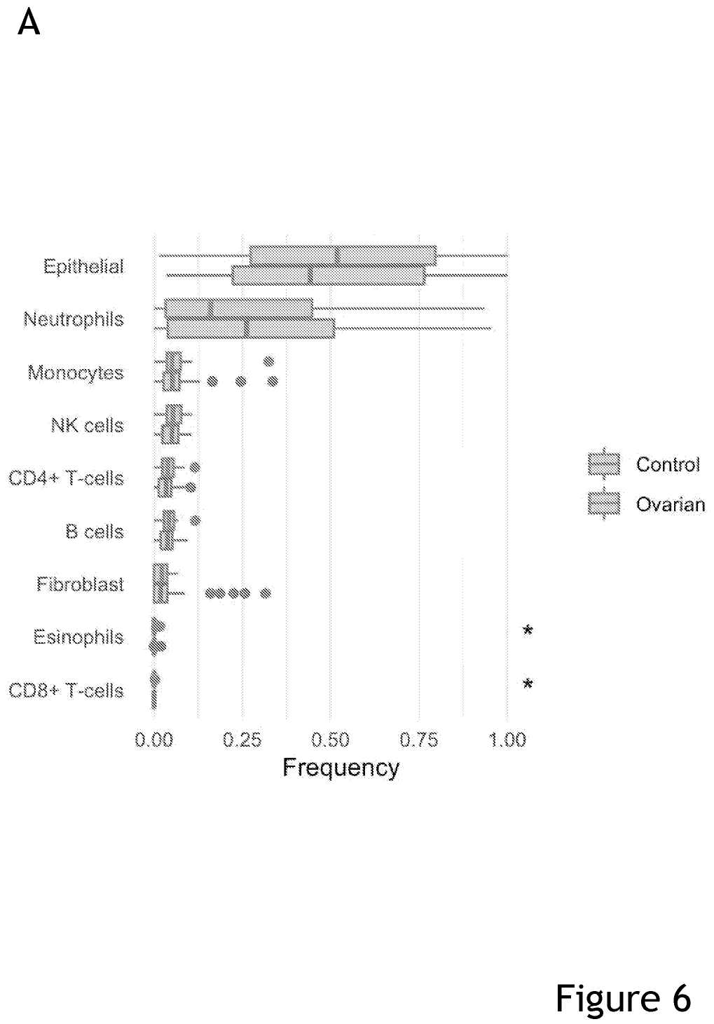

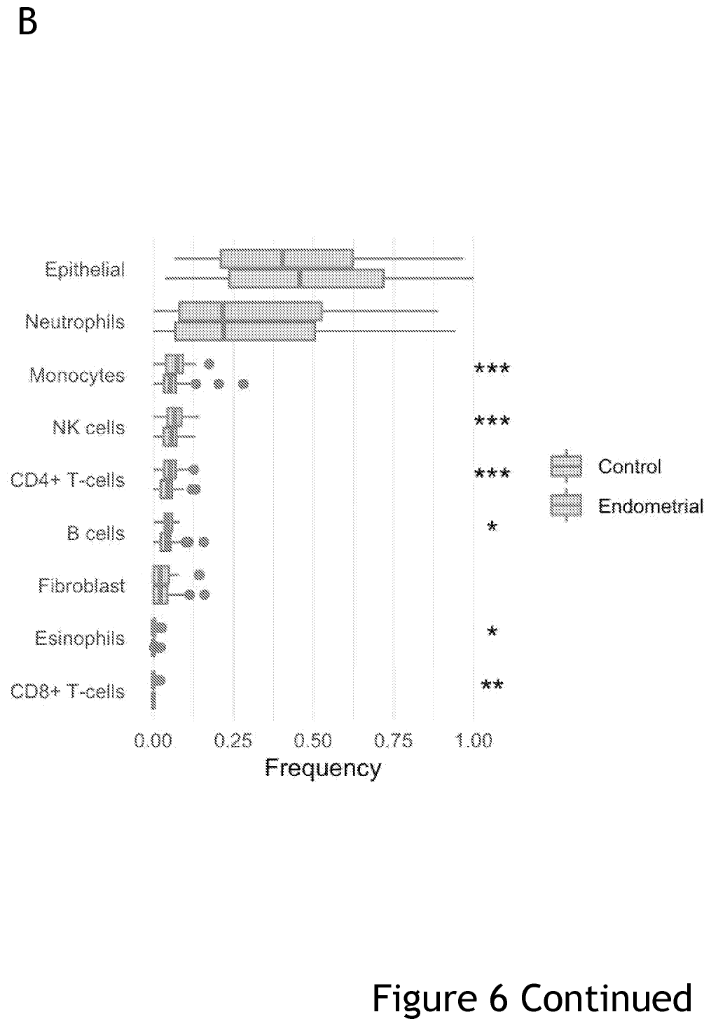

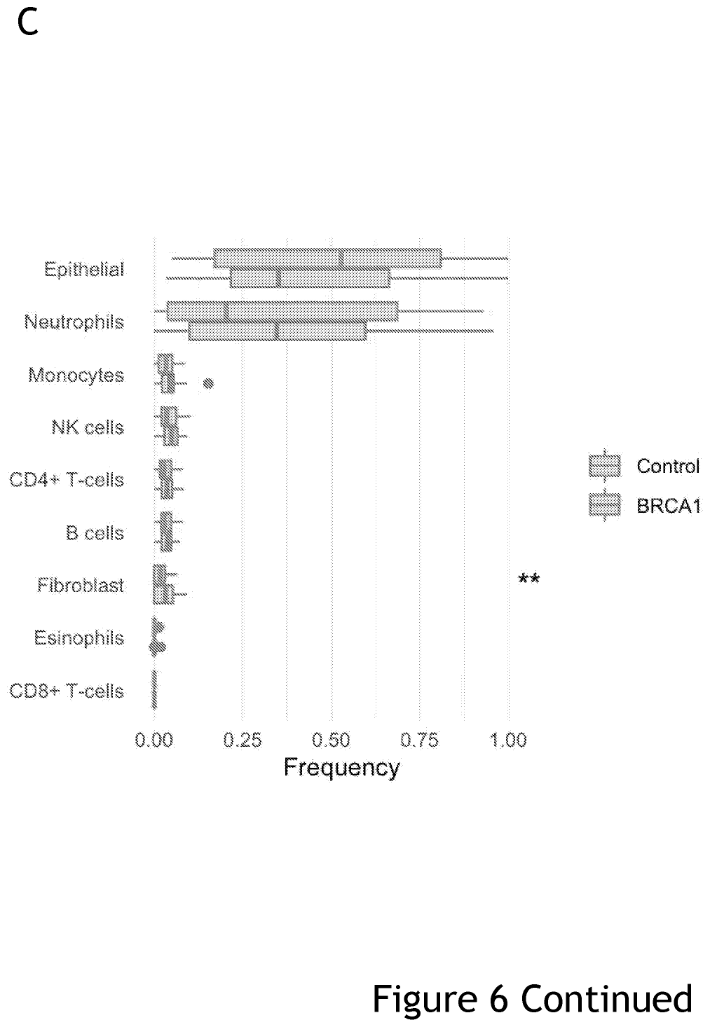

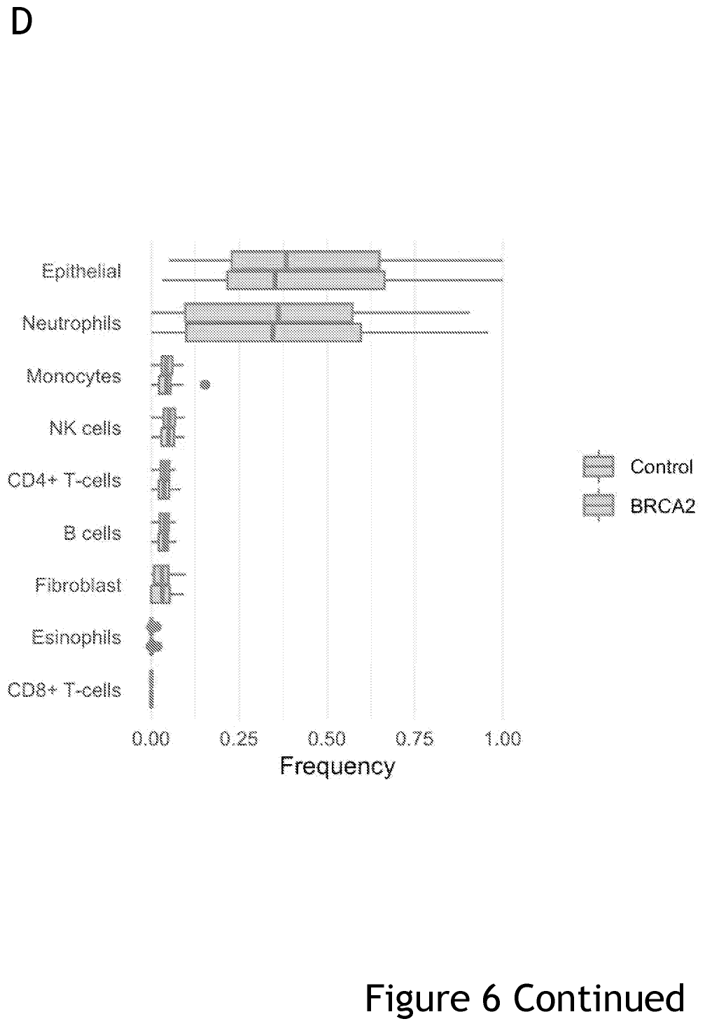

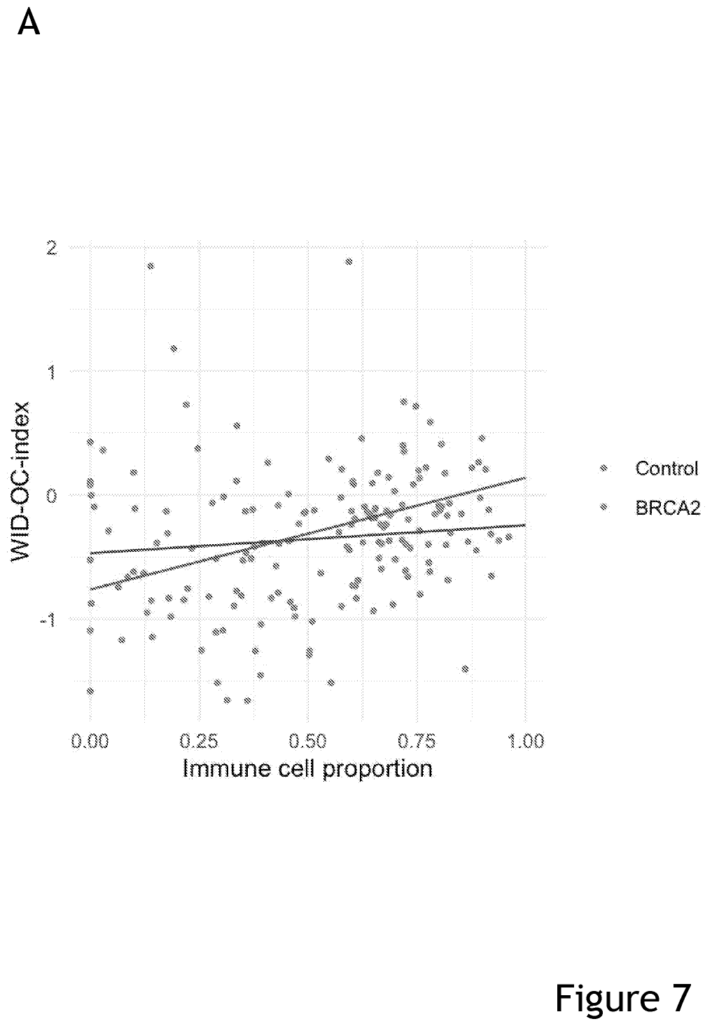

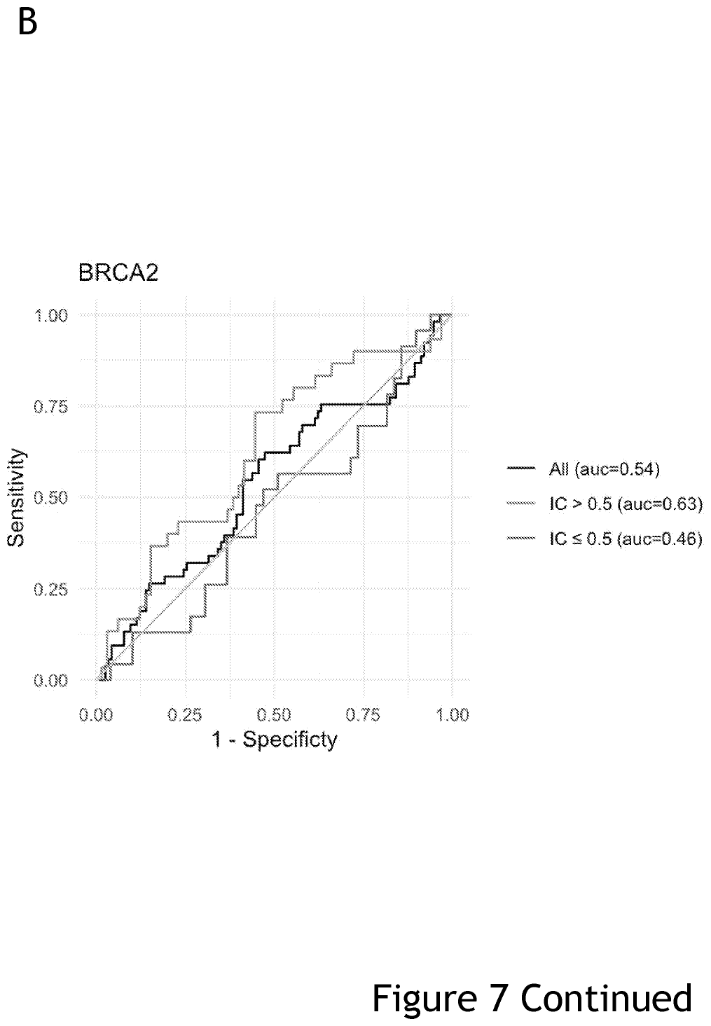

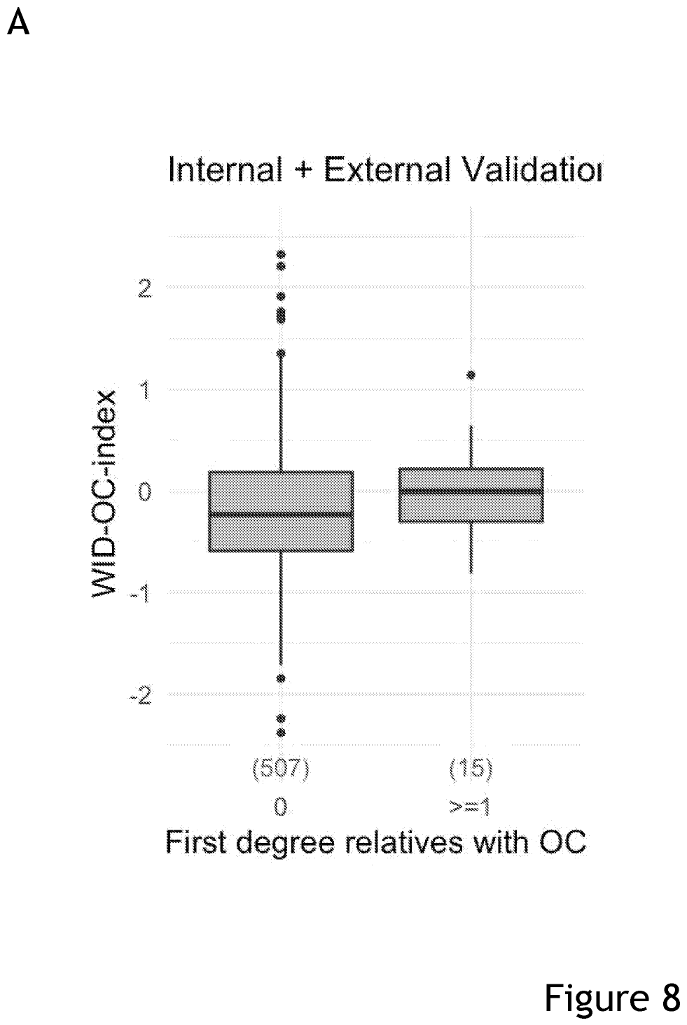

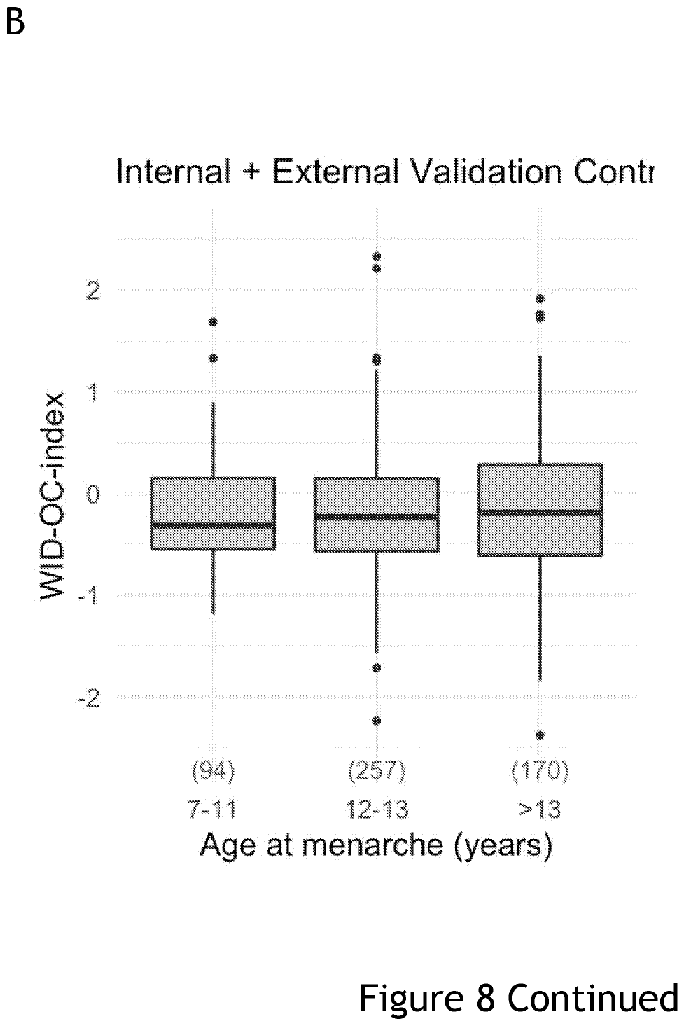

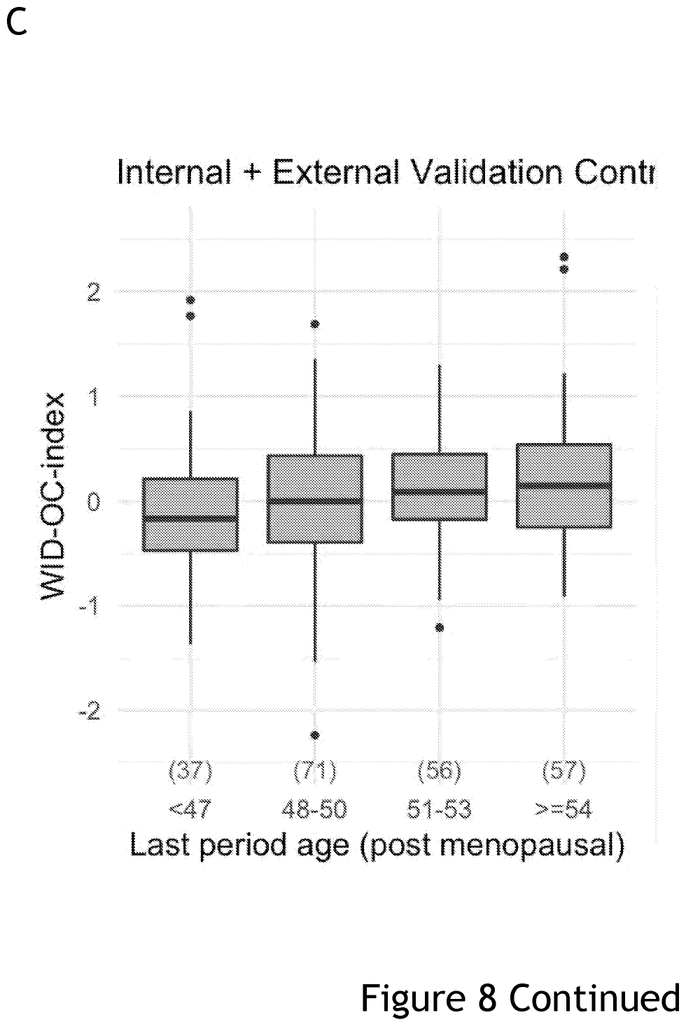

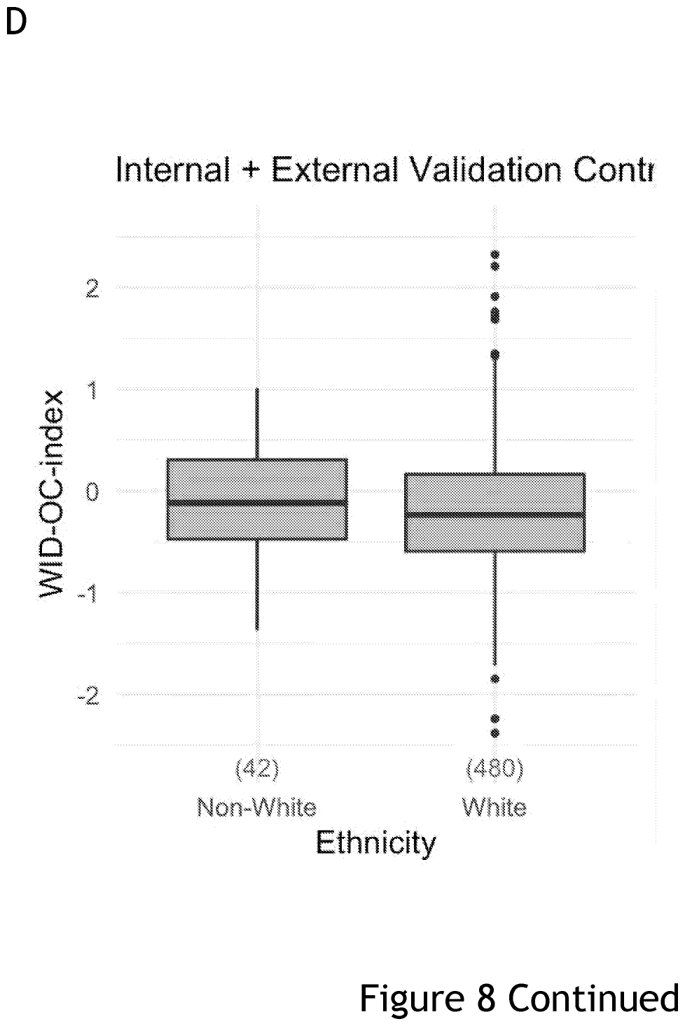

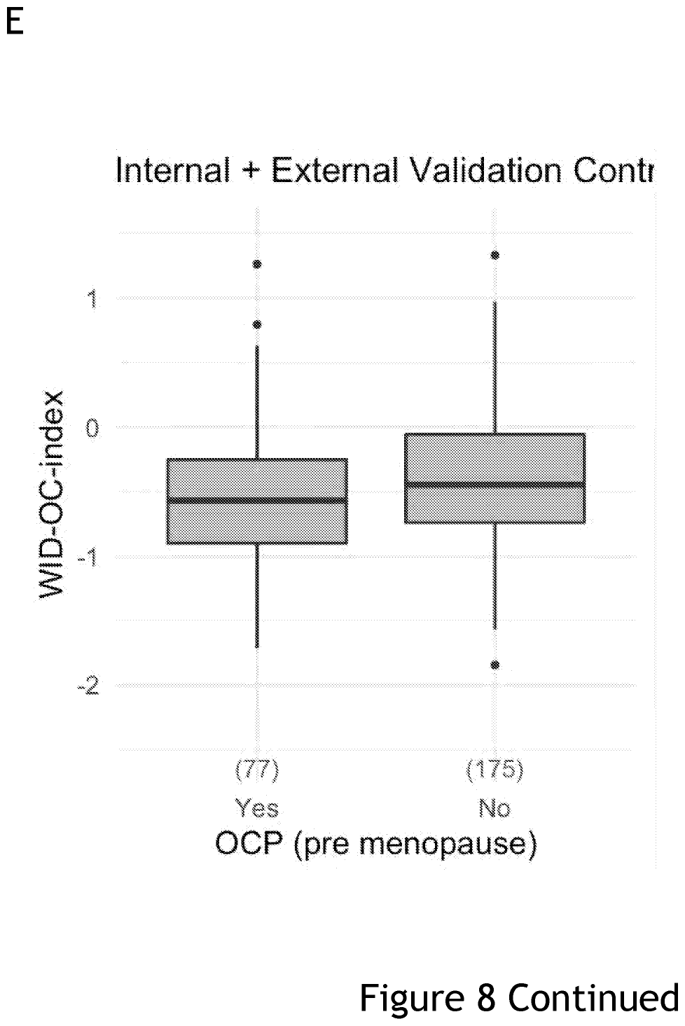

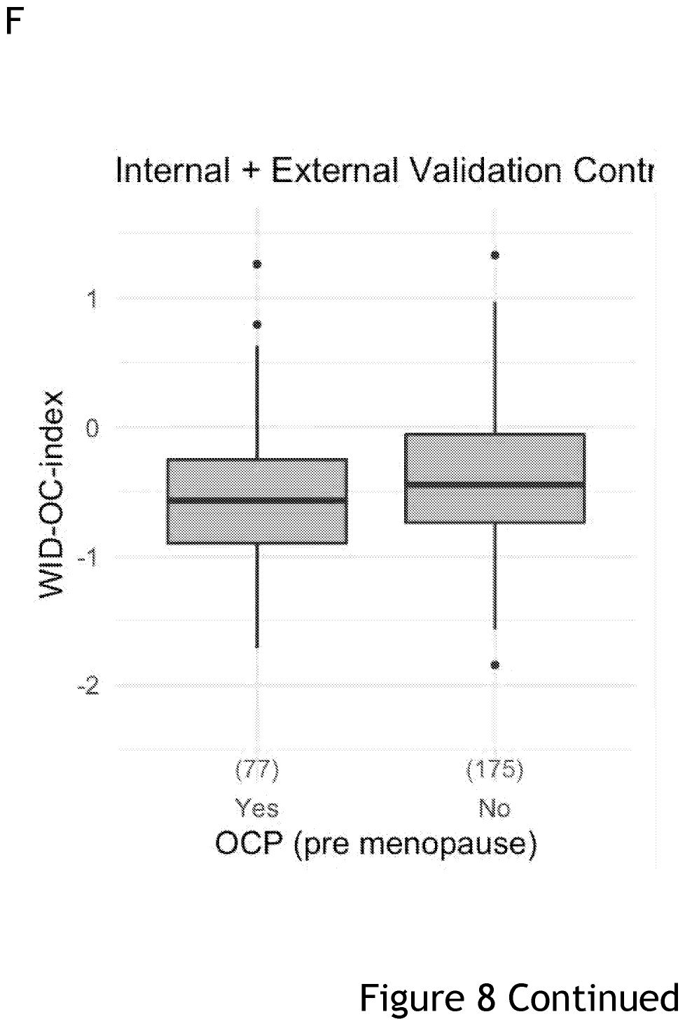

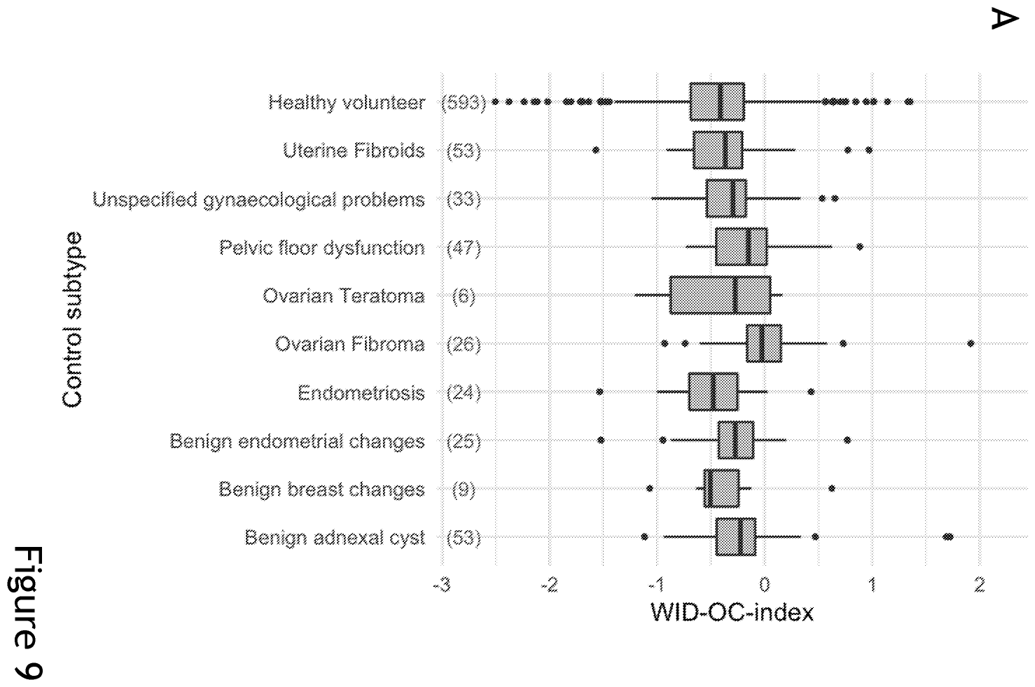

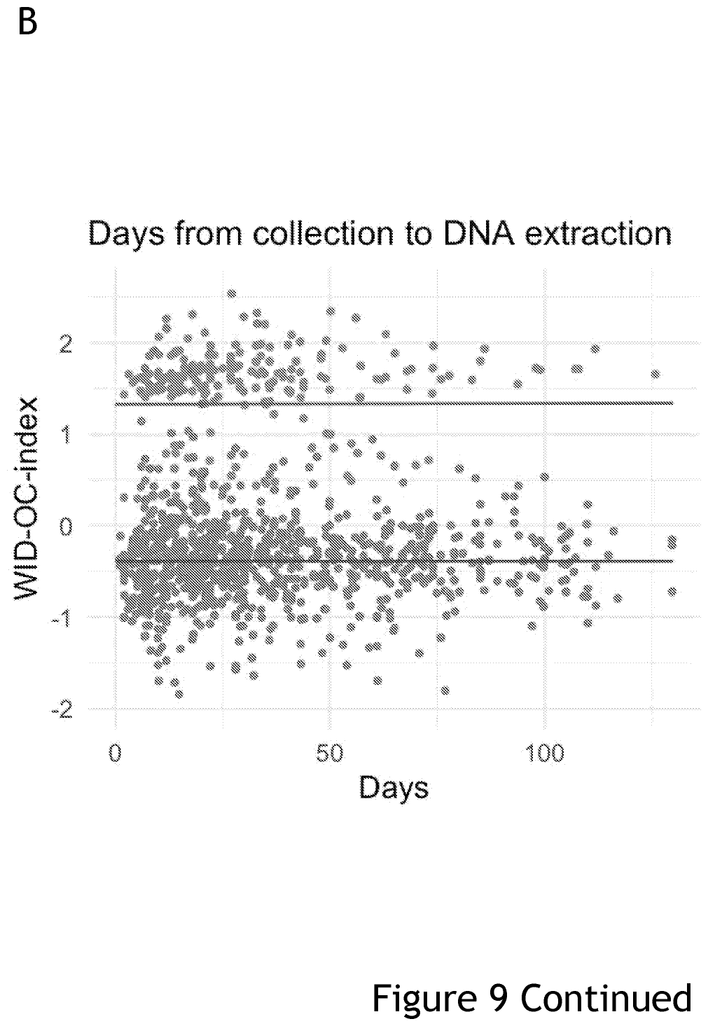

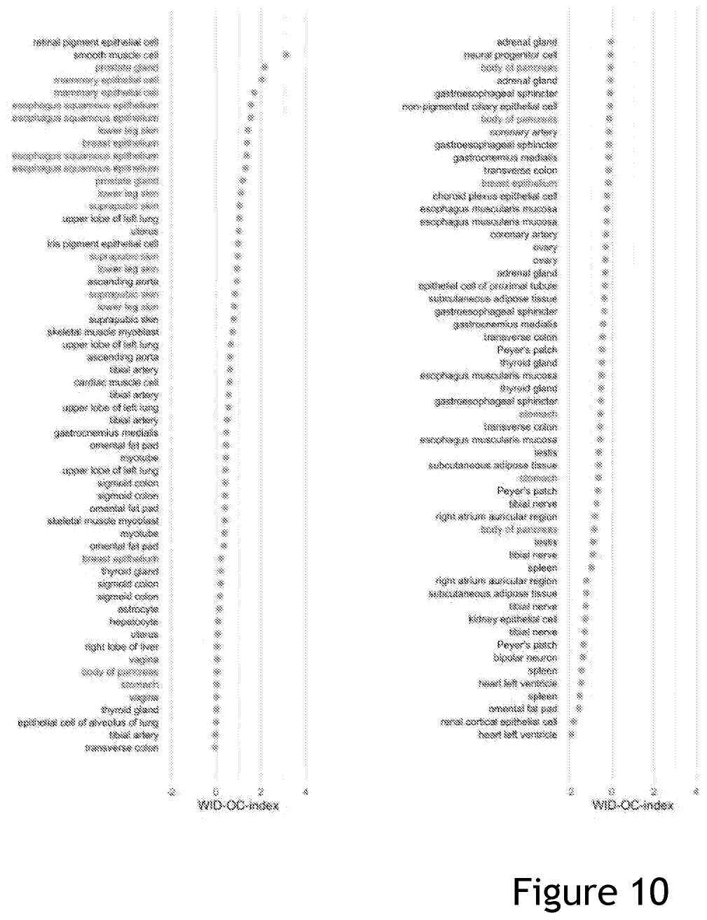

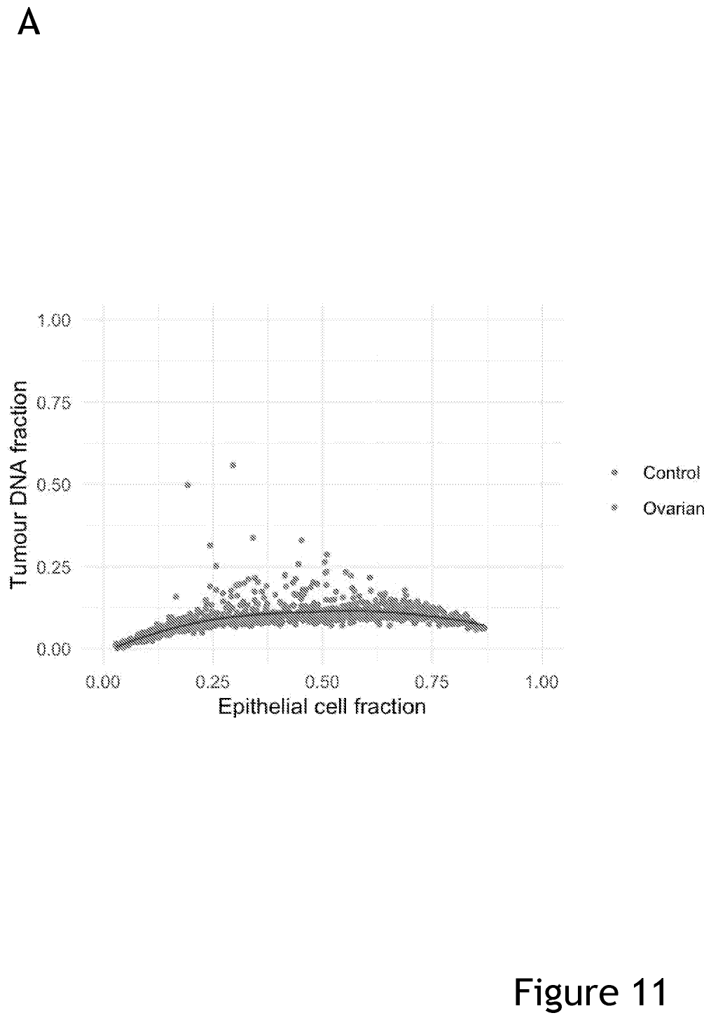

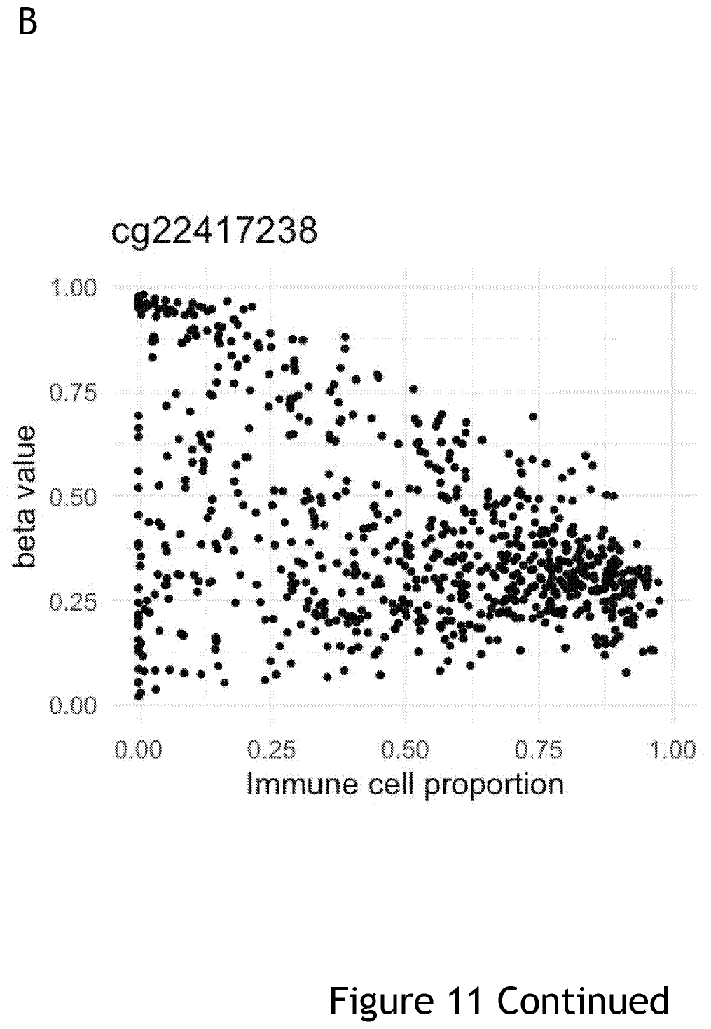

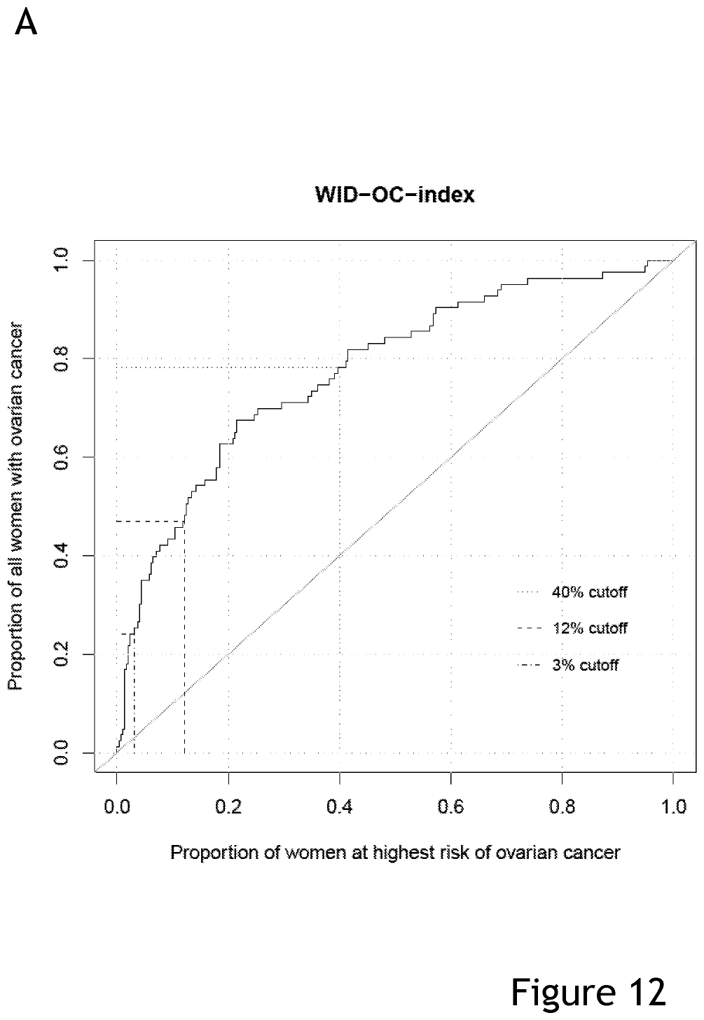

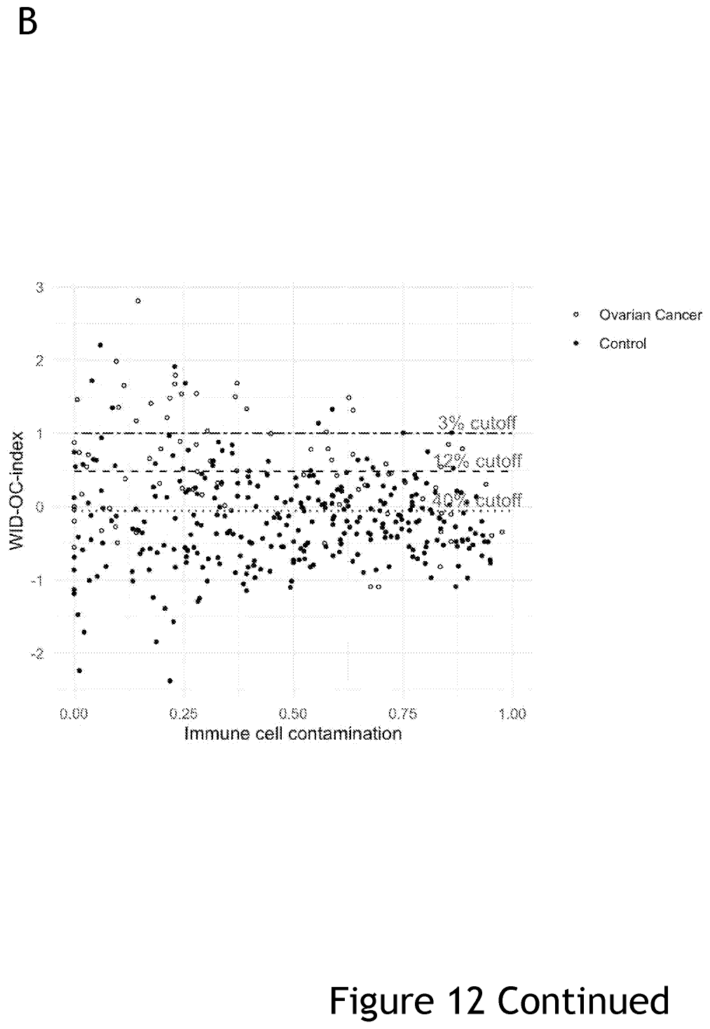

The inventors assessed the number of CpGs which were significantly differentially methylated between cancer cases and controls (FIG. 1A); after Bonferonni multiple test adjustment, 91 CpGs showed significantly differential methylation. Previously the inventors found that methylation differences may vary due to immune cell-type composition in cases compared to controls (A. E. Teschendorff et al., An epigenetic signature in peripheral blood predicts active ovarian cancer. PLoS. One 4, e8274 (2009). Hence, the inventors assessed the level of cell type heterogeneity in each cervical smear sample using HepiDISH, an algorithm that infers the relative proportion of epithelial cells, fibroblasts, and seven subtypes of immune cells (IC) in each sample. The cell-type distributions were broadly similar between cancer cases and controls although there was a significantly greater proportion of epithelial cells in cancer cases (FIG. 1B; this remained significant after adjusting for age and menopausal status). A similar trend was observed in the external validation dataset but was not significant (FIG. 6A).

Identification of CpGs with differential methylation between cases and controls was hampered by contaminating ICs, since any differential methylation in epithelial cells was greatly diminished in samples with high IC. In order to infer which CpGs may contain a potential discriminatory signal the inventors developed a statistical protocol to estimate the delta-beta (i.e. difference in mean proportion of methylated cells) between cases and controls in epithelial and immune cells. The inventors linearly regressed beta values on IC fraction in both cases and controls separately. The difference between the two points where these lines intercept the y-axis at IC=0 gives an estimate of the delta-beta between cases and controls in pure epithelial cells. Conversely, the difference between intercept points at the IC=1 axis gives a delta-beta estimate in immune cells.

### Development of Discriminatory Index

In order to derive a diagnostic methylation signature, termed the WID-OC-index, the inventors used ridge and lasso regression to classify individuals as cases or controls. Classifiers were trained on two thirds of the discovery dataset (572 cancer-free controls, 159 ovarian cancer cases) and the remaining one third was used as an internal validation set (297 controls, 83 cases) with the intention of evaluating their performance as a function of the number of CpGs used to construct the index. The area under the receiver operator characteristic curve (AUC) was used as a measure of predictive performance. CpGs were ranked according to their epithelial delta-beta.

Predictive performance was evaluated as a function of the number of CpGs used to train the classifier using the internal validation dataset and optimal performance of 0.78 (95% CI: 0.72-0.84) was achieved using 14,000 CpGs with ridge regression (FIG. 1C). In samples with an immune cell proportion <0.5 the AUC was 0.82 (FIG. 1D; 95% CI: 0.75-0.88), and in those with a proportion >0.5 the AUC was 0.71 (95% CI: 0.60-0.82). The WID-OC-index was not associated with IC fraction in controls (FIG. 1E, linear regression coefficients of 0.03, p=0.81), but a strong negative association was observed in cancer cases (linear regression coefficient of −0.76, p-value=0.005). Classifiers were also developed after ranking CpGs according to immune delta-betas and a combined ranking based on both epithelial and immune delta-betas but these approaches offered inferior performance.

The inventors developed a statistical model to infer the variance in epithelial and immune cells at each of the 14,000 CpG sites used in the WID-OC-index, and classified each CpG as “epithelial” (92.4%), “shared” (7.1%), or “immune” (0.5%) as shown in FIG. 1F. These findings suggest that the discriminatory signal originates primarily in epithelial cells and the discriminatory power is diminished in samples with higher levels of immune cells.

The inventors found that the index was highly depleted of CpG islands and enriched for Open Sea regions (FIG. 1G). Ridge regression combines information from all input CpGs in contrast to lasso regression, which typically selects a small subset of inputs. Ridge regression offered consistently superior performance suggesting that the discriminatory signal is most robustly extracted by combining a large number of comparatively weak signals from multiple CpG sites. The inventors ranked the 14,000 CpGs used to define the WID-OC-index according to the absolute value of the regression coefficients from the ridge model. In order to assess how informative the top CpG sites are the inventors trained sub-classifiers on the top n sites (FIG. 1H). The inventors observed that AUCs of 0.74 and 0.76 can be achieved with the top 500 and 3,000 CpGs respectively indicating that these subsets are particularly informative. The inventors also trained sub-classifiers after removing the top n CpGs, and on subsets of 500 CpGs after partitioning the ranked list into bins of size 500. In both cases, the inventors found that a substantial predictive signal is present. These observations suggest that the predictive signal is widely distributed among Open Sea CpGs with a high degree of redundancy.

### External Validation

A separate independent external validation dataset consisting of 47 ovarian cancer cases and 225 controls was used to validate the index performance (Table 6). The WID-OC-index was computed for each woman (FIG. 2A) resulting in an AUC of 0.76 (95% CI: 0.68-0.84) and 0.77 (95% CI: 0.67-0.88) and 0.75 (95% CI: 0.63-0.87) for IC ≤0.5 and >0.5 samples respectively (FIG. 2B).

The vast majority of ovarian cancers arise from the Fallopian Tube—a part of the Müllerian Duct system. In order to assess whether the WID-OC-index reflects cancer predisposition of other parts of the Müllerian Duct like the endometrium, the inventors assessed its performance to discriminate women with and without endometrial cancer. Analysing 73 cervical samples from women with endometrial cancer, and the 297 control samples from the ovarian cancer internal validation, the inventors obtained an AUC of 0.92 (CI: 0.88-0.95; FIGS. 2C and 2D).

In order to assess whether the WID-OC-index is also informative in healthy women who are known to have a very high probability of developing ovarian cancer in the future, the inventors analysed a separate dataset consisting of cervical smear samples from 57 healthy BRCA1 mutation carriers (who have an up to 40 fold increased cancer risk (L. C. Hartmann, N. M. Lindor, Risk-Reducing Surgery in Hereditary Breast and Ovarian Cancer. N Engl J Med 374, 2404 (2016))) and 114 controls. The inventors observed an AUC of 0.62 (95% CI: 0.52-0.71) overall with 0.61 (95% CI: 0.48-0.74) and 0.65 (95% CI: 0.52-0.78) for IC ≤0.5 and >0.5 samples respectively. The inventors also analysed 53 women with a BRCA2 mutation and found that the discriminatory performance was poorer (0.54; 95% CI: 0.45-0.64; FIG. 7).

For each of the validation datasets the inventors computed odds ratios corresponding to quartiles defined on the internal validation dataset (Table 5). The cell-type composition of these three datasets was broadly similar to the discovery dataset used to develop the index and did not show any significant differences between cases and controls (FIG. 6).

Association with Epidemiological, Clinical and Technical Factors

The inventors investigated the relationship between the WID-OC-index and various epidemiological and clinical variables. A statistically significant association was found between the WID-OC-index and age in controls (correlation=0.52, p=10⁻³⁷). The Illumina 650k Infinium Global Screening Array was used to genotype matched blood samples from a subset of 74 cases and 255 controls in our internal validation dataset. The inventors computed a polygenic risk score (PRS; described in methods) for ovarian cancer prediction. The inventors found a correlation close to zero (−0.04, p=0.48) between the PRS and the WID-OC-index (FIG. 3B). The inventors compared different histologies and observed a significantly lower index in seven mucinous cancers compared to serous high grade and those classified as other (invasive epithelial ovarian cancers which do not belong to the four main categories; FIG. 3C). The WID-OC-index was significantly higher in stage III/IV cancers compared to stage I/II cancers (FIG. 3D). No significant association was found between the WID-OC-index and family history (FIG. 8A), age at menarche (FIG. 8B), oral contraceptive pill use (FIG. 8C), or ethnicity (FIG. 8D). The inventors observed a trend of increasing WID-OC-index values with respect to age at last period (FIG. 8E; linear regression p-value=0.01), and parity in post-menopausal women (FIG. 8F; not significant).

The inventors investigated whether any association existed between the WID-OC-index and various technical parameters including date of sample processing, plate number (samples were processed on 96 sample plates) and sentrix position but no significant associations were found. The inventors compared the 593 control samples from healthy volunteers to 276 control samples taken from women presenting with benign women-specific conditions but did not find any significant differences (FIG. 9A). The inventors also observed no significant dependence on the time from sample collection to DNA extraction (FIG. 9B).

### Inferred Proportion of Tumour DNA

Due to the anatomical proximity between the cancer (i.e. ovary/Fallopian Tube) and the area from which the sample was taken (i.e. cervix), the inventors investigated whether the signal which discriminates between a case and a control is driven by tumour DNA draining from the peritoneal cavity via the Fallopian Tube and the uterus to the cervix or whether the signal is a generic risk signal retained in cervical epithelial cells. The inventors used 11 epithelial, 7 fibroblast, 42 immune cell, and 11 high grade serous ovarian cancer cell line samples (Additional Data 1) in order to develop a new reference panel for use with the EpiDISH algorithm (see Material and Methods). For each sample, the inventors obtained estimates of the proportion of DNA from each of the four cell types. The inventors observed that the proportion of tumour DNA in both cases and controls is close to zero, with the exception of two cases that were composed of approximately 50% tumour DNA (FIG. 4A).

In order to assess further the absence of tumour DNA in the cervical smear samples the inventors used MethyLight, a real time PCR based method, to amplify methylated ZNF 154, a pan-cancer marker primarily discovered in ovarian cancer. The inventors detected a strong signal in cervical smear samples from 20 endometrial cancer cases but not from 20 ovarian cancer patients or 20 cancer-free women (FIG. 4B), providing further evidence that the signal in cancer patients is not driven by tumour DNA.

### Cell Line Data

In order to evaluate further the nature and meaning of the DNA methylation signature which forms the WID-OC-index the inventors calculated the index in the fimbriae of the Fallopian Tube, the organ from which the vast majority of ovarian cancers arise and which originates from the Müllerian Duct, the embryological structure which also gives rise to the cervix. Due to the fact that the tissue contains a heterogenous set of various cells, the inventors isolated and cultured pure fimbrial cells (without any modification, see Methods) from surgical specimens. Interestingly the WID-OC-index was comparatively high in the normal fimbrial cells, as well as fimbrial cells from BRCA mutation carriers (FIG. 4C). Two cancer cell lines had higher WID-OC-index values compared to both the cervix-based WID-OC-index and the fimbrial cells. The observation that the discriminatory DNAme signature observed in the cervixes of ovarian cancer patients is more pronounced in the Fallopian Tube is suggestive of an epigenetic field defect in which DNAme patterns in the cervix resemble the epigenome of the cell of origin, the Fallopian Tube.

### ENCODE Samples

In order to assess additionally whether the WID-OC-index is reflective of a cell-specific program the inventors analysed all ENCODE samples for which EPIC array data were available (Additional Data 2). The inventors ranked and plotted the WID-OC-index in all primary cell samples and in vitro differentiated cell samples (FIG. 10). The inventors observed that those tissues at the highest risk of becoming cancerous in BRCA carriers—such as Fallopian Tube, breast, pancreas and prostate—had the highest WID-OC-index. In order to quantify this observation, the inventors correlated the proportion of cancers per organ which arise in BRCA1 and BRCA2 mutation carriers (P. Jonsson et al., Tumour lineage shapes BRCA-mediated phenotypes. Nature 571, 576-579 (2019)) with the WID-OC-index of the respective normal tissue and found correlations of 0.23 (p=0.22) and 0.43 (p=0.019) in BRCA1- and BRCA2-driven cancers respectively (FIGS. 4D and 4E).

eFORGE and Gene Set Enrichment Analysis

The eFORGE tool was used to search for enrichment of cell-type specific CpGs in the top 1,000 hyper- and hypo-methylated CpGs (FIGS. 4F and 4G). Hypo-methylated CpGs were consistent with a foetal-like epigenetic program with enrichment for foetal intestine, stem cells, and foetal lung. A gene set enrichment analysis was performed using the Broad Institute's Molecular Signatures database but no significantly enriched pathways were detected (Table S2).

### Cancer Index Values and Clinical Actions

Four sub-groups defined by ranges of cancer index values are specified in Table 7 as corresponding to preferred clinical actions, comprising intensified screening, administration of therapeutics and surgery. The subgroups are quartiles based on control samples from the internal validation set. That is, these values of the index split the control samples into four equally sized groups. Odds ratio values are calculated by comparing the number of cases and controls in a given quartile to the first quartile (which is taken as a reference). Odds ratio values are determined for ovarian cancer risk, endometrial cancer risk and BRCA1 mutation risk. For the endometrial cancers these estimates are based on the internal validation dataset. For example, a woman in the fourth quartile is roughly 15 times more likely to have endometrial cancer than a woman in the first quartile.

### Discussion

To date, the best performing risk prediction model which incorporates 17 established epidemiologic risk factors and 17 genome-wide significant SNPs using data from 11 case-control studies provided an AUC of 0.66 (M. A. Clyde et al., Risk Prediction for Epithelial Ovarian Cancer in 11 United States-Based Case-Control Studies: Incorporation of Epidemiologic Risk Factors and 17 Confirmed Genetic Loci. Am J Epidemiol 184, 579-589 (2016)). Here the inventors demonstrate that the WID-OC-index—an index that is purely based on a DNAme signature in cervical smear samples—is strongly associated with ovarian cancer risk and provides an AUC of 0.76. The inventors neither observed a striking association between the WID-OC-index and any of the known epidemiological risk factors for ovarian cancer (apart for a BRCA1 germline mutation), nor did the inventors find any evidence that the WID-OC-index is triggered by tumour DNA draining from the peritoneal cavity via the uterine cavity and detected by the cervical smear. Therefore, the inventors speculate that the striking ovarian cancer risk reflected by the WID-OC-index is due to an epigenetic Müllerian Duct differentiation defect assessed at the level of the uterine cervix (a part of the Müllerian Duct) using a DNAme signature. Several lines of evidence support this view: (i) the WID-OC-index is high in Fallopian Tube fimbrial cells and reflective of organs that are at high risk of a BRCA-mediated cancer; (ii) the index also identifies women with other cancers arising from the Müllerian Duct (i.e. endometrial cancer); (iii) HOXA 9, 10 and 11 genes regulate the differentiation of the Müllerian Duct into Fallopian Tube, uterus and cervix, respectively, and given that serous, clear cell/endometrioid and mucinous cancers express these genes differentially, may reflect their origin. The fact that the inventors observe a decrease of the WID-OC-index from serous to mucinous cancers again supports the view that a “shift” of epigenetic differentiation (i.e. differentiation of Fallopian Tube reflected in cervical epithelial cells) predisposes individuals to ovarian cancer formation; (iv) Given that the index is more discriminatory in samples with a high epithelial cell content and that the index almost exclusively consists of CpGs which are highly variable in epithelial but not in immune cells attests to this.

Overall, the inventors provide substantial evidence that an epigenetic differentiation defect in easy-to-access epithelial samples is strongly associated with cancer risk. The inventors' findings need to be further validated in a cohort-based setting; our observation (unpublished) that long-term storage (i.e. several years) of smear samples within the fluid which is used for liquid based cytology (e.g. Preservcyt) strongly impacts on DNA methylation with the least impact on CpG within a CpG islands and the strongest impact on Open Sea CpGs (i.e. CpGs which are the main contributors to our signature) requires prospectively collected cohorts with samples processed and DNA extracted ideally within a few weeks of sample collection.

TABLE 5A

| | Con- | Can- | | | | |
|---|---|---|---|---|---|---|
| Quantile | trol | cer | OR (unadjusted) | | OR (adjusted) | |
| (−2.38, −0.57) | 75 | 3 | 1.00 | (reference) | 1.00 | (reference) |
| (−0.57, −0.21) | 74 | 10 | 3.25 | (0.93, 15.68) | 2.38 | (0.56, 12.99) |
| (−0.21, 0.17) | 74 | 13 | 4.2 | (1.27, 19.79) | 3.65 | (0.87, 19.42) |
| (0.17, 2.21) | 74 | 57 | 18.2 | (6.33, 79.95) | 10.26 | (2.89, 49.1) |

TABLE 5B

| | Con- | Can- | | |
|---|---|---|---|---|
| Quantile | trol | cer | OR (unadjusted) | OR (adjusted) |
| (−2.38, −0.57) | 59 | 2 | 1.00 (reference) | 1.00 (reference) |
| (−0.57, −0.21) | 57 | 6 | 2.94 (0.62, 22.94) | 4.88 (0.85, 41.76) |
| (−0.21, 0.17) | 49 | 6 | 3.42 (0.72, 26.71) | 4.57 (0.77, 40.12) |
| (0.17, 2.21) | 60 | 33 | 14.99 (4.26, 103.18) | 26.25 (5.89, 194.92) |

TABLE 5C

| Quantile | Control | Cancer | OR (unadjusted) | | OR (adjusted) |
|---|---|---|---|---|---|
| (−2.38, −0.57) | 75 | 0 | 1.00 | (reference) | 1.00 (reference) |
| (−0.57, −0.21) | 74 | 2 | 1.9 | (0.15, 60.54) | NA |
| (−0.21, 0.17) | 74 | 6 | 5.41 | (0.86, 142.55) | NA |
| (0.17, 2.21) | 74 | 65 | 57.12 | (12.24, 1351.82) | NA |

TABLE 5D

| | Con- | | | | | |
|---|---|---|---|---|---|---|
| Quantile | trol | Case | OR (unadjusted) | | OR (adjusted) | |
| (−2.38, −0.57) | 41 | 12 | 1.00 | (reference) | 1.00 | (reference) |
| (−0.57, −0.21) | 26 | 15 | 1.95 | (0.79, 4.95) | 1.66 | (0.64, 4.31) |
| (−0.21, 0.17) | 31 | 12 | 1.32 | (0.51, 3.39) | 1.2 | (0.42, 3.4) |
| (0.17, 2.21) | 16 | 18 | 3.76 | (1.49, 9.88) | 2.59 | (0.84, 8.08) |

TABLE 6A
External validation

| Factor | Group | Control | | Cancer | | OR | | p-value |
|---|---|---|---|---|---|---|---|---|
| Age (years) | <52 | 91 | (40%) | 15 | (32%) | 1.00 | (reference) | — |
| | 52-64 | 85 | (38%) | 15 | (32%) | 1.07 | (0.49, 2.35) | 0.86 |
| | >64 | 49 | (22%) | 17 | (36%) | 2.09 | (0.96, 4.62) | 0.06 |
| Age at menarche (years) | <12 | 34 | (15%) | 4 | (9%) | 0.62 | (0.16, 1.91) | 0.42 |
| | Dec-13 | 119 | (53%) | 29 | (62%) | 1.25 | (0.62, 2.59) | 0.54 |
| | >13 | 72 | (32%) | 14 | (30%) | 1.00 | (reference) | — |
| Parous | Yes | 166 | (74%) | 35 | (74%) | 1.00 | (reference) | — |
| | No | 59 | (26%) | 12 | (26%) | 0.97 | (0.45, 1.96) | 0.94 |
| Age at birth of first live child | <21 | 16 | (10%) | 3 | (9%) | 1.00 | (reference) | — |
| (years) | 21-27 | 61 | (38%) | 21 | (60%) | 1.76 | (0.51, 8.54) | 0.39 |
| | >27 | 84 | (52%) | 11 | (31%) | 0.68 | (0.18, 3.43) | 0.61 |
| Menopausal status | Pre | 79 | (35%) | 12 | (26%) | 1.00 | (reference) | — |
| | Post | 146 | (65%) | 35 | (74%) | 1.56 | (0.78, 3.32) | 0.21 |
| Age at menopause (years) | <46 | 82 | (37%) | 12 | (27%) | 1.13 | (0.41, 3.5) | 0.82 |
| | 46-52 | 91 | (41%) | 27 | (60%) | 2.27 | (0.92, 6.54) | 0.08 |
| | >52 | 47 | (21%) | 6 | (13%) | 1.00 | (reference) | — |
| BMI (kg/m2) | <25 | 117 | (52%) | 28 | (60%) | 1.00 | (reference) | — |
| | 25-30 | 57 | (25%) | 13 | (28%) | 0.96 | (0.45, 1.97) | 0.91 |
| | >30 | 51 | (23%) | 6 | (13%) | 0.5 | (0.18, 1.22) | 0.13 |
| Oral contraceptive use (pre | No | 29 | (39%) | 4 | (27%) | 1.00 | (reference) | — |
| menopausal only) | Yes | 46 | (61%) | 11 | (73%) | 1.69 | (0.51, 6.78) | 0.4 |
| Hormone replacement therapy | No | 71 | (93%) | 15 | (100%) | 1.00 | (reference) | — |
| (post menopausal only) | Yes | 5 | (7%) | 0 | (0%) | | | |
| Smoking | No | 190 | (85%) | 40 | (85%) | 1.00 | (reference) | — |
| | Yes | 34 | (15%) | 7 | (15%) | 0.99 | (0.38, 2.3) | 0.99 |
| First degree relatives with | 0 | 218 | (97%) | 46 | (98%) | 1.00 | (reference) | — |
| ovarian cancer | 1 | 7 | (3%) | 1 | (2%) | 0.76 | (0.03, 4.53) | 0.8 |
| | 2+ | 0 | (0%) | 0 | (0%) | | | |

TABLE 6B
Internal validation

| Factor | Group | Set | |
|---|---|---|---|
| Age (years) | <52 | 57 | (28%) |
| | 52-64 | 74 | (36%) |
| | >64 | 75 | (36%) |
| Menopausal status | Pre | 49 | (24%) |
| | Post | 157 | (76%) |
| Stage | T1 | 40 | (19%) |
| | T2 | 27 | (13%) |
| | T3 | 104 | (50%) |
| | T4 | 28 | (14%) |
| Grade | I | 24 | (12%) |
| | II | 10 | (5%) |
| | III | 159 | (77%) |
| Histology | Endometrioid | 15 | (7%) |
| | Mucinous | 12 | (6%) |
| | Clear cell | 15 | (7%) |
| | High grade serous | 142 | (69%) |
| | Other | 22 | (11%) |

TABLE 6C
External validation

| Factor | Group | Set | |
|---|---|---|---|
| Age (years) | <52 | 57 | (28%) |
| | 52-64 | 74 | (36%) |
| | >64 | 75 | (36%) |
| Menopausal status | Pre | 49 | (24%) |
| | Post | 157 | (76%) |
| Stage | T1 | 40 | (19%) |
| | T2 | 27 | (13%) |
| | T3 | 104 | (50%) |
| | T4 | 28 | (14%) |
| Grade | I | 24 | (12%) |
| | II | 10 | (5%) |
| | III | 159 | (77%) |
| Histology | Endometrioid | 15 | (7%) |
| | Mucinous | 12 | (6%) |
| | Clear cell | 15 | (7%) |
| | High grade serous | 142 | (69%) |
| | Other | 22 | (11%) |

TABLE 7

| WID-OC | Ovarian | Endometrial | BRCA1 | |
|---|---|---|---|---|
| ranges | cancer risk | cancer risk | mutation risk | Clinical Action |
| (−2.38, −0.57) | 1 | 1 | 1 | No specific screening or preventive measures required. |
| | | | | Repeat test in 2 years time. |
| (−0.57, −0.21) | 2.94 | 1.9 | 1.95 | (1) Testing for a BRCA1 or BRCA2 germline mutation; |
| | (0.62, 22.94) | (0.15, 60.54) | (0.79, 4.95) | (2) in postmenopaual women: Annual CA125 and cell- |
| | | | | free tumour DNA methylation in plasma/serum for early |
| | | | | detection of ovarian cancer and annual testing for cell- |
| | | | | free tumour DNA methylation in vaginal fluid. Repeat |
| | | | | test in 2 years time. |
| (−0.21, 0.17) | 3.42 | 5.41 | 1.32 | (1) Testing for a BRCA1 or BRCA2 germline mutation; |
| | (0.72, 26.71) | (0.86, 142.55) | (0.51, 3.39) | (2) Annual CA125 and cell-free tumour DNA methylation |
| | | | | in plasma/serum for early detection of ovarian cancer |
| | | | | and annual testing for cell-free tumour DNA methylation |
| | | | | in vaginal fluid. (3) Annual pelvic MRI in postmenopausal |
| | | | | women. (4) To consider taking any of the following risk |
| | | | | preventive drugs: Aspirin, oral contraceptive pill, |
| | | | | “selective estrogen receptor modulators” (SERMs) and |
| | | | | “selective progesterone receptor modulators” (SPRMs). |
| | | | | SERMs may include Anordin, Bazedoxifene, |
| | | | | Broparestrol, Clomifene, Cyclofenil, Lasofoxifene, |
| | | | | Ormeloxifene, Ospemifene, Raloxifene, Tamoxifen. |
| | | | | Preferably, the SERMs include Tamoxifen, Bazedoxifene |
| | | | | and Raloxifene. Preferably, the SPRMs include |
| | | | | Mifepristone, Ulipristal, Asoprisnil, Proellex, Onapristone, |
| | | | | Asoprisnil and Lonaprisan. (5) Repeat test annually |
| (0.17, 2.21) | 14.99 | 57.12 | 3.76 | (1) Testing for a BRCA1 or BRCA2 germline mutation; |
| | (4.26, 103.18) | (12.24, 1351.82) | (1.49, 9.88) | (2) Intensified ovarian cancer screening: Three monthly |
| | | | | CA125 and cell-free tumour DNA methylation in |
| | | | | plasma/serum for early detection of ovarian cancer and |
| | | | | testing for cell-free tumour DNA methylation in vaginal |
| | | | | fluid; annual pelvic MRI in all women. (3) To consider |
| | | | | taking any of the following risk preventive drugs: Aspirin, |
| | | | | oral contraceptive pill, “selective estrogen receptor |
| | | | | modulators” (SERMs) and “selective progesterone |
| | | | | receptor modulators” (SPRMs). SERMs may include |
| | | | | Anordin, Bazedoxifene, Broparestrol, Clomifene, |
| | | | | Cyclofenil, Lasofoxifene, Ormeloxifene, Ospemifene, |
| | | | | Raloxifene, Tamoxifen. Preferably, the SERMs include |
| | | | | Tamoxifen, Bazedoxifene and Raloxifene. Preferably, |
| | | | | the SPRMs include Mifepristone, Ulipristal, Asoprisnil, |
| | | | | Proellex, Onapristone, Asoprisnil and Lonaprisan. (4) To |
| | | | | consider risk reducing blateral salpingoophorectomy and |
| | | | | total hysterectomy. |

1. A method comprising assaying from a sample from an individual:
the methylation status of a panel of one or more CpGs selected from a panel of CpGs identified at nucleotide positions 61 to 62 in SEQ ID NOs 1 to 14,000.

2. A method according to claim 1, wherein:
(i) the panel of one or more CpGs comprises at least 500 CpGs selected from the CpGs identified at nucleotide positions 61 to 62 in SEQ ID NOs 1 to 14,000 and optionally wherein the panel of one or more CpGs comprises at least the CpGs identified in SEQ ID NOs 1 to 500 and identified at nucleotide positions 61 to 62; or
(ii) the panel of one or more CpGs comprises at least 1000 CpGs selected from the CpGs identified at nucleotide positions 61 to 62 in SEQ ID NOs 1 to 14,000, and optionally wherein the panel of one or more CpGs comprises at least the CpGs identified in SEQ ID NOs 1 to 1000 and identified at nucleotide positions 61 to 62; or,
ii) the panel of one or more CpGs comprises at least 1000 CpGs selected from the CpGs identified at nucleotide positions 61 to 62 in SEQ ID NOs 1 to 14,000, and optionally wherein the panel of one or more CpGs comprises at least the CpGs identified in SEQ ID NOs 1 to 1000 and identified at nucleotide positions 61 to 62; or
iii) the panel of one or more CpGs comprises at least 2000 CpGs selected from the CpGs identified at nucleotide positions 61 to 62 in SEQ ID NOs 1 to 14,000, and optionally wherein the panel of one or more CpGs comprises at least the CpGs identified in SEQ ID NOs 1 to 2000 and identified at nucleotide positions 61 to 62; or
iv) the panel of one or more CpGs comprises at least 10,000 CpGs selected from the CpGs identified at nucleotide positions 61 to 62 in SEQ ID NOs 1 to 14,000, and optionally wherein the panel of one or more CpGs comprises at least the CpGs identified in SEQ ID NOs 1 to 10,000 and identified at nucleotide positions 61 to 62; or
v) the panel of one or more CpGs comprises at least the 14,000 CpGs identified at nucleotide positions 61 to 62 in SEQ ID NOs 1 to 14,000.

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. A method according to claim 1, wherein the step of assaying the methylation status of each CpG in the panel of one or more CpGs comprises:
a. performing a sequencing step to determine the sequence of each CpG;
b. hybridising DNA to an array comprising probes capable of discriminating between methylated and non-methylated forms of the CpGs and applying a detection system to the array so as to determine the methylation status of each CpG; and/or
c. performing a PCR step using methylation-specific primers, wherein the methylation status of the CpG is determined by the presence or absence of a PCR product.

19. A method according to claim 1, wherein the step of assaying the methylation status of each CpG in the panel of one or more CpGs comprises:
a. bisulphite converting the DNA; or
b. performing the steps of oxidising 5-methylcytosine bases (5mC) to 5-carboxylcytosine bases (5caC), preferably by ten-eleven translocation (TET), and/or oxidising 5-hydroxymethylcytosine bases (5hmC) to 5-carboxylcytosine bases (5caC), preferably by ten-eleven translocation (TET); followed by reducing 5-carboxylcytosine bases (5caC) to dihydrouracil bases (DHU), optionally with pyridine borane.

20. A method of treating or preventing cancer in an individual, the method comprising administering one or more treatments to the individual determined to have cancer by the step of performing the method of claim 1 on a sample from the individual.

21.-41. (canceled)

42. A method according to claim 20, further defined as comprising a step of stratifying the individual according to their cancer risk or of having cancer.

43. A method according to claim 42, wherein the individual is stratified as not having cancer or as having a low risk of cancer development, and wherein the individual is subjected to one or more treatments according to their stratification, wherein the one or more treatments comprise intensified screening, preferably wherein the intensified screening comprises any one or more of:
a. a test for a BRCA1 and/or BRCA2 germline mutation;
b. a test for CA125, preferably wherein the test is repeated annually;
c. a test for cell-free tumour DNA methylation in plasma/serum, preferably wherein the test is repeated annually;
d. a test for cell-free tumour DNA methylation in vaginal fluid, preferably wherein the test is repeated annually;
e. a repeat method according to claim 1, preferably wherein the repeat method is performed about two years after the previous method,
preferably wherein the individual being tested by any one or more of b. to d. is post-menopausal.

44. A method according to claim 42, wherein the individual is stratified as having a moderate risk of having cancer or as having a moderate risk of cancer development, and wherein the individual is subjected to one or more treatments according to their stratification, wherein the one or more treatments comprise any one or more of:
a. intensified screening, preferably wherein the intensified screening comprises one or more of:
i. a test for a BRCA1 and/or BRCA2 germline mutation;
ii. a test for CA125, preferably wherein the test is repeated annually;
iii. a test for cell-free tumour DNA methylation in plasma/serum, preferably wherein the test is repeated annually;
iv. a test for cell-free tumour DNA methylation in vaginal fluid, preferably wherein the test is repeated annually
v. a pelvic MRI scan, preferably wherein the individual being subjected to the pelvic MRI scan is post-menopausal, and preferably wherein the scan is repeated annually;
vi. a repeat method according to claim 1, preferably wherein the repeat method is performed about one year after the previous method;
b. administration of one or more of Aspirin, oral contraceptive pill, selective estrogen receptor modulators (SERMS) optionally comprising Anordin, Bazedoxifene, Broparestrol, Broparestrol, Clomifene, Cyclofenil, Lasofoxifene, Ormeloxifene, Ospemifene, Raloxifene or Tamoxifen, and selective progesterone receptor modulators (SPRMs) optionally comprising Mifepristone, Ulipristal, Asoprisnil, Proellex, Onapristone, Asoprisnil and Lonaprisan.

45. A method according to claim 42, wherein the individual is stratified as having cancer or as having a high risk of cancer development, and wherein the individual is subjected to one or more treatments according to their stratification, wherein the one or more treatments comprise any one or more of:
a. intensified screening, preferably wherein the intensified screening comprises one or more of:
i. a test for a BRCA1 and/or BRCA2 germline mutation;
ii. a test for CA125, preferably wherein the test is repeated three-monthly;
iii. a test for cell-free tumour DNA methylation in plasma/serum, preferably wherein the test is repeated annually;
iv. a test for cell-free tumour DNA methylation in vaginal fluid, preferably wherein the test is repeated annually
v. a pelvic MRI scan, preferably wherein the scan is repeated annually;
vi. a repeat method according to claim 1, preferably wherein the repeat method is performed about one year after the previous method;
b. administration of one or more of Aspirin, oral contraceptive pill, selective estrogen receptor modulators (SERMS) optionally comprising Anordin, Bazedoxifene, Broparestrol, Broparestrol, Clomifene, Cyclofenil, Lasofoxifene, Ormeloxifene, Ospemifene, Raloxifene or Tamoxifen, and selective progesterone receptor modulators (SPRMs) optionally comprising Mifepristone, Ulipristal, Asoprisnil, Proellex, Onapristone, Asoprisnil and Lonaprisan; and/or
c. a total hysterectomy and bilateral salpingo-oophorectomy.

46. A method according to claim 42, wherein the one or more treatments that the individual is subjected to are repeated on a monthly, three monthly, six monthly, yearly or two yearly basis following an initial administration.

47. A method of assaying methylation in an individual at multiple time points, the method comprising: (a) performing the method according to claim 1 at a first time point; (b) performing the method according to claim 1 at one or more further time points; and (c) detecting differential methylation status between (a) and (b).

48. A method according to claim 47, wherein the further time points are monthly, three monthly, six monthly, yearly or two yearly basis following an initial assessment; and/or wherein one or more treatments are administered to the individual according to claim 45.

49. A method according to claim 1, wherein the sample is obtained from a tissue comprising epithelial cells, preferably wherein the sample is not obtained from ovarian or endometrial tissue.

50. A method according to claim 49, wherein the sample is obtained from:
a. cervical tissue;
b. vaginal tissue;
c. cervicovaginal tissue; and/or
d. buccal tissue;
preferably wherein the sample is obtained from cervical tissue, most preferably wherein the sample is obtained from tissue from a cervical smear.

51. An array for discriminating between methylated and non-methylated forms of CpGs; the array comprising oligonucleotide probes specific for a methylated form of each CpG in a CpG panel and oligonucleotide probes specific for a non-methylated form of each CpG in the panel; wherein the panel comprises at least 500 CpGs selected from the CpGs identified at nucleotide position 61 to 62 in SEQ ID NOs 1 to 14,000.

52. An array according to claim 51, provided that the array is not an Infinium MethylationEPIC BeadChip array or an Infinium HumanMethylation450, and/or provided that the number of CpG-specific oligonucleotide probes of the array is 482,000 or less, 480,000 or less, 450,000 or less, 440,000 or less, 430,000 or less, 420,000 or less, 410,000 or less, or 400,000 or less; and/or wherein the panel comprises one or more CpGs selected from the CpGs identified at nucleotide positions 61 to 62 in SEQ ID NOs 1 to 14,000; and or wherein the array further comprises one or more oligonucleotides comprising any set of CpGs one or more CpGs selected from the CpGs identified at nucleotide positions 61 to 62 in SEQ ID NOs 1 to 14,000, wherein the one or more oligonucleotides are hybridized to corresponding oligonucleotide probes of the array.

53. A hybridized array, wherein the array is obtainable by hybridizing to an array according to claim 51 a group of oligonucleotides comprising any set of CpGs selected from the CpGs identified at nucleotide positions 61 to 62 in SEQ ID NOs 1 to 14,000.

54. A process for making the hybridized array according to claim 53, comprising contacting an array comprising oligonucleotide probes specific for a methylated form of each CpG in a CpG panel and oligonucleotide probes specific for a non-methylated form of each CpG in the panel; wherein the panel comprises at least 500 CpGs selected from the CpGs identified at nucleotide position 61 to 62 in SEQ ID NOs 1 to 14,000 with a group of oligonucleotides comprising any set of CpGs selected from the CpGs identified at nucleotide positions 61 to 62 in SEQ ID NOs 1 to 14,000.
